(19) United States
(12) Patent Application Publication
(10) Pub. No.: US 2014/0179620 A1
(43) Pub. Date: Jun. 26, 2014

(54) GENE EXPRESSION MARKERS FOR INFLAMMATORY BOWEL DISEASE

(71) Applicant: GENENTECH, INC., South San Francisco, CA (US)

(72) Inventors: Alexander R. Abbas, San Carlos, CA (US); Hilary Clark, San Francisco, CA (US); Lauri Diehl, Los Altos, CA (US); Charles Lees, Linlithgow (GB); Colin L. Noble, Edinburgh (GB); Jack Satsangi, Edinburgh (GB)

(73) Assignee: GENENTECH, INC., South San Francisco, CA (US)

(21) Appl. No.: 13/964,861

(22) Filed: Aug. 12, 2013

Related U.S. Application Data
(63) Continuation of application No. 12/324,783, filed on Nov. 26, 2008, now abandoned.
Application No. 12/324,783 is a continuation-in-part of application No. 12/125,724, filed on May 22, 2008, now abandoned.

(51) Int. Cl. C12Q 1/68; G01N 33/564

(52) U.S. Cl. CPC C12Q 1/6883; G01N 33/564
USPC 514/20.5; 435/7.1; 435/6.11; 435/6.12; 435/7.21; 435/7.4; 435/7.92; 506/9; 506/2; 514/353

(57) ABSTRACT

The present invention relates to methods of gene expression profiling for inflammatory bowel disease pathogenesis, in which the differential expression in a test sample from a mammalian subject of one or more IBD markers relative to a control is determined, wherein the differential expression in the test sample is indicative of an IBD in the mammalian subject from which the test sample was obtained.

### CROSS-REFERENCE TO RELATED APPLICATIONS

This application claims priority under Section 119(e) and the benefit of U.S. Provisional Application Ser. No. 60/991,203 filed Nov. 29, 2007, U.S. Provisional Application Ser. No. 61/192,268 filed Sep. 17, 2008, the entire disclosures of which are incorporated herein by reference in their entirety. The application also claims priority under Section 119(e) and the benefit of U.S. Provisional Application Ser. No. 60/190,689 filed Aug. 28, 2008.

This application is a continuation application of U.S. patent application Ser. No. 12/324,783, filed Nov. 26, 2008 (co-pending), which claims priority under Section 119(e) and the benefit of U.S. Provisional Application Ser. No. 60/991,203 filed Nov. 29, 2007, U.S. Provisional Application Ser. No. 61/190,689 filed Aug. 28, 2008, and U.S. Provisional Application Ser. No. 61/192,268 filed Sep. 17, 2008. This application also claims priority under 35 USC §120 to, and is a continuation-in-part of, U.S. Non-provisional application Ser. No. 12/125,724 filed May 22, 2008.

### SEQUENCE LISTING

The instant application contains a Sequence Listing which has been submitted in ASCII format via EFS-Web and is hereby incorporated by reference in its entirety. Said ASCII copy, created on Dec. 8, 2011, is named GNE238US.txt and is 807,225 bytes in size.

### BACKGROUND OF THE INVENTION

1. Field of the Invention

The present invention relates to gene expression profiles in inflammatory bowel disease pathogenesis, including use in the detection and diagnosis of inflammatory bowel disease.

2. Description of Related Art

Immune related and inflammatory diseases are the manifestation or consequence of fairly complex, often multiple interconnected biological pathways which in normal physiology are critical to respond to insult or injury, initiate repair from insult or injury, and mount innate and acquired defense against foreign organisms. Disease or pathology occurs when these normal physiological pathways cause additional insult or injury either as directly related to the intensity of the response, as a consequence of abnormal regulation or excessive stimulation, as a reaction to self, or as a combination of these.

Though the genesis of these diseases often involves multistep pathways and often multiple different biological systems/pathways, intervention at critical points in one or more of these pathways can have an ameliorative or therapeutic effect. Therapeutic intervention can occur by either antagonism of a detrimental process/pathway or stimulation of a beneficial process/pathway.

Many immune related diseases are known and have been extensively studied. Such diseases include immune-mediated inflammatory diseases, non-immune-mediated inflammatory diseases, infectious diseases, immunodeficiency diseases, neoplasia, etc.

The term inflammatory bowel disorder (“IBD”) describes a group of chronic inflammatory disorders of unknown causes in which the intestine (bowel) becomes inflamed, often causing recurring cramps or diarrhea. The prevalence of IBD in the US is estimated to be about 200 per 100,000 population. Patients with IBD can be divided into two major groups, those with ulcerative colitis (“UC”) and those with Crohn's disease (“CD”). Both UC and CD are chronic relapsing diseases and are complex clinical entities that occur in genetically susceptible individuals who are exposed to as yet poorly defined environmental stimuli. (Bonen and Cho, Gastroenterology. 2003; 124:521-536; Gaya et al. Lancet. 2006; 367:1271-1284).

Although the cause of IBD remains unknown, several factors such as genetic, infectious and immunologic susceptibility have been implicated. IBD is much more common in Caucasians, especially those of Jewish descent. The chronic inflammatory nature of the condition has prompted an intense search for a possible infectious cause. Although agents have been found which stimulate acute inflammation, none has been found to cause the chronic inflammation associated with IBD. The hypothesis that IBD is an autoimmune disease is supported by the previously mentioned extraintestinal manifestation of IBD as joint arthritis, and the known positive response to IBD by treatment with therapeutic agents such as adrenal glucocorticoids, cyclosporine and azathioprine, which are known to suppress immune response. In addition, the GI tract, more than any other organ of the body, is continuously exposed to potential antigenic substances such as proteins from food, bacterial byproducts (LPS), etc.

There is sufficient overlap in the diagnostic criteria for UC and CD that it is sometimes impossible to say which a given patient has; however, the type of lesion typically seen is different, as is the localization. UC mostly appears in the colon, proximal to the rectum, and the characteristic lesion is a superficial ulcer of the mucosa; CD can appear anywhere in the bowel, with occasional involvement of stomach, esophagus and duodenum, and the lesions are usually described as extensive linear fissures.

The current therapy of IBD usually involves the administration of antiinflammatory or immunosuppressive agents, such as sulfasalazine, corticosteroids, 6-mercaptopurine/azathioprine, or cyclosporine, which usually bring only partial results. If anti-inflammatory/immunosuppressive therapies fail, colectomies are the last line of defense. The typical operation for CD not involving the rectum is resection (removal of a diseased segment of bowel) and anastomosis (reconnection) without an ostomy. Sections of the small or large intestine may be removed. About 30% of CD patients will need surgery within the first year after diagnosis. In the subsequent years, the rate is about 5% per year. Unfortunately, CD is characterized by a high rate of recurrence; about 5% of patients need a second surgery each year after initial surgery.

Refining a diagnosis of inflammatory bowel disease involves evaluating the progression status of the diseases using standard classification criteria. The classification systems used in IBD include the Truelove and Witts Index (Truelove S. C. and Witts, L. J. Br Med J. 1955; 2:1041-1048), which classifies colitis as mild, moderate, or severe, as well as Lennard-Jones. (Lennard-Jones J E. Scand J Gastroenterol Suppl 1989; 170:2-6) and the simple clinical colitis activity index (SCCAI). (Walmsley et. al. Gut. 1998; 43:29-32) These systems track such variables as daily bowel movements, rectal bleeding, temperature, heart rate, hemoglobin levels, erythrocyte sedimentation rate, weight, hematocrit score, and the level of serum albumin.

In approximately 10-15% of cases, a definitive diagnosis of ulcerative colitis or Crohn's disease cannot be made and such cases are often referred to as “indeterminate colitis.” Two antibody detection tests are available that can help the diagnosis, each of which assays for antibodies in the blood. The antibodies are “perinuclear anti-neutrophil antibody” (pANCA) and “anti-Saccharomyces cervisiae antibody” (ASCA). Most patients with ulcerative colitis have the pANCA antibody but not the ASCA antibody, while most patients with Crohn's disease have the ASCA antibody but not the pANCA antibody. However, these two tests have shortcomings as some patients have neither antibody and some Crohn's disease patients may have only the pANCA antibody. For clinical practice, a reliable test that would indicate the presence and/or progression of an IBD based on molecular markers rather than the measurement of a multitude of variables would be useful for identifying and/or treating individuals with an IBD. Hypothesis free, linkage and association studies have identified genetic loci that have been associated with UC, notably the MHC region on chromosome 6, (Rioux et al. Am J Hum Genet. 2000; 66:1863-1870; Stokkers et al. Gut. 1999; 45:395-401; Van Heel et al. Hum Mol. Genet. 2004; 13:763-770) the IBD2 locus on chromosome 12 (Parkes et al. Am J Hum Genet. 2000; 67:1605-1610; Satsangi et al. Nat. Genet. 1996; 14:199-202) and the IBD5 locus on chromosome 5. (Giallourakis et. al. Am J. Hum Genet. 2003; 73:205-211; Palmieri et. al Aliment Pharmacol Ther. 2006; 23:497-506; Russell et. al. Gut. 2006; 55:1114-1123; Waller et. al. Gut. 2006; 55:809-814) Following a UK wide linkage scan identifying a putative loci of association for UC on chromosome 7q, further studies have implicated variants in the ABCB1 (MDR1) gene which is involved in cellular detoxification with UC. (Satsangi et. al. Nat. Genet. 1996; 14:199-202; Brant et. al. Am J Hum Genet. 2003; 73:1282-1292; Ho et. al. Gastroenterology. 2005; 128:288-296)

A complementary approach towards the identification and understanding of the complex gene-gene and gene-environment relationships that result in the chronic intestinal inflammation observed in inflammatory bowel disease (IBD) is microarray gene expression analysis. Microarrays allow a comprehensive picture of gene expression at the tissue and cellular level, thus helping understand the underlying patho-physiological processes. (Stoughton et. al. Annu Rev Biochem. 2005; 74:53-82) Microarray analysis was first applied to patients with IBD in 1997, comparing expression of 96 genes in surgical resections of patients with CD to synovial tissue of patients with rheumatoid arthritis. (Heller et. al. Proc Natl Acad Sci USA. 1997; 94:2150-2155) Further studies using microarray platforms to interrogate surgical specimens from patients with IBD identified an number of novel genes that were differentially regulated when diseased samples were compared to controls. (Dieckgraefe et. al. Physiol Genomics. 2000; 4:1-11; Lawrance et. al. Hum Mol. Genet. 2001; 10:445-456)

A complementary approach towards the identification and understanding of the complex gene-gene and gene-environment relationships that result in the chronic intestinal inflammation observed in inflammatory bowel disease (IBD) is microarray gene expression analysis. Microarrays allow a comprehensive picture of gene expression at the tissue and cellular level, thus helping understand the underlying patho-physiological processes. (Stoughton et. al. Annu Rev Biochem. 2005; 74:53-82) Microarray analysis was first applied to patients with IBD in 1997, comparing expression of 96 genes in surgical resections of patients with CD to synovial tissue of patients with rheumatoid arthritis. (Heller et. al. Proc Natl Acad Sci USA. 1997; 94:2150-2155) Further studies using microarray platforms to interrogate surgical specimens from patients with IBD identified a number of novel genes that were differentially regulated when diseased samples were compared to controls. (Dieckgraefe et. al. Physiol Genomics. 2000; 4:1-11; Lawrance et. al. Hum Mol. Genet. 2001; 10:445-456)

Endoscopic pinch mucosal biopsies have allowed investigators to microarray tissue from a larger range of patients encompassing those with less severe disease. Langmann et. al. used microarray technology to analyze 22,283 genes in biopsy specimens from macroscopically non affected areas of the colon and terminal ileum. (Langmann et. al. Gastroenterology. 2004; 127:26-40) Genes which were involved in cellular detoxification and biotransformation (Pregnane X receptor and MDR1) were significantly downregulated in the colon of patients with UC, however, there was no change in the expression of these genes in the biopsies from patients with CD. Costello and colleagues (Costello et. al. PLoS Med. 2005; 2:e199) looked at the expression of 33792 sequences in endoscopic sigmoid colon biopsies obtained from healthy controls, patients with CD and UC. A number of sequences representing novel proteins were differentially regulated and in silico analysis suggested that these proteins had putative functions related to disease pathogenesis—transcription factors, signaling molecules and cell adhesion.

In a study of patients with UC, Okahara et al. (Aliment Pharmacol Ther. 2005; 21:1091-1097) observed that (migration inhibitory factor-related protein 14 (MRP14), growth-related oncogene gamma (GROγ) and serum amyloid A1 (SAA1) were upregulated where as TIMP1 and elfin were down regulated in the inflamed biopsies when compared to the non-inflamed biopsies. When observing 41 chemokines and 21 chemokine receptors, Puleston et al demonstrated that chemokines CXCLs 1-3 and 8 and CCL20 were upregulated in active colonic CD and UC. (Aliment Pharmacol Ther. 2005; 21:109-120) Overall these studies illustrate the heterogeneity of early microarray platforms and tissue collection. However, despite these problems differential expression of a number of genes was consistently observed.

Despite the above identified advances in IBD research, there is a great need for additional diagnostic and therapeutic agents capable of detecting IBD in a mammal and for effectively treating this disorder. Accordingly, the present invention provides polynucleotides and polypeptides that are overexpressed in IBD as compared to normal tissue, and methods of using those polypeptides, and their encoding nucleic acids, for to detect or diagnose the presence of an IBD in mammalian subjects and subsequently to treat those subjects in which an IBD is detected with suitable IBD therapeutic agents.

The present invention provides methods for detecting the presence of and determining the progression of inflammatory bowel disease (IBD), including ulcerative colitis (UC) and Crohn's disease (CD).

The invention disclosed herein provides methods and assays examining expression of one or more gene expression markers in a mammalian tissue or cell sample, wherein the expression of one or more such biomarkers is predictive of whether the mammalian subject from which the tissue or cell sample was taken is more likely to have an IBD. In various embodiments of the invention, the methods and assays examine the expression of gene expression markers such as those listed in Tables 1, 2, and 3 and determine whether expression is higher or lower than a control sample.

These and further embodiments of the present invention will be apparent to those of ordinary skill in the art.

### SUMMARY OF THE INVENTION

In one aspect, the invention concerns a method of detecting or diagnosing an inflammatory bowel disease (IBD) in a mammalian subject comprising determining, in a biological sample obtained from the subject, that expression levels of (i) one or more nucleic acids encoding one or more polypeptides selected from Tables 1, 2, or 3, or (ii) RNA transcripts or their expression products of one or more genes selected from Tables 1, 2, or 3, is different relative to the expression level in a control, wherein the difference in expression indicates the subject is more likely to have an IBD.

In one embodiment, the methods of diagnosing or detecting the presence of an IBD in a mammalian subject comprise determining that the expression level of (i) one or more nucleic acids encoding one or more polypeptides selected from Tables 1A, 2, or 3A; or (ii) RNA transcripts or expression products thereof of one or more genes selected from Tables 1A, 2 or 3A in a test sample obtained from the subject is higher relative to the level of expression in a control, wherein the higher level of expression is indicative of the presence of an IBD in the subject from which the test sample was obtained.

In another embodiment, the methods of diagnosing or detecting the presence of an IBD in a mammalian subject comprise determining that the expression level of (i) one or more nucleic acids encoding one or more polypeptides selected from Tables 1B or 3B; or (ii) RNA transcripts or expression products thereof of one or more genes selected from Tables 1B or 3B in a test sample obtained from the subject is lower relative to the level of expression in a control, wherein the lower level of expression is indicative of the presence of an IBD in the subject from which the test sample was obtained.

In one aspect, the methods are directed to diagnosing or detecting a flare-up of an IBD in mammalian subject that was previously diagnosed with an IBD and is currently in remission. The subject may have completed treatment for the IBD or is currently undergoing treatment for the IBD. In one embodiment, the methods comprise determining, in a biological sample obtained from the mammalian subject, that the expression level of (i) one or more nucleic acids encoding one or more polypeptides selected from Tables 1, 2, or 3; or (ii) RNA transcripts or expression products thereof of one or more genes selected from Tables 1, 2 or 3 is different relative to the expression level in a control, wherein the difference in expression indicates the subject is more likely to have an IBD flareup. Alternatively, the test sample may be compared to a prior test sample of the mammalian subject, if available, obtained before, after, or at the time of the initial IBD diagnosis.

In all aspects, the mammalian subject preferably is a human patient, such as a human patient diagnosed with or at risk of developing an IBD. The subject may also be an IBD patient who has received prior treatment for an IBD but is at risk of a recurrence of the IBD.

For all aspects of the method of the invention, determining the expression level of one or more genes described herein (or one or more nucleic acids encoding polypeptide(s) expressed by one or more of such genes) may be obtained, for example, by a method of gene expression profiling. The method of gene expression profiling may be, for example, a PCR-based method.

In various embodiments, the diagnosis includes quantification of the expression level of (i) one or more nucleic acids encoding one or more polypeptides selected from Tables 1, 2, or 3; or (ii) RNA transcripts or expression products thereof of one or more genes selected from Tables 1, 2 or 3, such as by immunohistochemistry (1HC) and/or fluorescence in situ hybridization (FISH).

For all aspects of the invention, the expression levels of the genes may be normalized relative to the expression levels of one or more reference genes, or their expression products.

For all aspects of the invention, the method may further comprise determining evidence of the expression levels of at least two, three, four, five, six, seven, eight, nine, ten, eleven, twelve, thirteen, fourteen, fifteen, sixteen, seventeen, eighteen, nineteen, or twenty of said genes, or their expression products.

In another aspect, the methods of present invention also contemplate the use of a “panel” of such genes (i.e. IBD markers as disclosed herein) based on the evidence of their level of expression. In some embodiments, the panel of IBD markers will include at least one, two, three, four, five, six, seven, eight, nine, ten, eleven, twelve, thirteen, fourteen, fifteen, sixteen, seventeen, eighteen, nineteen or twenty IBD markers. The panel may include an IBD marker that is overexpressed in IBD relative to a control, an IBD marker that is underexpressed in IBD relative to a control, or IBD markers that are both overexpressed and underexpressed in IBD relative to a control. Such panels may be used to screen a mammalian subject for the differential expression of one or more IBD markers in order to make a determination on whether an IBD is present in the subject.

In one embodiment, the IBD markers that make up the panel are selected from Tables 1, 2, and 3. In a preferred embodiment, the methods of diagnosing or detecting the presence of an IBD in a mammalian subject comprise determining a differential expression level of RNA transcripts or expression products thereof from a panel of IBD markers in a test sample obtained from the subject relative to the level of expression in a control, wherein the differential level of expression is indicative of the presence of an IBD in the subject from which the test sample was obtained. The differential expression in the test sample may be higher and/or lower relative to a control as discussed herein.

For all aspects of the invention, the method may further comprise the step of creating a report summarizing said prediction.

For all aspects, the IBD diagnosed or detected according to the methods of the present invention is Crohn's disease (CD), ulcerative colitis (UC), or both CD and UC.

For all aspects of the invention, the test sample obtained from a mammalian subject may be derived from a colonic tissue biopsy. In a preferred embodiment, the biopsy is a tissue selected from the group consisting of terminal ileum, the ascending colon, the descending colon, and the sigmoid colon. In other preferred embodiments, the biopsy is from an inflamed colonic area or from a non-inflamed colonic area. The inflamed colonic area may be acutely inflamed or chronically inflamed.

For all aspects, determination of expression levels may occur at more than one time. For all aspects of the invention, the determination of expression levels may occur before the patient is subjected to any therapy before and/or after any surgery. In some embodiments, the determining step is indicative of a recurrence of an IBD in the mammalian subject following surgery or indicative of a flare-up of said IBD in said mammalian subject. In a preferred embodiment, the IBD is Crohn's disease.

In another aspect, the present invention concerns methods of treating a mammalian subject in which the presence of an IBD has been detected by the methods described herein. For example, following a determination that a test sample obtained from the mammalian subject exhibits differential expression relative to a control of one or more of the RNA transcripts or the corresponding gene products of an IBD marker described herein, the mammalian subject may be administered an IBD therapeutic agent.

In one embodiment, the methods of treating an IBD in a mammalian subject in need thereof, comprise (a) determining a differential level of expression of (i) one or more nucleic acids encoding one or more polypeptides selected from Tables 1, 2, or 3; or (ii) RNA transcripts or expression products thereof of one or more genes selected from Tables 1, 2 or 3 in a test sample obtained from said subject relative to the level of expression in a control, wherein said differential level of expression is indicative of the presence of an IBD in the subject from which the test sample was obtained; and (b) administering to said subject an effective amount of an IBD therapeutic agent. In a preferred embodiment, the methods of treating an IBD comprise (a) determining that the expression level of (i) one or more nucleic acids encoding one or more polypeptides selected from Tables 1A, 2, or 3A; or (ii) RNA transcripts or expression products thereof of one or more genes selected from Tables 1A, 2 or 3A in a test sample obtained from the subject is higher relative to the level of expression in a control, wherein the higher level of expression is indicative of the presence of an IBD in the subject from which the test sample was obtained; and (b) administering to said subject an effective amount of an IBD therapeutic agent. In another preferred embodiment, the methods of treating an IBD comprise (a) determining that the expression level of (i) one or more nucleic acids encoding one or more polypeptides selected from Tables 1B or 3B; or (ii) RNA transcripts or expression products thereof of one or more genes selected from Tables 1B or 3B in a test sample obtained from the subject is lower relative to the level of expression in a control, wherein the lower level of expression is indicative of the presence of an IBD in the subject from which the test sample was obtained. In some preferred embodiments, the IBD therapeutic agent is one or more of an aminosalicylate, a corticosteroid, and an immunosuppressive agent.

In one aspect, the panel of IBD markers discussed above is useful in methods of treating an IBD in a mammalian subject. In one embodiment, the mammalian subject is screened against the panel of markers and if the presence of an IBD is determined, IBD therapeutic agent(s) may be administered as discussed herein.

In a different aspect the invention concerns a kit comprising one or more of (1) extraction buffer/reagents and protocol; (2) reverse transcription buffer/reagents and protocol; and (3) qPCR buffer/reagents and protocol suitable for performing the methods of this invention. The kit may comprise data retrieval and analysis software.

In one embodiment, the gene whose differential expression is indicative of an IBD is GLI1. In another embodiment, the GLI1 gene is a GLI1 variant. In a preferred embodiment, the GLI1 variant is rs2228226C→G (Q1100E) as described in Example 4.

All publications mentioned herein are incorporated herein by reference to disclose and describe the methods and/or materials in connection with which the publications are cited. Publications cited herein are cited for their disclosure prior to the filing date of the present application. Nothing here is to be construed as an admission that the inventors are not entitled to antedate the publications by virtue of an earlier priority date or prior date of invention. Further the actual publication dates may be different from those shown and require independent verification.

### BRIEF DESCRIPTION OF DRAWINGS

FIG. 1 shows the histologically normal biopsies from control patients that were analysed by unsupervised hierarchical clustering.

FIGS. 2A and 2B shows the expression of defensins alpha 5 (A) and alpha 6 (B) in ulcerative colitis patients and controls.

FIGS. 3A and 3B shows the expression of the matrix metalloproteinases (MMPs) 3 (A) and 7 (B) in ulcerative colitis and controls.

FIG. 4A-D shows the real time PCR expression of SAA1(A), IL-8(B), defensin alpha 5(C), and defensin alpha 6(D) in control and ulcerative colitis inflamed and non-inflamed sigmoid colon biopsies.

FIG. 5A-D shows the real time PCR expression of MMP3(A), MMP7(B), S100A8(C), and TLR4(D) in control and ulcerative colitis inflamed and non-inflamed sigmoid colon biopsies.

FIG. 6 shows in situ hybridization of defensin alpha 5 in the terminal ileum and colon of patients with ulcerative colitis and controls.

FIG. 7 shows in situ hybridization of defensin alpha 6 in the terminal ileum and colon of patients with ulcerative colitis and controls.

FIGS. 8A and 8B depict the nucleic acid sequence (SEQ ID NO:1) encoding human DEFA6 polypeptide and the amino acid sequence of human DEFA6 polypeptide (SEQ ID NO:2).

FIGS. 9A and 9B depict the nucleic acid sequence (SEQ ID NO:3) encoding human DEFAS polypeptide and the amino acid sequence of human DEFAS polypeptide (SEQ ID NO:4). FIGS. 9C and 9D depict the nucleic acid sequence (SEQ ID NO:210) encoding human DEFB14 polypeptide and the amino acid sequence of human DEFB14 polypeptide (SEQ ID NO:211).

FIGS. 10A and 10B depict the nucleic acid sequence (SEQ ID NO:5) encoding human IL3RA polypeptide and the amino acid sequence of human IL3RA polypeptide (SEQ ID NO:6).

FIGS. 11A and 11B depict the nucleic acid sequence (SEQ ID NO:7) encoding human IL2RA polypeptide and the amino acid sequence of human IL2RA polypeptide (SEQ ID NO:8).

FIGS. 12A and 12B depict the nucleic acid sequence (SEQ ID NO:9) encoding human REG3G polypeptide and the amino acid sequence of human REG3G polypeptide (SEQ ID NO:10).

FIGS. 13A and 13B depict the nucleic acid sequence (SEQ ID NO:11) encoding human REG1B polypeptide and the amino acid sequence of human REG1B polypeptide (SEQ ID NO:12).

FIGS. 14A and 14B depict the nucleic acid sequence (SEQ ID NO:13) encoding human KCND3 polypeptide and the amino acid sequence of human KCND3 polypeptide (SEQ ID NO:14).

FIGS. 15A and 15B depict the nucleic acid sequence (SEQ ID NO:15) encoding human MIP-3a polypeptide and the amino acid sequence of human MIP-3a polypeptide (SEQ ID NO:16).

FIGS. 16A and 16B depict the nucleic acid sequence (SEQ ID NO:17) encoding human ECGF1 polypeptide and the amino acid sequence of human ECGF1 polypeptide (SEQ ID NO:18).

FIGS. 17A and 17B depict the nucleic acid sequence (SEQ ID NO:19) encoding human IL1B polypeptide and the amino acid sequence of human IL1B polypeptide (SEQ ID NO:20).

FIGS. 18A and 18B depict the nucleic acid sequence (SEQ ID NO:21) encoding human MIP2BGRO-g polypeptide and the amino acid sequence of human MIP2BGRO-g polypeptide (SEQ ID NO:22).

FIGS. 19A and 19B depict the nucleic acid sequence (SEQ ID NO:23) encoding human CXCL1 polypeptide and the amino acid sequence of human CXCL1 polypeptide (SEQ ID NO:24).

FIGS. 20A and 20B depict the nucleic acid sequence (SEQ ID NO:25) encoding human IAP1 polypeptide and the amino acid sequence of human IAP1 polypeptide (SEQ ID NO:26).

FIGS. 21A and 21B depict the nucleic acid sequence (SEQ ID NO:27) encoding human CASP5 polypeptide and the amino acid sequence of human CASP5 polypeptide (SEQ ID NO:28).

FIGS. 22A and 22B depict the nucleic acid sequence (SEQ ID NO:29) encoding human DMBT1 polypeptide and the amino acid sequence of human DMBT1 polypeptide (SEQ ID NO:30).

FIGS. 23A and 23B depict the nucleic acid sequence (SEQ ID NO:31) encoding human PCDH17 polypeptide and the amino acid sequence of human PCDH17 polypeptide (SEQ ID NO:32).

FIGS. 24A and 24B depict the nucleic acid sequence (SEQ ID NO:33) encoding human IFITM1 polypeptide and the amino acid sequence of human IFITM1 polypeptide (SEQ ID NO:34).

FIGS. 25A and 25B depict the nucleic acid sequence (SEQ ID NO:35) encoding human PDZK1IP1 polypeptide and the amino acid sequence of human PDZK1IP1 polypeptide (SEQ ID NO:36).

FIGS. 26A and 26B depict the nucleic acid sequence (SEQ ID NO:37) encoding human IRTA2 polypeptide and the amino acid sequence of human IRTA2 polypeptide (SEQ ID NO:38).

FIGS. 27A and 27B depict the nucleic acid sequence (SEQ ID NO:39) encoding human SLC40A1 polypeptide and the amino acid sequence of human SLC40A1 polypeptide (SEQ ID NO:40).

FIGS. 28A and 28B depict the nucleic acid sequence (SEQ ID NO:41) encoding human IGHV4-4 polypeptide and the amino acid sequence of human IGHV4-4 polypeptide (SEQ ID NO:42).

FIGS. 29A and 29B depict the nucleic acid sequence (SEQ ID NO:43) encoding human REG3G polypeptide and the amino acid sequence of human REG3G polypeptide (SEQ ID NO:44).

FIGS. 30A and 30B depict the nucleic acid sequence (SEQ ID NO:45) encoding human AQP9 polypeptide and the amino acid sequence of human AQP9 polypeptide (SEQ ID NO:46).

FIGS. 31A and 31B depict the nucleic acid sequence (SEQ ID NO:47) encoding human OLFM4 polypeptide and the amino acid sequence of human OLFM4 polypeptide (SEQ ID NO:48).

FIGS. 32A and 32B depict the nucleic acid sequence (SEQ ID NO:49) encoding human S100A9 polypeptide and the amino acid sequence of human S100A9 polypeptide (SEQ ID NO:50).

FIGS. 33A and 33B depict the nucleic acid sequence (SEQ ID NO:51) encoding human UNC5CL polypeptide and the amino acid sequence of human UNC5CL polypeptide (SEQ ID NO:52).

FIGS. 34A and 34B depict the nucleic acid sequence (SEQ ID NO:53) encoding human GPR110 polypeptide and the amino acid sequence of human GPR110 polypeptide (SEQ ID NO:54).

FIGS. 35A and 35B depict the nucleic acid sequence (SEQ ID NO:55) encoding human HLA-G polypeptide and the amino acid sequence of human HLA-G polypeptide (SEQ ID NO:56).

FIGS. 36A and 36B depict the nucleic acid sequence (SEQ ID NO:57) encoding human TAP1 polypeptide and the amino acid sequence of human TAP1 polypeptide (SEQ ID NO:58).

FIGS. 37A and 37B depict the nucleic acid sequence (SEQ ID NO:59) encoding human MAP3K8 polypeptide and the amino acid sequence of human MAP3K8 polypeptide (SEQ ID NO:60).

FIGS. 38A and 38B depict the nucleic acid sequence (SEQ ID NO:61) encoding human UBD|GABBR1 polypeptide and the amino acid sequence of human UBD|GABBR1 polypeptide (SEQ ID NO:62).

FIGS. 39A and 39B depict the nucleic acid sequence (SEQ ID NO:63) encoding human DHX57 polypeptide and the amino acid sequence of human DHX57 polypeptide (SEQ ID NO:64).

FIGS. 40A and 40B depict the nucleic acid sequence (SEQ ID NO:65) encoding human MA polypeptide and the amino acid sequence of human MApolypeptide (SEQ ID NO:66).

FIGS. 41A and 41B depict the nucleic acid sequence (SEQ ID NO:67) encoding human IGLJCOR18 polypeptide and the amino acid sequence of human IGUCOR18 polypeptide (SEQ ID NO:68).

FIGS. 42A and 42B depict the nucleic acid sequence (SEQ ID NO:69) encoding human TNF R superfamily, member 6b, decoy (TNFRSF6B) polypeptide and the amino acid sequence of human TNF R superfamily, member 6b, decoy (TNFRSF6B) polypeptide (SEQ ID NO:70).

FIGS. 43A and 43B depict the nucleic acid sequence (SEQ ID NO:71) encoding human SAA1 polypeptide and the amino acid sequence of human SAA1 polypeptide (SEQ ID NO:72).

FIGS. 44A and 44B depict the nucleic acid sequence (SEQ ID NO:73) encoding human TAP2 polypeptide and the amino acid sequence of human TAP2 polypeptide (SEQ ID NO:74).

FIGS. 45A and 45B depict the nucleic acid sequence (SEQ ID NO:75) encoding human PCAA17448 polypeptide and the amino acid sequence of human PCAA17448 polypeptide (SEQ ID NO:76).

FIGS. 46A and 46B depict the nucleic acid sequence (SEQ ID NO:77) encoding human LCN2 polypeptide and the amino acid sequence of human LCN2 polypeptide (SEQ ID NO:78).

FIGS. 47A and 47B depict the nucleic acid sequence (SEQ ID NO:79) encoding human ZBP1 polypeptide and the amino acid sequence of human ZBP1 polypeptide (SEQ ID NO:80).

FIGS. 48A and 48B depict the nucleic acid sequence (SEQ ID NO:81) encoding human TNIP3 polypeptide and the amino acid sequence of human TNIP3 polypeptide (SEQ ID NO:82).

FIGS. 49A and 49B depict the nucleic acid sequence (SEQ ID NO:83) encoding human ZC3H12A polypeptide and the amino acid sequence of human ZC3H12A polypeptide (SEQ ID NO:84).

FIGS. 50A and 50B depict the nucleic acid sequence (SEQ ID NO:85) encoding human CHI3L1 polypeptide and the amino acid sequence of human CHI3L1 polypeptide (SEQ ID NO:86).

FIGS. 51A and 51B depict the nucleic acid sequence (SEQ ID NO:87) encoding human FCGR3A polypeptide and the amino acid sequence of human FCGR3A polypeptide (SEQ ID NO:88).

FIGS. 52A and 52B depict the nucleic acid sequence (SEQ ID NO:89) encoding human SAMD9L polypeptide and the amino acid sequence of human SAMD9L polypeptide (SEQ ID NO:90).

FIGS. 53A and 53B depict the nucleic acid sequence (SEQ ID NO:91) encoding human MMP9 polypeptide and the amino acid sequence of human MMP9 polypeptide (SEQ ID NO:92).

FIGS. 54A and 54B depict the nucleic acid sequence (SEQ ID NO:93) encoding human MMP7 polypeptide and the amino acid sequence of human MMP7 polypeptide (SEQ ID NO:94).

FIGS. 55A and 55B depict the nucleic acid sequence (SEQ ID NO:95) encoding human BF polypeptide and the amino acid sequence of human BF polypeptide (SEQ ID NO:96).

FIGS. 56A and 56B depict the nucleic acid sequence (SEQ ID NO:97) encoding human S100P polypeptide and the amino acid sequence of human S100P polypeptide (SEQ ID NO:98).

FIGS. 57A and 57B depict the nucleic acid sequence (SEQ ID NO:99) encoding human GRO polypeptide and the amino acid sequence of human GRO polypeptide (SEQ ID NO:100).

FIGS. 58A and 58B depict the nucleic acid sequence (SEQ ID NO:101) encoding human INDO polypeptide and the amino acid sequence of human INDO polypeptide (SEQ ID NO:102).

FIGS. 59A and 59B depict the nucleic acid sequence (SEQ ID NO:103) encoding human TRIM22 polypeptide and the amino acid sequence of human TRIM22 polypeptide (SEQ ID NO:104).

FIGS. 60A and 60B depict the nucleic acid sequence (SEQ ID NO:105) encoding human SAA2 polypeptide and the amino acid sequence of human SAA2 polypeptide (SEQ ID NO:106).

FIGS. 61A and 61B depict the nucleic acid sequence (SEQ ID NO:107) encoding human NEU4 polypeptide and the amino acid sequence of human NEU4 polypeptide (SEQ ID NO:108).

FIGS. 62A and 62B depict the nucleic acid sequence (SEQ ID NO:109) encoding human IRTA2/FCRH5 polypeptide and the amino acid sequence of human IRTA2/FCRH5 polypeptide (SEQ ID NO:110).

FIGS. 63A and 63B depict the nucleic acid sequence (SEQ ID NO:111) encoding human IGLJCOR18 polypeptide and the amino acid sequence of human IGUCOR18 polypeptide (SEQ ID NO:112).

FIGS. 64A and 64B depict the nucleic acid sequence (SEQ ID NO:113) encoding human IGHV4-4 polypeptide and the amino acid sequence of human IGHV4-4 polypeptide (SEQ ID NO:114).

FIGS. 65A and 65B depict the nucleic acid sequence (SEQ ID NO:115) encoding human MMP9 polypeptide and the amino acid sequence of human MMP9 polypeptide (SEQ ID NO:116).

FIGS. 66A and 66B depict the nucleic acid sequence (SEQ ID NO:117) encoding human GRO polypeptide and the amino acid sequence of human GRO polypeptide (SEQ ID NO:118).

FIGS. 67A and 67B depict the nucleic acid sequence (SEQ ID NO:119) encoding human MIP2BGRO-g polypeptide and the amino acid sequence of human MIP2BGRO-g polypeptide (SEQ ID NO:120).

FIGS. 68A and 68B depict the nucleic acid sequence (SEQ ID NO:121) encoding human IL1B polypeptide and the amino acid sequence of human IL1B polypeptide (SEQ ID NO:122).

FIGS. 69A and 69B depict the nucleic acid sequence (SEQ ID NO:123) encoding human IL3RA polypeptide and the amino acid sequence of human IL3RA polypeptide (SEQ ID NO:124).

FIGS. 70A and 70B depict the nucleic acid sequence (SEQ ID NO:125) encoding human CASP1 polypeptide and the amino acid sequence of human CASP1 polypeptide (SEQ ID NO:126).

FIGS. 71A and 71B depict the nucleic acid sequence (SEQ ID NO:127) encoding human BV8 polypeptide and the amino acid sequence of human BV8 polypeptide (SEQ ID NO:128).

FIGS. 72A and 72B depict the nucleic acid sequence (SEQ ID NO:129) encoding human HDAC7A polypeptide and the amino acid sequence of human HDAC7A polypeptide (SEQ ID NO:130).

FIGS. 73A and 73B depict the nucleic acid sequence (SEQ ID NO:131) encoding human ACVRL1 polypeptide and the amino acid sequence of human ACVRL1 polypeptide (SEQ ID NO:132).

FIGS. 74A and 74B depict the nucleic acid sequence (SEQ ID NO:133) encoding human NR4A1 polypeptide and the amino acid sequence of human NR4A1 polypeptide (SEQ ID NO:134).

FIGS. 75A and 75B depict the nucleic acid sequence (SEQ ID NO:135) encoding human K5B polypeptide and the amino acid sequence of human K5B polypeptide (SEQ ID NO:136).

FIGS. 76A and 76B depict the nucleic acid sequence (SEQ ID NO:137) encoding human SILV polypeptide and the amino acid sequence of human SILV polypeptide (SEQ ID NO:138).

FIGS. 77A and 77B depict the nucleic acid sequence (SEQ ID NO:139) encoding human IRAK3 polypeptide and the amino acid sequence of human IRAK3 polypeptide (SEQ ID NO:140).

FIGS. 78A and 78B depict the nucleic acid sequence (SEQ ID NO:141) encoding human IL-4 polypeptide and the amino acid sequence of human IL-4 polypeptide (SEQ ID NO:142).

FIGS. 79A and 79B depict the nucleic acid sequence (SEQ ID NO:143) encoding human IL-13 polypeptide and the amino acid sequence of human IL-13 polypeptide (SEQ ID NO:144).

FIGS. 80A and 80B depict the nucleic acid sequence (SEQ ID NO:145) encoding human RAD50 polypeptide and the amino acid sequence of human RAD50 polypeptide (SEQ ID NO:146).

FIGS. 81A and 81B depict the nucleic acid sequence (SEQ ID NO:147) encoding human IL-5 polypeptide and the amino acid sequence of human IL-5 polypeptide (SEQ ID NO:148).

FIGS. 82A and 82B depict the nucleic acid sequence (SEQ ID NO:149) encoding human IRF1 polypeptide and the amino acid sequence of human IRF1 polypeptide (SEQ ID NO:150).

FIGS. 83A and 83B depict the nucleic acid sequence (SEQ ID NO:151) encoding human PDLIM4 polypeptide and the amino acid sequence of human PDLIM4 polypeptide (SEQ ID NO:152).

FIGS. 84A and 84B depict the nucleic acid sequence (SEQ ID NO:153) encoding human CSF2 polypeptide and the amino acid sequence of human CSF2 polypeptide (SEQ ID NO:154).

FIGS. 85A and 85B depict the nucleic acid sequence (SEQ ID NO:155) encoding human IL-3 polypeptide and the amino acid sequence of human IL-3 polypeptide (SEQ ID NO:156).

FIGS. 86A and 86B depict the nucleic acid sequence (SEQ ID NO:157) encoding human MMP3 polypeptide and the amino acid sequence of human MMP3 polypeptide (SEQ ID NO:158).

FIGS. 87A and 87B depict the nucleic acid sequence (SEQ ID NO:159) encoding human IL-8 polypeptide and the amino acid sequence of human IL-8 polypeptide (SEQ ID NO:160).

FIGS. 88A and 88B depict the nucleic acid sequence (SEQ ID NO:161) encoding human TLR4 polypeptide and the amino acid sequence of human TLR4 polypeptide (SEQ ID NO:162).

FIGS. 89A and 89B depict the nucleic acid sequence (SEQ ID NO:163) encoding human HLA-DRB 1 polypeptide and the amino acid sequence of human HLA-DRB 1 polypeptide (SEQ ID NO:164).

FIGS. 90A and 90B depict the nucleic acid sequence (SEQ ID NO:165) encoding human MMP19 polypeptide and the amino acid sequence of human MMP19 polypeptide (SEQ ID NO:166).

FIGS. 91A and 91B depict the nucleic acid sequence (SEQ ID NO:167) encoding human TIMP1 polypeptide and the amino acid sequence of human TIMP1 polypeptide (SEQ ID NO:168).

FIGS. 92A and 92B depict the nucleic acid sequence (SEQ ID NO:169) encoding human Elfin polypeptide and the amino acid sequence of human Elfin polypeptide (SEQ ID NO:170).

FIGS. 93A and 93B depict the nucleic acid sequence (SEQ ID NO:171) encoding human CXCL1 polypeptide and the amino acid sequence of human CXCL1 polypeptide (SEQ ID NO:172).

FIGS. 94A and 94B depict the nucleic acid sequence (SEQ ID NO:173) encoding human DKFZP586A0522 polypeptide and the amino acid sequence of human DFKZP586A0522 polypeptide (SEQ ID NO:174).

FIGS. 95A and 95B depict the nucleic acid sequence (SEQ ID NO:175) encoding human SLC39A5 polypeptide and the amino acid sequence of human SLC39A5 polypeptide (SEQ ID NO:176).

FIGS. 96A and 96B depict the nucleic acid sequence (SEQ ID NO:177) encoding human GLI-1 polypeptide and the amino acid sequence of human GLI-1 polypeptide (SEQ ID NO:178).

FIGS. 97A and 97B depict the nucleic acid sequence (SEQ ID NO:179) encoding human HMGA2 polypeptide and the amino acid sequence of human HMGA2 polypeptide (SEQ ID NO:180).

FIGS. 98A and 98B depict the nucleic acid sequence (SEQ ID NO:181) encoding human SLC22A5 polypeptide and the amino acid sequence of human SLC22A5 polypeptide (SEQ ID NO:182).

FIGS. 99A and 99B depict the nucleic acid sequence (SEQ ID NO:183) encoding human SLC22A4 polypeptide and the amino acid sequence of human SLC22A4 polypeptide (SEQ ID NO:184).

FIGS. 100A and 100B depict the nucleic acid sequence (SEQ ID NO:185) encoding human P4HA2 polypeptide and the amino acid sequence of human P4HA2 polypeptide (SEQ ID NO:186).

FIGS. 101A and 101B depict the nucleic acid sequence (SEQ ID NO:187) encoding human TSLP polypeptide and the amino acid sequence of human TSLP polypeptide (SEQ ID NO:188).

FIGS. 102A and 102B depict the nucleic acid sequence (SEQ ID NO:189) encoding human tubulin alpha 5/alpha 3 polypeptide and the amino acid sequence of human tubulin alpha 5/alpha 3 polypeptide (SEQ ID NO:190).

FIGS. 103A and 103B depict the nucleic acid sequence (SEQ ID NO:191) encoding human tubulin alpha 6 polypeptide and the amino acid sequence of human tubulin alpha 6 polypeptide (SEQ ID NO:192).

FIG. 104 shows a meta-analysis of non-synonymous GLI1 SNP rs2228226 in Scotland, Cambridge and Sweden using Mantel-Haenszel method.

FIG. 105A-E shows Q1100E disrupts a conserved region of the GLI1 protein (SEQ ID NOS:245-250) and reduces GLI1 transcriptional activity.

FIG. 106A-C shows expression of hedgehog (HH) signalling components in the healthy human adult colon (HC) and ulcerative colitis (UC).

FIG. 107A-C shows the results in which Gli1+/− animals show mortality, severe clinical symptoms, and profound weight loss after DSS treatment.

FIG. 108A-F shows Gli1+/− animals demonstrate more severe intestinal inflammation than WT littermates in response to DSS treatment.

FIG. 109A-B shows cytokine analysis of Gli1+/− and WT mice after DSS treatments demonstrates robust pro-inflammatory cytokine activation.

FIG. 110A-B shows the (A) nucleic acid sequence encoding human arrestin domain containing 5 (ARRDCS) (LOC342959) (SEQ ID NO:193) and the (B) amino acid sequence of human ARRDCS polypeptide (SEQ ID NO:194).

FIG. 111 shows the nucleic acid sequence corresponding to human ataxin 3-like (ATXN3L) (SEQ ID NO:195).

FIG. 112A-B shows the (A) nucleic acid sequence encoding human follicle stimulating hormone receptor (FSHR) (LOC92552) (SEQ ID NO:196) and (B) the amino acid sequence of human FSHR polypeptide (SEQ ID NO:197).

FIG. 113A-B shows the (A) nucleic acid sequence encoding human Platelet-derived growth factor receptor, alpha polypeptide (PDGFRA) (SEQ ID NO:198) and (B) the amino acid sequence of human PDGFRA polypeptide (SEQ ID NO:199).

FIG. 114A-B shows the (A) nucleic acid sequence encoding human transforming growth factor beta 3 (TGFB3) (SEQ ID NO:200) and (B) the amino acid sequence of human TGFB3 polypeptide (SEQ ID NO:201).

FIG. 115A-B shows the (A) nucleic acid sequence encoding human potassium channel tetramerisation domain containing 8 (KCTD8) (SEQ ID NO:202) and (B) the amino acid sequence of human KCTD8 polypeptide (SEQ ID NO:203).

FIG. 116A-B shows the (A) nucleic acid sequence encoding human transglutaminase 4 (TGM4) (SEQ ID NO:204) and (B) the amino acid sequence of human TGM4 polypeptide (SEQ ID NO:205).

FIG. 117A-B shows the (A) nucleic acid sequence encoding human TPD52L3 tumor protein D52-like 3 (NYD-SP25) (SEQ ID NO:206) and (B) the amino acid sequence of human NYD-SP25 polypeptide (SEQ ID NO:207).

FIG. 118 shows the nucleic acid sequence corresponding to misc_RNA (C3orf53), FLJ33651 (SEQ ID NO:208).

FIG. 119 shows the nucleic acid sequence corresponding to EMX2 opposite strand (non-protein coding) (EMX2OS) on chromosome 10 (SEQ ID NO:209).

FIG. 120A-B shows the (A) nucleic acid sequence encoding human wingless-type MMTV integration site family, member 16 (WNT16) (SEQ ID NO:210) and (B) the amino acid sequence of human WNT16 polypeptide (SEQ ID NO:211).

FIG. 121A-C shows the (A-B) nucleic acid sequences encoding human sprouty-related, EVH1 domain containing 2 (SPRED2) (SEQ ID NO:212, 270) and (C) the amino acid sequence of human SPRED2 (SEQ ID NO:213).

FIG. 122A-C shows the (A-B) nucleic acid sequences encoding human chromosome 16 open reading frame 65 (C16orf65) (SEQ ID NO:214, 271) and (C) the amino acid sequence of human chromosome 16 open reading frame 65 (C16orf65) (SEQ ID NO:215).

FIG. 123A-B shows the (A) nucleic acid sequence encoding human chromosome 12 open reading frame 2 (C12orf2) (SEQ ID NO:216) and (B) the amino acid sequence of human chromosome 12 open reading frame 2 (C12orf2) (SEQ ID NO:217).

FIG. 124A-B shows the (A) nucleic acid sequence encoding human multiple PDZ domain protein (MPDZ) (SEQ ID NO:218) and (B) the amino acid sequence of human MPDZ (SEQ ID NO:219).

FIG. 125A-B shows the (A) nucleic acid sequence encoding human phenylalanine-tRNA synthetase 2 (FARS2) (SEQ ID NO:220) and (B) the amino acid sequence of human FARS2 (SEQ ID NO:221).

FIG. 126A-B shows the (A) nucleic acid sequence encoding human caspase 8, apoptosis-related cysteine protease (CASP8) (SEQ ID NO:222) and (B) the amino acid sequence of human CASP8 (SEQ ID NO:223).

FIG. 127A-B shows the (A) nucleic acid sequence encoding human 5′-nucleotidase, ecto (CD73) (NT5E) (SEQ ID NO:224) and (B) the amino acid sequence of human NT5E (SEQ ID NO:225).

FIG. 128 shows the nucleic acid sequence corresponding to human teratocarcinoma-derived growth factor 3 (TDGF3) (SEQ ID NO:226).

FIG. 129A-B shows the (A) nucleic acid sequence encoding human butyrophilin-like 3 (BTNL3) (SEQ ID NO:227) and (B) the amino acid sequence of human BTNL3 (SEQ ID NO:228).

FIG. 130A-B shows the (A) nucleic acid sequence encoding human S100A8 and (B) the amino acid sequence of human S100A8.

FIG. 131A-B shows the (A) nucleic acid sequence encoding human CCL20 and (B) the amino acid sequence of human CCL20.

### DETAILED DESCRIPTION OF THE INVENTION

### A. Definitions

Unless defined otherwise, technical and scientific terms used herein have the same meaning as commonly understood by one of ordinary skill in the art to which this invention belongs. Singleton et al., Dictionary of Microbiology and Molecular Biology 2nd ed., J. Wiley & Sons (New York, N.Y. 1994), and March, Advanced Organic Chemistry Reactions, Mechanisms and Structure 4th ed., John Wiley & Sons (New York, N.Y. 1992), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

The term “inflammatory bowel disease” or “IBD” is used as a collective term for ulcerative colitis and Crohn's disease. Although the two diseases are generally considered as two different entities, their common characteristics, such as patchy necrosis of the surface epithelium, focal accumulations of leukocytes adjacent to glandular crypts, and an increased number of intraepithelial lymphocytes (IEL) and certain macrophage subsets, justify their treatment as a single disease group.

The term “Crohn's disease” or “CD” is used herein to refer to a condition involving chronic inflammation of the gastrointestinal tract. Crohn's-related inflammation usually affects the intestines, but may occur anywhere from the mouth to the anus. CD differs from UC in that the inflammation extends through all layers of the intestinal wall and involves mesentery as well as lymph nodes. The disease is often discontinuous, i.e., severely diseased segments of bowel are separated from apparently disease-free areas. In CD, the bowel wall also thickens which can lead to obstructions, and the development of fistulas and fissures are not uncommon As used herein, CD may be one or more of several types of CD, including without limitation, ileocolitis (affects the ileum and the large intestine); ileitis (affects the ileum); gastroduodenal CD (inflammation in the stomach and the duodenum); jejunoileitis (spotty patches of inflammation in the jejunum); and Crohn's (granulomatous) colitis (only affects the large intestine).

The term “ulcerative colitis” or “UC” is used herein to refer to a condition involving inflammation of the large intestine and rectum. In patients with UC, there is an inflammatory reaction primarily involving the colonic mucosa. The inflammation is typically uniform and continuous with no intervening areas of normal mucosa. Surface mucosal cells as well as crypt epithelium and submucosa are involved in an inflammatory reaction with neutrophil infiltration. Ultimately, this reaction typically progresses to epithelial damage and loss of epithelial cells resulting in multiple ulcerations, fibrosis, dysplasia and longitudinal retraction of the colon.

The term “inactive” IBD is used herein to mean an IBD that was previously diagnosed in an individual but is currently in remission. This is in contrast to an “active” IBD in which an individual has been diagnosed with and IBD but has not undergone treatment. In addition, the active IBD may be a recurrence of a previously diagnosed and treated IBD that had gone into remission (i.e. become an inactive IBD). Such recurrences may also be referred to herein as “flare-ups” of an IBD. Mammalian subjects having an active autoimmune disease, such as an IBD, may be subject to a flare-up, which is a period of heightened disease activity or a return of corresponding symptoms. Flare-ups may occur in response to severe infection, allegic reactions, physical stress, emotional trauma, surgery, or environmental factors.

The term “modulate” is used herein to mean that the expression of the gene, or level of RNA molecule or equivalent RNA molecules encoding one or more proteins or protein subunits, or activity of one or more proteins or protein subunits is up regulated or down regulated, such that expression, level, or activity is greater than or less than that observed in the absence of the modulator.

The terms “inhibit”, “down-regulate”, “underexpress” and “reduce” are used interchangeably and mean that the expression of a gene, or level of RNA molecules or equivalent RNA molecules encoding one or more proteins or protein subunits, or activity of one or more proteins or protein subunits, is reduced relative to one or more controls, such as, for example, one or more positive and/or negative controls.

The term “up-regulate” or “overexpress” is used to mean that the expression of a gene, or level of RNA molecules or equivalent RNA molecules encoding one or more proteins or protein subunits, or activity of one or more proteins or protein subunits, is elevated relative to one or more controls, such as, for example, one or more positive and/or negative controls.

The term “diagnosis” is used herein to refer to the identification of a molecular or pathological state, disease or condition, such as the identification of IBD.

The term “prognosis” is used herein to refer to the prediction of the likelihood of IBD development or progression, including autoimmune flare-ups and recurrences following surgery. Prognostic factors are those variables related to the natural history of IBD, which influence the recurrence rates and outcome of patients once they have developed IBD. Clinical parameters that may be associated with a worse prognosis include, for example, an abdominal mass or tenderness, skin rash, swollen joints, mouth ulcers, and borborygmus (gurgling or splashing sound over the intestine). Prognostic factors may be used to categorize patients into subgroups with different baseline recurrence risks.

The “pathology” of an IBD includes all phenomena that compromise the well-being of the patient. IBD pathology is primarily attributed to abnormal activation of the immune system in the intestines that can lead to chronic or acute inflammation in the absence of any known foreign antigen, and subsequent ulceration. Clinically, IBD is characterized by diverse manifestations often resulting in a chronic, unpredictable course. Bloody diarrhea and abdominal pain are often accompanied by fever and weight loss. Anemia is not uncommon, as is severe fatigue. Joint manifestations ranging from arthralgia to acute arthritis as well as abnormalities in liver function are commonly associated with IBD. During acute “attacks” of IBD, work and other normal activity are usually impossible, and often a patient is hospitalized.

The aetiology of these diseases is unknown and the initial lesion has not been clearly defined; however, patchy necrosis of the surface epithelium, focal accumulations of leukocytes adjacent to glandular crypts, and an increased number of intraepithelial lymphocytes and certain macrophage subsets have been described as putative early changes, especially in Crohn's disease.

The term “treatment” refers to both therapeutic treatment and prophylactic or preventative measures for IBD, wherein the object is to prevent or slow down (lessen) the targeted pathologic condition or disorder. Those in need of treatment include those already with an IBD as well as those prone to have an IBD or those in whom the IBD is to be prevented. Once the diagnosis of an IBD has been made by the methods disclosed herein, the goals of therapy are to induce and maintain a remission.

Various agents that are suitable for use as an “IBD therapeutic agent” are known to those of ordinary skill in the art. As described herein, such agents include without limitation, aminosalicylates, corticosteroids, and immunosuppressive agents.

The term “test sample” refers to a sample from a mammalian subject suspected of having an IBD, known to have an IBD, or known to be in remission from an IBD. The test sample may originate from various sources in the mammalian subject including, without limitation, blood, semen, serum, urine, bone marrow, mucosa, tissue, etc.

The term “control” or “control sample” refers a negative control in which a negative result is expected to help correlate a positive result in the test sample. Controls that are suitable for the present invention include, without limitation, a sample known to have normal levels of gene expression, a sample obtained from a mammalian subject known not to have an IBD, and a sample obtained from a mammalian subject known to be normal. A control may also be a sample obtained from a subject previously diagnosed and treated for an IBD who is currently in remission; and such a control is useful in determining any recurrence of an IBD in a subject who is in remission. In addition, the control may be a sample containing normal cells that have the same origin as cells contained in the test sample. Those of skill in the art will appreciate other controls suitable for use in the present invention.

The term “microarray” refers to an ordered arrangement of hybridizable array elements, preferably polynucleotide probes, on a substrate.

The term “polynucleotide,” when used in singular or plural, generally refers to any polyribonucleotide or polydeoxyribonucleotide, which may be unmodified RNA or DNA or modified RNA or DNA. Thus, for instance, polynucleotides as defined herein include, without limitation, single- and double-stranded DNA, DNA including single- and double-stranded regions, single- and double-stranded RNA, and RNA including single- and double-stranded regions, hybrid molecules comprising DNA and RNA that may be single-stranded or, more typically, double-stranded or include single- and double-stranded regions. In addition, the term “polynucleotide” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. The term “polynucleotide” specifically includes cDNAs. The term includes DNAs (including cDNAs) and RNAs that contain one or more modified bases. Thus, DNAs or RNAs with backbones modified for stability or for other reasons are “polynucleotides” as that term is intended herein. Moreover, DNAs or RNAs comprising unusual bases, such as inosine, or modified bases, such as tritiated bases, are included within the term “polynucleotides” as defined herein. In general, the term “polynucleotide” embraces all chemically, enzymatically and/or metabolically modified forms of unmodified polynucleotides, as well as the chemical forms of DNA and RNA characteristic of viruses and cells, including simple and complex cells.

The term “oligonucleotide” refers to a relatively short polynucleotide, including, without limitation, single-stranded deoxyribonucleotides, single- or double-stranded ribonucleotides, RNA:DNA hybrids and double-stranded DNAs. Oligonucleotides, such as single-stranded DNA probe oligonucleotides, are often synthesized by chemical methods, for example using automated oligonucleotide synthesizers that are commercially available. However, oligonucleotides can be made by a variety of other methods, including in vitro recombinant DNA-mediated techniques and by expression of DNAs in cells and organisms.

The terms “differentially expressed gene,” “differential gene expression” and their synonyms, which are used interchangeably, refer to a gene whose expression is activated to a higher or lower level in a subject suffering from a disease, specifically an IBD, such as UC or CD, relative to its expression in a normal or control subject. The terms also include genes whose expression is activated to a higher or lower level at different stages of the same disease. It is also understood that a differentially expressed gene may be either activated or inhibited at the nucleic acid level or protein level, or may be subject to alternative splicing to result in a different polypeptide product. Such differences may be evidenced by a change in mRNA levels, surface expression, secretion or other partitioning of a polypeptide, for example. Differential gene expression may include a comparison of expression between two or more genes or their gene products, or a comparison of the ratios of the expression between two or more genes or their gene products, or even a comparison of two differently processed products of the same gene, which differ between normal subjects and subjects suffering from a disease, specifically an IBD, or between various stages of the same disease. Differential expression includes both quantitative, as well as qualitative, differences in the temporal or cellular expression pattern in a gene or its expression products among, for example, normal and diseased cells, or among cells which have undergone different disease events or disease stages. For the purpose of this invention, “differential gene expression” is considered to be present when there is at least an about two-fold, preferably at least about four-fold, more preferably at least about six-fold, most preferably at least about ten-fold difference between the expression of a given gene in normal and diseased subjects, or in various stages of disease development in a diseased subject.

The term “over-expression” with regard to an RNA transcript is used to refer to the level of the transcript determined by normalization to the level of reference mRNAs, which might be all transcripts detected in the specimen or a particular reference set of mRNAs.

The phrase “gene amplification” refers to a process by which multiple copies of a gene or gene fragment are formed in a particular cell or cell line. The duplicated region (a stretch of amplified DNA) is often referred to as “amplicon”. Usually, the amount of the messenger RNA (mRNA) produced, i.e., the level of gene expression, also increases in the proportion of the number of copies made of the particular gene expressed.

In general, the term “marker” or “biomarker” or refers to an identifiable physical location on a chromosome, such as a restriction endonuclease recognition site or a gene, whose inheritance can be monitored. The marker may be an expressed region of a gene referred to as a “gene expression marker”, or some segment of DNA with no known coding function. An “IBD marker” as used herein refers those genes listed in Tables 1, 2, and 3.

“Stringency” of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures. Hybridization generally depends on the ability of denatured DNA to reanneal when complementary strands are present in an environment below their melting temperature. The higher the degree of desired homology between the probe and hybridizable sequence, the higher the relative temperature which can be used. As a result, it follows that higher relative temperatures would tend to make the reaction conditions more stringent, while lower temperatures less so. For additional details and explanation of stringency of hybridization reactions, see Ausubel et al., Current Protocols in Molecular Biology, Wiley Interscience Publishers, (1995).

“Stringent conditions” or “high stringency conditions”, as defined herein, typically: (1) employ low ionic strength and high temperature for washing, for example 0.015 M sodium chloride/0.0015 M sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with washes at 42° C. in 0.2×SSC (sodium chloride/sodium citrate), 50% formamide, followed by a high-stringency wash consisting of 0.1×SSC containing EDTA at 55° C.

“Moderately stringent conditions” may be identified as described by Sambrook et al., Molecular Cloning: A Laboratory Manual, New York: Cold Spring Harbor Press, 1989, and include the use of washing solution and hybridization conditions (e.g., temperature, ionic strength and % SDS) less stringent that those described above. An example of moderately stringent conditions is overnight incubation at 37° C. in a solution comprising: 20% formamide, 5×SSC (150 mM NaCl, 15 mM trisodium citrate), 50 mM sodium phosphate (pH 7.6), 5×Denhardt's solution, 10% dextran sulfate, and 20 mg/ml denatured sheared salmon sperm DNA, followed by washing the filters in 1×SSC at about 37-50° C. The skilled artisan will recognize how to adjust the temperature, ionic strength, etc. as necessary to accommodate factors such as probe length and the like.

In the context of the present invention, reference to “at least one,” “at least two,” “at least five,” etc. of the genes listed in any particular gene set means any one or any and all combinations of the genes listed.

The terms “splicing” and “RNA splicing” are used interchangeably and refer to RNA processing that removes introns and joins exons to produce mature mRNA with continuous coding sequence that moves into the cytoplasm of an eukaryotic cell.

In theory, the term “exon” refers to any segment of an interrupted gene that is represented in the mature RNA product (B. Lewin. Genes IV Cell Press, Cambridge Mass. 1990). In theory the term “intron” refers to any segment of DNA that is transcribed but removed from within the transcript by splicing together the exons on either side of it. Operationally, exon sequences occur in the mRNA sequence of a gene as defined by Ref. SEQ ID numbers. Operationally, intron sequences are the intervening sequences within the genomic DNA of a gene, bracketed by exon sequences and having GT and AG splice consensus sequences at their 5′ and 3′ boundaries.

An “interfering RNA” or “small interfering RNA (siRNA)” is a double stranded RNA molecule usually less than about 30 nucleotides in length that reduces expression of a target gene. Interfering RNAs may be identified and synthesized using known methods (Shi Y., Trends in Genetics 19(1):9-12 (2003), WO/2003056012 and WO2003064621), and siRNA libraries are commercially available, for example from Dharmacon, Lafayette, Colo.

A “native sequence” polypeptide is one which has the same amino acid sequence as a polypeptide derived from nature, including naturally occurring or allelic variants. Such native sequence polypeptides can be isolated from nature or can be produced by recombinant or synthetic means. Thus, a native sequence polypeptide can have the amino acid sequence of naturally occurring human polypeptide, murine polypeptide, or polypeptide from any other mammalian species.

The term “antibody” herein is used in the broadest sense and specifically covers monoclonal antibodies, polyclonal antibodies, multispecific antibodies (e.g. bispecific antibodies), and antibody fragments, so long as they exhibit the desired biological activity. The present invention particularly contemplates antibodies against one or more of the IBD markers disclosed herein. Such antibodies may be referred to as “anti-IBD marker antibodies”.

The term “monoclonal antibody” as used herein refers to an antibody from a population of substantially homogeneous antibodies, i.e., the individual antibodies comprising the population are identical and/or bind the same epitope(s), except for possible variants that may arise during production of the monoclonal antibody, such variants generally being present in minor amounts. Such monoclonal antibody typically includes an antibody comprising a polypeptide sequence that binds a target, wherein the target-binding polypeptide sequence was obtained by a process that includes the selection of a single target binding polypeptide sequence from a plurality of polypeptide sequences.

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., Proc. Natl. Acad. Sci. USA, 81:6851-6855 (1984)). Chimeric antibodies of interest herein include “primatized” antibodies comprising variable domain antigen-binding sequences derived from a non-human primate (e.g. Old World Monkey, Ape etc) and human constant region sequences, as well as “humanized” antibodies.

“Humanized” forms of non-human (e.g., rodent) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity.

An “intact antibody” herein is one which comprises two antigen binding regions, and an Fc region. Preferably, the intact antibody has a functional Fc region.

“Antibody fragments” comprise a portion of an intact antibody, preferably comprising the antigen binding region thereof. Examples of antibody fragments include Fab, Fab′, F(ab′)₂, and Fv fragments; diabodies; linear antibodies; single-chain antibody molecules; and multispecific antibodies formed from antibody fragment(s).

“Native antibodies” are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (V_{H}) followed by a number of constant domains. Each light chain has a variable domain at one end (V_{L}) and a constant domain at its other end. The constant domain of the light chain is aligned with the first constant domain of the heavy chain, and the light-chain variable domain is aligned with the variable domain of the heavy chain. Particular amino acid residues are believed to form an interface between the light chain and heavy chain variable domains.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called hypervariable regions both in the light chain and the heavy chain variable domains. The more highly conserved portions of variable domains are called the framework regions (FRs). The variable domains of native heavy and light chains each comprise four FRs, largely adopting a β-sheet configuration, connected by three hypervariable regions, which form loops connecting, and in some cases forming part of, the β-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991)).

The term “hypervariable region,” “HVR,” or “HV,” when used herein refers to the regions of an antibody-variable domain that are hypervariable in sequence and/or form structurally defined loops. Generally, antibodies comprise six HVRs; three in the VH(H1, H2, H3), and three in the VL (L1, L2, L3). In native antibodies, H3 and L3 display the most diversity of the six HVRs, and H3 in particular is believed to play a unique role in conferring fine specificity to antibodies. See, e.g., Xu et al. Immunity 13:37-45 (2000); Johnson and Wu in Methods in Molecular Biology 248:1-25 (Lo, ed., Human Press, Totowa, N.J., 2003)). Indeed, naturally occurring camelid antibodies consisting of a heavy chain only are functional and stable in the absence of light chain. See, e.g., Hamers-Casterman et al., Nature 363:446-448 (1993) and Sheriff et al., Nature Struct. Biol. 3:733-736 (1996).

A number of hypervariable region delineations are in use and are encompassed herein. The Kabat Complementarity Determining Regions (CDRs) are based on sequence variability and are the most commonly used (Kabat et al., Sequences of Proteins of Immunological Interest, 5th Ed. Public Health Service, National Institutes of Health, Bethesda, Md. (1991)). Chothia refers instead to the location of the structural loops (Chothia and Lesk J. Mol. Biol. 196:901-917 (1987)). The end of the Chothia CDR-H1 loop when numbered using the Kabat numbering convention varies between H32 and H34 (see below) depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops, and are used by Oxford Molecular's AbM antibody modeling software. The “contact” hypervariable regions are based on an analysis of the available complex crystal structures. The residues from each of these hypervariable regions are noted below.

| Loop | Kabat | AbM | Chothia | Contact |
|---|---|---|---|---|
| L1 | L24-L34 | L24-L34 | L24-L34 | L30-L36 |
| L2 | L50-L56 | L50-L56 | L50-L56 | L46-L55 |
| L3 | L89-L97 | L89-L97 | L89-L97 | L89-L96 |
| H1 | H31-H35B | H26-H35B | H26-H32, | H30-H35B |
| | | | 33 or 34 | |

(Kabat Numbering)

| | | | | |
|---|---|---|---|---|
| H1 | H31-H35 | H26-H35 | H26-H32 | H30-H35 |

(Chothia Numbering)

| | | | | |
|---|---|---|---|---|
| H2 | H50-H65 | H50-H58 | H52-H56 | H47-H58 |
| H3 | H95-H102 | H95-H102 | H95-H102 | H93-H101. |

Hypervariable regions may comprise “extended hypervariable regions” as follows: 24-36 or 24-34 (L1), 46-56 or 50-56 (L2) and 89-97 (L3) in the VL and 26-35B (H1), 50-65, 47-65 or 49-65 (H2) and 93-102, 94-102 or 95-102 (H3) in the VH. These extended hypervariable regions are typically combinations of the Kabat and Chothia definitions, which may optionally further include residues identified using the Contact definition. The variable domain residues are numbered according to Kabat et al., supra for each of these definitions.

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two antigen-binding sites and is still capable of cross-linking antigen.

“Fv” is the minimum antibody fragment which contains a complete antigen-recognition and antigen-binding site. This region consists of a dimer of one heavy chain and one light chain variable domain in tight, non-covalent association. It is in this configuration that the three hypervariable regions of each variable domain interact to define an antigen-binding site on the surface of the V_{H}-V_{L} dimer. Collectively, the six hypervariable regions confer antigen-binding specificity to the antibody. However, even a single variable domain (or half of an Fv comprising only three hypervariable regions specific for an antigen) has the ability to recognize and bind antigen, although at a lower affinity than the entire binding site.

The Fab fragment also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear at least one free thiol group. F(ab′)₂ antibody fragments originally were produced as pairs of Fab′ fragments which have hinge cysteines between them. Other chemical couplings of antibody fragments are also known.

The “light chains” of antibodies from any vertebrate species can be assigned to one of two clearly distinct types, called kappa (x) and lambda (λ), based on the amino acid sequences of their constant domains.

The term “Fc region” herein is used to define a C-terminal region of an immunoglobulin heavy chain, including native sequence Fc regions and variant Fc regions. Although the boundaries of the Fc region of an immunoglobulin heavy chain might vary, the human IgG heavy chain Fc region is usually defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue.

Unless indicated otherwise, herein the numbering of the residues in an immunoglobulin heavy chain is that of the EU index as in Kabat et al., Sequences of Proteins of Immunological Interest, 5th Ed. Public Health Service, National Institutes of Health, Bethesda, Md. (1991), expressly incorporated herein by reference. The “EU index as in Kabat” refers to the residue numbering of the human IgG1 EU antibody.

A “native sequence Fc region” comprises an amino acid sequence identical to the amino acid sequence of an Fc region found in nature. Native sequence human Fc regions include a native sequence human IgG1 Fc region (non-A and A allotypes); native sequence human IgG2 Fc region; native sequence human IgG3 Fc region; and native sequence human IgG4 Fc region as well as naturally occurring variants thereof.

A “variant Fc region” comprises an amino acid sequence which differs from that of a native sequence Fc region by virtue of at least one amino acid modification, preferably one or more amino acid substitution(s). Preferably, the variant Fc region has at least one amino acid substitution compared to a native sequence Fc region or to the Fc region of a parent polypeptide, e.g. from about one to about ten amino acid substitutions, and preferably from about one to about five amino acid substitutions in a native sequence Fc region or in the Fc region of the parent polypeptide. The variant Fc region herein will preferably possess at least about 80% homology with a native sequence Fc region and/or with an Fc region of a parent polypeptide, and most preferably at least about 90% homology therewith, more preferably at least about 95% homology therewith.

Depending on the amino acid sequence of the constant domain of their heavy chains, intact antibodies can be assigned to different “classes”. There are five major classes of intact antibodies: IgA, IgD, IgE, IgG, and IgM, and several of these may be further divided into “subclasses” (isotypes), e.g., IgG1, IgG2, IgG3, IgG4, IgA, and IgA2. The heavy-chain constant domains that correspond to the different classes of antibodies are called α, δ, ε, γ, and μ, respectively. The subunit structures and three-dimensional configurations of different classes of immunoglobulins are well known.

“Single-chain Fv” or “scFv” antibody fragments comprise the V_{H} and V_{L} domains of antibody, wherein these domains are present in a single polypeptide chain. Preferably, the Fv polypeptide further comprises a polypeptide linker between the V_{H} and V_{L} domains which enables the scFv to form the desired structure for antigen binding. For a review of scFv see Plückthun in The Pharmacology of Monoclonal Antibodies, vol. 113, Rosenburg and Moore eds., Springer-Verlag, New York, pp. 269-315 (1994).

The term “diabodies” refers to small antibody fragments with two antigen-binding sites, which fragments comprise a variable heavy domain (V_{H}) connected to a variable light domain (V_{L}) in the same polypeptide chain (V_{H}-V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA, 90:6444-6448 (1993).

A “naked antibody” is an antibody that is not conjugated to a heterologous molecule, such as a small molecule or radiolabel.

An “isolated” antibody is one which has been identified and separated and/or recovered from a component of its natural environment. Contaminant components of its natural environment are materials which would interfere with diagnostic or therapeutic uses for the antibody, and may include enzymes, hormones, and other proteinaceous or nonproteinaceous solutes. In preferred embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

An “affinity matured” antibody is one with one or more alterations in one or more hypervariable regions thereof which result an improvement in the affinity of the antibody for antigen, compared to a parent antibody which does not possess those alteration(s). Preferred affinity matured antibodies will have nanomolar or even picomolar affinities for the target antigen. Affinity matured antibodies are produced by procedures known in the art. Marks et al. Bio/Technology 10:779-783 (1992) describes affinity maturation by VH and VL domain shuffling. Random mutagenesis of HVR and/or framework residues is described by: Barbas et al. Proc Nat. Acad. Sci, USA 91:3809-3813 (1994); Schier et al. Gene 169:147-155 (1995); Yelton et al. J. Immunol. 155:1994-2004 (1995); Jackson et al., J. Immunol. 154(7):3310-9 (1995); and Hawkins et al, J. Mol. Biol. 226:889-896 (1992).

An “amino acid sequence variant” antibody herein is an antibody with an amino acid sequence which differs from a main species antibody. Ordinarily, amino acid sequence variants will possess at least about 70% homology with the main species antibody, and preferably, they will be at least about 80%, more preferably at least about 90% homologous with the main species antibody. The amino acid sequence variants possess substitutions, deletions, and/or additions at certain positions within or adjacent to the amino acid sequence of the main species antibody. Examples of amino acid sequence variants herein include an acidic variant (e.g. deamidated antibody variant), a basic variant, an antibody with an amino-terminal leader extension (e.g. VHS-) on one or two light chains thereof, an antibody with a C-terminal lysine residue on one or two heavy chains thereof, etc., and includes combinations of variations to the amino acid sequences of heavy and/or light chains. The antibody variant of particular interest herein is the antibody comprising an amino-terminal leader extension on one or two light chains thereof, optionally further comprising other amino acid sequence and/or glycosylation differences relative to the main species antibody.

A “glycosylation variant” antibody herein is an antibody with one or more carbohydrate moieties attached thereto which differ from one or more carbohydrate moieties attached to a main species antibody. Examples of glycosylation variants herein include antibody with a G1 or G2 oligosaccharide structure, instead a G0 oligosaccharide structure, attached to an Fc region thereof, antibody with one or two carbohydrate moieties attached to one or two light chains thereof, antibody with no carbohydrate attached to one or two heavy chains of the antibody, etc., and combinations of glycosylation alterations.

Where the antibody has an Fc region, an oligosaccharide structure may be attached to one or two heavy chains of the antibody, e.g. at residue 299 (298, Eu numbering of residues). For pertuzumab, G0 was the predominant oligosaccharide structure, with other oligosaccharide structures such as G0-F, G-1, ManS, Man6, G1-1, G1(1-6), G1(1-3) and G2 being found in lesser amounts in the pertuzumab composition.

Unless indicated otherwise, a “G1 oligosaccharide structure” herein includes G-1, G1-1, G1(1-6) and G1(1-3) structures.

An “amino-terminal leader extension” herein refers to one or more amino acid residues of the amino-terminal leader sequence that are present at the amino-terminus of any one or more heavy or light chains of an antibody. An exemplary amino-terminal leader extension comprises or consists of three amino acid residues, VHS, present on one or both light chains of an antibody variant.

A “deamidated” antibody is one in which one or more asparagine residues thereof has been derivatized, e.g. to an aspartic acid, a succinimide, or an iso-aspartic acid.

### B.1 GENERAL DESCRIPTION OF THE INVENTION

The practice of the present invention will employ, unless otherwise indicated, conventional techniques of molecular biology (including recombinant techniques), microbiology, cell biology, and biochemistry, which are within the skill of the art. Such techniques are explained fully in the literature, such as, “Molecular Cloning: A Laboratory Manual”, 2^{nd} edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Handbook of Experimental Immunology”, 4^{th} edition (D. M. Weir & C. C. Blackwell, eds., Blackwell Science Inc., 1987); “Gene Transfer Vectors for Mammalian Cells” (J. M. Miller & M. P. Calos, eds., 1987); “Current Protocols in Molecular Biology” (F. M. Ausubel et al., eds., 1987); and “PCR: The Polymerase Chain Reaction”, (Mullis et al., eds., 1994).

As discussed above, the detection or diagnosis of IBD is currently obtained by various classification systems that rely on a number of variables observed in a patient. The present invention is based on the identification of genes that are associated with IBD. Accordingly, the expression levels of such genes can serve as diagnostic markers to identify patients with IBD. As described in the Examples, the differential expression of a number of genes in IBD patients has been observed. Thus, according to the present invention, the genes listed in Tables 1A-B, 2, and 3A-B have been identified as differentially expressed in IBD.

Table 1A provides a list of genes that are upregulated in IBD.

TABLE 1A

| | | SEQ | |
|---|---|---|---|
| | | ID NO | |
| | | nucleic | |
| | | acid, | |
| | Indi- | amino | Fig- |
| Gene | cation(s) | acid | ure |
| Defensin, alpha 6 (DEFA6) | CD, UC | 1, 2 | 8 |
| Defensin, alpha 5 (DEFA5) | UC | 3, 4 | 9A-B |
| Defensin beta 14 (DEFB14) | UC | 229, 230 | 9C-D |
| IL-3 receptor, alpha low affinity | CD | 5, 6 | 10 |
| (IL3RA) | | | |
| IL-2 receptor, alpha (IL2RA) | CD, UC | 7, 8 | 11 |
| Regenerating islet-derived 3 | CD, UC | 9, 10 | 12 |
| gamma (REG3G) | | | |
| Regenerating islet-derived 1 beta | CD, UC | 11, 12 | 13 |
| pancreatic stone protein (REG1B) | | | |
| Potassium voltage-gated channel, | CD, UC | 13, 14 | 14 |
| Shal-related subfamily (KCND3) | | | |
| Human macrophage inflammatory | CD, UC | 15, 16 | 15 |
| protein 3 (MIP-3a) | | | |
| Endothelial cell growth factor 1 | CD, UC | 17, 18 | 16 |
| platelet-derived (ECGF1) | | | |
| Interleukin 1 beta (IL1B) | CD, UC | 19, 20 | 17 |
| Growth regulated protein gamma | CD, UC | 21, 22 | 18 |
| (MIP2BGRO-g) | | | |
| Chemokine C-X-C motif ligand 1 | CD, UC | 23, 24 | 19 |
| (CXCL1) | | | |
| Inhibitor of apoptosis protein 1 | CD, UC | 25, 26 | 20 |
| (IAP1) | | | |
| Caspase 5, apoptosis-related cyste- | CD, UC | 27, 28 | 21 |
| ine protease (CASP5) | | | |
| Deleted in malignant brain tumors | CD, UC | 29, 30 | 22 |
| 1 (DMBT1) | | | |
| Protocadherin 17 (PCDH17) | CD, UC | 31, 32 | 23 |
| Interferon-inducible protein 9-27 | CD, UC | 33, 34 | 24 |
| (IFITM1) | | | |
| PDZK1 interacting protein 1 | CD, UC | 35, 36 | 25 |
| (PDZK1IP1) | | | |
| IRTA2/FCRH5 (IRTA2) | CD | 37, 38 | 26 |
| Solute carrier family 40 iron- | CD, UC | 39, 40 | 27 |
| regulated transporter, member 1 | | | |
| (SLC40A1) | | | |
| Immunoglobulin heavy variable | CD, UC | 41, 42 | 28 |
| 4-4 (IGHV4-4) | | | |
| Regenerating islet-derived 3 | CD, UC | 43, 44 | 29 |
| gamma (REG3G) | | | |
| Aquaporin 9 (AQP9) | CD, UC | 45, 46 | 30 |
| Olfactomedin 4 (OLFM4) | CD, UC | 47, 48 | 31 |
| S100 calcium binding protein A9 | CD, UC | 49, 50 | 32 |
| calgranulin B (S100A9) | | | |
| unc-5 homolog C C. elegans-like | CD, UC | 51, 52 | 33 |
| (UNC5CL) | | | |
| G protein-coupled receptor 110 | CD, UC | 53, 54 | 34 |
| (GPR110) | | | |
| HLA-G histocompatibility antigen, | CD, UC | 55, 56 | 35 |
| class I, G (HLA-G) | | | |
| Transporter 1, ATP-binding cas- | CD, UC | 57, 58 | 36 |
| sette, sub-family B MDR/TAP | | | |
| (TAP1) | | | |
| Mitogen-activated protein kinase | CD, UC | 59, 60 | 37 |
| kinase kinase 8 (MAP3K8) | | | |
| Ubiquitin D\| | CD, UC | 61, 62 | 38 |
| Gamma-aminobutyric acid GABA | | | |
| B receptor, 1 (UBD\|GABBR1) | | | |
| DEAH Asp-Glu-Ala-Asp/His | CD, UC | 63, 64 | 39 |
| box polypeptide 57 (DHX57) | | | |
| Metastasis-associate (MA) | CD, UC | 65, 66 | 40 |
| LY6/PLAUR domain containing 5 | | | |
| (LYPD5) | | | |
| Immunoglobulin lambda joining- | CD, UC | 67, 68 | 41 |
| constant/OR18 (IGLJCOR18) | | | |
| TNF R superfamily, member 6b, | CD, UC | 69, 70 | 42 |
| decoy (TNFRSF6B) | | | |
| serum amyloid A1 (SAA1) | CD, UC | 71, 72 | 43 |
| Transporter 2, ATP-binding cas- | CD, UC | 73, 74 | 44 |
| sette, sub-family B MDR/TAP | | | |
| (TAP2) | | | |
| PCAA17448 | CD, UC | 75, 76 | 45 |
| lipocalin 2 oncogene 24p3 (LCN2) | CD, UC | 77, 78 | 46 |
| Z-D binding protein 1 (ZBP1) | CD, UC | 79, 80 | 47 |
| TNFAIP3 interacting protein 3 | CD, UC | 81, 82 | 48 |
| (TNIP3) | | | |
| Zinc finger CCCH-type containing | CD, UC | 83, 84 | 49 |
| 12A (ZC3H12A) | | | |
| Chitinase 3-like 1 cartilage glyco- | CD, UC | 85, 86 | 50 |
| protein-39 (CHI3L1) | | | |
| Fc fragment of IgG, low affinity | CD, UC | 87, 88 | 51 |
| IIIa, receptor CD16a (FCGR3A) | | | |
| Sterile alpha motif domain contain- | CD, UC | 89, 90 | 52 |
| ing 9-like (SAMD9L) | | | |
| Matrix metalloproteinase 9 | CD | 91, 92 | 53 |
| gelatinase B, 92 kDa gelatinase, | | | |
| 92 kDa type IV collagenase | | | |
| (MMP9) | | | |
| Matrix metalloproteinase 7 | CD, UC | 93, 94 | 54 |
| matrilysin, uterine (MMP7) | | | |
| B-factor, properdin (BF) | CD, UC | 95, 96 | 55 |
| S100 calcium binding protein P | CD, UC | 97, 98 | 56 |
| (S100P) | | | |
| Growth regulated protein (GRO) | CD, UC | 99, 100 | 57 |
| Indoleamine-pyrrole 2,3 | CD, UC | 101, 102 | 58 |
| dioxygenase INDO (INDO) | | | |
| Tripartite motif-containing 22 | CD, UC | 103, 104 | 59 |
| (TRIM22) | | | |
| Serum amyloid A2 (SAA2) | CD, UC | 105, 106 | 60 |
| Arrestin domain containing 5 | CD, UC | 193, 194 | 110 |
| (ARRDC5) (LOC342959) | | | |
| ataxin 3-like (ATXN3L) | CD, UC | 195 | 111 |
| (A_24_P910246) | | | |
| follicle stimulating hormone recep- | CD, UC | 196, 197 | 112 |
| tor (FSHR) (LOC92552) | | | |
| platelet-derived growth factor re- | CD, UC | 198, 199 | 113 |
| ceptor, alpha polypeptide | | | |
| (PDGFRA) | | | |
| transforming growth factor beta 3 | CD, UC | 200, 201 | 114 |
| (TGFB3) | | | |
| potassium channel tetramerisation | CD, UC | 202, 203 | 115 |
| domain containing 8 (KCTD8) | | | |
| transglutaminase 4 (TGM4) | CD, UC | 204, 205 | 116 |
| TPD52L3 tumor protein D52-like 3 | CD, UC | 206, 207 | 117 |
| (NYD-SP25) | | | |
| misc_RNA (C3orf53) (FLJ33651) | CD, UC | 208 | 118 |
| EMX2 opposite strand (non-protein | CD, UC | 209 | 119A |
| coding) (EMX2OS) on chromo- | | | |
| some 10 | | | |
| S100A8 | UC | 231, 232 | 130 |
| CCL20 | UC | 233, 234 | 131 |

Table 1B provides a list of genes that are downregulated in IBD.

| | | SEQ | |
|---|---|---|---|
| | | ID NO | |
| | | nucleic | |
| | | acid, | |
| | Indi- | amino | Fig- |
| Gene | cation(s) | acid | ure |
| Sialidase 4 (NEU4) | CD, UC | 107, 108 | 61 |
| wingless-type MMTV integration site | CD, UC | 210, 211 | 120 |
| family, member 16 (WNT16) | | | |
| sprouty-related, EVH1 domain con- | CD, UC | 212, 213 | 121 |
| taining 2 (SPRED2) | | | |
| chromosome 16 open reading frame 65 | CD, UC | 214, 215 | 122 |
| (C16orf65) (MGC50721) | | | |
| chromosome 12 open reading frame 2 | CD, UC | 216, 217 | 123 |
| (C12orf2) | | | |
| multiple PDZ domain protein (MPDZ) | CD, UC | 218, 219 | 124 |
| phenylalanine-tRNA synthetase 2 | CD, UC | 220, 221 | 125 |
| (FARS2) | | | |
| caspase 8, apoptosis-related cysteine | CD, UC | 222, 223 | 126 |
| protease (CASP8) | | | |
| 5′-nucleotidase, ecto (CD73) (NT5E) | CD, UC | 224, 225 | 127 |
| teratocarcinoma-derived growth factor | CD, UC | 226 | 128 |
| 3 (TDGF3) | | | |
| butyrophilin-like 3 (BTNL3) | CD, UC | 227, 228 | 129 |

Table 2 provides a list of genes that are upregulated in IBD. These genes were identified from the immune response in silico (IRIS) collection of genes (Abbas, A. et al. Genes and Immunity, 6:319-331 (2005) hereby incorporated by reference in its entirety).

TABLE 2

| | | SEQ | |
|---|---|---|---|
| | | ID NO | |
| | | nucleic | |
| | | acid, | |
| | Indi- | amino | Fig- |
| Gene | cation(s) | acid | ure |
| Immunoglobulin domain IRTA2/ | CD, UC | 109, 110 | 62 |
| FCRH5 | | | |
| Immunoglobulin lambda joining-con- | CD, UC | 111, 112 | 63 |
| stant/OR18 (IGLJCOR18) | | | |
| Immunoglobulin heavy variable 4-4 | CD, UC | 113, 114 | 64 |
| (IGHV4-4) | | | |
| Matrix metalloproteinase 9 gelatinase B, | CD, UC | 115, 116 | 65 |
| 92 kDa gelatinase, 92 kDa type IV | | | |
| collagenase (MMP9) | | | |
| Growth regulated protein (GRO) | CD, UC | 117, 118 | 66 |
| Growth regulated protein gamma | CD, UC | 119, 120 | 67 |
| (MIP2BGRO-g) | | | |
| interleukin 1, beta (IL1B) | CD, UC | 121, 122 | 68 |
| IL-3 receptor, alpha low affinity | CD, UC | 123, 124 | 69 |
| (IL3RA) | | | |
| Caspase 1, apoptosis-related cysteine | CD, UC | 125, 126 | 70 |
| protease interleukin 1, beta, convertase | | | |
| (CASP1) | | | |
| Bv8 protein (BV8) | CD, UC | 127, 128 | 71 |

Table 3A provides a list of genes that are upregulated in IBD and were identified based on the experiments described in Example 2. In some instances, the locus/chromosome of the gene is provided.

TABLE 3A

| | | Locus/ | SEQ ID NO | |
|---|---|---|---|---|
| | Indi- | Chromosome | nucleic acid, | |
| Gene | cation(s) | (if known) | amino acid | Figure |
| HDAC7A | UC | IBD2/12 | 129, 130 | 72 |
| ACVRL1 | UC | IBD2/12 | 131, 132 | 73 |
| NR4A1 | UC | IBD2/12 | 133, 134 | 74 |
| K5B | UC | IBD2/12 | 135, 136 | 75 |
| SILV | UC | IBD2/12 | 137, 138 | 76 |
| IRAK3 | UC | IBD2/12 | 139, 140 | 77 |
| IL-4 | UC | IBD5/5 | 141, 142 | 78 |
| IL-13 | UC | IBD5/5 | 143, 144 | 79 |
| RAD50 | UC | IBD5/5 | 145, 146 | 80 |
| IL-5 | UC | IBD5/5 | 147, 148 | 81 |
| IRF1 | UC | IBD5/5 | 149, 150 | 82 |
| PDLIM4 | UC | IBD5/5 | 151, 152 | 83 |
| CSF2 | UC | IBD5/5 | 153, 154 | 84 |
| IL-3 | UC | IBD5/5 | 155, 156 | 85 |
| MMP3 | UC | | 157, 158 | 86 |
| IL-8 | UC | | 159, 160 | 87 |
| TLR4 | UC | | 161, 162 | 88 |
| HLA-DRB1 | UC | | 163, 164 | 89 |
| MMP19 | UC | | 165, 166 | 90 |
| TIMP1 | UC | | 167, 168 | 91 |
| Elfin | UC | | 169, 170 | 92 |
| CXCL1 | UC | | 171, 172 | 93 |

Table 3B provides a list of genes that are down-regulated in IBD and were identified based on the experiments described in Example 2. In some instances, the locus/chromosome of the gene is provided.

TABLE 3B

| | | | SEQ ID NO | |
|---|---|---|---|---|
| | Indi- | Locus/ | nucleic acid, | |
| Gene | cation(s) | Chromosome | amino acid | Figure |
| DFKZP586A0522 | UC | IBD2/12 | 173, 174 | 94 |
| SLC39A5 | UC | IBD2/12 | 175, 176 | 95 |
| GLI-1 | UC | IBD2/12 | 177, 178 | 96 |
| HMGA2 | UC | IBD2/12 | 179, 180 | 97 |
| SLC22A5 | UC | IBD5/5 | 181, 182 | 98 |
| SLC22A4 | UC | IBD5/5 | 183, 184 | 99 |
| P4HA2 | UC | IBD5/5 | 185, 186 | 100 |
| TSLP | UC | | 187, 188 | 101 |
| tubulin alpha 5/alpha 3 | UC | | 189, 190 | 102 |
| tubulin alpha 6 | UC | | 191, 192 | 103 |

a. Biomarkers of the Invention

The present invention provides numerous gene expression markers or biomarkers for IBD listed in Tables 1A, 1B, 2, 3A, and 3B. In one embodiment of the present invention, the biomarkers are suitable for use in a panel of markers (as described herein). Such panels may include one or more markers from Table 1A; one or more markers from Table 1B; the marker from Table 2; one or markers from Table 3A; and one or more markers from Table 3B. In addition, the present invention also contemplates panels of markers selected from two or more of Tables 1A, 1B, 2, 3A, and 3B. For example, a panel might contain one or more markers from Table 1A and one or more markers from Table 1B; or one or more markers from Table 1A and the marker from Table 2; or one or more markers from Table 1B and the marker from Table 2; or one or more markers from Table 1A and one or more markers from Table 3A, etc. Those of ordinary skill in the art will appreciate the various combinations of biomarkers from Tables 1-3 that are suitable for use in the panels described herein.

In one embodiment of the present invention, a preferred set of IBD markers identified by microarray analysis, includes markers that are upregulated in an IBD. Preferably, the set of upregulated markers includes DEFA5 (SEQ ID NOS:3-4), DEFA6 (SEQ ID NO:1-2), TNIP3 (SEQ ID NO:81-82), REG3-gamma (SEQ ID NO:9-10), MMP7 (SEQ ID NO:93-94), and SAA1 (SEQ ID NO:71-72) in Table 1A; and IL-8 (SEQ ID NO:159-160), Keratin 5B (K5B) (SEQ ID NO:135-136), SLC22A4 (SEQ ID NO:183-184), SLC22A5 (SEQ ID NO:181-182), MMP3 (SEQ ID NO:157-158), and MMP19 (SEQ ID NO:165-166) in Tables 3A. A preferred downregulated marker is GLI-1 (SEQ ID NO: 175-176) in Table 3B. A panel of biomarkers as described herein may include one of, more than one of, or all of these markers. Alternatively, the set of markers include 1, 2, 3, 4, 5, 6 of the indicated markers from Table 1A, and/or 1, 2, 3, 4, 5, 6 of the indicated markers from Table 3A and/or 1 or 2 of the indicated markers in Table 3B.

Members of lists provided above, as single markers or in any combination, are preferred for use in prognostic and diagnostic assays of the present invention. The IBD markers of the present invention are differentially expressed genes or regions of genes. A differential level of expression of one or more markers in a test sample from a mammalian subject relative to a control can determined from the level of RNA transcripts or expression products detected by one or more of the methods described in further detail below.

Based on evidence of differential expression of RNA transcripts in normal cells and cells from a mammalian subject having IBD, the present invention provides gene markers for IBD. The IBD markers and associated information provided by the present invention allow physicians to make more intelligent treatment decisions, and to customize the treatment of IBD to the needs of individual patients, thereby maximizing the benefit of treatment and minimizing the exposure of patients to unnecessary treatments, which do not provide any significant benefits and often carry serious risks due to toxic side-effects.

Multi-analyte gene expression tests can measure the expression level of one or more genes involved in each of several relevant physiologic processes or component cellular characteristics. In some instances the predictive power of the test, and therefore its utility, can be improved by using the expression values obtained for individual genes to calculate a score which is more highly correlated with outcome than is the expression value of the individual genes. For example, the calculation of a quantitative score (recurrence score) that predicts the likelihood of recurrence in estrogen receptor-positive, node-negative breast cancer is describe in U.S. Published Patent Application No. 20050048542. The equation used to calculate such a recurrence score may group genes in order to maximize the predictive value of the recurrence score. The grouping of genes may be performed at least in part based on knowledge of their contribution to physiologic functions or component cellular characteristics such as discussed above. The formation of groups, in addition, can facilitate the mathematical weighting of the contribution of various expression values to the recurrence score. The weighting of a gene group representing a physiological process or component cellular characteristic can reflect the contribution of that process or characteristic to the pathology of the IBD and clinical outcome. Accordingly, in an important aspect, the present invention also provides specific groups of the genes identified herein, that together are more reliable and powerful predictors of outcome than the individual genes or random combinations of the genes identified.

In addition, based on the determination of a recurrence score, one can choose to partition patients into subgroups at any particular value(s) of the recurrence score, where all patients with values in a given range can be classified as belonging to a particular risk group. Thus, the values chosen will define subgroups of patients with respectively greater or lesser risk.

The utility of a gene marker in predicting the development or progression of an IBD may not be unique to that marker. An alternative marker having a expression pattern that is closely similar to a particular test marker may be substituted for or used in addition to a test marker and have little impact on the overall predictive utility of the test. The closely similar expression patterns of two genes may result from involvement of both genes in a particular process and/or being under common regulatory control. The present invention specifically includes and contemplates the use of such substitute genes or gene sets in the methods of the present invention.

The markers and associated information provided by the present invention predicting the development and/or progression of an IBD also have utility in screening patients for inclusion in clinical trials that test the efficacy of drug compounds for the treatment of patients with IBD.

The markers and associated information provided by the present invention predicting the presence, development and/or progression of an IBD are useful as criterion for determining whether IBD treatment is appropriate. For example, IBD treatment may be appropriate where the results of the test indicate that an IBD marker is differentially expressed in a test sample from an individual relative to a control sample. The individual may be an individual not known to have an IBD, an individual known to have an IBD, an individual previously diagnosed with an IBD undergoing treatment for the IBD, or an individual previously diagnosed with an IBD and having had surgery to address the IBD. In addition, the present invention contemplates methods of treating an IBD. As described below, the diagnostic methods of the present invention may further comprise the step of administering an IBD therapeutic agent to the mammalian subject that provided the test sample in which the differential expression of one or more IBD markers was observed relative to a control. Such methods of treatment would therefore comprise (a) determining the presence of an IBD in a mammalian subject, and (b) administering an IBD therapeutic agent to the mammalian subject.

In another embodiment, the IBD markers and associated information are used to design or produce a reagent that modulates the level or activity of the gene's transcript or its expression product. Said reagents may include but are not limited to an antisense RNA, a small inhibitory RNA (siRNA), a ribozyme, a monoclonal or polyclonal antibody. In a further embodiment, said gene or its transcript, or more particularly, an expression product of said transcript is used in an (screening) assay to identify a drug compound, wherein said drug compounds is used in the development of a drug to treat an IBD.

In various embodiments of the inventions, various technological approaches described below are available for determination of expression levels of the disclosed genes. In particular embodiments, the expression level of each gene may be determined in relation to various features of the expression products of the gene including exons, introns, protein epitopes and protein activity. In other embodiments, the expression level of a gene may be inferred from analysis of the structure of the gene, for example from the analysis of the methylation pattern of gene's promoter(s).

b. Diagnostic Methods of the Invention

The present invention provides methods of detecting or diagnosing an IBD in a mammalian subject based on differential expression of an IBD marker. In a one embodiment, the methods comprise the use of a panel of IBD markers as discussed above. The panels may include one or more IBD markers selected from Tables 1-3.

In some embodiments, the panel of IBD markers will include at least 1 IBD marker, at least two IBD markers, at least three IBD markers, at least 4 IBD markers, at least five IBD markers, at least 6 IBD markers, at least 7 IBD marker, at least 8 IBD markers, at least 9 IBD markers, at least 10 IBD markers, at least 11 IBD markers, at least 12 IBD markers, at least 13 IBD markers, at least 14 IBD markers, at least 15 IBD markers, at least 16 IBD markers, at least 17 IBD markers, at least 18 IBD markers, at least 19 IBD markers, or at least 20 IBD markers. In one embodiment, the panel includes markers in increments of five. In another embodiment, the panel includes markers in increments of ten. The panel may include an IBD marker that is overexpressed in IBD relative to a control, an IBD marker that is underexpressed in IBD relative to a control, or IBD markers that are both overexpressed and underexpressed in IBD relative to a control. In a preferred embodiment, the panel includes one or more markers that are upregulated in CD and one or more markers that are downregulated in CD. In another preferred embodiment, the panel includes one or more markers that are upregulated in UC and one or more markers that are downregulated in UC.

In another embodiment, the panels of the present invention may include an IBD marker that is overexpressed in an active IBD relative to a control, underexpressed in an active IBD relative to a control, or IBD markers that are both overexpressed and underexpressed in an active IBD relative to a control. In another embodiment, the panels of the present invention may include an IBD marker that is overexpressed in an inactive IBD relative to a control, underexpressed in an inactive IBD relative to a control, or IBD markers that are both overexpressed and underexpressed in an inactive IBD relative to a control. In a preferred embodiment, the active IBD is CD. In another preferred embodiment, the inactive IBD is CD.

In a preferred embodiment, the methods of diagnosing or detecting the presence of an IBD in a mammalian subject comprise determining a differential expression level of RNA transcripts or expression products thereof from a panel of IBD markers in a test sample obtained from the subject relative to the level of expression in a control, wherein the differential level of expression is indicative of the presence of an IBD in the subject from which the test sample was obtained. The differential expression in the test sample may be higher and/or lower relative to a control as discussed herein.

Differential expression or activity of one or more of the genes provided in the lists above, or the corresponding RNA molecules or encoded proteins in a biological sample obtained from the patient, relative to control, indicates the presence of an IBD in the patient. The control can, for example, be a gene, present in the same cell, which is known to be up-regulated (or down-regulated) in an IBD patient (positive control). Alternatively, or in addition, the control can be the expression level of the same gene in a normal cell of the same cell type (negative control). Expression levels can also be normalized, for example, to the expression levels of housekeeping genes, such as glyceraldehyde-3-phosphate-dehydrogenase (GAPDH) and/or β-actin, or to the expression levels of all genes in the sample tested. In one embodiment, expression of one or more of the above noted genes is deemed positive expression if it is at the median or above, e.g. compared to other samples of the same type. The median expression level can be determined essentially contemporaneously with measuring gene expression, or may have been determined previously. These and other methods are well known in the art, and are apparent to those skilled in the art.

Methods for identifying IBD patients are provided herein. Of this patient population, patients with an IBD can be identified by determining the expression level of one or more of the genes, the corresponding RNA molecules or encoded proteins in a biological sample comprising cells obtained from the patient. The biological sample can, for example, be a tissue biopsy as described herein.

The methods of the present invention concern IBD diagnostic assays, and imaging methodologies. In one embodiment, the assays are performed using antibodies as described herein. The invention also provides various immunological assays useful for the detection and quantification of proteins. These assays are performed within various immunological assay formats well known in the art, including but not limited to various types of radioimmunoassays, enzyme-linked immunosorbent assays (ELISA), enzyme-linked immunofluorescent assays (ELIFA), and the like. In addition, immunological imaging methods capable of detecting an IBD characterized by expression of a molecule described herein are also provided by the invention, including but not limited to radioscintigraphic imaging methods using labeled antibodies. Such assays are clinically useful in the detection, monitoring, diagnosis and prognosis of IBD characterized by expression of one or more molecules described herein.

Another aspect of the present invention relates to methods for identifying a cell that expresses a molecule described herein. The expression profile of a molecule(s) described herein make it a diagnostic marker for IBD. Accordingly, the status of the expression of the molecule(s) provides information useful for predicting a variety of factors including susceptibility to advanced stages of disease, rate of progression, and/or sudden and severe onset of symptoms in an active IBD or an inactive IBD, i.e. flare-ups.

In one embodiment, the present invention provides methods of detecting an IBD. A test sample from a mammalian subject and a control sample from a known normal mammal are each contacted with an anti-IBD marker antibody or a fragment thereof. The level of IBD marker expression is measured and a differential level of expression in the test sample relative to the control sample is indicative of an IBD in the mammalian subject from which the test sample was obtained. In some embodiments, the level of IBD marker expression in the test sample is determined to be higher than the level of expression in the control, wherein the higher level of expression indicates the presence of an IBD in the subject from which the test sample was obtained. In another embodiments, the level of IBD marker expression in the test sample is determined to be lower than the level of expression in the control, wherein the lower level of expression indicates the presence of an IBD in the subject from which the test sample was obtained.

In another embodiment, the IBD detected by the methods of the present invention is the recurrence or flareup of an IBD in the mammalian subject.

In preferred embodiments, the methods are employed to detect the flare-up of an IBD or a recurrence of an IBD in a mammalian subject previously determined to have an IBD who underwent treatment for the IBD, such as drug therapy or a surgical procedure. Following initial detection of an IBD, additional test samples may be obtained from the mammalian subject found to have an IBD. The additional sample may be obtained hours, days, weeks, or months after the initial sample was taken. Those of skill in the art will appreciate the appropriate schedule for obtaining such additional samples, which may include second, third, fourth, fifth, sixth, etc. test samples. The initial test sample and the additional sample (and alternately a control sample as described herein) are contacted with an anti-IBD marker antibody. The level of IBD marker expression is measured and a differential level of expression in the additional test sample as compared to the initial test sample is indicative of a flare-up in or a recurrence of an IBD in the mammalian subject from which the test sample was obtained.

In one aspect, the methods of the present invention are directed to a determining step. In one embodiment, the determining step comprises measuring the level of expression of one or more IBD markers in a test sample relative to a control. Typically, measuring the level of IBD marker expression, as described herein, involves analyzing a test sample for differential expression of an IBD marker relative to a control by performing one or more of the techniques described herein. The expression level data obtained from a test sample and a control are compared for differential levels of expression. In another embodiment, the determining step further comprises an examination of the test sample and control expression data to assess whether an IBD is present in the subject from which the test sample was obtained.

In some embodiments, the determining step comprises the use of a software program executed by a suitable processor for the purpose of (i) measuring the differential level of IBD marker expression in a test sample and a control; and/or (ii) analyzing the data obtained from measuring differential level of IBD marker expression in a test sample and a control. Suitable software and processors are well known in the art and are commercially available. The program may be embodied in software stored on a tangible medium such as CD-ROM, a floppy disk, a hard drive, a DVD, or a memory associated with the processor, but persons of ordinary skill in the art will readily appreciate that the entire program or parts thereof could alternatively be executed by a device other than a processor, and/or embodied in firmware and/or dedicated hardware in a well known manner.

Following the determining step, the measurement results, findings, diagnoses, predictions and/or treatment recommendations are typically recorded and communicated to technicians, physicians and/or patients, for example. In certain embodiments, computers will be used to communicate such information to interested parties, such as, patients and/or the attending physicians. In some embodiments, the assays will be performed or the assay results analyzed in a country or jurisdiction which differs from the country or jurisdiction to which the results or diagnoses are communicated.

In a preferred embodiment, a diagnosis, prediction and/or treatment recommendation based on the level of expression of one or more IBD markers disclosed herein measured in a test subject of having one or more of the IBD markers herein is communicated to the subject as soon as possible after the assay is completed and the diagnosis and/or prediction is generated. The results and/or related information may be communicated to the subject by the subject's treating physician. Alternatively, the results may be communicated directly to a test subject by any means of communication, including writing, electronic forms of communication, such as email, or telephone. Communication may be facilitated by use of a computer, such as in case of email communications. In certain embodiments, the communication containing results of a diagnostic test and/or conclusions drawn from and/or treatment recommendations based on the test, may be generated and delivered automatically to the subject using a combination of computer hardware and software which will be familiar to artisans skilled in telecommunications. One example of a healthcare-oriented communications system is described in U.S. Pat. No. 6,283,761; however, the present invention is not limited to methods which utilize this particular communications system. In certain embodiments of the methods of the invention, all or some of the method steps, including the assaying of samples, diagnosing of diseases, and communicating of assay results or diagnoses, may be carried out in diverse (e.g., foreign) jurisdictions.

The invention provides assays for detecting the differential expression of an IBD marker in tissues associated with the gastrointestinal tract including, without limitation, ascending colon tissue, descending colon tissue, sigmoid colon tissue, and terminal ileum tissue; as well expression in other biological samples such as serum, semen, bone, prostate, urine, cell preparations, and the like. Methods for detecting differential expression of an IBD marker are also well known and include, for example, immunoprecipitation, immunohistochemical analysis, Western blot analysis, molecular binding assays, ELISA, ELIFA and the like. For example, a method of detecting the differential expression of an IBD marker in a biological sample comprises first contacting the sample with an anti-IBD marker antibody, an IBD marker-reactive fragment thereof, or a recombinant protein containing an antigen-binding region of an anit-IBD marker antibody; and then detecting the binding of an IBD marker protein in the sample.

In various embodiments of the inventions, various technological approaches are available for determination of expression levels of the disclosed genes, including, without limitation, RT-PCR, microarrays, serial analysis of gene expression (SAGE) and Gene Expression Analysis by Massively Parallel Signature Sequencing (MPSS), which will be discussed in detail below. In particular embodiments, the expression level of each gene may be determined in relation to various features of the expression products of the gene including exons, introns, protein epitopes and protein activity. In other embodiments, the expression level of a gene may be inferred from analysis of the structure of the gene, for example from the analysis of the methylation pattern of gene's promoter(s).

c. Therapeutic Methods of the Invention

The present invention provides therapeutic methods of treating an IBD in a subject in need that comprise detecting the presence of an IBD in a mammalian subject by the diagnostic methods described herein and then administering to the mammalian subject an IBD therapeutic agent. Those of ordinary skill in the art will appreciate the various IBD therapeutic agents that may be suitable for use in the present invention (see St Clair Jones, Hospital Pharmacist, May 2006, Vol. 13; pages 161-166, hereby incorporated by reference in its entirety). The present invention contemplates methods of IBD treatment in which one or more IBD therapeutic agents are administered to a subject in need. In one embodiment, the IBD therapeutic agent is one or more of an aminosalicylate, a corticosteroid, and an immunosuppressive agent. In a preferred embodiment, the aminosalicylate is one of sulfasalazine, olsalazine, mesalamine, balsalazide, and asacol. In another preferred embodiment, multiple aminosalicylates are co-administered, such as a combination of sulfasalazine and olsalazine. In other preferred embodiments, the corticosteroid may be budesonide, prednisone, prednisolone, methylprednisolone, 6-mercaptopurine (6-MP), azathioprine, methotrexate, and cyclosporin. In other preferred embodiments, the IBD therapeutic agent may an antibiotic, such as ciprofloxacin and/or metronidazole; or an antibody-based agent such as infliximab (Remicade®).

The least toxic IBD therapeutic agents which patients are typically treated with are the aminosalicylates. Sulfasalazine (Azulfidine), typically administered four times a day, consists of an active molecule of aminosalicylate (5-ASA) which is linked by an azo bond to a sulfapyridine. Anaerobic bacteria in the colon split the azo bond to release active 5-ASA. However, at least 20% of patients cannot tolerate sulfapyridine because it is associated with significant side-effects such as reversible sperm abnormalities, dyspepsia or allergic reactions to the sulpha component. These side effects are reduced in patients taking olsalazine. However, neither sulfasalazine nor olsalazine are effective for the treatment of small bowel inflammation. Other formulations of 5-ASA have been developed which are released in the small intestine (e.g. mesalamine and asacol). Normally it takes 6-8 weeks for 5-ASA therapy to show full efficacy. Patients who do not respond to 5-ASA therapy, or who have a more severe disease, are prescribed corticosteroids. However, this is a short term therapy and cannot be used as a maintenance therapy. Clinical remission is achieved with corticosteroids within 2-4 weeks, however the side effects are significant and include Cushing goldface, facial hair, severe mood swings and sleeplessness. The response to sulfasalazine and 5-aminosalicylate preparations is poor in CD, fair to mild in early ulcerative colitis and poor in severe UC. If these agents fail, powerful immunosuppressive agents such as cyclosporine, prednisone, 6-mercaptopurine or azathioprine (converted in the liver to 6-mercaptopurine) are typically tried. For CD patients, the use of corticosteroids and other immunosuppressives must be carefully monitored because of the high risk of intra-abdominal sepsis originating in the fistulas and abscesses common in this disease. Approximately 25% of IBD patients will require surgery (colectomy) during the course of the disease.

Treatment of an IBD may include a surgical procedure, including without limitation, a bowel resection, anastomosis, a colectomy, a proctocolectomy, and an ostomy, or any combination thereof.

In addition to pharmaceutical medicine and surgery, nonconventional treatments for IBD such as nutritional therapy have also been attempted. For example, Flexical®, a semi-elemental formula, has been shown to be as effective as the steroid prednisolone. Sanderson et al., Arch. Dis. Child. 51:123-7 (1987). However, semi-elemental formulas are relatively expensive and are typically unpalatable—thus their use has been restricted. Nutritional therapy incorporating whole proteins has also been attempted to alleviate the symptoms of IBD. Giafer et al., Lancet 335: 816-9 (1990). U.S. Pat. No. 5,461,033 describes the use of acidic casein isolated from bovine milk and TGF-2. Beattie et al., Aliment. Pharmacol. Ther. 8: 1-6 (1994) describes the use of casein in infant formula in children with IBD. U.S. Pat. No. 5,952,295 describes the use of casein in an enteric formulation for the treatment of IBD. However, while nutrional therapy is non-toxic, it is a palliative treatment and does not treat the underlying cause of the disease.

The present invention contemplates methods of IBD treatment, including for example, in vitro, ex vivo and in vivo therapeutic methods. The invention provides methods useful for treating an IBD in a subject in need upon the detection of an IBD disease state in the subject associated with the expression of one or more IBD markers disclosed herein, such as increased and/or decreased IBD marker expression. In one preferred embodiment, the method comprises (a) determining that the level of expression of (i) one or more nucleic acids encoding one or more polypeptides selected from Tables 1, 2, or 3; or (ii) RNA transcripts or expression products thereof of one or more genes listed in Tables 1, 2, and 3 in a test sample obtained from said subject is higher and/or lower relative to the level of expression in a control, wherein said higher and/or lower level of expression is indicative of the presence of an IBD in the subject from which the test sample was obtained; and (b) administering to said subject an effective amount of an IBD therapeutic agent. The determining step (a) may comprise the measurement of the expression of multiple IBD marker

The method of treatment comprises detecting the IBD and administering an effective amount of an IBD therapeutic agent to a subject in need of such treatment. In some embodiments, the IBD disease state is associated with an increased and/or decrease in expression of one or more IBD markers.

In one aspect, the invention provides methods for treating or preventing an IBD, the methods comprising detecting the presence of an IBD in a subject and administering an effective amount of an IBD therapeutic agent to the subject. It is understood that any suitable IBD therapeutic agent may be used in the methods of treatment, including aminosalicylates, corticosteroids, and immunosuppressive agents as discussed herein.

In any of the methods herein, one may administer to the subject or patient along with a single IBD therapeutic agent discussed herein an effective amount of a second medicament (where the single IBD therapeutic agent herein is a first medicament), which is another active agent that can treat the condition in the subject that requires treatment. For instance, an aminosalicylate may be co-administered with a corticosteroid, an immunosuppressive agent, or another aminosalicylate. The type of such second medicament depends on various factors, including the type of IBD, its severity, the condition and age of the patient, the type and dose of first medicament employed, etc.

Such treatments using first and second medicaments include combined administration (where the two or more agents are included in the same or separate formulations), and separate administration, in which case, administration of the first medicament can occur prior to, and/or following, administration of the second medicament. In general, such second medicaments may be administered within 48 hours after the first medicaments are administered, or within 24 hours, or within 12 hours, or within 3-12 hours after the first medicament, or may be administered over a pre-selected period of time, which is preferably about 1 to 2 days, about 2 to 3 days, about 3 to 4 days, about 4 to 5 days, about 5 to 6 days, or about 6 to 7 days.

The first and second medicaments can be administered concurrently, sequentially, or alternating with the first and second medicament or upon non-responsiveness with other therapy. Thus, the combined administration of a second medicament includes co-administration (concurrent administration), using separate formulations or a single pharmaceutical formulation, and consecutive administration in either order, wherein preferably there is a time period while both (or all) medicaments simultaneously exert their biological activities. All these second medicaments may be used in combination with each other or by themselves with the first medicament, so that the express “second medicament” as used herein does not mean it is the only medicament besides the first medicament, respectively. Thus, the second medicament need not be one medicament, but may constitute or comprise more than one such drug. These second medicaments as set forth herein are generally used in the same dosages and with administration routes as the first medicaments, or about from 1 to 99% of the dosages of the first medicaments. If such second medicaments are used at all, preferably, they are used in lower amounts than if the first medicament were not present, especially in subsequent dosings beyond the initial dosing with the first medicament, so as to eliminate or reduce side effects caused thereby.

Where the methods of the present invention comprise administering one or more IBD therapeutic agent to treat or prevent an IBD, it may be particularly desirable to combine the administering step with a surgical procedure that is also performed to treat or prevent the IBD. The IBD surgical procedures contemplated by the present invention include, without limitation, a bowel resection, anastomosis, a colectomy, a proctocolectomy, and an ostomy, or any combination thereof. For instance, an IBD therapeutic agent described herein may be combined with a colectomy in a treatment scheme, e.g. in treating an IBD. Such combined therapies include and separate administration, in which case, administration of the IBD therapeutic agent can occur prior to, and/or following, the surgical procedure.

Treatment with a combination of one or more IBD therapeutic agents; or a combination of one or more IBD therapeutic agents and a surgical procedure described herein preferably results in an improvement in the signs or symptoms of an IBD. For instance, such therapy may result in an improvement in the subject receiving the IBD therapeutic agent treatment regimen and a surgical procedure, as evidenced by a reduction in the severity of the pathology of the IBD.

The IBD therapeutic agent(s) is/are administered by any suitable means, including parenteral, subcutaneous, intraperitoneal, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g. by injections, such as intravenous or subcutaneous injections, depending in part on whether the administration is brief or chronic.

The IBD therapeutic agent(s) compositions administered according to the methods of the invention will be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The first medicament(s) need not be, but is optionally formulated with one or more additional medicament(s) (e.g. second, third, fourth, etc. medicaments) described herein. The effective amount of such additional medicaments depends on the amount of the first medicament present in the formulation, the type of disorder or treatment, and other factors discussed above. These are generally used in the same dosages and with administration routes as used hereinbefore or about from 1 to 99% of the heretofore employed dosages.

For the prevention or treatment of an IBD, the appropriate dosage of an IBD therapeutic agent (when used alone or in combination with other agents) will depend on the type of disease to be treated, the type of IBD therapeutic agent(s), the severity and course of the disease, whether the IBD therapeutic agent is administered for preventive or therapeutic purposes, previous therapy, the patient's clinical history and response to the IBD therapeutic agent, and the discretion of the attending physician. The IBD therapeutic agent is suitably administered to the patient at one time or over a series of treatments. Depending on the type and severity of the disease, about 1 ug/kg to 15 mg/kg (e.g. 0.1 mg/kg-10 mg/kg) of IBD therapeutic agent is an initial candidate dosage for administration to the patient, whether, for example, by one or more separate administrations, or by continuous infusion. One typical daily dosage might range from about 1 ug/kg to 100 mg/kg or more, depending on the factors mentioned above. For repeated administrations over several days or longer, depending on the condition, the treatment is sustained until a desired suppression of disease symptoms occurs. One exemplary dosage of the IBD therapeutic agent would be in the range from about 0.05 mg/kg to about 10 mg/kg. Thus, one or more doses of about 0.5 mg/kg, 2.0 mg/kg, 4.0 mg/kg or 10 mg/kg (or any combination thereof) may be administered to the patient. Such doses may be administered intermittently, e.g. every week or every three weeks (e.g. such that the patient receives from about two to about twenty, e.g. about six doses of the IBD therapeutic agent). An initial higher loading dose, followed by one or more lower doses may be administered. An exemplary dosing regimen comprises administering an initial loading dose of about 4 mg/kg, followed by a weekly maintenance dose of about 2 mg/kg of the IBD therapeutic agent. However, other dosage regimens may be useful. The progress of this therapy is easily monitored by conventional techniques and assays.

### B.2. GENE EXPRESSION PROFILING

In general, methods of gene expression profiling can be divided into two large groups: methods based on hybridization analysis of polynucleotides, and other methods based on biochemical detection or sequencing of polynucleotides. The most commonly used methods known in the art for the quantification of mRNA expression in a sample include northern blotting and in situ hybridization (Parker & Barnes, Methods in Molecular Biology 106:247-283 (1999)); RNAse protection assays (Hod, Biotechniques 13:852-854 (1992)); and reverse transcription polymerase chain reaction (RT-PCR) (Weis et al., Trends in Genetics 8:263-264 (1992)). Alternatively, antibodies may be employed that can recognize specific duplexes, including DNA duplexes, RNA duplexes, and DNA-RNA hybrid duplexes or DNA-protein duplexes. Various methods for determining expression of mRNA or protein include, but are not limited to, gene expression profiling, polymerase chain reaction (PCR) including quantitative real time PCR (qRT-PCR), microarray analysis that can be performed by commercially available equipment, following manufacturer's protocols, such as by using the Affymetrix GenChip technology, serial analysis of gene expression (SAGE) (Velculescu et al., Science 270:484-487 (1995); and Velculescu et al., Cell 88:243-51 (1997)), MassARRAY, Gene Expression Analysis by Massively Parallel Signature Sequencing (MPSS) (Brenner et al., Nature Biotechnology 18:630-634 (2000)), proteomics, immunohistochemistry (1HC), etc. Preferably mRNA is quantified. Such mRNA analysis is preferably performed using the technique of polymerase chain reaction (PCR), or by microarray analysis. Where PCR is employed, a preferred form of PCR is quantitative real time PCR (qRT-PCR).

a. Reverse Transcriptase PCR(RT-PCR)

Of the techniques listed above, the most sensitive and most flexible quantitative method is RT-PCR, which can be used to compare mRNA levels in different sample populations, in normal and test sample tissues, to characterize patterns of gene expression, to discriminate between closely related mRNAs, and to analyze RNA structure.

The first step is the isolation of mRNA from a target sample. The starting material is typically total RNA isolated from colonic tissue biopsies. Thus, RNA can be isolated from a variety of tissues, including without limitation, the terminal ileum, the ascending colon, the descending colon, and the sigmoid colon. In addition, the colonic tissue from which a biopsy is obtained may be from an inflamed and/or a non-inflamed colonic area.

In one embodiment, the mRNA is obtained from a biopsy as defined above wherein the biopsy is obtained from the left colon or from the right colon. As used herein, the “left colon” refers to the sigmoideum and rectosigmoideum and the “right colon” refers to the cecum.

General methods for mRNA extraction are well known in the art and are disclosed in standard textbooks of molecular biology, including Ausubel et al., Current Protocols of Molecular Biology, John Wiley and Sons (1997). In particular, RNA isolation can be performed using purification kit, buffer set and protease from commercial manufacturers, such as Qiagen, according to the manufacturer's instructions. Total RNA from tissue samples can be isolated using RNA Stat-60 (Tel-Test). RNA prepared from a biopsy can be isolated, for example, by cesium chloride density gradient centrifugation.

As RNA cannot serve as a template for PCR, the first step in gene expression profiling by RT-PCR is the reverse transcription of the RNA template into cDNA, followed by its exponential amplification in a PCR reaction. The two most commonly used reverse transcriptases are avilo myeloblastosis virus reverse transcriptase (AMV-RT) and Moloney murine leukemia virus reverse transcriptase (MMLV-RT). The reverse transcription step is typically primed using specific primers, random hexamers, or oligo-dT primers, depending on the circumstances and the goal of expression profiling. For example, extracted RNA can be reverse-transcribed using a GeneAmp RNA PCR kit (Perkin Elmer, Calif., USA), following the manufacturer's instructions. The derived cDNA can then be used as a template in the subsequent PCR reaction.

Although the PCR step can use a variety of thermostable DNA-dependent DNA polymerases, it typically employs the Taq DNA polymerase, which has a 5′-3′ nuclease activity but lacks a 3′-5′ proofreading endonuclease activity. Thus, TaqMan° PCR typically utilizes the 5′-nuclease activity of Taq or Tth polymerase to hydrolyze a hybridization probe bound to its target amplicon, but any enzyme with equivalent 5′ nuclease activity can be used. Two oligonucleotide primers are used to generate an amplicon typical of a PCR reaction. A third oligonucleotide, or probe, is designed to detect nucleotide sequence located between the two PCR primers. The probe is non-extendible by Taq DNA polymerase enzyme, and is labeled with a reporter fluorescent dye and a quencher fluorescent dye. Any laser-induced emission from the reporter dye is quenched by the quenching dye when the two dyes are located close together as they are on the probe. During the amplification reaction, the Taq DNA polymerase enzyme cleaves the probe in a template-dependent manner. The resultant probe fragments disassociate in solution, and signal from the released reporter dye is free from the quenching effect of the second fluorophore. One molecule of reporter dye is liberated for each new molecule synthesized, and detection of the unquenched reporter dye provides the basis for quantitative interpretation of the data.

TaqMan® RT-PCR can be performed using commercially available equipment, such as, for example, ABI PRISM 7700™ Sequence Detection System™ (Perkin-Elmer-Applied Biosystems, Foster City, Calif., USA), or Lightcycler (Roche Molecular Biochemicals, Mannheim, Germany). In a preferred embodiment, the 5′ nuclease procedure is run on a real-time quantitative PCR device such as the ABI PRISM 7700™ Sequence Detection System™. The system consists of a thermocycler, laser, charge-coupled device (CCD), camera and computer. The system amplifies samples in a 96-well format on a thermocycler. During amplification, laser-induced fluorescent signal is collected in real-time through fiber optics cables for all 96 wells, and detected at the CCD. The system includes software for running the instrument and for analyzing the data.

5′-Nuclease assay data are initially expressed as Ct, or the threshold cycle. As discussed above, fluorescence values are recorded during every cycle and represent the amount of product amplified to that point in the amplification reaction. The point when the fluorescent signal is first recorded as statistically significant is the threshold cycle (Ct).

To minimize errors and the effect of sample-to-sample variation, RT-PCR is usually performed using an internal standard. The ideal internal standard is expressed at a constant level among different tissues, and is unaffected by the experimental treatment. RNAs most frequently used to normalize patterns of gene expression are mRNAs for the housekeeping genes glyceraldehyde-3-phosphate-dehydrogenase (GAPDH) and β-actin.

A more recent variation of the RT-PCR technique is the real time quantitative PCR, which measures PCR product accumulation through a dual-labeled fluorigenic probe (i.e., TaqMan® probe). Real time PCR is compatible both with quantitative competitive PCR, where internal competitor for each target sequence is used for normalization, and with quantitative comparative PCR using a normalization gene contained within the sample, or a housekeeping gene for RT-PCR. For further details see, e.g. Held et al., Genome Research 6:986-994 (1996).

According to one aspect of the present invention, PCR primers and probes are designed based upon intron sequences present in the gene to be amplified. In this embodiment, the first step in the primer/probe design is the delineation of intron sequences within the genes. This can be done by publicly available software, such as the DNA BLAT software developed by Kent, W. J., Genome Res. 12(4):656-64 (2002), or by the BLAST software including its variations. Subsequent steps follow well established methods of PCR primer and probe design.

In order to avoid non-specific signals, it is important to mask repetitive sequences within the introns when designing the primers and probes. This can be easily accomplished by using the Repeat Masker program available on-line through the Baylor College of Medicine, which screens DNA sequences against a library of repetitive elements and returns a query sequence in which the repetitive elements are masked. The masked intron sequences can then be used to design primer and probe sequences using any commercially or otherwise publicly available primer/probe design packages, such as Primer Express (Applied Biosystems); MGB assay-by-design (Applied Biosystems); Primer3 (Steve Rozen and Helen J. Skaletsky (2000) Primer3 on the WWW for general users and for biologist programmers. In: Krawetz S, Misener S (eds) Bioinformatics Methods and Protocols: Methods in Molecular Biology. Humana Press, Totowa, N.J., pp 365-386).

The most important factors considered in PCR primer design include primer length, melting temperature (Tm), and G/C content, specificity, complementary primer sequences, and 3′-end sequence. In general, optimal PCR primers are generally 17-30 bases in length, and contain about 20-80%, such as, for example, about 50-60% G+C bases. Tm's between 50 and 80° C., e.g. about 50 to 70° C. are typically preferred.

For further guidelines for PCR primer and probe design see, e.g. Dieffenbach, C. W. et al., “General Concepts for PCR Primer Design” in: PCR Primer, A Laboratory Manual, Cold Spring Harbor Laboratory Press, New York, 1995, pp. 133-155; Innis and Gelfand, “Optimization of PCRs” in: PCR Protocols, A Guide to Methods and Applications, CRC Press, London, 1994, pp. 5-11; and Plasterer, T. N. Primerselect: Primer and probe design. Methods Mol. Biol. 70:520-527 (1997), the entire disclosures of which are hereby expressly incorporated by reference.

Further PCR-based techniques include, for example, differential display (Liang and Pardee, Science 257:967-971 (1992)); amplified fragment length polymorphism (iAFLP) (Kawamoto et al., Genome Res. 12:1305-1312 (1999)); BeadArray™ technology (Illumina, San Diego, Calif.; Oliphant et al., Discovery of Markers for Disease (Supplement to Biotechniques), June 2002; Ferguson et al., Analytical Chemistry 72:5618 (2000)); BeadsArray for Detection of Gene Expression (BADGE), using the commercially available Luminex 100 LabMAP system and multiple color-coded microspheres (Luminex Corp., Austin, Tex.) in a rapid assay for gene expression (Yang et al., Genome Res. 11:1888-1898 (2001)); and high coverage expression profiling (HiCEP) analysis (Fukumura et al., Nucl. Acids. Res. 31(16) e94 (2003)).

b. Microarrays

Differential gene expression can also be identified, or confirmed using the microarray technique. Thus, the expression profile of IBD-associated genes can be measured in either fresh or paraffin-embedded tissue, using microarray technology. In this method, polynucleotide sequences of interest (including cDNAs and oligonucleotides) are plated, or arrayed, on a microchip substrate. The arrayed sequences are then hybridized with specific DNA probes from cells or tissues of interest. Just as in the RT-PCR method, the source of mRNA typically is total RNA isolated from biopsy tissue or cell lines derived from cells obtained from a subject having an IBD, and corresponding normal tissues or cell lines. Thus RNA can be isolated from a variety of colonic tissues or colonic tissue-based cell lines.

In a specific embodiment of the microarray technique, PCR amplified inserts of cDNA clones are applied to a substrate in a dense array. Preferably at least 10,000 nucleotide sequences are applied to the substrate. The microarrayed genes, immobilized on the microchip at 10,000 elements each, are suitable for hybridization under stringent conditions. Fluorescently labeled cDNA probes may be generated through incorporation of fluorescent nucleotides by reverse transcription of RNA extracted from tissues of interest. Labeled cDNA probes applied to the chip hybridize with specificity to each spot of DNA on the array. After stringent washing to remove non-specifically bound probes, the chip is scanned by confocal laser microscopy or by another detection method, such as a CCD camera. Quantitation of hybridization of each arrayed element allows for assessment of corresponding mRNA abundance. With dual color fluorescence, separately labeled cDNA probes generated from two sources of RNA are hybridized pairwise to the array. The relative abundance of the transcripts from the two sources corresponding to each specified gene is thus determined simultaneously. The miniaturized scale of the hybridization affords a convenient and rapid evaluation of the expression pattern for large numbers of genes. Such methods have been shown to have the sensitivity required to detect rare transcripts, which are expressed at a few copies per cell, and to reproducibly detect at least approximately two-fold differences in the expression levels (Schena et al., Proc. Natl. Acad. Sci. USA 93(2):106-149 (1996)). Microarray analysis can be performed by commercially available equipment, following manufacturer's protocols, such as by using the Affymetrix GenChip technology, or Incyte's microarray technology, or Agilent's Whole Human Genome microarray technology.

c. Serial Analysis of Gene Expression (SAGE)

Serial analysis of gene expression (SAGE) is a method that allows the simultaneous and quantitative analysis of a large number of gene transcripts, without the need of providing an individual hybridization probe for each transcript. First, a short sequence tag (about 10-14 bp) is generated that contains sufficient information to uniquely identify a transcript, provided that the tag is obtained from a unique position within each transcript. Then, many transcripts are linked together to form long serial molecules, that can be sequenced, revealing the identity of the multiple tags simultaneously. The expression pattern of any population of transcripts can be quantitatively evaluated by determining the abundance of individual tags, and identifying the gene corresponding to each tag. For more details see, e.g. Velculescu et al., Science 270:484-487 (1995); and Velculescu et al., Cell 88:243-51 (1997).

d. MassARRAY Technology

In the MassARRAY-based gene expression profiling method, developed by Sequenom, Inc. (San Diego, Calif.) following the isolation of RNA and reverse transcription, the obtained cDNA is spiked with a synthetic DNA molecule (competitor), which matches the targeted cDNA region in all positions, except a single base, and serves as an internal standard. The cDNA/competitor mixture is PCR amplified and is subjected to a post-PCR shrimp alkaline phosphatase (SAP) enzyme treatment, which results in the dephosphorylation of the remaining nucleotides. After inactivation of the alkaline phosphatase, the PCR products from the competitor and cDNA are subjected to primer extension, which generates distinct mass signals for the competitor- and cDNA-derives PCR products. After purification, these products are dispensed on a chip array, which is pre-loaded with components needed for analysis with matrix-assisted laser desorption ionization time-of-flight mass spectrometry (MALDI-TOF MS) analysis. The cDNA present in the reaction is then quantified by analyzing the ratios of the peak areas in the mass spectrum generated. For further details see, e.g. Ding and Cantor, Proc. Natl. Acad. Sci. USA 100:3059-3064 (2003).

e. Gene Expression Analysis by Massively Parallel Signature Sequencing (MPSS)

This method, described by Brenner et al., Nature Biotechnology 18:630-634 (2000), is a sequencing approach that combines non-gel-based signature sequencing with in vitro cloning of millions of templates on separate 5 μm diameter microbeads. First, a microbead library of DNA templates is constructed by in vitro cloning. This is followed by the assembly of a planar array of the template-containing microbeads in a flow cell at a high density (typically greater than 3×10⁶ microbeads/cm²). The free ends of the cloned templates on each microbead are analyzed simultaneously, using a fluorescence-based signature sequencing method that does not require DNA fragment separation. This method has been shown to simultaneously and accurately provide, in a single operation, hundreds of thousands of gene signature sequences from a yeast cDNA library.

The steps of a representative protocol for profiling gene expression using fixed, paraffin-embedded tissues as the RNA source, including mRNA isolation, purification, primer extension and amplification are given in various published journal articles (for example: Godfrey et al. J. Molec. Diagnostics 2: 84-91 (2000); Specht et al., Am. J. Pathol. 158: 419-29 (2001)). Briefly, a representative process starts with cutting about 10 microgram thick sections of paraffin-embedded tissue samples. The mRNA is then extracted, and protein and DNA are removed. General methods for mRNA extraction are well known in the art and are disclosed in standard textbooks of molecular biology, including Ausubel et al., Current Protocols of Molecular Biology, John Wiley and Sons (1997). Methods for RNA extraction from paraffin embedded tissues are disclosed, for example, in Rupp and Locker, Lab Invest. 56:A67 (1987), and De Andrés et al., BioTechniques 18:42044 (1995). In particular, RNA isolation can be performed using purification kit, buffer set and protease from commercial manufacturers, such as Qiagen, according to the manufacturer's instructions. For example, total RNA from cells in culture can be isolated using Qiagen RNeasy mini-columns. Other commercially available RNA isolation kits include MasterPure™ Complete DNA and RNA Purification Kit (EPICENTRE®, Madison, Wis.), and Paraffin Block RNA Isolation Kit (Ambion, Inc.). Total RNA from tissue samples can be isolated using RNA Stat-60 (Tel-Test). RNA prepared from tissues can be isolated, for example, by cesium chloride density gradient centrifugation. After analysis of the RNA concentration, RNA repair and/or amplification steps may be included, if necessary, and RNA is reverse transcribed using gene specific promoters followed by PCR. Preferably, real time PCR is used, which is compatible both with quantitative competitive PCR, where internal competitor for each target sequence is used for normalization, and with quantitative comparative PCR using a normalization gene contained within the sample, or a housekeeping gene for RT-PCR. For further details see, e.g. “PCR: The Polymerase Chain Reaction”, Mullis et al., eds., 1994; and Held et al., Genome Research 6:986-994 (1996). Finally, the data are analyzed to identify the best treatment option(s) available to the patient on the basis of the characteristic gene expression pattern identified in the sample examined.

f. Immunohistochemistry

Immunohistochemistry methods are also suitable for detecting the expression levels of the IBD markers of the present invention. Thus, antibodies or antisera, preferably polyclonal antisera, and most preferably monoclonal antibodies specific for each marker are used to detect expression. The antibodies can be detected by direct labeling of the antibodies themselves, for example, with radioactive labels, fluorescent labels, hapten labels such as, biotin, or an enzyme such as horse radish peroxidase or alkaline phosphatase. Alternatively, unlabeled primary antibody is used in conjunction with a labeled secondary antibody, comprising antisera, polyclonal antisera or a monoclonal antibody specific for the primary antibody. Immunohistochemistry protocols and kits are well known in the art and are commercially available.

Expression levels can also be determined at the protein level, for example, using various types of immunoassays or proteomics techniques.

In immunoassays, the target diagnostic protein marker is detected by using an antibody specifically binding to the marks. The antibody typically will be labeled with a detectable moiety. Numerous labels are available which can be generally grouped into the following categories:

Radioisotopes, such as 35S, 14C, 125I, 3H, and 131I. The antibody can be labeled with the radioisotope using the techniques described in Current Protocols in Immunology, Volumes 1 and 2, Coligen et al. (1991) Ed. Wiley-Interscience, New York, N.Y., Pubs. for example and radioactivity can be measured using scintillation counting.

Fluorescent labels such as rare earth chelates (europium chelates) or fluorescein and its derivatives, rhodamine and its derivatives, dansyl, Lissamine, phycoerythrin and Texas Red are available. The fluorescent labels can be conjugated to the antibody using the techniques disclosed in Current Protocols in Immunology, supra, for example. Fluorescence can be quantified using a fluorimeter.

Various enzyme-substrate labels are available and U.S. Pat. No. 4,275,149 provides a review of some of these. The enzyme generally catalyzes a chemical alteration of the chromogenic substrate which can be measured using various techniques. For example, the enzyme may catalyze a color change in a substrate, which can be measured spectrophotometrically. Alternatively, the enzyme may alter the fluorescence or chemiluminescence of the substrate. Techniques for quantifying a change in fluorescence are described above. The chemiluminescent substrate becomes electronically excited by a chemical reaction and may then emit light which can be measured (using a chemiluminometer, for example) or donates energy to a fluorescent acceptor. Examples of enzymatic labels include luciferases (e.g., firefly luciferase and bacterial luciferase; U.S. Pat. No. 4,737,456), luciferin, 2,3-dihydrophthalazinediones, malate dehydrogenase, urease, peroxidase such as horseradish peroxidase (HRPO), alkaline phosphatase, β-galactosidase, glucoamylase, lysozyme, saccharide oxidases (e.g., glucose oxidase, galactose oxidase, and glucose-6-phosphate dehydrogenase), heterocyclic oxidases (such as uricase and xanthine oxidase), lactoperoxidase, microperoxidase, and the like. Techniques for conjugating enzymes to antibodies are described in O'Sullivan et al. (1981) Methods for the Preparation of Enzyme-Antibody Conjugates for use in Enzyme Immunoassay, in Methods in Enzym. (ed J. Langone & H. Van Vunakis), Academic press, New York 73:147-166.

Examples of enzyme-substrate combinations include, for example: horseradish peroxidase (HRPO) with hydrogen peroxidase as a substrate, wherein the hydrogen peroxidase oxidizes a dye precursor (e.g., orthophenylene diamine (OPD) or 3,3′,5,5′-tetramethyl benzidine hydrochloride (TMB)); alkaline phosphatase (AP) with para-Nitrophenyl phosphate as chromogenic substrate; and β-D-galactosidase (β-D-Gal) with a chromogenic substrate (e.g., p-nitrophenyl-β-D-galactosidase) or fluorogenic substrate 4-methylumbelliferyl-β-D-galactosidase.

Numerous other enzyme-substrate combinations are available to those skilled in the art. For a general review of these, see U.S. Pat. Nos. 4,275,149 and 4,318,980.

Sometimes, the label is indirectly conjugated with the antibody. The skilled artisan will be aware of various techniques for achieving this. For example, the antibody can be conjugated with biotin and any of the three broad categories of labels mentioned above can be conjugated with avidin, or vice versa. Biotin binds selectively to avidin and thus, the label can be conjugated with the antibody in this indirect manner. Alternatively, to achieve indirect conjugation of the label with the antibody, the antibody is conjugated with a small hapten (e.g., digoxin) and one of the different types of labels mentioned above is conjugated with an anti-hapten antibody (e.g., anti-digoxin antibody). Thus, indirect conjugation of the label with the antibody can be achieved.

In other versions of immunoassay techniques, the antibody need not be labeled, and the presence thereof can be detected using a labeled antibody which binds to the antibody.

Thus, the diagnostic immunoassays herein may be in any assay format, including, for example, competitive binding assays, direct and indirect sandwich assays, and immunoprecipitation assays. Zola, Monoclonal Antibodies: A Manual of Techniques, pp. 147-158 (CRC Press, Inc. 1987).

Competitive binding assays rely on the ability of a labeled standard to compete with the test sample analyze for binding with a limited amount of antibody. The amount of antigen in the test sample is inversely proportional to the amount of standard that becomes bound to the antibodies. To facilitate determining the amount of standard that becomes bound, the antibodies generally are insolubilized before or after the competition, so that the standard and analyze that are bound to the antibodies may conveniently be separated from the standard and analyze which remain unbound.

Sandwich assays involve the use of two antibodies, each capable of binding to a different immunogenic portion, or epitope, of the protein to be detected. In a sandwich assay, the test sample analyze is bound by a first antibody which is immobilized on a solid support, and thereafter a second antibody binds to the analyze, thus forming an insoluble three-part complex. See, e.g., U.S. Pat. No. 4,376,110. The second antibody may itself be labeled with a detectable moiety (direct sandwich assays) or may be measured using an anti-immunoglobulin antibody that is labeled with a detectable moiety (indirect sandwich assay). For example, one type of sandwich assay is an ELISA assay, in which case the detectable moiety is an enzyme.

g. Proteomics

The term “proteome” is defined as the totality of the proteins present in a sample (e.g. tissue, organism, or cell culture) at a certain point of time. Proteomics includes, among other things, study of the global changes of protein expression in a sample (also referred to as “expression proteomics”). Proteomics typically includes the following steps: (1) separation of individual proteins in a sample by 2-D gel electrophoresis (2-D PAGE); (2) identification of the individual proteins recovered from the gel, e.g. my mass spectrometry or N-terminal sequencing, and (3) analysis of the data using bioinformatics. Proteomics methods are valuable supplements to other methods of gene expression profiling, and can be used, alone or in combination with other methods, to detect the products of the markers of the present invention.

h. 5′-Multiplexed Gene Specific Priming of Reverse Transcription

RT-PCR requires reverse transcription of the test RNA population as a first step. The most commonly used primer for reverse transcription is oligo-dT, which works well when RNA is intact. However, this primer will not be effective when RNA is highly fragmented.

The present invention includes the use of gene specific primers, which are roughly 20 bases in length with a Tm optimum between about 58° C. and 60° C. These primers will also serve as the reverse primers that drive PCR DNA amplification.

An alternative approach is based on the use of random hexamers as primers for cDNA synthesis. However, we have experimentally demonstrated that the method of using a multiplicity of gene-specific primers is superior over the known approach using random hexamers.

i. Promoter Methylation Analysis

A number of methods for quantization of RNA transcripts (gene expression analysis) or their protein translation products are discussed herein. The expression level of genes may also be inferred from information regarding chromatin structure, such as for example the methylation status of gene promoters and other regulatory elements and the acetylation status of histones.

In particular, the methylation status of a promoter influences the level of expression of the gene regulated by that promoter. Aberrant methylation of particular gene promoters has been implicated in expression regulation, such as for example silencing of tumor suppressor genes. Thus, examination of the methylation status of a gene's promoter can be utilized as a surrogate for direct quantization of RNA levels.

Several approaches for measuring the methylation status of particular DNA elements have been devised, including methylation-specific PCR (Herman J. G. et al. (1996) Methylation-specific PCR: a novel PCR assay for methylation status of CpG islands. Proc. Natl. Acad. Sci. USA. 93, 9821-9826) and bisulfite DNA sequencing (Frommer M. et al. (1992) A genomic sequencing protocol that yields a positive display of 5-methylcytosine residues in individual DNA strands. Proc. Natl. Acad. Sci. USA. 89, 1827-1831). More recently, microarray-based technologies have been used to characterize promoter methylation status (Chen C. M. (2003) Methylation target array for rapid analysis of CpG island hypermethylation in multiple tissue genomes. Am. J. Pathol. 163, 37-45).

j. Coexpression of Genes

A further aspect of the invention is the identification of gene expression clusters. Gene expression clusters can be identified by analysis of expression data using statistical analyses known in the art, including pairwise analysis of correlation based on Pearson correlation coefficients (Pearson K. and Lee A. (1902) Biometrika 2, 357).

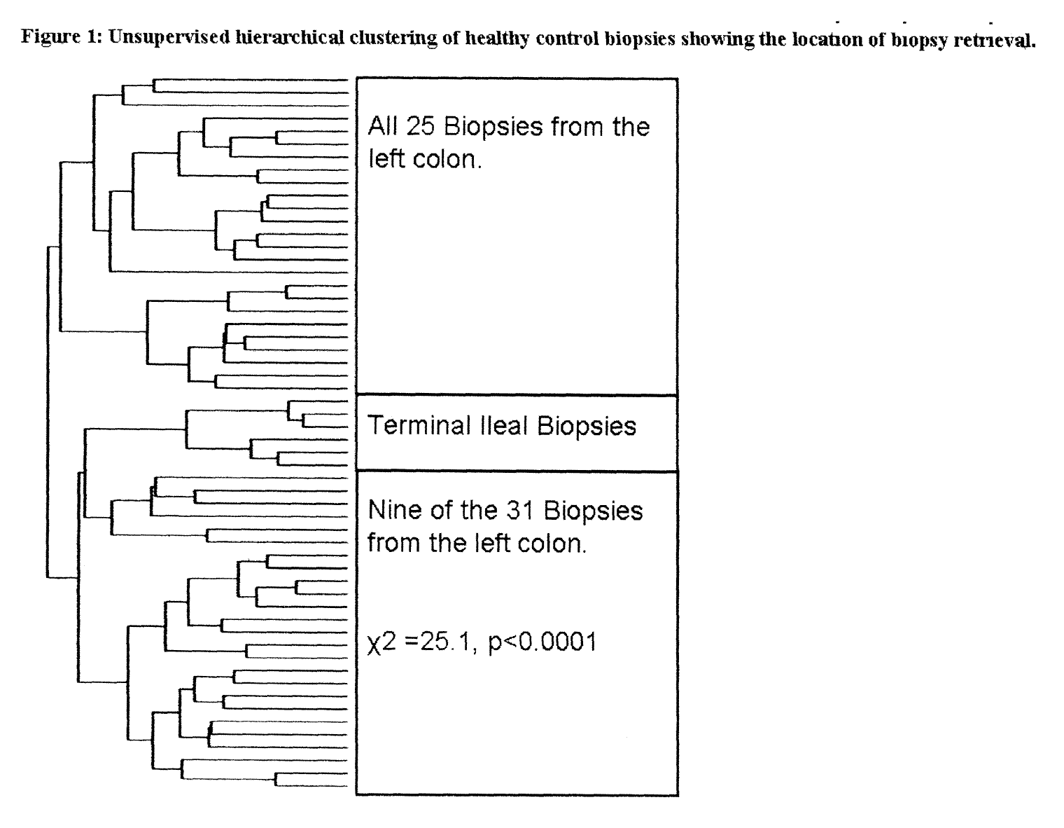

In one embodiment, an expression cluster identified herein includes genes upregulated in the left colon (FIG. 1).

In another embodiment, an expression cluster identified herein includes genes upregulated in the right colon (FIG. 1).

In one other embodiment, an expression cluster identified herein includes genes upregulated in the terminal ileum (FIG. 1).

In other embodiments, the expression cluster identified herein includes genes in the IBD2 locus (Table 7); or in the IBDS locus (Table 8).

In some embodiments, the expression cluster identified herein includes genes classified under an immune response.

In other embodiments, the expression cluster identified herein includes genes classified under a response to wounding.

k. Design of Intron-Based PCR Primers and Probes

According to one aspect of the present invention, PCR primers and probes are designed based upon intron sequences present in the gene to be amplified. Accordingly, the first step in the primer/probe design is the delineation of intron sequences within the genes. This can be done by publicly available software, such as the DNA BLAT software developed by Kent, W. J., Genome Res. 12(4):656-64 (2002), or by the BLAST software including its variations. Subsequent steps follow well established methods of PCR primer and probe design.

In order to avoid non-specific signals, it is important to mask repetitive sequences within the introns when designing the primers and probes. This can be easily accomplished by using the Repeat Masker program available on-line through the Baylor College of Medicine, which screens DNA sequences against a library of repetitive elements and returns a query sequence in which the repetitive elements are masked. The masked intron sequences can then be used to design primer and probe sequences using any commercially or otherwise publicly available primer/probe design packages, such as Primer Express (Applied Biosystems); MGB assay-by-design (Applied Biosystems); Primer3 (Steve Rozen and Helen J. Skaletsky (2000) Primer3 on the WWW for general users and for biologist programmers. In: Krawetz S, Misener S (eds) Bioinformatics Methods and Protocols: Methods in Molecular Biology. Humana Press, Totowa, N.J., pp 365-386).

The most important factors considered in PCR primer design include primer length, melting temperature (Tm), and G/C content, specificity, complementary primer sequences, and 3′-end sequence. In general, optimal PCR primers are generally 17-30 bases in length, and contain about 20-80%, such as, for example, about 50-60% G+C bases. Tm's between 50 and 80° C., e.g. about 50 to 70° C. are typically preferred.

For further guidelines for PCR primer and probe design see, e.g. Dieffenbach, C. W. et al., “General Concepts for PCR Primer Design” in: PCR Primer, A Laboratory Manual, Cold Spring Harbor Laboratory Press, New York, 1995, pp. 133-155; Innis and Gelfand, “Optimization of PCRs” in: PCR Protocols, A Guide to Methods and Applications, CRC Press, London, 1994, pp. 5-11; and Plasterer, T. N. Primerselect: Primer and probe design. Methods Mol. Biol. 70:520-527 (1997), the entire disclosures of which are hereby expressly incorporated by reference.

l. IBD Gene Set, Assayed Gene Subsequences, and Clinical Application of Gene Expression Data

An important aspect of the present invention is to use the measured expression of certain genes by colonic issue to provide diagnostic information. For this purpose it is necessary to correct for (normalize away) both differences in the amount of RNA assayed and variability in the quality of the RNA used. Therefore, the assay typically measures and incorporates the expression of certain normalizing genes, including well known housekeeping genes, such as GAPDH and Cyp1. Alternatively, normalization can be based on the mean or median signal (Ct) of all of the assayed genes or a large subset thereof (global normalization approach). On a gene-by-gene basis, measured normalized amount of a patient colonic tissue mRNA is compared to the amount found in an appropriate tissue reference set. The number (N) of tissues in this reference set should be sufficiently high to ensure that different reference sets (as a whole) behave essentially the same way. If this condition is met, the identity of the individual colonic tissues present in a particular set will have no significant impact on the relative amounts of the genes assayed. Usually, the tissue reference set consists of at least about 30, preferably at least about 40 different IBD tissue specimens. Unless noted otherwise, normalized expression levels for each mRNA/tested tissue/patient will be expressed as a percentage of the expression level measured in the reference set. More specifically, the reference set of a sufficiently high number (e.g. 40) of IBD samples yields a distribution of normalized levels of each mRNA species. The level measured in a particular sample to be analyzed falls at some percentile within this range, which can be determined by methods well known in the art. Below, unless noted otherwise, reference to expression levels of a gene assume normalized expression relative to the reference set although this is not always explicitly stated.

m. Production of Antibodies

The present invention further provides anti-IBD marker antibodies. Exemplary antibodies include polyclonal, monoclonal, humanized, bispecific, and heteroconjugate antibodies. As discussed herein, the antibodies may be used in the diagnostic methods for IBD, and in some cases in methods of treatment of IBD.

(1) Polyclonal Antibodies

Polyclonal antibodies are preferably raised in animals by multiple subcutaneous (sc) or intraperitoneal (ip) injections of the relevant antigen and an adjuvant. It may be useful to conjugate the relevant antigen to a protein that is immunogenic in the species to be immunized, e.g., keyhole limpet hemocyanin, serum albumin, bovine thyroglobulin, or soybean trypsin inhibitor using a bifunctional or derivatizing agent, for example, maleimidobenzoyl sulfosuccinimide ester (conjugation through cysteine residues), N-hydroxysuccinimide (through lysine residues), glutaraldehyde, succinic anhydride, SOCl2, or R1N═C═NR, where R1 and R1 are different alkyl groups.

Animals are immunized against the antigen, immunogenic conjugates, or derivatives by combining, e.g., 100 μg or 5 μg of the protein or conjugate (for rabbits or mice, respectively) with 3 volumes of Freund's complete adjuvant and injecting the solution intradermally at multiple sites. One month later the animals are boosted with ⅕ to 1/10 the original amount of peptide or conjugate in Freund's complete adjuvant by subcutaneous injection at multiple sites. Seven to 14 days later the animals are bled and the serum is assayed for antibody titer. Animals are boosted until the titer plateaus. Preferably, the animal is boosted with the conjugate of the same antigen, but conjugated to a different protein and/or through a different cross-linking reagent. Conjugates also can be made in recombinant cell culture as protein fusions. Also, aggregating agents such as alum are suitably used to enhance the immune response.

(2) Monoclonal Antibodies

Various methods for making monoclonal antibodies herein are available in the art. For example, the monoclonal antibodies may be made using the hybridoma method first described by Kohler et al., Nature, 256:495 (1975), by recombinant DNA methods (U.S. Pat. No. 4,816,567).

In the hybridoma method, a mouse or other appropriate host animal, such as a hamster, is immunized as hereinabove described to elicit lymphocytes that produce or are capable of producing antibodies that will specifically bind to the protein used for immunization. Alternatively, lymphocytes may be immunized in vitro. Lymphocytes then are fused with myeloma cells using a suitable fusing agent, such as polyethylene glycol, to form a hybridoma cell (Goding, Monoclonal Antibodies: Principles and Practice, pp. 59-103 (Academic Press, 1986)).

The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Preferred myeloma cells are those that fuse efficiently, support stable high-level production of antibody by the selected antibody-producing cells, and are sensitive to a medium such as HAT medium. Among these, preferred myeloma cell lines are murine myeloma lines, such as those derived from MOPC-21 and MPC-11 mouse tumors available from the Salk Institute Cell Distribution Center, San Diego, Calif. USA, and SP-2 or X63-Ag8-653 cells available from the American Type Culture Collection, Rockville, Md. USA. Human myeloma and mouse-human heteromyeloma cell lines also have been described for the production of human monoclonal antibodies (Kozbor, J. Immunol., 133:3001 (1984); and Brodeur et al., Monoclonal Antibody Production Techniques and Applications, pp. 51-63 (Marcel Dekker, Inc., New York, 1987)).

Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as radioimmunoassay (RIA) or enzyme-linked immunoabsorbent assay (ELISA).

The binding affinity of the monoclonal antibody can, for example, be determined by the Scatchard analysis of Munson et al., Anal. Biochem., 107:220 (1980).

After hybridoma cells are identified that produce antibodies of the desired specificity, affinity, and/or activity, the clones may be subcloned by limiting dilution procedures and grown by standard methods (Goding, Monoclonal Antibodies: Principles and Practice, pp. 59-103 (Academic Press, 1986)). Suitable culture media for this purpose include, for example, D-MEM or RPMI-1640 medium. In addition, the hybridoma cells may be grown in vivo as ascites tumors in an animal.

The monoclonal antibodies secreted by the subclones are suitably separated from the culture medium, ascites fluid, or serum by conventional antibody purification procedures such as, for example, protein A-Sepharose, hydroxylapatite chromatography, gel electrophoresis, dialysis, or affinity chromatography.

DNA encoding the monoclonal antibodies is readily isolated and sequenced using conventional procedures (e.g., by using oligonucleotide probes that are capable of binding specifically to genes encoding the heavy and light chains of murine antibodies). The hybridoma cells serve as a preferred source of such DNA. Once isolated, the DNA may be placed into expression vectors, which are then transfected into host cells such as E. coli cells, simian COS cells, Chinese Hamster Ovary (CHO) cells, or myeloma cells that do not otherwise produce antibody protein, to obtain the synthesis of monoclonal antibodies in the recombinant host cells. Review articles on recombinant expression in bacteria of DNA encoding the antibody include Skerra et al., Curr. Opinion in Immunol., 5:256-262 (1993) and Plückthun, Immunol Revs., 130:151-188 (1992).

In a further embodiment, monoclonal antibodies or antibody fragments can be isolated from antibody phage libraries generated using the techniques described in McCafferty et al., Nature, 348:552-554 (1990). Clackson et al., Nature, 352:624-628 (1991) and Marks et al., J. Mol. Biol., 222:581-597 (1991) describe the isolation of murine and human antibodies, respectively, using phage libraries. Subsequent publications describe the production of high affinity (nM range) human antibodies by chain shuffling (Marks et al., Bio/Technology, 10:779-783 (1992)), as well as combinatorial infection and in vivo recombination as a strategy for constructing very large phage libraries (Waterhouse et al., Nuc. Acids. Res., 21:2265-2266 (1993)). Thus, these techniques are viable alternatives to traditional monoclonal antibody hybridoma techniques for isolation of monoclonal antibodies.

The DNA also may be modified, for example, by substituting the coding sequence for human heavy chain and light chain constant domains in place of the homologous murine sequences (U.S. Pat. No. 4,816,567; and Morrison, et al., Proc. Natl. Acad. Sci. USA, 81:6851 (1984)), or by covalently joining to the immunoglobulin coding sequence all or part of the coding sequence for a non-immunoglobulin polypeptide.

Typically such non-immunoglobulin polypeptides are substituted for the constant domains of an antibody, or they are substituted for the variable domains of one antigen-combining site of an antibody to create a chimeric bivalent antibody comprising one antigen-combining site having specificity for an antigen and another antigen-combining site having specificity for a different antigen.

(3) Humanized Antibodies

Methods for humanizing non-human antibodies have been described in the art. Preferably, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting hypervariable region sequences for the corresponding sequences of a human antibody. Accordingly, such “humanized” antibodies are chimeric antibodies (U.S. Pat. No. 4,816,567) wherein substantially less than an intact human variable domain has been substituted by the corresponding sequence from a non-human species. In practice, humanized antibodies are typically human antibodies in which some hypervariable region residues and possibly some FR residues are substituted by residues from analogous sites in rodent antibodies. An example of a humanized antibody used to treat IBD is infliximab (Remicade®), an engineered murine-human chimeric monoclonal antibody. The antibody binds the cytokine TNF-alpha and prevents it from binding its receptors to trigger and sustain an inflammatory response. Infliximab is used to treat both CD and UC.

The choice of human variable domains, both light and heavy, to be used in making the humanized antibodies is very important to reduce antigenicity. According to the so-called “best-fit” method, the sequence of the variable domain of a rodent antibody is screened against the entire library of known human variable-domain sequences. The human sequence which is closest to that of the rodent is then accepted as the human framework region (FR) for the humanized antibody (Sims et al., J. Immunol., 151:2296 (1993); Chothia et al., J. Mol. Biol., 196:901 (1987)). Another method uses a particular framework region derived from the consensus sequence of all human antibodies of a particular subgroup of light or heavy chains. The same framework may be used for several different humanized antibodies (Carter et al., Proc. Natl. Acad. Sci. USA, 89:4285 (1992); Presta et al., J. Immunol., 151:2623 (1993)).

It is further important that antibodies be humanized with retention of high affinity for the antigen and other favorable biological properties. To achieve this goal, according to a preferred method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the hypervariable region residues are directly and most substantially involved in influencing antigen binding.

Various forms of the humanized antibody are contemplated. For example, the humanized antibody may be an antibody fragment, such as a Fab, which is optionally conjugated with one or more cytotoxic agent(s) in order to generate an immunoconjugate. Alternatively, the humanized antibody may be an intact antibody, such as an intact IgG1 antibody.

(4) Human Antibodies

As an alternative to humanization, human antibodies can be generated. For example, it is now possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region (JH) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g., Jakobovits et al., Proc. Natl. Acad. Sci. USA, 90:2551 (1993); Jakobovits et al., Nature, 362:255-258 (1993); Bruggermann et al., Year in Immuno., 7:33 (1993); and U.S. Pat. Nos. 5,591,669, 5,589,369 and 5,545,807. Alternatively, phage display technology (McCafferty et al., Nature 348:552-553 (1990)) can be used to produce human antibodies and antibody fragments in vitro, from immunoglobulin variable (V) domain gene repertoires from unimmunized donors. According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B-cell. Phage display can be performed in a variety of formats; for their review see, e.g., Johnson, Kevin S, and Chiswell, David J., Current Opinion in Structural Biology 3:564-571 (1993). Several sources of V-gene segments can be used for phage display. Clackson et al., Nature, 352:624-628 (1991) isolated a diverse array of anti-oxazolone antibodies from a small random combinatorial library of V genes derived from the spleens of immunized mice. A repertoire of V genes from unimmunized human donors can be constructed and antibodies to a diverse array of antigens (including self-antigens) can be isolated essentially following the techniques described by Marks et al., J. Mol. Biol. 222:581-597 (1991), or Griffith et al., EMBO J. 12:725-734 (1993). See, also, U.S. Pat. Nos. 5,565,332 and 5,573,905.

As discussed above, human antibodies may also be generated by in vitro activated B cells (see U.S. Pat. Nos. 5,567,610 and 5,229,275).

(5) Antibody Fragments

Various techniques have been developed for the production of antibody fragments comprising one or more antigen binding regions. Traditionally, these fragments were derived via proteolytic digestion of intact antibodies (see, e.g., Morimoto et al., Journal of Biochemical and Biophysical Methods 24:107-117 (1992); and Brennan et al., Science, 229:81 (1985)). However, these fragments can now be produced directly by recombinant host cells. For example, the antibody fragments can be isolated from the antibody phage libraries discussed above. Alternatively, Fab′-SH fragments can be directly recovered from E. coli and chemically coupled to form F(ab′)2 fragments (Carter et al., Bio/Technology 10:163-167 (1992)). According to another approach, F(ab′)2 fragments can be isolated directly from recombinant host cell culture. Other techniques for the production of antibody fragments will be apparent to the skilled practitioner. In other embodiments, the antibody of choice is a single chain FIT fragment (scFv). See WO 93/16185; U.S. Pat. No. 5,571,894; and U.S. Pat. No. 5,587,458. The antibody fragment may also be a Alinear antibody@, e.g., as described in U.S. Pat. No. 5,641,870 for example. Such linear antibody fragments may be monospecific or bispecific.

(6) Bispecific Antibodies

Bispecific antibodies are antibodies that have binding specificities for at least two different epitopes. Exemplary bispecific antibodies may bind to two different epitopes of an IBD marker protein. Bispecific antibodies may also be used to localize agents to cells which express an IBD marker protein.

These antibodies possess an IBD marker-binding arm and an arm which binds an agent (e.g. an aminosalicylate). Bispecific antibodies can be prepared as full length antibodies or antibody fragments (e.g. F(ab′)2 bispecific antibodies).

Methods for making bispecific antibodies are known in the art. Traditional production of full length bispecific antibodies is based on the coexpression of two immunoglobulin heavy chain-light chain pairs, where the two chains have different specificities (Millstein et al., Nature, 305:537-539 (1983)). Because of the random assortment of immunoglobulin heavy and light chains, these hybridomas (quadromas) produce a potential mixture of 10 different antibody molecules, of which only one has the correct bispecific structure. Purification of the correct molecule, which is usually done by affinity chromatography steps, is rather cumbersome, and the product yields are low. Similar procedures are disclosed in WO 93/08829, and in Traunecker et al., EMBO J., 10:3655-3659 (1991).

According to a different approach, antibody variable domains with the desired binding specificities (antibody-antigen combining sites) are fused to immunoglobulin constant domain sequences. The fusion preferably is with an immunoglobulin heavy chain constant domain, comprising at least part of the hinge, CH2, and CH3 regions. It is preferred to have the first heavy-chain constant region (CH1) containing the site necessary for light chain binding, present in at least one of the fusions. DNAs encoding the immunoglobulin heavy chain fusions and, if desired, the immunoglobulin light chain, are inserted into separate expression vectors, and are co-transfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance.

In a preferred embodiment of this approach, the bispecific antibodies are composed of a hybrid immunoglobulin heavy chain with a first binding specificity in one arm, and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in WO 94/04690. For further details of generating bispecific antibodies see, for example, Suresh et al., Methods in Enzymology, 121:210 (1986).

According to another approach described in U.S. Pat. No. 5,731,168, the interface between a pair of antibody molecules can be engineered to maximize the percentage of heterodimers which are recovered from recombinant cell culture. The preferred interface comprises at least a part of the C_{H}3 domain of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Such antibodies have, for example, been proposed to target immune system cells to unwanted cells (U.S. Pat. No. 4,676,980), and for treatment of HIV infection (WO 91/00360, WO 92/200373, and EP 03089). Heteroconjugate antibodies may be made using any convenient cross-linking methods. Suitable cross-linking agents are well known in the art, and are disclosed in U.S. Pat. No. 4,676,980, along with a number of cross-linking techniques.

Techniques for generating bispecific antibodies from antibody fragments have also been described in the literature. For example, bispecific antibodies can be prepared using chemical linkage. Brennan et al., Science, 229: 81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Various techniques for making and isolating bispecific antibody fragments directly from recombinant cell culture have also been described. For example, bispecific antibodies have been produced using leucine zippers. Kostelny et al., J. Immunol., 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA, 90:6444-6448 (1993) has provided an alternative mechanism for making bispecific antibody fragments. The fragments comprise a heavy-chain variable domain (V_{H}) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See Gruber et al., J. Immunol., 152:5368 (1994).

Antibodies with more than two valencies are contemplated. For example, trispecific antibodies can be prepared. Tuft et al. J. Immunol. 147: 60 (1991).

(7) Other Amino Acid Sequence Modifications

Amino acid sequence modification(s) of the antibodies described herein are contemplated. For example, it may be desirable to improve the binding affinity and/or other biological properties of the antibody. Amino acid sequence variants of the antibody are prepared by introducing appropriate nucleotide changes into the antibody nucleic acid, or by peptide synthesis. Such modifications include, for example, deletions from, and/or insertions into and/or substitutions of, residues within the amino acid sequences of the antibody. Any combination of deletion, insertion, and substitution is made to arrive at the final construct, provided that the final construct possesses the desired characteristics. The amino acid changes also may alter post-translational processes of the antibody, such as changing the number or position of glycosylation sites.

A useful method for identification of certain residues or regions of the antibody that are preferred locations for mutagenesis is called “alanine scanning mutagenesis” as described by Cunningham and Wells Science, 244:1081-1085 (1989). Here, a residue or group of target residues are identified (e.g., charged residues such as arg, asp, his, lys, and glu) and replaced by a neutral or negatively charged amino acid (most preferably alanine or polyalanine) to affect the interaction of the amino acids with antigen. Those amino acid locations demonstrating functional sensitivity to the substitutions then are refined by introducing further or other variants at, or for, the sites of substitution. Thus, while the site for introducing an amino acid sequence variation is predetermined, the nature of the mutation per se need not be predetermined. For example, to analyze the performance of a mutation at a given site, ala scanning or random mutagenesis is conducted at the target codon or region and the expressed antibody variants are screened for the desired activity.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include antibody with an N-terminal methionyl residue or the antibody fused to a cytotoxic polypeptide. Other insertional variants of the antibody molecule include the fusion to the N- or C-terminus of the antibody to an enzyme (e.g. for ADEPT) or a polypeptide which increases the serum half-life of the antibody.

Another type of variant is an amino acid substitution variant. These variants have at least one amino acid residue in the antibody molecule replaced by a different residue. The sites of greatest interest for substitutional mutagenesis include the hypervariable regions, but FR alterations are also contemplated. Conservative substitutions are shown in Table 1 under the heading of “preferred substitutions”. If such substitutions result in a change in biological activity, then more substantial changes, denominated “exemplary substitutions” in the following table, or as further described below in reference to amino acid classes, may be introduced and the products screened.

| Original | Exemplary | Preferred |
|---|---|---|
| Residue | Substitutions | Substitutions |
| Ala (A) | val; leu; ile | val |
| Arg (R) | lys; gln; asn | lys |
| Asn (N) | gln; his; lys; arg | gln |
| Asp (D) | glu | glu |
| Cys (C) | ser | ser |
| Gln (Q) | asn | asn |
| Glu (E) | asp | asp |
| Gly (G) | pro; ala | ala |
| His (H) | asn; gln; lys; arg | arg |
| Ile (I) | leu; val; met; ala; phe; | leu |
| | norleucine | |
| Leu (L) | norleucine; ile; val; met; ala; | ile |
| | phe | |
| Lys (K) | arg; gln; asn | arg |
| Met (M) | leu; phe; ile | leu |
| Phe (F) | leu; val; ile; ala; tyr | leu |
| Pro (P) | ala | ala |
| Ser (S) | thr | thr |
| Thr (T) | ser | ser |
| Trp (W) | tyr; phe | tyr |
| Tyr (Y) | trp; phe; thr; ser | phe |
| Val (V) | ile; leu; met; phe; ala; | leu |
| | norleucine | |

Substantial modifications in the biological properties of the antibody are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Amino acids may be grouped according to similarities in the properties of their side chains (in A. L. Lehninger, in Biochemistry, second ed., pp. 73-75, Worth Publishers, New York (1975)): non-polar: Ala (A), Val (V), Leu (L), Ile (I), Pro (P), Phe (F), Trp (W), Met (M); uncharged polar: Gly (G), Ser (S), Thr (T), Cys (C), Tyr (Y), Asn (N), Gln (O); acidic: Asp (D), Glu (E); and basic: Lys (K), Arg (R), His (H).

Alternatively, naturally occurring residues may be divided into groups based on common side-chain properties: hydrophobic: Norleucine, Met, Ala, Val, Leu, Ile; neutral hydrophilic: Cys, Ser, Thr, Asn, Gln; acidic: Asp, Glu; basic: H is, Lys, Arg; residues that influence chain orientation: Gly, Pro; and aromatic: Trp, Tyr, Phe.

Non-conservative substitutions will entail exchanging a member of one of these classes for another class.

Any cysteine residue not involved in maintaining the proper conformation of the antibody also may be substituted, generally with serine, to improve the oxidative stability of the molecule and prevent aberrant crosslinking. Conversely, cysteine bond(s) may be added to the antibody to improve its stability (particularly where the antibody is an antibody fragment such as an Fv fragment).

A particularly preferred type of substitutional variant involves substituting one or more hypervariable region residues of a parent antibody (e.g. a humanized or human antibody). Generally, the resulting variant(s) selected for further development will have improved biological properties relative to the parent antibody from which they are generated. A convenient way for generating such substitutional variants involves affinity maturation using phage display. Briefly, several hypervariable region sites (e.g. 6-7 sites) are mutated to generate all possible amino substitutions at each site. The antibody variants thus generated are displayed in a monovalent fashion from filamentous phage particles as fusions to the gene III product of M13 packaged within each particle. The phage-displayed variants are then screened for their biological activity (e.g. binding affinity) as herein disclosed. In order to identify candidate hypervariable region sites for modification, alanine scanning mutagenesis can be performed to identify hypervariable region residues contributing significantly to antigen binding. Alternatively, or additionally, it may be beneficial to analyze a crystal structure of the antigen-antibody complex to identify contact points between the antibody and an IBD marker protein. Such contact residues and neighboring residues are candidates for substitution according to the techniques elaborated herein. Once such variants are generated, the panel of variants is subjected to screening as described herein and antibodies with superior properties in one or more relevant assays may be selected for further development.

Engineered antibodies with three or more (preferably four) functional antigen binding sites are also contemplated (U.S. Published Patent Application No. US2002/0004587 A1, Miller et al.).

Nucleic acid molecules encoding amino acid sequence variants of the antibody are prepared by a variety of methods known in the art. These methods include, but are not limited to, isolation from a natural source (in the case of naturally occurring amino acid sequence variants) or preparation by oligonucleotide-mediated (or site-directed) mutagenesis, PCR mutagenesis, and cassette mutagenesis of an earlier prepared variant or a non-variant version of the antibody.

### B.3 KITS OF THE INVENTION

The materials for use in the methods of the present invention are suited for preparation of kits produced in accordance with well known procedures. The invention thus provides kits comprising agents, which may include gene-specific or gene-selective probes and/or primers, for quantitating the expression of the disclosed genes for IBD. Such kits may optionally contain reagents for the extraction of RNA from samples, in particular fixed paraffin-embedded tissue samples and/or reagents for RNA amplification. In addition, the kits may optionally comprise the reagent(s) with an identifying description or label or instructions relating to their use in the methods of the present invention. The kits may comprise containers (including microtiter plates suitable for use in an automated implementation of the method), each with one or more of the various reagents (typically in concentrated form) utilized in the methods, including, for example, pre-fabricated microarrays, buffers, the appropriate nucleotide triphosphates (e.g., dATP, dCTP, dGTP and dTTP; or rATP, rCTP, rGTP and UTP), reverse transcriptase, DNA polymerase, RNA polymerase, and one or more probes and primers of the present invention (e.g., appropriate length poly(T) or random primers linked to a promoter reactive with the RNA polymerase).

### B.4 REPORTS OF THE INVENTION

The methods of this invention, when practiced for commercial diagnostic purposes generally produce a report or summary of the normalized expression levels of one or more of the selected genes. The methods of this invention will produce a report comprising a prediction of the clinical outcome of a subject diagnosed with an IBD before and after any surgical procedure to treat the IBD. The methods and reports of this invention can further include storing the report in a database. Alternatively, the method can further create a record in a database for the subject and populate the record with data. In one embodiment the report is a paper report, in another embodiment the report is an auditory report, in another embodiment the report is an electronic record. It is contemplated that the report is provided to a physician and/or the patient. The receiving of the report can further include establishing a network connection to a server computer that includes the data and report and requesting the data and report from the server computer.

The methods provided by the present invention may also be automated in whole or in part.

All aspects of the present invention may also be practiced such that a limited number of additional genes that are co-expressed with the disclosed genes, for example as evidenced by high Pearson correlation coefficients, are included in a prognostic or predictive test in addition to and/or in place of disclosed genes.

Having described the invention, the same will be more readily understood through reference to the following Examples, which is provided by way of illustration, and is not intended to limit the invention in any way.

### EXAMPLES

### Example 1

### Microarray Analysis

Clinically, IBD is characterized by diverse manifestations often resulting in a chronic, unpredictable course. Bloody diarrhea and abdominal pain are often accompanied by fever and weight loss. Anemia is common, as is severe fatigue. Joint manifestations ranging from arthralgia to acute arthritis as well as abnormalities in liver function are commonly associated with IBD. Patients with IBD also have an increased risk of colon carcinomas compared to the general population. During acute “attacks” of IBD, work and other normal activity are usually impossible, and often a patient is hospitalized.

Although the cause of IBD remains unknown, several factors such as genetic, infectious and immunologic susceptibility have been implicated. IBD is much more common in Caucasians, especially those of Jewish descent. The chronic inflammatory nature of the condition has prompted an intense search for a possible infectious cause. Although agents have been found which stimulate acute inflammation, none has been found to cause the chronic inflammation associated with IBD. The hypothesis that IBD is an autoimmune disease is supported by the previously mentioned extraintestinal manifestation of IBD as joint arthritis, and the known positive response to IBD by treatment with therapeutic agents such as adrenal glucocorticoids, cyclosporine and azathioprine, which are known to suppress immune response. In addition, the GI tract, more than any other organ of the body, is continuously exposed to potential antigenic substances such as proteins from food, bacterial byproducts (LPS), etc. The subtypes of IBD are UC and CD.

CD differs from UC in that the inflammation extends through all layers of the intestinal wall and involves mesentery as well as lymph nodes. CD may affect any part of the alimentary canal from mouth to anus. The disease is often discontinuous, i.e., severely diseased segments of bowel are separated from apparently disease-free areas. In CD, the bowel wall also thickens which can lead to obstructions. In addition, fistulas and fissures are not uncommon.

Both UC and CD are typically diagnosed by endoscopy, which shows the afflicted areas. However, the use of microarray technology has shed light on the molecular pathology of UC. A study published in 2000 used microarray technology to analyze eight UC patients. (Dieckgraefe et al., Physiol. Genomics 2000; 4:1-11). 6500 genes were analyzed in this study and the results confirmed increased expression of genes previously implicated in UC pathogenesis, namely IL-1, IL-IRA and IL-8. Endoscopic diagnosis coupled with the ability to take pinch mucosal biopsies have allowed investigators to further diagnose UC using microarray analysis, and allowed investigators to analyze afflicted tissue against normal tissue and to analyze tissue from a larger range of patients with varying degrees of severity. Biopsies of macroscopically unaffected areas of the colon and terminal ileum were microarrayed. (Langman et al., Gastroent. 2004; 127:26-40). Langman analyzed 22,283 genes and found that genes involved in cellular detoxification, such as pregnane X receptor and MDR1 were significantly downregulated in the colon of patients with UC, but there was no change in expression of these genes in CD patients.

Nucleic acid microarrays, often containing thousands of gene sequences, are useful for identifying differentially expressed genes in diseased tissues as compared to their normal counterparts. Using nucleic acid microarrays, test and control mRNA samples from test and control tissue samples are reverse transcribed and labeled to generate cDNA probes. The cDNA probes are then hybridized to an array of nucleic acids immobilized on a solid support. The array is configured such that the sequence and position of each member of the array is known. For example, a selection of genes known to be expressed in certain disease states may be arrayed on a solid support. Hybridization of a labeled probe with a particular array member indicates that the sample from which the probe was derived expresses that gene. If the hybridization signal of a probe from a test (for example, disease tissue) sample is greater than hybridization signal of a probe from a control, normal tissue sample, the gene or genes overexpressed in the disease tissue are identified. The implication of this result is that an overexpressed protein in a disease tissue is useful not only as a diagnostic marker for the presence of the disease condition, but also as a therapeutic target for treatment of the disease condition.

The methodology of hybridization of nucleic acids and microarray technology is well known in the art. In one example, the specific preparation of nucleic acids for hybridization and probes, slides, and hybridization conditions are all detailed in PCT Patent Application Serial No. PCT/US01/10482, filed on Mar. 30, 2001 and which is herein incorporated by reference.

Microarray analysis was used to find genes that are overexpressed in CD as compared to normal bowel tissue. For this study, sixty seven patients with CD and thirty-one control patients undergoing colonoscopy were recruited. Patient symptoms were evaluated at the time of colonoscopy using the simple clinical colitis activity index (SCCAI). (Walmsley et al., Gut. 1998; 43:29-32). Quiescent disease showing no histological inflammation was defined as a SCCAI of 2 or less. Active disease with histologially acute or chronic inflammation was defined as a SCCAI of greater than 2. The severity of the CD itself was determined by the criteria of Leonard-Jones. (Lennard-Jones Scand. J. Gastroent. 1989; 170:2-6). The CD patients provided well phenotyped biopsies for analysis of inflammatory pathways of CD at the molecular level, thus identifying novel candidate genes and potential pathways for therapeutic intervention. Paired biopsies were taken from each anatomical location.

All biopsies were stored at −70° C. until ready for RNA isolation. The biopsies were homogenized in 600 μl of RLT buffer (+BME) and RNA was isolated using Qiagen™ Rneasy Mini columns (Qiagen) with on-column DNase treatment following the manufacturer's guidelines. Following RNA isolation, RNA was quantitated using RiboGreen™ (Molecular Probes) following the manufacturer's guidelines and checked on agarose gels for integrity. Appropriate amounts of RNA were labeled for microarray analysis and samples were run on proprietary Genentech microarray and Affymetrics™ microarrays. Genes were compared whose expression was upregulated in UC tissue vs normal bowel, matching biopsies from normal bowel and CD tissue from the same patient. The results of this experiment showed that the nucleic acids as shown in Tables 1A, 1B, and 2A (provided above) are differentially expressed in CD and/or UC tissue in comparison to normal tissue.

The genes listed in Table 1A-1B demonstrated a minimum 1.5 fold difference in expression and also acceptable probe hybridization strength was observed. The genes listed in Table 2 were found to have a minimum within-group Pearson correlation of approximately 0.65 and a three-fold upregulation of gene expression was observed.

More specifically, the SEQ ID NOS listed in Tables 1A and 2 represent polynucleotides and their encoded polypeptide which are significantly up-regulated/overexpressed in CD and/or UC.

The SEQ ID NO listed in Table 1B represents a polynucleotide and its encoded polypeptide which is significantly down-regulated/under-expressed in CD and/or UC.

SEQ ID NOS: listed in Table 3A represent polynucleotides and their encoded polypeptide which are significantly up-regulated/overexpressed in UC.

SEQ ID NOS: listed in Table 3B represent polynucleotides and their encoded polypeptide which are significantly down-regulated/underexpressed in UC.

### Example 2

### Characterisation of Distinct Intestinal Gene Expression Profiles in Ulcerative Colitis by Microarray Analysis

Microarray analysis allows a comprehensive picture of gene expression at the cellular level. The aim of this study was to investigate differential intestinal gene expression in patients with ulcerative colitis (UC) and controls.

Methods: 67 UC and 31 control subjects-23 normal and 8 inflamed non-inflammatory bowel disease patients were studied. Paired endoscopic biopsies were taken from 5 specific anatomical locations for RNA extraction and histology. 41058 expression sequence tags were analyzed in 215 biopsies using the Agilent platform. Confirmation of results was undertaken by real time PCR and immunohistochemistry. Results: In healthy control biopsies, cluster analysis showed differences in gene expression between the right and left colon. (χ²=25.1, p<0.0001). Developmental genes HOXA13, (p=2.3×10⁻¹⁶), HOXB13 (p<1×10⁻⁴⁵), GLI1 (p=4.0×10⁻²⁴), and GLI3 (p=2.1×10⁻²⁸) primarily drove this separation. When all UC biopsies and control biopsies were compared, 143 sequences had a fold change of >1.5 in the UC biopsies (0.01>p>10⁻⁴⁵) and 54 sequences had a fold change of <−1.5 (0.01>p>10⁻²⁰). Differentially upregulated in UC genes included SAA1 (p<10⁻⁴⁵) the alpha defensins, DEFAS&6 (p=0.00003 and p=6.95×10⁻⁷ respectively), MMP3 (p=5.6×10⁻¹⁰) and MMP7 (p=2.3×10⁻⁷). Increased DEFAS&6 expression was further characterized to Paneth cell metaplasia by immunohistochemistry and in-situ hybridization. Sub-analysis of the IBD2 & IBDS loci, and the ABC transporter genes revealed a number of differentially regulated genes in the UC biopsies. Conclusions: These data implicate a number of novel gene families, as well as established candidate genes in the pathogenesis of UC, and may allow characterisation of potential therapeutic targets.

The aim of the current study was to use microarray gene expression analysis to investigate genome wide expression in endoscopic mucosal biopsies of patients with UC and controls. In order to resolve previous inconsistencies and to further delineate inflammatory pathways in UC, substantially more patients and biopsies were included than in previous studies.

Materials and Methods

Patients and Controls. Sixty seven patients with UC and 31 control patients who were undergoing colonoscopy were recruited. Their demographics are shown in Table 4.

TABLE 4

| | UC |
|---|---|
| Number of patients | 67 |
| Male/Female | 33/34 |
| Median age at diagnosis (years) | 37 |
| Median duration of follow up (years) | 7.8 |
| Disease Group | |
| New Diagnosis (1) | 8 |
| Quiescent disease (2) | 41 |
| Active disease (3) | 18 |
| Disease extent at time of Endoscopy | |
| Proctitis | 15 |
| L sided colitis | 27 |
| Extensive colitis | 25 |
| Current Smoker | 6 |
| Family history of IBD | 5 |
| 5 ASA Therapy | 40 |
| Corticosteroid therapy | 10 |
| Immunosuppressant therapy (AZA, 6MP, MTX, MMF) | 11 |

Sixty seven patients with UC and 31 control patients who were undergoing colonoscopy were recruited (Table 4). All UC patients attended the clinic at the Western General Hospital, Edinburgh and the diagnosis of UC adhered to the criteria of Lennard-Jones. (Lennard-Jones J E. Scand J Gastroenterol Suppl 1989; 170:2-6) Phenotypic data were collected by interview and case-note review and comprised of demographics, date of diagnosis, disease location, disease behavior, progression, extra-intestinal manifestations, surgical operations, current medication, smoking history, joint symptoms, family history and ethnicity. At the time of colonoscopy patients symptoms were evaluated using the simple clinical colitis activity index (SCCAI). (Walmsley et. al. Gut. 1998; 43:29-32)

Patients were recorded as having a ‘new diagnosis’ of UC if the colonoscopy took place at the time of their index presentation and they had had less than 24 hours of oral/IV therapy. Quiescent disease was defined as a SCCAI of 2 or less and histology showing no inflammation or mild chronic inflammation and active disease was defined as a SCCAI of greater than 2 and histology showing acute or chronic inflammation.

Eleven of the controls were male, 20 were female with a median age of 43 at the time of endoscopy. Six of the controls had normal colonoscopies for colon cancer screening, 9 controls had symptoms consistent with irritable bowel syndrome and had a normal colonoscopic investigation and 7 patients had a colonoscopy for another indication and histologically normal biopsies were obtained. Eight control patients had abnormal inflamed colonic biopsies (1 pseudomembranous colitis, 1 diverticulitis, 1 amoebiasis, 2 microscopic colitis, 1 eosoinophilic infiltrate, 2 scattered lymphoid aggregates and a history of gastroenteritis). Written informed consent was obtained from all patients. Lothian Local Research Ethics Committee approved the study protocol: REC 04/S 1103/22.

Biopsy Collection. Anatomical location was confirmed by an experienced operator, distance of endoscope insertion and endoscope configuration using a Scope Guide™. Paired biopsies were taken from each anatomical location. One biopsy was sent for histological examination and the other was snap frozen in liquid nitrogen for RNA extraction. Each biopsy was graded histologically, by an experienced gastrointestinal pathologist as having no evidence on inflammation, biopsies with evidence of chronic inflammation and predominately chronic inflammatory cell infiltrate or simply those with acute inflammation and an acute inflammatory cell infiltrate. One hundred and thirty nine paired UC biopsies and 76 paired control biopsies were collected. The number of paired biopsies in UC patients and controls from each anatomical location are shown in Table 5.

TABLE 5

| | UC (n = 67) | Controls (n = 31) |
|---|---|---|
| Total number of paired biopsies | 139 | 76 |
| Terminal Ileum | 4 | 6 |
| Ascending colon | 33 | 17 |
| Descending colon | 35 | 23 |
| Sigmoid colon biopsies. | 57 | 27 |
| Removed from analysis | 10 | 3 |

RNA Isolation. The biopsies weighed between 0.2 mg and 16.5 mg with a median weight of 5.5 mg. Total RNA was extracted from each biopsy using the micro total RNA isolation from animal tissues protocol (Qiagen, Valencia, Calif.), according to the manufacturer's instructions. To evaluate purity and integrity 1 μL of total RNA was assessed each sample with the Agilent technologies 2100 bioanalyzer using the Pico LabChip reagent set (Agilent Technologies, Palo Alto, Calif.).

Microarray Analysis.

1 μg of total RNA was amplified using the Low RNA Input Fluorescent Linear Amplification protocol (Agilent Technologies, Palo Alto, Calif.). A T7 RNA polymerase single round of linear amplification was carried out to incorporate Cyanine-3 and Cyanine-5 label into cRNA. The cRNA was purified using the RNeasy Mini Kit (Qiagen, Valencia, Calif.). 1 μl of cRNA was quantified using the NanoDrop ND-1000 spectrophotometer (NanoDrop Technologies, Wilmington, Del.).

750 ng of Universal Human Reference (Stratagene, La Jolla, Calif.) cRNA labeled with Cyanine-3 and 750 ng of the test sample cRNA labeled with Cyanine-5 were fragmented for 30 minutes at 60° C. before loading onto Agilent Whole Human Genome microarrays (Agilent technologies, Palo Alto, Calif.). The samples were hybridized for 18 hours at 60° C. with constant rotation. Microarrays were washed, dried and scanned on the Agilent scanner according to the manufacturer's protocol (Agilent technologies, Palo Alto, Calif.). Microarray image files were analyzed using Agilent's Feature Extraction software version 7.5 (Agilent Technologies, Palo Alto, Calif.). The distribution of log intensities for each sample was plotted and outlier samples (i.e. greater than 2 standard deviations from the mean) were excluded from analysis. 10 UC samples and 3 control samples were designated as outliers using these criteria.

Real Time PCR.

Confirmation real time PCR analysis was carried out on 8 genes—SAA1, IL8, DEFA5, DEFA6, MMP3, MMP1, S100A8 and TLR4. Ten healthy control sigmoid colon biopsies with normal histology, 9 quiescent UC sigmoid biopsies and 11 UC sigmoid biopsies with an acute (6 biopsies) or chronic (5 biopsies) inflammatory cell infiltrate were selected to represent the different disease groups after stratifying to represent a range of SAA1 and IL-8 expression.

Prior to RTPCR analysis 1 RNA amplification cycle was carried out using the MessageAmp™ II aRNA Amplification Kit protocol (Ambion technologies, Austin, Tex.). Reverse transcription PCR was then performed on 50 ng of RNA using Stratagene model MX4000 (La Jolla, Calif., USA). TaqMan primers and probes were manufactured in house (Genentech Inc, South San Francisco, Calif.). The sequences for the forward probe, the reverse probe and the TaqMan probe were as follows—

SAA1,
[SEQ ID NO: 235]
forward-agcgatgccagagagaata,
reverse-ggaagtgattggggtctttg,
Taq-ctttggccatggtgcggagg,
IL-8,
[SEQ ID NO: 236]
forward-actcccagtcttgtcattgc,
reverse-caagtttcaaccagcaagaa,
Taq-tgtgttggtagtgctgtgttgaattacgg,
DEFA5,
[SEQ ID NO: 237]
forward-gctacccgtgagtccctct,
reverse-tcttgcactgctttggtttc,
Taq-tgtgtgaaatcagtggccgcct,
DEFA6,
[SEQ ID NO: 238]
forward-agagctttgggctcaacaag,
reverse-atgacagtgcaggtcccata,
Taq-cacttgccattgcagaaggtcctg,
MMP3,
[SEQ ID NO: 239]
forward-aagggaacttgagcgtgaat,
reverse-gagtgcttccccttctcttg,
Taq-ggcattcaaatgggctgctgc,
MMP7,
[SEQ ID NO: 240]
forward-cacttcgatgaggatgaacg,
reverse-gtcccatacccaaagaatgg,
Taq-ctggacggatggtagcagtctaggga,
S100A8,
[SEQ ID NO: 241]
forward-ttgaccgagctggagaaag,
treverse-tcaggtcatccctgtagacg,
Taq-ccctgataaaggggaatttccatgc
and
TLR4,
[SEQ ID NO: 242]
forward-agagccgctggtgtatcttt,
reverse-ccttctgcaggacaatgaag,
Taq-tggcagtttctgagcagtcgtgc.

PCR conditions comprised of 48° C. for 30 minutes, 95° C. hold for 10 minutes, followed by 40 cycles of 30 second 95° C. melt and 1 minute 60° C. anneal/extend. Absolute quantification of product was calculated by normalizing to RPL19. Results were analyzed using SAS and JMP software (SAS, North Carolina).

In Situ Hybridization for Defensin Alpha 5.

PCR primers were designed to amplify a 318 bp fragment of DEFA5 spanning from nt 55-372 of NM_{—}021010 (upper-5′ catcccttgctgccattct [SEQ ID NO:243] and lower-5′ gaccttgaactgaatcttgc [SEQ ID NO:244]). Primers included extensions encoding 27-nucleotide T7 or T3 RNA polymerase initiation sites to allow in vitro transcription of sense or antisense probes, respectively, from the amplified products. Endoscopic biopsies were fixed in 10% neutral buffered formalin and paraffin-embedded. Sections 5 μm thick were deparaffinized, deproteinated in 10 ug/ml Proteinase K (Amresco) for 45 minutes at 37° C., and further processed for in situ hybridization as previously described. (Jubb et. al. Methods Mol. Biol. 2006; 326:255-264) ³³P-UTP labeled sense and antisense probes were hybridized to the sections at 55° C. overnight. Unhybridized probe was removed by incubation in 20 μg/ml RNase A for 30 min at 37° C., followed by a high stringency wash at 55° C. in 0.1×SSC for 2 hours and dehydration through graded ethanols. The slides were dipped in NTB nuclear track emulsion (Eastman Kodak), exposed in sealed plastic slide boxes containing desiccant for 4 weeks at 4° C., developed and counterstained with hematoxylin and eosin.

Immunohistochemistry for Rabbit Anti-Human Lysozyme and Rabbit Anti-Human Defensin Alpha 6

Formalin fixed paraffin embedded tissue sections were rehydrated prior to quenching of endogenous peroxidase activity (KPL, Gathersburg, Md.) and blocking of avidin and biotin (Vector, Burlingame, Calif.). Sections were blocked for 30 minutes with 10% normal goat serum in PBS with 3% BSA. Tissue sections were then incubated with primary antibodies for 60 minutes at room temperature, biotinylated secondary antibodies for 30 min, and incubated in ABC reagent (Vector, Burlingame, Calif.) for 30 minutes followed by a 5 minute incubation in metal enhanced DAB (Pierce, Rockford, Ill.). The sections were then counterstained with Mayer's hematoxylin. Primary antibodies used were rabbit anti-human lysozyme at 5.0 μg/m1 (Dako, Carpinteria, Calif.) and rabbit anti-human DEFA6 at 5.0 μg/ml (Alpha Diagnostics, SanAntonio, Tex.). Secondary antibody used was biotinylated goat anti-rabbit IgG at 7.5 μg/m1 (Vector, Burlingame, Calif.). DEFA6 alpha staining required pre-treatment with Target Retrieval High pH (Dako, Carpenteria, Calif.) at 99° C. for 20 minutes, lysozyme staining did not require pretreatment. All other steps were performed at room temperature.

Data Analysis. Microarray data were analyzed using the Rosetta Resolver software (Rosetta Inpharmatics, Seattle). Statistical significance of the microarray data was determined by Student's unpaired t test. p<0.01 and a fold change of greater or less than 1.5 were considered statistically significant. Fold change data was calculated using the Rosetta Resolver software. Gene ontology was analyzed using Ingenuity software (Ingenuity Systems, Mountain View, Calif.). The Mann-Whitney U test was used to analyze the real time PCR data. p<0.05 was considered significant.

Results

Influence of anatomical location on gene expression in the healthy colon and terminal ileum. 56 histologically normal biopsies from control patients were analyzed by unsupervised hierarchical clustering. Clear separation by anatomical location was observed on one side of the dendrogram 25/25 biopsies were from the left colon (descending colon or sigmoid colon) where as on the other side of the dendrogram 20/31 biopsies were from the ascending colon (χ²=25.1, p<0.0001) (FIG. 1). 6/6 of the terminal ileal biopsies were clustered together. Biopsies from individual patients did not cluster together. The genes driving the differential expression between the right and left colon that were causing the observed clustering were predominately involved in the embryological development of the GI tract—HOXA13, (FC +4.93, p=2.3×10⁻¹⁶), HOXB13 (FC +16.96, p<1×10⁻⁴⁵), GLI1 (FC +2.2, p=4.0×10⁻²⁴), and GLI3 (FC +2.3, p=2.1×10⁻²⁸) were all upregulated in the left colon.

Analysis of Expression in UC and Control Biopsies.

Using unsupervised hierarchical clustering we were unable to differentiate between biopsies from UC patients and controls patients. In addition no clustering based on the inflammation status of the biopsies was observed. The only clustering that was observed was with biopsies from the terminal ileum where both UC and control biopsies clustered together. When all of the UC biopsies (129) and control biopsies (73) were compared, 143 sequences had a fold change of greater than 1.5 in the UC biopsies (0.01>p>10⁻⁴⁵) and 54 sequences had a fold change of less than 1.5 (0.01>p>10⁻²⁰) (data not shown).

Serum amyloid A1 (SAA1) was the most up regulated gene (Fold change (FC) +8.19, p<10⁻⁴⁵). Other notably upregulated genes were S100A8 (FC +3.50, p=2.3×10⁻¹⁷), S100A9 (FC +3.06, p=4.1×10⁻¹³), the alpha defensins, alpha 5 (DEFAS) (FC +3.25, p=0.00003), alpha 6 (DEFA6) (FC +2.18, p=6.95×10⁻⁷) and the matrix metalloproteinases MMP3 (FC +2.17, p=5.6×10⁻¹⁰) and MMP7 (FC +2.29, p=2.3×10⁻⁷).

A list of the genes found to be differentially expressed in UC patients when compared to normal patients can be found in Tables 1A, 1B, and 2A as described above.

The differential gene expression of a number of candidate genes across more than one experiment is shown in Table 6. Table 6 shows fold changes and p values are shown in a number of different genes in four different experiments. The number of biopsies analyzed in each experiment is shown in brackets. Significant consistent changes in expression across more than one experiment were observed for the genes of interest in this table.

TABLE 6

| | | | Non-inflamed | | | | | |
|---|---|---|---|---|---|---|---|---|
| | | | UC | | Inflamed UC | | Inflamed UC | |
| | All UC (129 | | sigmoid (22) v | | sigmoid (35) v | | sigmoid (35) v | |
| | biopsies) v | | non-inflamed | | inflamed | | non-inflamed | |
| | controls (73 | | control sigmoid | | control sigmoid | | sigmoid UC | |
| Genes | biopsies) | | (18) | | (8) | | (22) | |
| Analyzed | Fold change | p value | Fold change | p value | Fold change | p value | Fold change | p value |
| SAA1 | +8.19 | <10⁻⁴⁵ | +2.0 | 0.00024 | +17.5 | 2.9 × 10⁻²¹ | +16.51 | <10⁻⁴⁵ |
| Def alpha 5 | +3.25 | 0.00003 | +1.02 | 0.89 | +7.27 | 6.3 × 10⁻³⁰ | +8.44 | <10⁻⁴⁵ |
| Def alpha 6 | +2.18 | 6.95 × 10⁻⁷ | −1.09 | 0.34 | +4.41 | 9.7 × 10⁻⁹ | +6.72 | 4.16 × 10⁻¹⁹ |
| S100A8 | +3.50 | 2.3 × 10⁻¹⁷ | +1.21 | 0.19 | +9.75 | 2.4 × 10⁻²⁴ | +6.84 | 1.16 × 10⁻¹⁹ |
| S100A9 | +3.06 | 4.1 × 10⁻¹³ | +1.05 | 0.16 | +7.53 | 6.4 × 10⁻¹² | +7.11 | 1.96 × 10⁻³² |
| MMP3 | +2.17 | 5.6 × 10⁻¹⁰ | −1.55 | 0.0088 | +11.0 | 1.22 × 10⁻³⁷ | +8.15 | 2.32 × 10⁻³⁵ |
| MMP7 | +2.29 | 2.3 × 10⁻⁷ | +1.16 | 0.080 | +7.31 | 4.9 × 10⁻²⁴ | +5.53 | 1.01 × 10⁻²³ |
| IL8 | +2.05 | 4.2 × 10⁻¹¹ | +1.10 | 0.26 | +6.36 | 9.27 × 10⁻¹⁷ | +7.24 | 8.42 × 10⁻¹⁹ |
| TLR4 | +1.34 | 4.5 × 10⁻⁷ | +1.15 | 0.18 | +1.50 | 0.0044 | +1.54 | 0.00073 |
| TNIP3 | +8.02 | 1.1 × 10⁻¹⁷ | −1.30 | 0.20 | +7.53 | 2.93 × 10⁻¹³ | +10.5 | 1 × 10⁻³⁸ |
| CCL20 | +1.30 | 0.00011 | +1.25 | 0.020 | +1.79 | 0.00002 | +2.36 | 4.68 × 10⁻¹¹ |
| ABCB1 | −1.32 | 0.00091 | +1.10 | 0.40 | −1.82 | 5.6 × 10⁻⁶ | −1.92 | 9.0 × 10⁻¹⁰ |
| HLA-DRB1 | +1.03 | 0.88 | −3.0 | 0.0010 | +3.30 | 0.033 | +2.67 | 0.0011 |
| TSLP | −1.12 | 0.31 | −2.73 | 2.7 × 10⁻¹⁰ | −1.15 | 0.61 | +1.23 | 0.092 |

Gene ontology analysis involving the genes differentially expressed between the UC and control biopsies showed a preponderance of differentially expressed genes were involved in immune response (48 genes out of a total of 679 genes classified under immune response, p=2.1×10⁻⁹, OR 2.61, CI 1.85-3.56) and response to wounding (30 genes out of a total of 359 genes classified under response to wounding, p=6.42×10⁻⁹, OR 3.14, CI 2.09-4.53) when biological systems were considered.

Analysis of expression in sigmoid colon biopsies in patients with quiescent UC and non-inflamed control biopsies. To compare expression in biopsies without an acute inflammatory signal and to remove the effect of anatomical variation, 22 biopsies from the sigmoid colon with no histological evidence of inflammation from patients with UC were compared to 18 histologically normal control sigmoid colon biopsies. 102 sequences had a fold change greater than 1.5 (0.01>p>4.77×10⁻¹³) and 84 sequences had a fold change of less than 1.5 (0.01>p>1.8×10⁻²¹) (data not shown).

Upregulated genes included defensin beta 14 (FC +2.11, p=0.00002) and SAA1 (FC +2.01, p=0.00024). Interesting genes that were down regulated included HLA-DRB1 (FC −3.0, p=0.0010) and TSLP (FC −2.73, p=2.7×10⁻¹⁰) (Table 6).

Analysis of expression in sigmoid colon biopsies in patients with active UC and inflamed control biopsies. Expression of 35 histologically inflamed sigmoid biopsies from patients with UC were compared to 8 histologically inflamed control sigmoid biopsies. Reflecting the more severe inflammation in the UC biopsies a number of genes involved in the acute inflammatory response were upregulated in the UC biopsies v the control biopsies—SAA1 (FC +17.5, p=2.9×10⁻²¹), MMP3 (FC +11.0, p=1.22×10⁻³⁷), MMP7 (FC +7.31, p=4.9×10⁻²⁴) and IL-8 (FC +6.36, p=9.27×10⁻¹⁷) (Table 6). Overall 623 sequences had a fold change of greater than 1.5 and 509 sequences had a fold change of −1.5 or less (p<0.01) (data not shown).

Inflamed versus non-inflamed UC sigmoid colon biopsies. When expression signals were compared between 35 histologically inflamed and 22 non-inflamed sigmoid colon UC biopsies 700 sequences had a fold change of greater than 1.5 (0.01>p>1×10⁻⁴⁵) and 518 sequences (0.01>p>1×10⁻⁴⁵) had a fold change of less than 1.5 in the inflamed biopsies (data not shown).

Notably upregulated genes included SAA1 (FC +16.51, p=<10⁻⁴⁵), TNFAIP3 interacting protein 3 (TNIP3) (FC +10.5, p=1×10⁻³⁸), DEFAS (FC +8.44, p=<10⁻⁴⁵), DEFA6 (FC +6.72, p=4.16×10⁻¹⁹) and regenerating islet-derived 3 gamma (REG3γ) (FC +6.99, p=<10⁻⁴⁵).

Analysis of Specific Gene Families-Alpha Defensins 5 and 6.

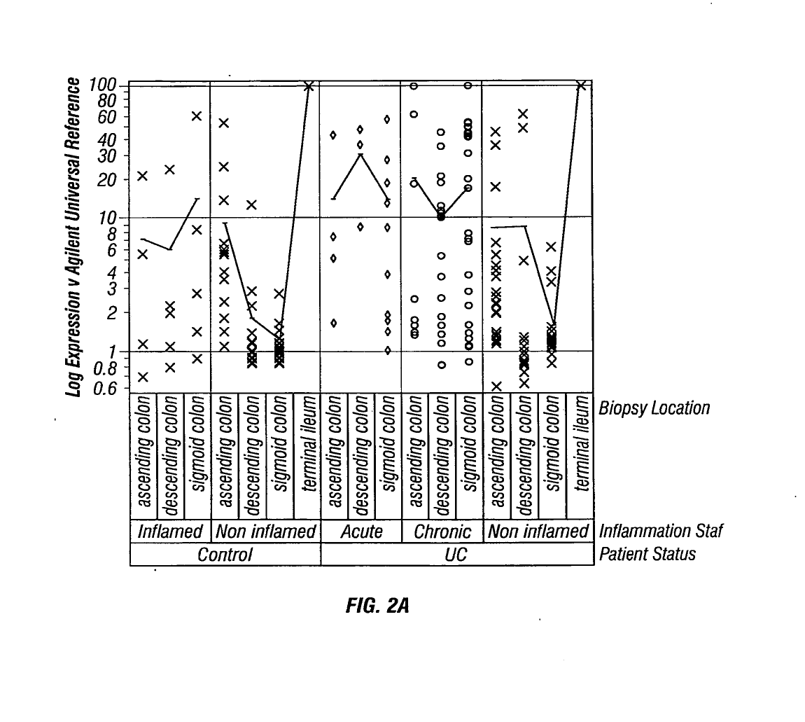

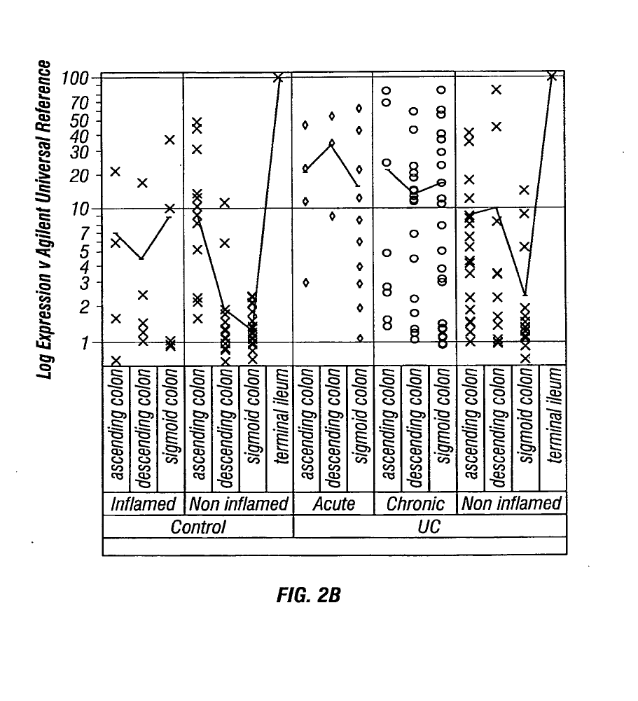

Expression of a number of genes of interest was further analysed, taking into consideration anatomical location and degree of inflammation in the UC samples. When DEFA5 and DEFA6 were analysed expression in the normal controls and the non-inflamed UC biopsies was similar across the different anatomical locations with there being high expression in the terminal ileum, and expression decreasing as the biopsy location became more distal in the colon (FIG. 2).

In FIG. 2, the expression of each array sample is plotted against the Agilent universal reference. Each endoscopic biopsy has been separated by patient status, biopsy inflammation status and anatomical location. The mean expression levels for each anatomical location are linked in blue. High alpha defensin 5 (panel A) and 6 (B) (DEFA5 and DEFA6) expression levels are seen in the terminal ileum of the controls and the non inflamed UC samples. The expression in these 2 groups decreased the more distally in the colon the biopsies were retrieved from. In the acute and chronically inflamed UC samples and to a lesser extent in the inflamed control samples there was a marked increase in DEFA5 and DEFA6 expression throughout the ascending, descending and sigmoid colon—sigmoid colon inflamed v non-inflamed UC samples (FC +8.44, p=<10⁻⁴⁵) for DEFA5, (FC +6.72, p=4.16×10⁻¹⁹) for DEFA6.

In the acute and chronically inflamed UC biopsies there was marked upregulation of DEFA5 and DEFA6 expression throughout the ascending, descending and sigmoid colon (Table 6).

Matrix Metalloproteinases 3 and 7.

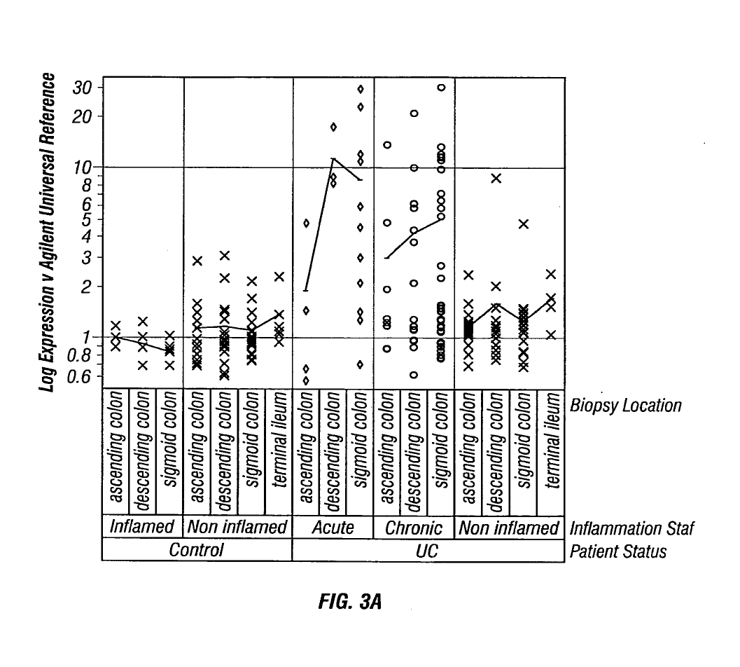

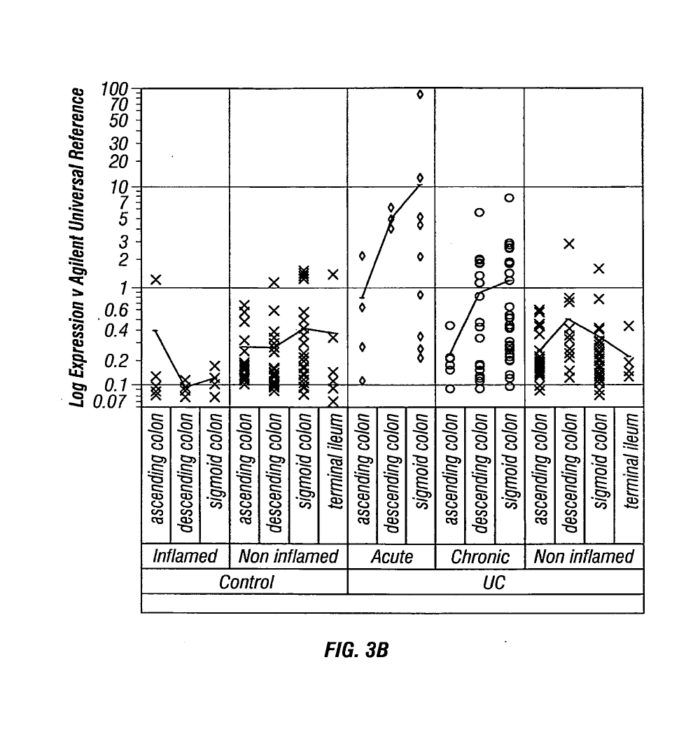

Increased expression of MMP3 and MMP7 was observed in the acutely and chronically inflamed UC biopsies when compared to the non-inflamed UC biopsies—sigmoid colon inflamed v non-inflamed UC samples MMP3 (FC +8.15, p=2.3×10⁻³⁵) and MMP7 (FC +5.53, p=1.0×10⁻²³) (FIG. 3).

In FIG. 3, the expression of each array sample is plotted against the Agilent universal reference. Each endoscopic biopsy has been separated by patient status, biopsy inflammation status and anatomical location. The mean expression levels for each anatomical location are linked in blue. Increased expression of MMP3 (panel A) and MMP7 (B) was observed in the acutely and chronically inflamed UC biopsies when compared to the non inflamed UC biopsies—sigmoid colon inflamed v non inflamed UC samples MMP3 (FC +8.15, p=2.3×10⁻³⁵) and MMP7 (FC +5.53, p=1.0×10⁻²³). In contrast when the inflamed and non-inflamed control samples were analysed, a decrease in the expression levels of MMP3 and MMP7 in the inflamed control biopsies was observed (FC −1.62, p=0.012 and FC −2.0, p=0.0002 respectively).

ATP-binding cassette (ABC) transporter family and the Xenobiotic-transcription regulators. To further investigate this gene family, expression patterns from probes representing 48 transcriptional genes and their key mediators (PXR, FXR, LXR and CAR) were analysed. When these genes were compared in all the UC and control biopsies, 7 genes were found to significantly down regulated in the UC samples when compared to the control samples-ABCA1 (p=0.01), ABCA8 (p=0.0064), ABCB1 (p=0.0091), ABCC6 (p=0.0050), ABCB7 (p=0.0068), ABCF1 (p=0.0005) and ABCF2 (p<0.00001). Only one probe representing ABCB2 was significantly upregulated in UC (p=0.0048).

A number of ABC genes were found to be down-regulated in IBD patients as compared to normal patients (data not shown). The changes observed in ABCB1 expression appeared to be primarily driven by the inflamed UC biopsies which were significantly downregulated when compared to the non-inflamed UC biopsies in the sigmoid colon (FC −1.82, p=5.6×10⁻⁶)(Table 6). Of interest, no difference in the expression of PXR between UC and controls was observed in any of the analysis including disease location and activity.

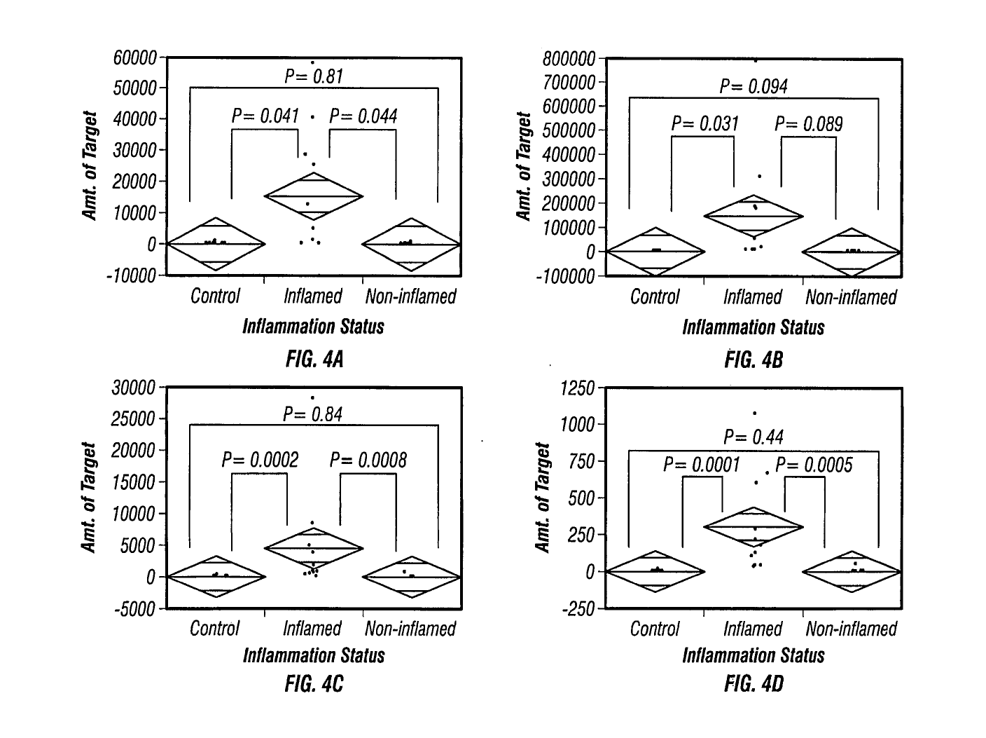

RTPCR Analysis. In the case of 8 genes implicated by microarray expression results, confirmatory real time PCR analysis using 10 healthy control colon sigmoid biopsies with normal histology, 9 quiescent UC sigmoid biopsies and 11 UC sigmoid biopsies with an acute (6 biopsies) or chronic (5 biopsies) inflammatory cell infiltrate was undertaken. Increased SAA1 expression in the inflamed UC sigmoid colon biopsies compared to the normal control sigmoid colon biopsies and the non-inflamed UC sigmoid colon biopsies (p=0.041 and p=0.044 respectively) was observed. Elevated IL-8 expression was also confirmed in the inflamed UC sigmoid biopsies when compared to the control sigmoid biopsies (p=0.031) and a trend was observed towards there being higher IL-8 expression in the inflamed UC sigmoid colon biopsies when compared to the non-inflamed UC biopsies (p=0.089) (FIG. 4).

FIG. 4 shows the real time PCR expression data comparing expression in 10 healthy control sigmoid biopsies with normal histology, 9 quiescent UC sigmoid biopsies and 11 UC sigmoid biopsies with an acute or chronic inflammatory cell infiltrate. Expression of SAA1 (A), IL-8 (B), defensin alpha 5 (C) and defensin alpha (D) were compared between the control and the inflamed and non-inflamed UC biopsies. Standard error bars are illustrated in green in each graph.

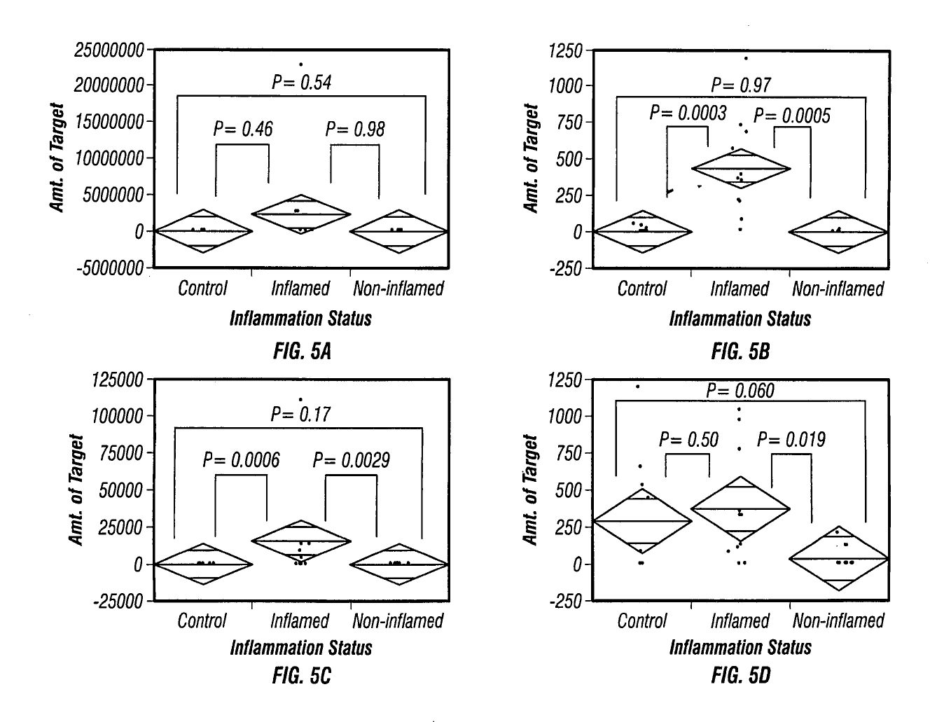

Increased expression of DEFAS and DEFA6 in the inflamed UC sigmoid colon biopsies when compared to the non-inflamed UC sigmoid colon biopsies (p=0.0008 and p=0.0005 respectively) and the control sigmoid colon biopsies (p=0.0002 and p=0.0001 respectively) was observed (FIG. 4). Increased expression in the inflamed UC sigmoid colon biopsies when compared to the non inflamed UC sigmoid colon biopsies was also observed when MMP7, (p=0.0005), S100A8, (p=0.0029) and TLR4, (p=0.019) were examined (FIG. 5).

FIG. 5 shows the real time PCR expression data comparing expression in 10 healthy control sigmoid biopsies with normal histology, 9 quiescent UC sigmoid biopsies and 11 UC sigmoid biopsies with an acute or chronic inflammatory cell infiltrate. Expression of MMP3 (A), MMP7 (B), S100A8 (C) and TLR4 (D) were compared between the different patient groups. Standard error bars are illustrated in green in each graph. No significant change in MMP3 expression was observed when the inflamed, non-inflamed UC sigmoid colon biopsies and the control sigmoid colon biopsies were analysed.

In-Situ Hybridization and Immunohistochemistry.

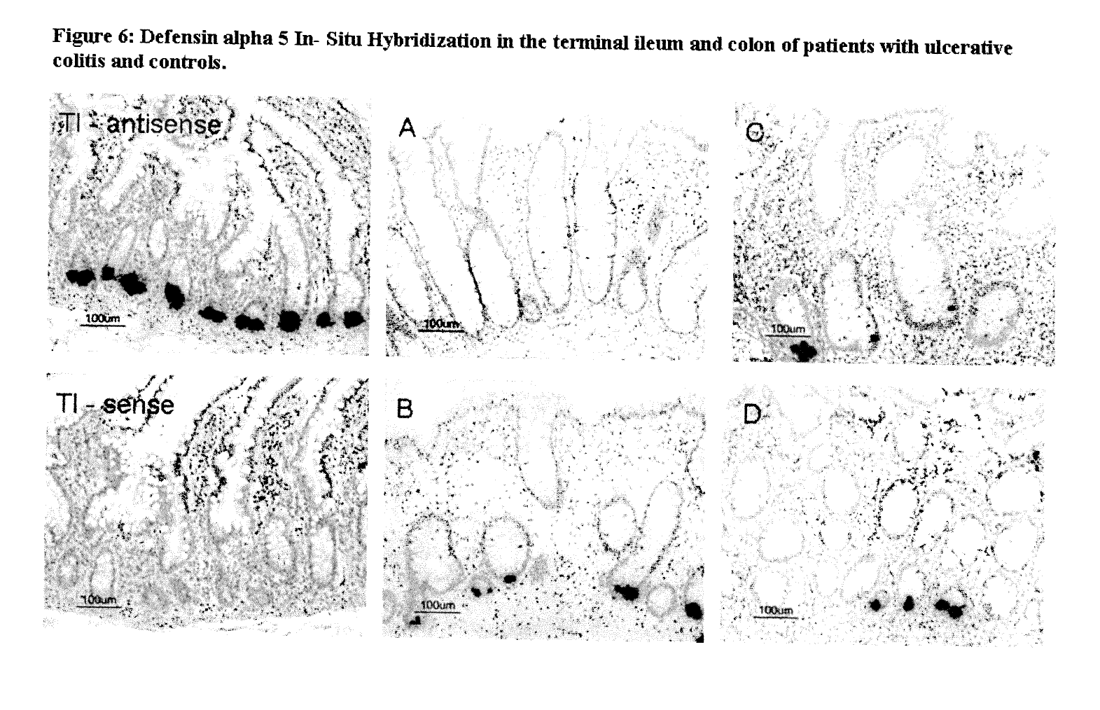

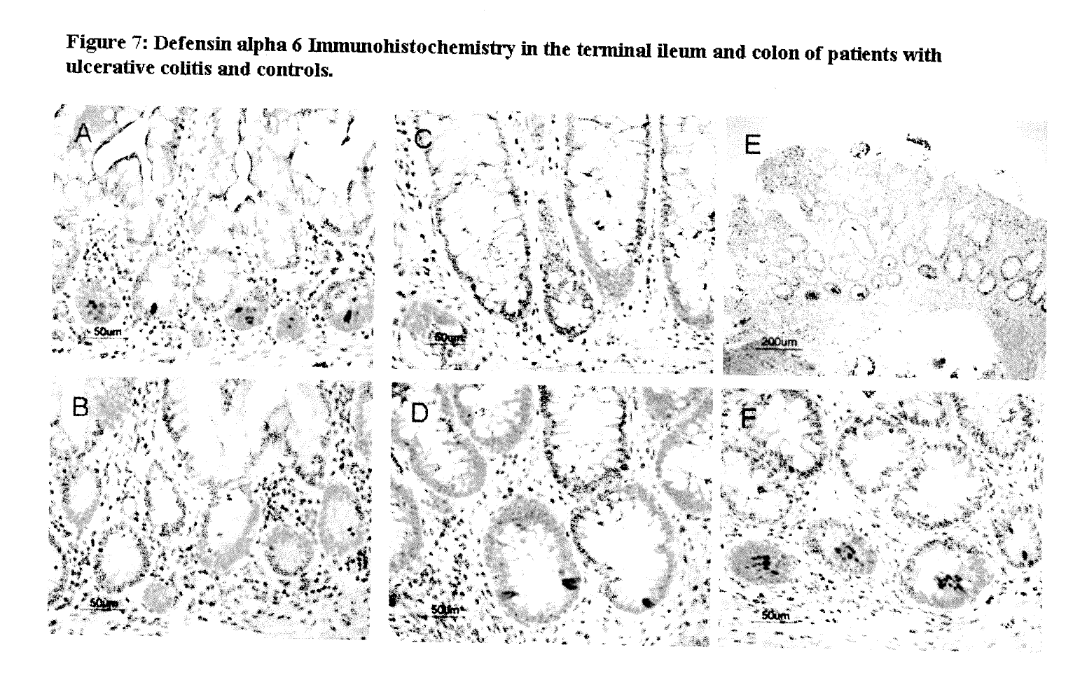

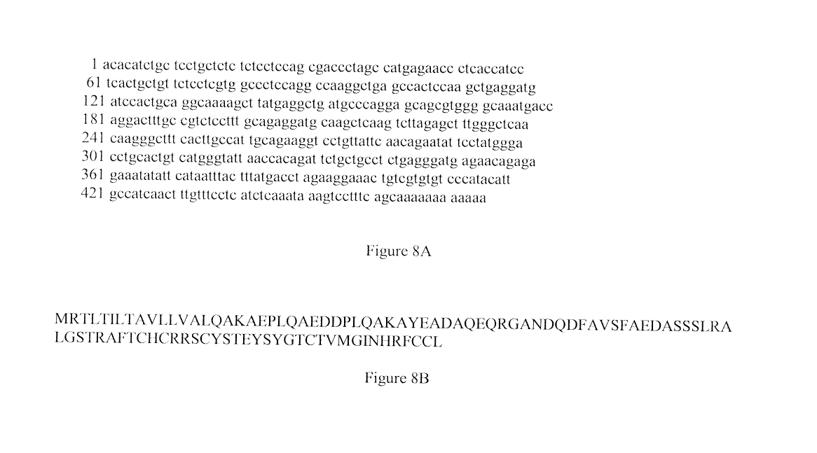

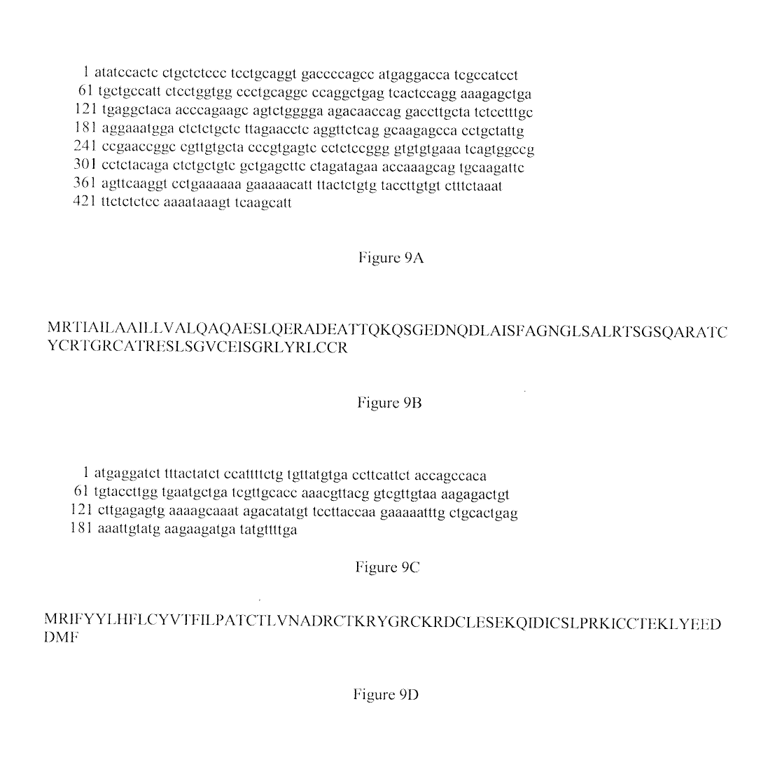

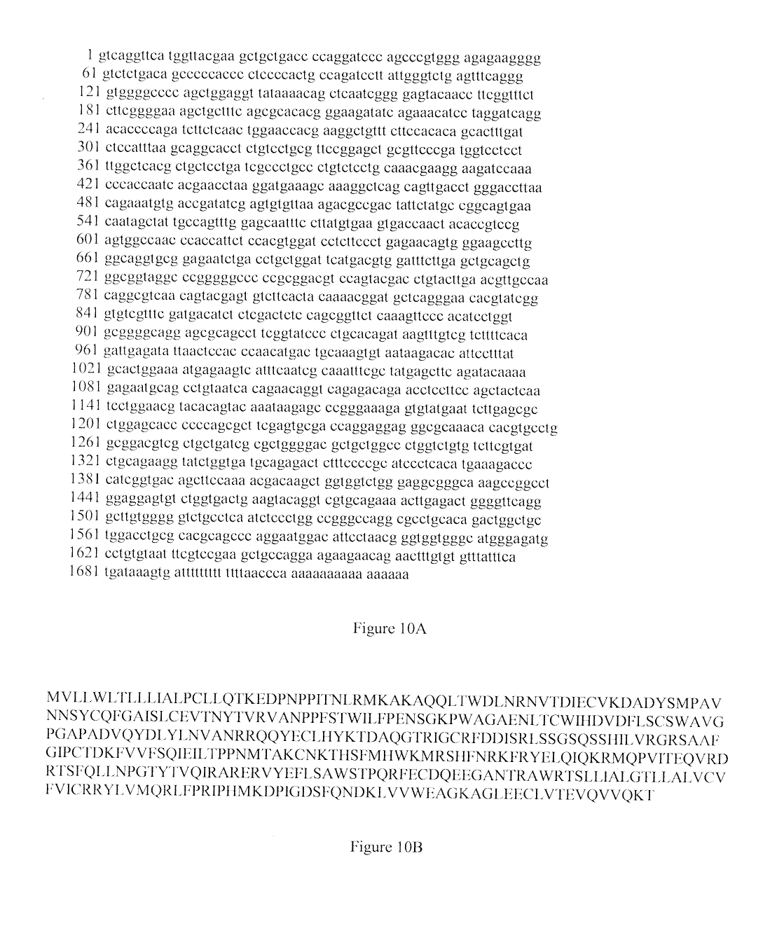

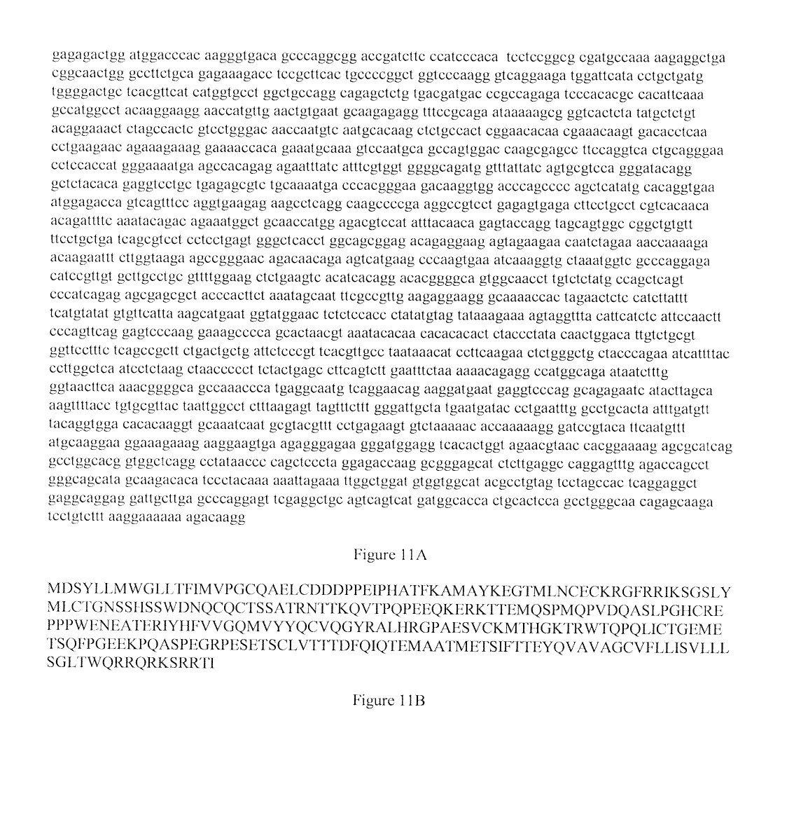

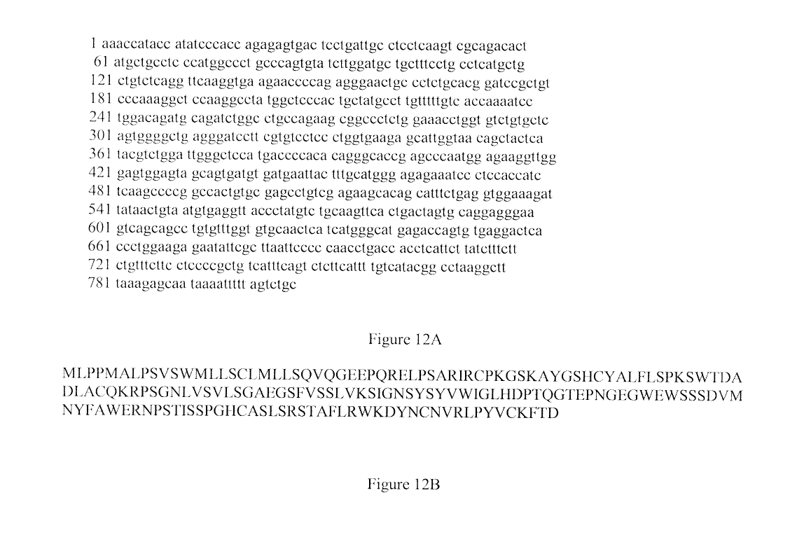

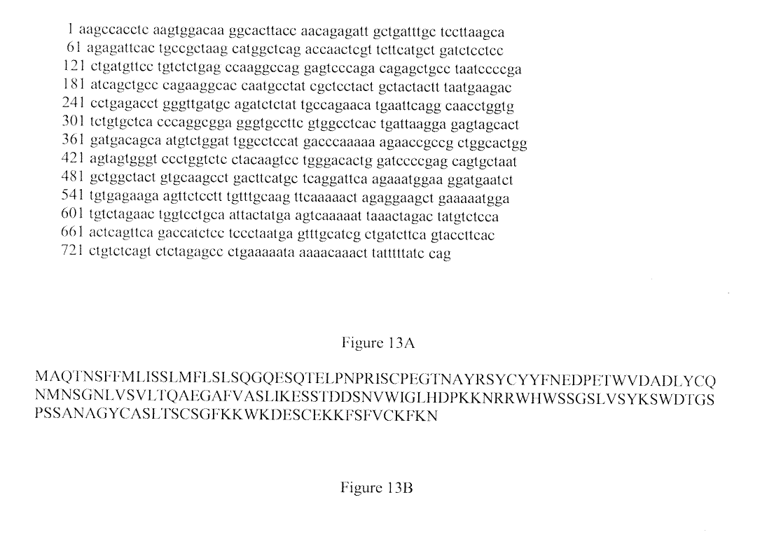

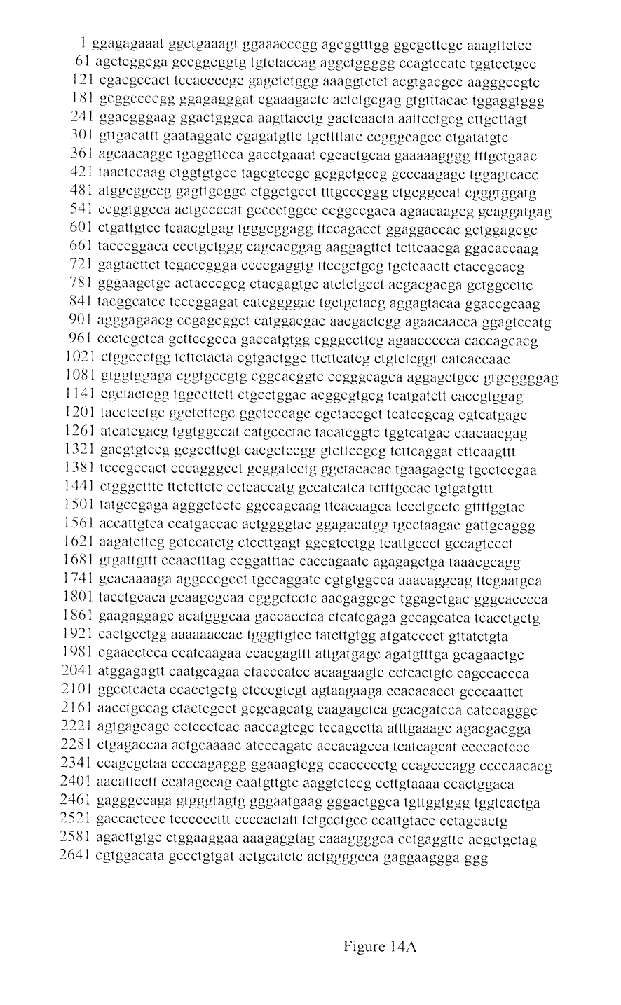

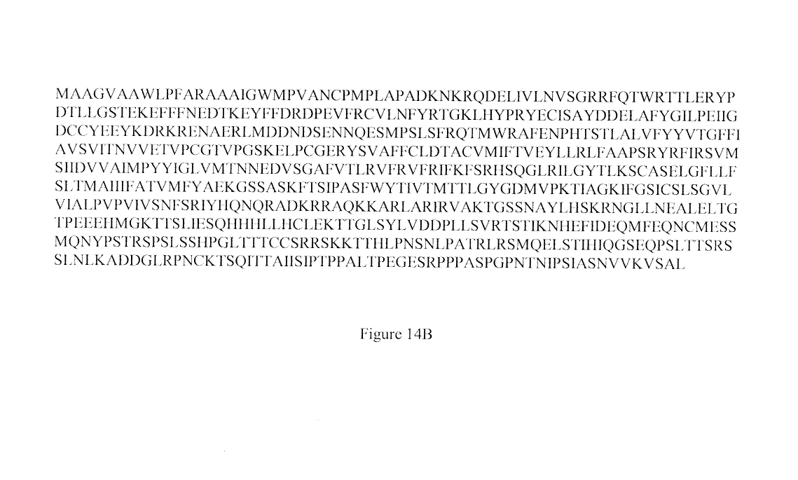

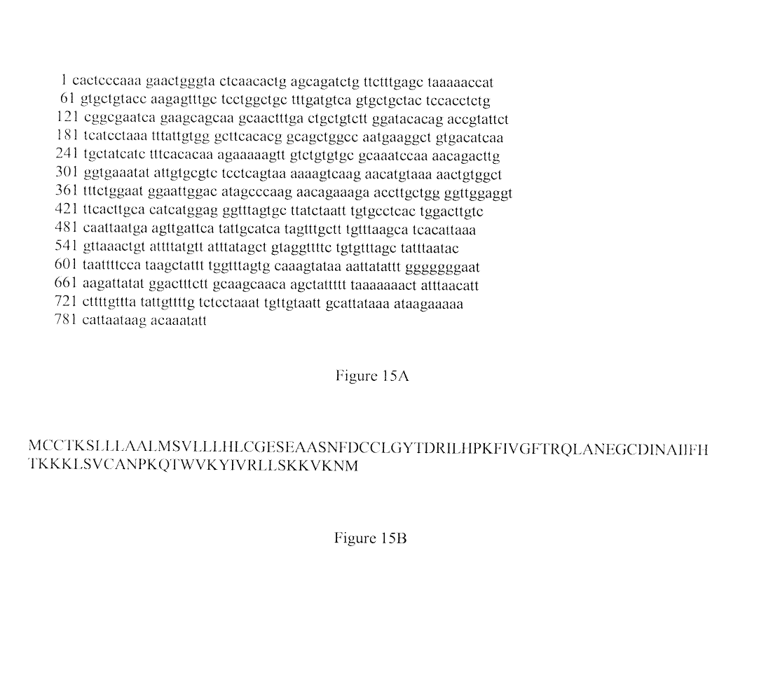

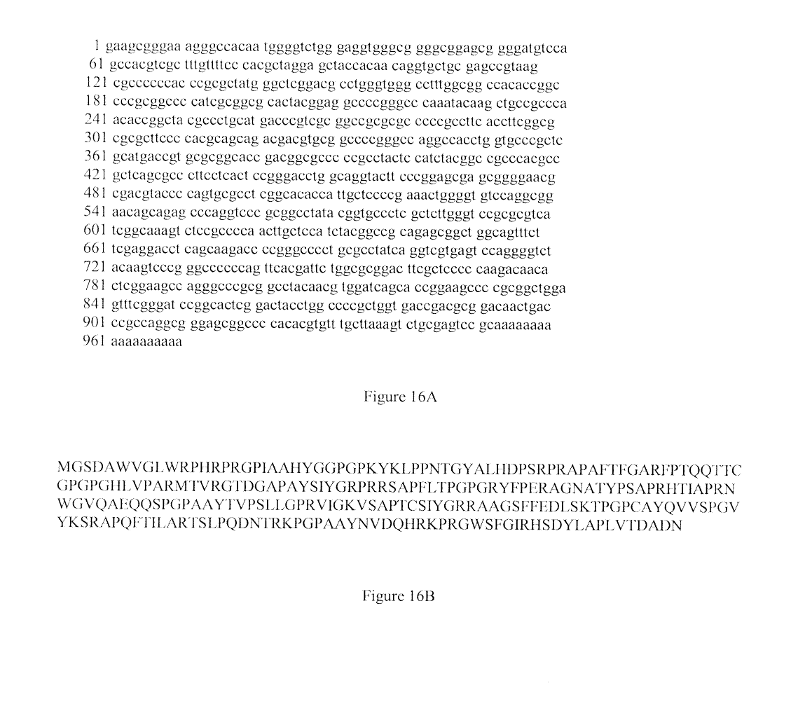

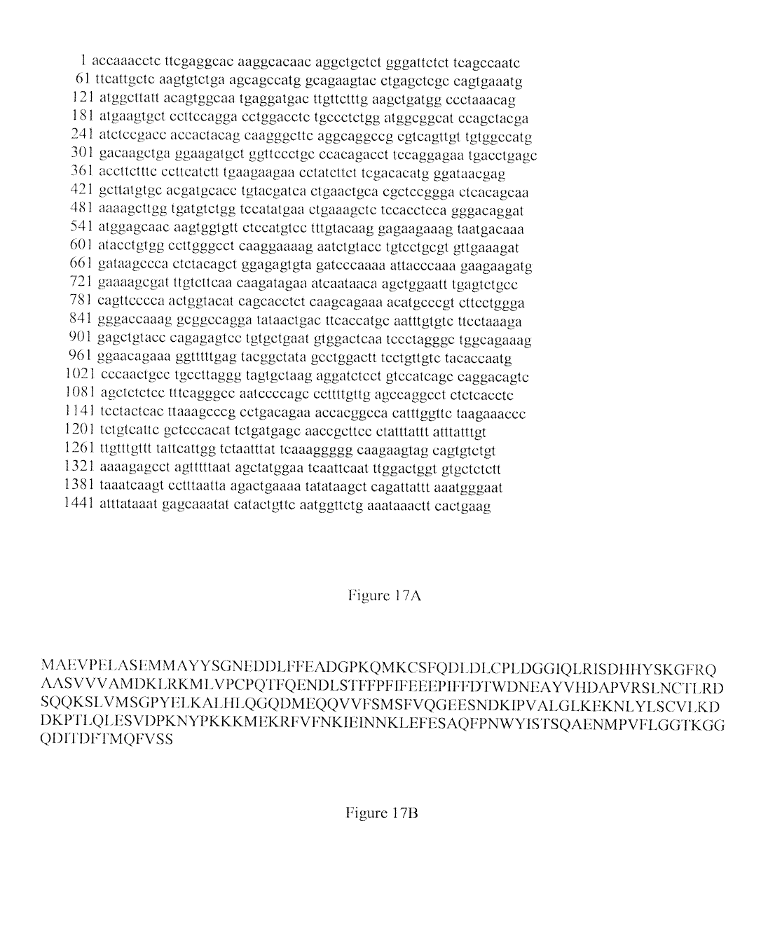

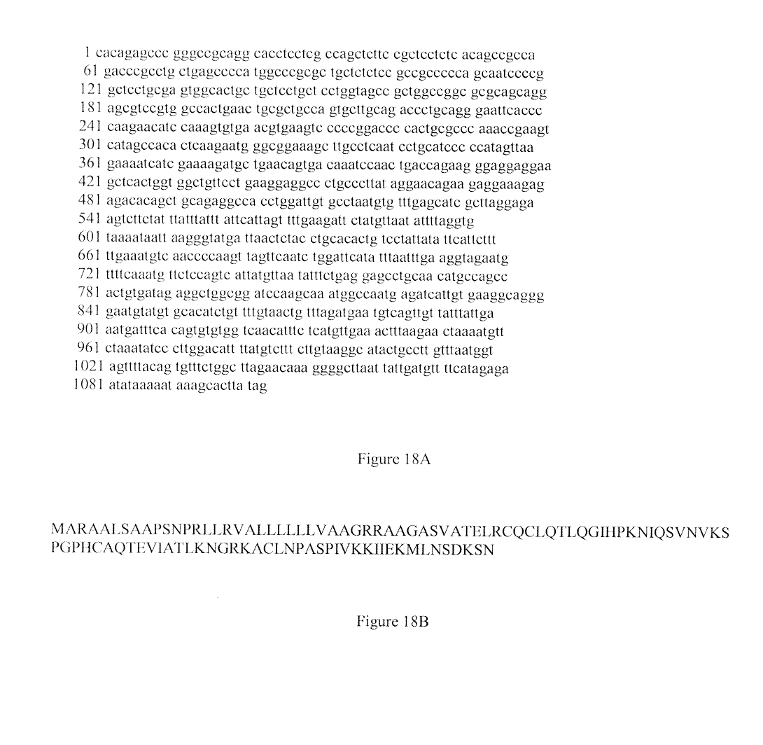

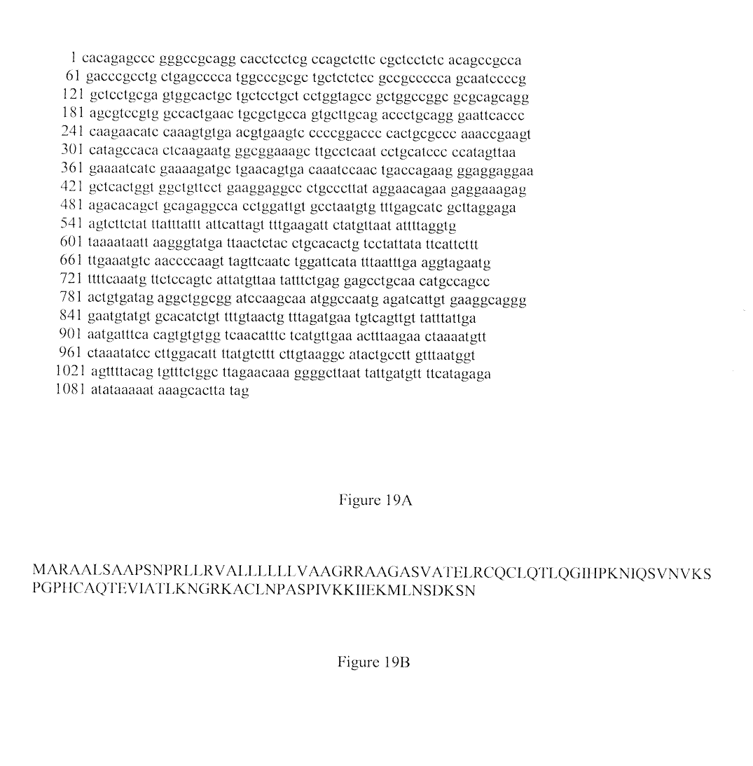

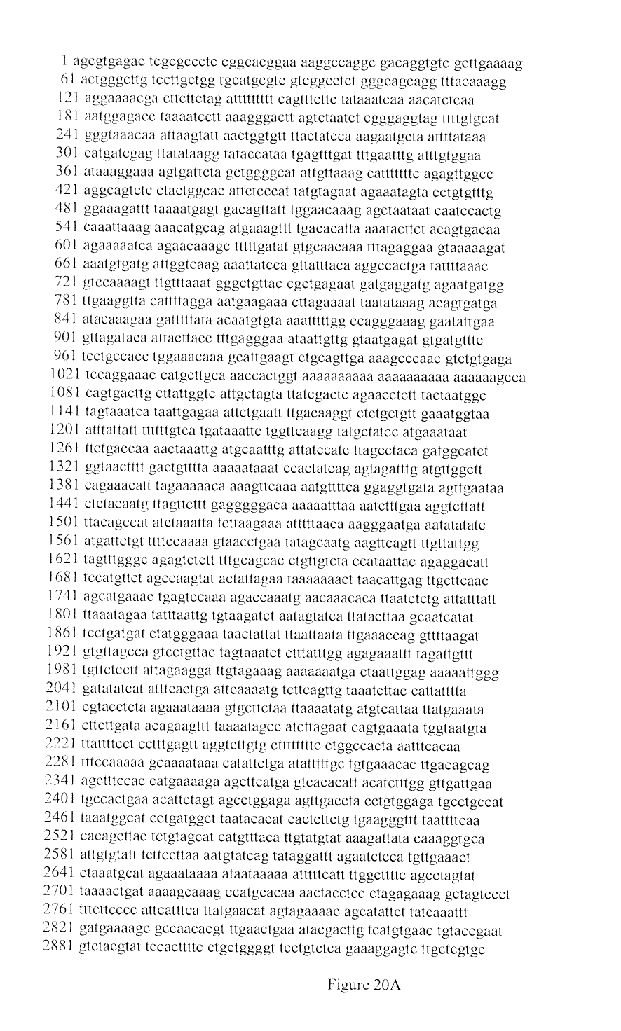

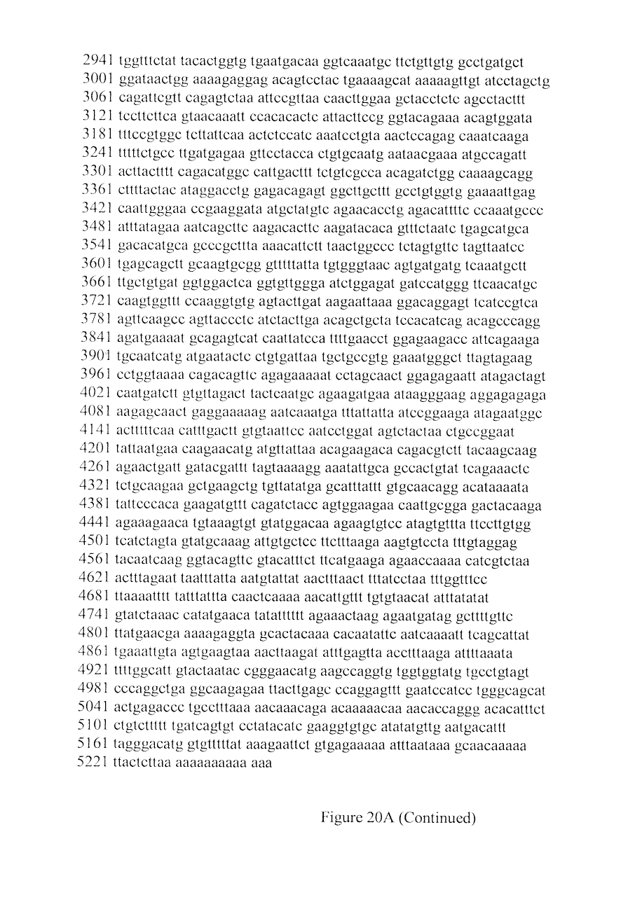

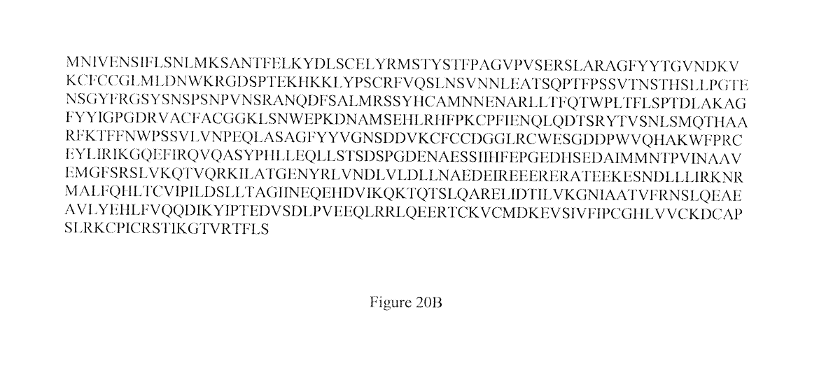

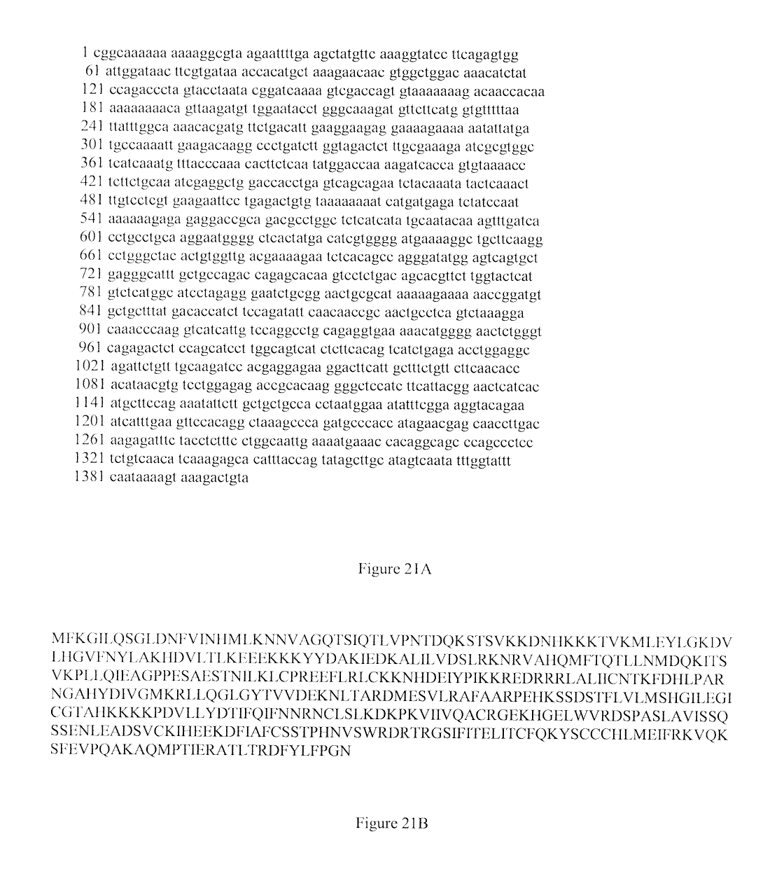

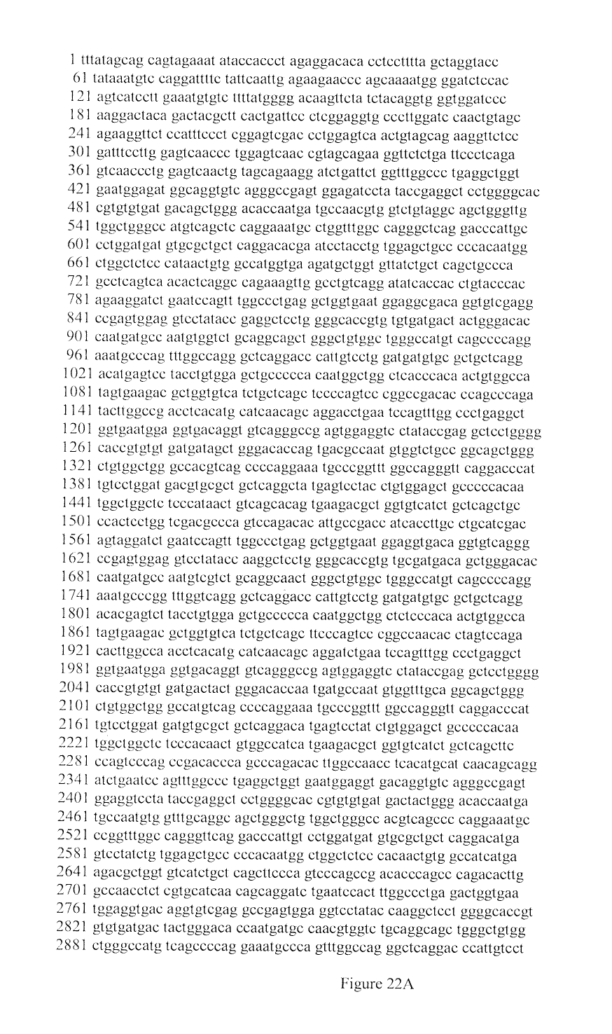

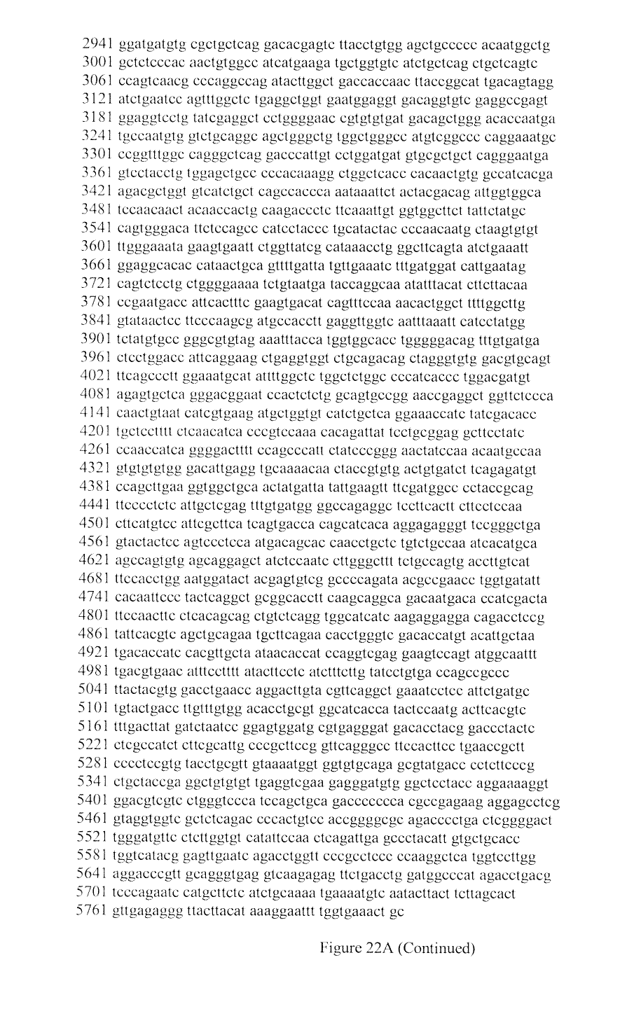

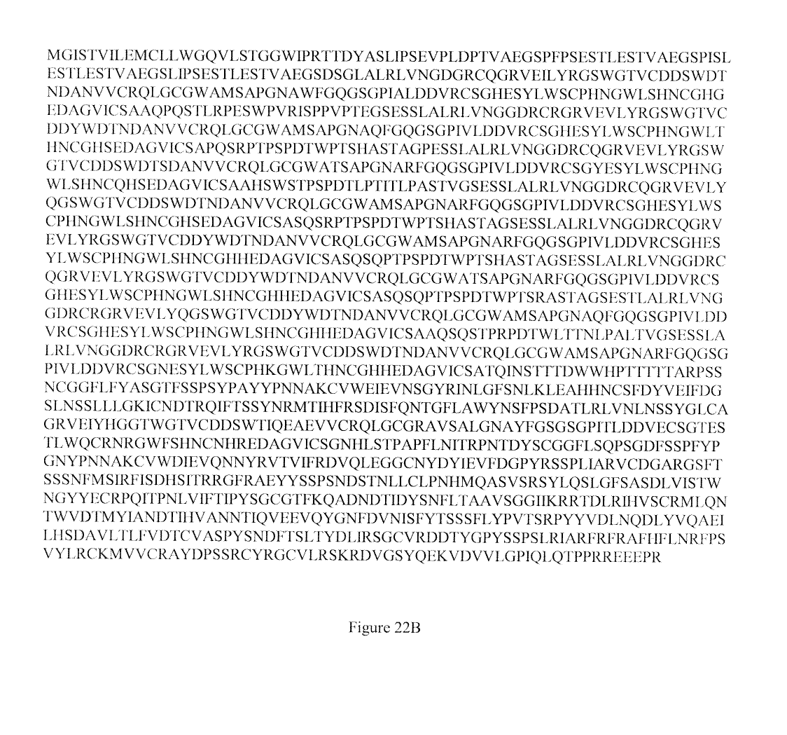

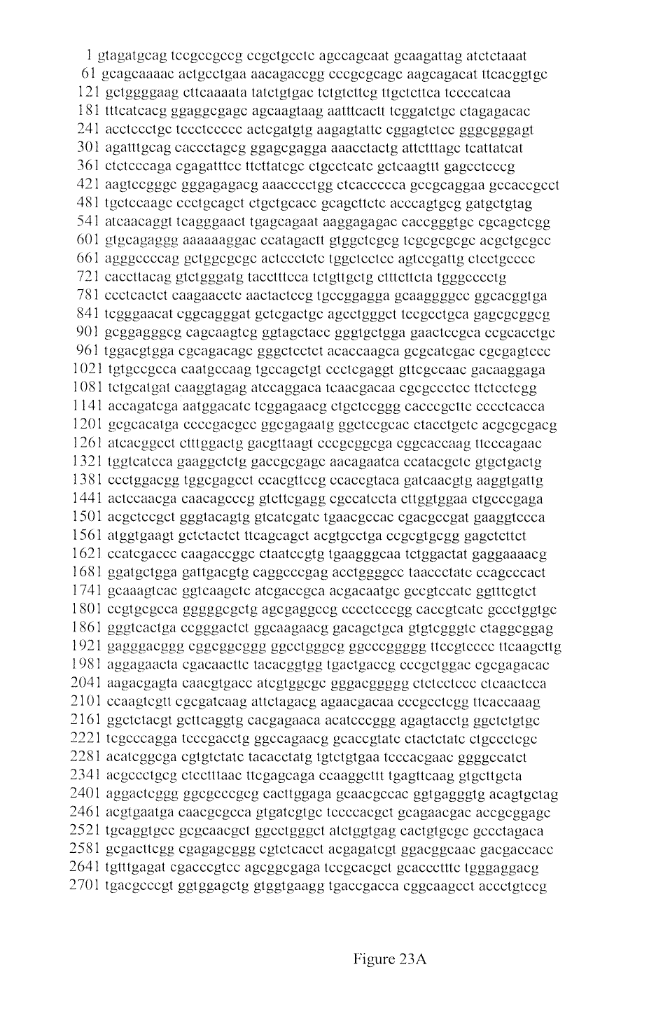

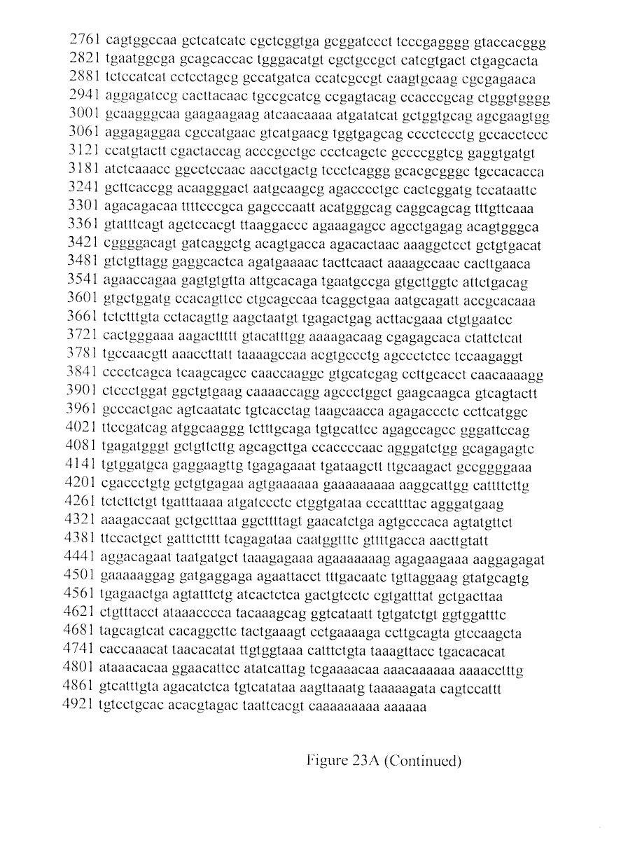

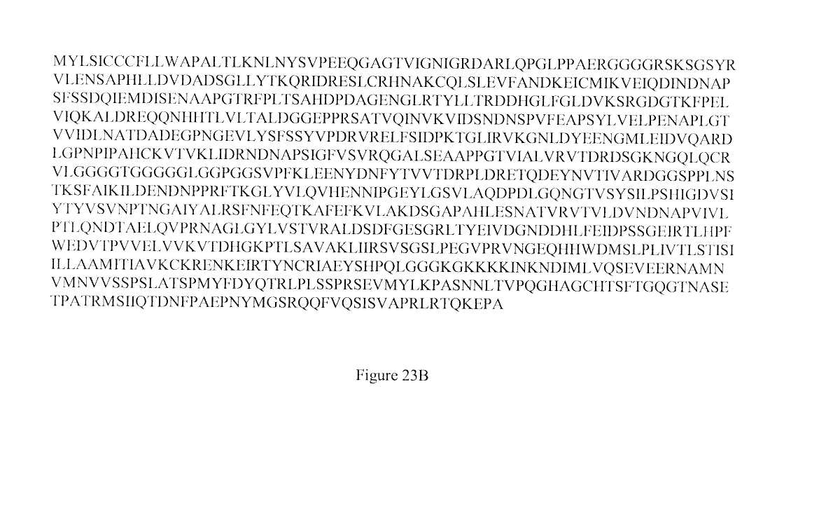

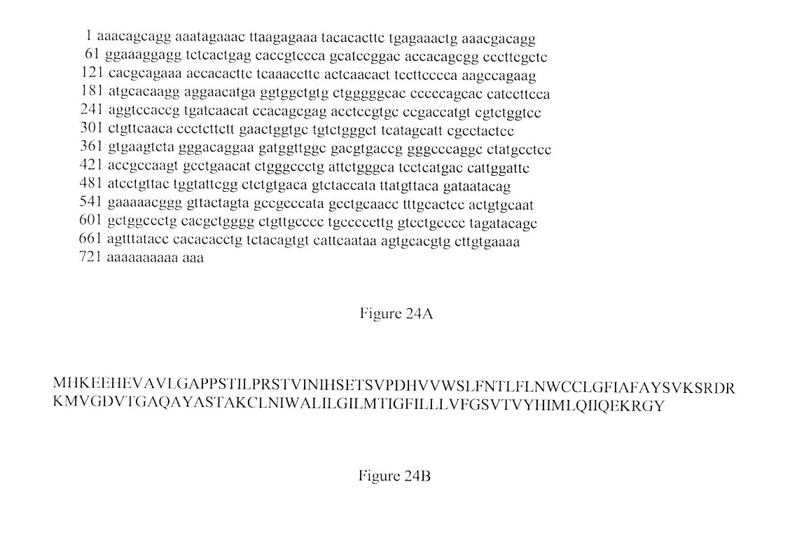

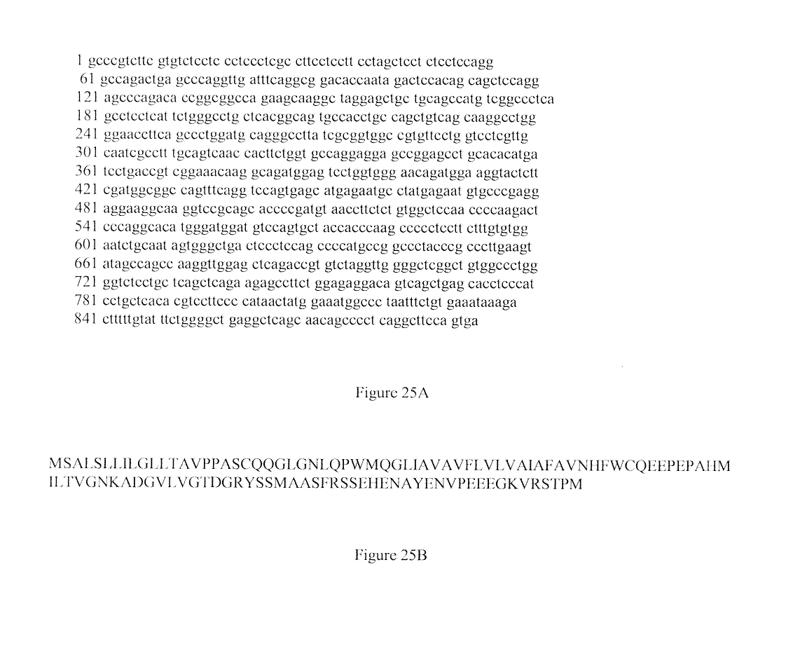

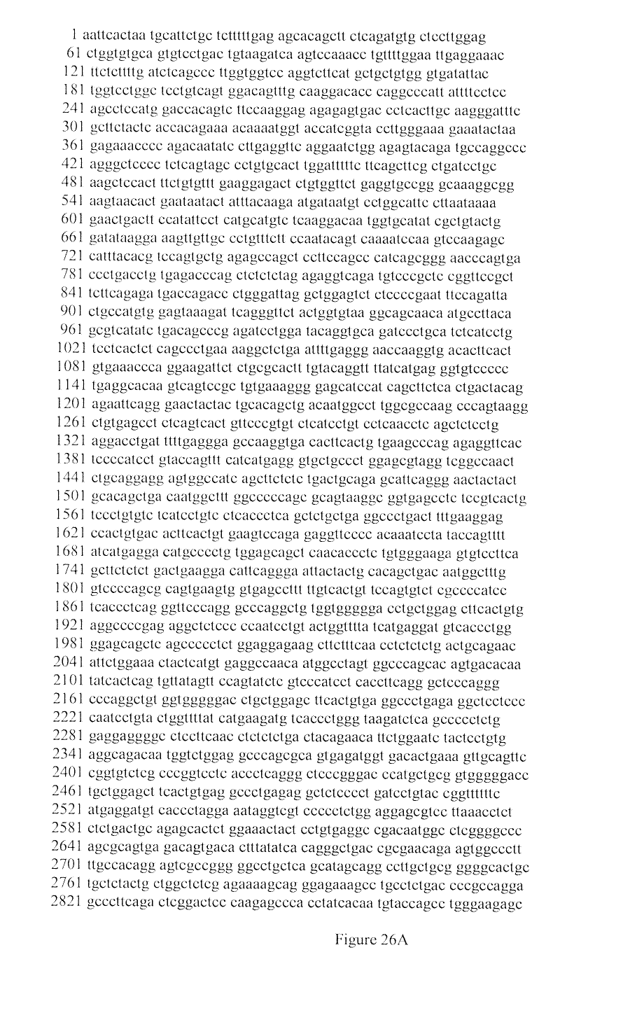

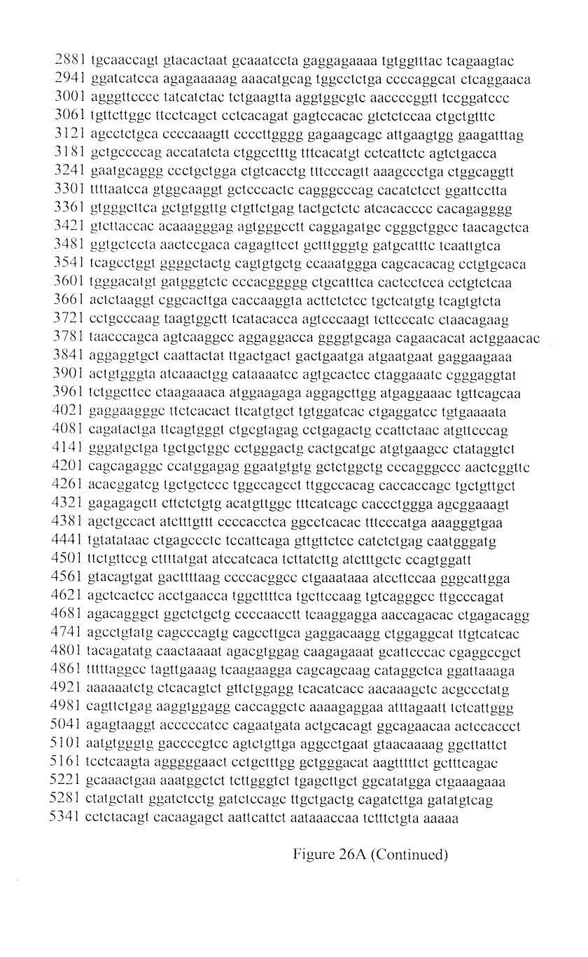

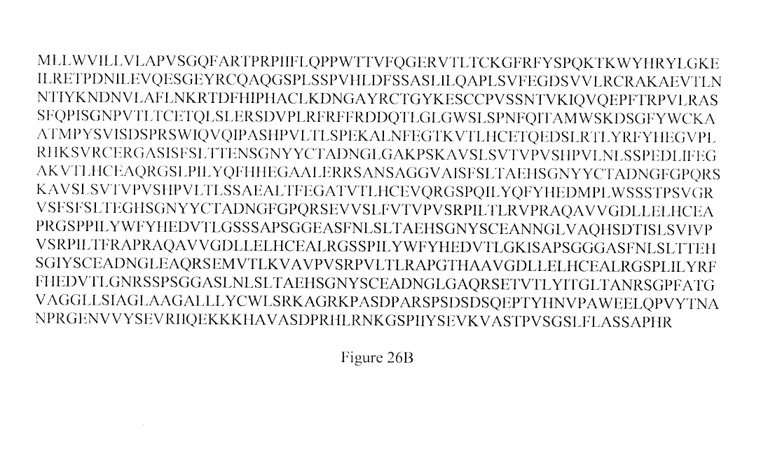

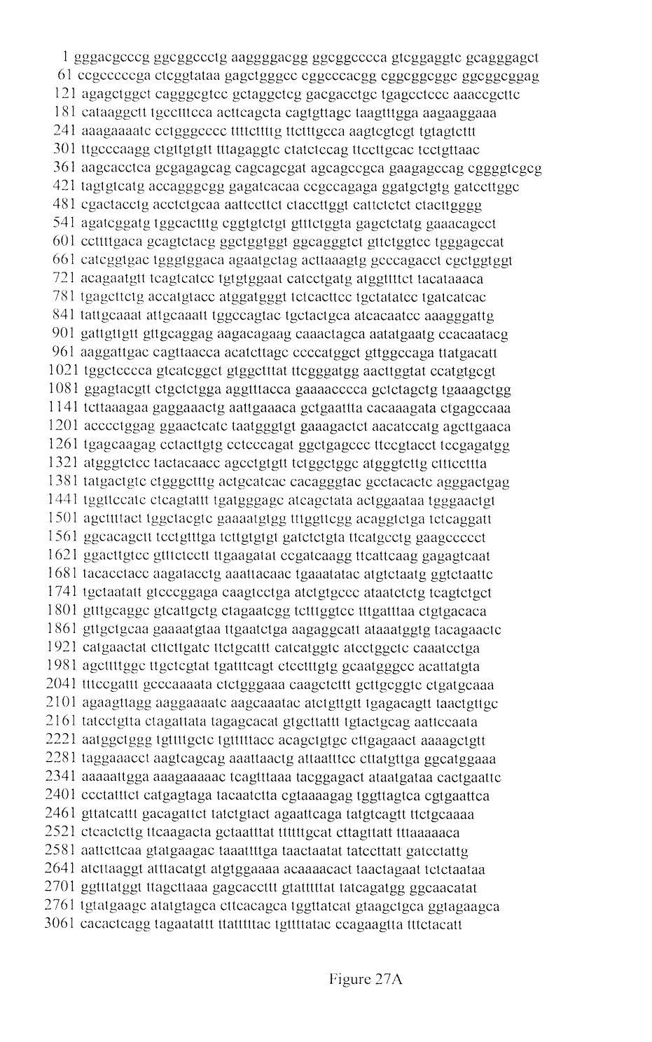

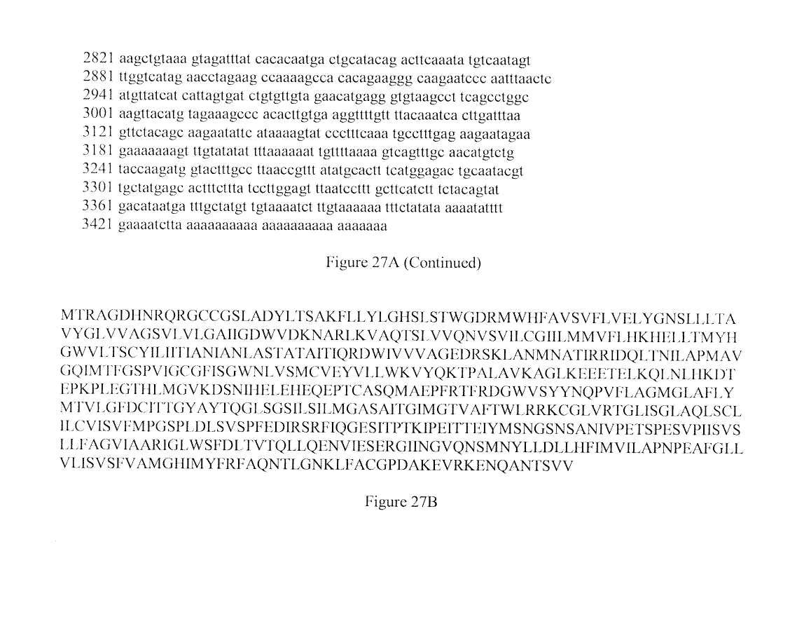

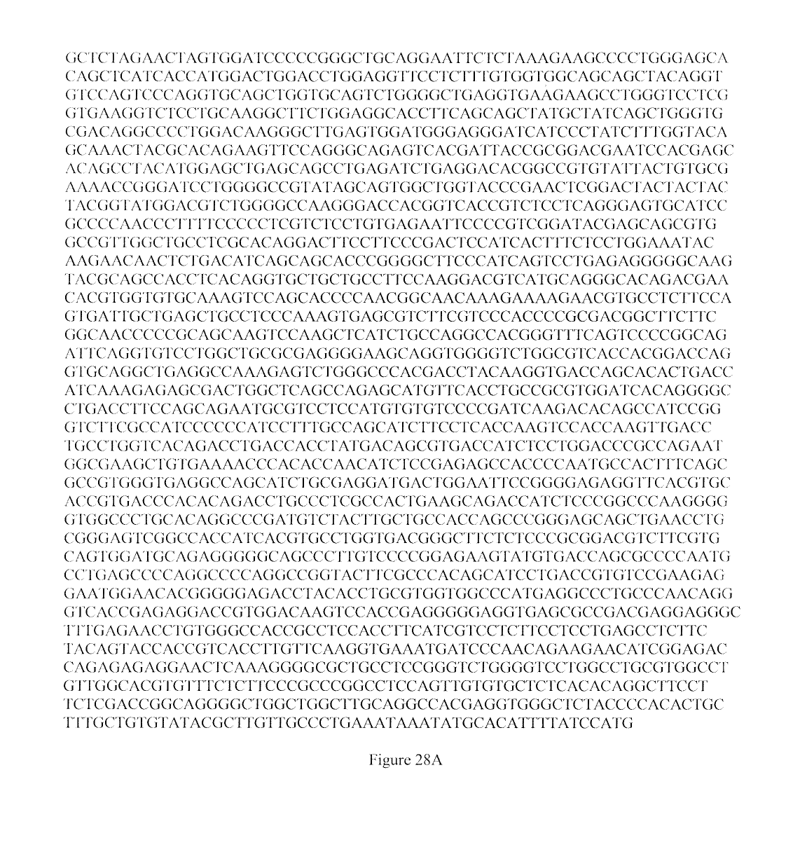

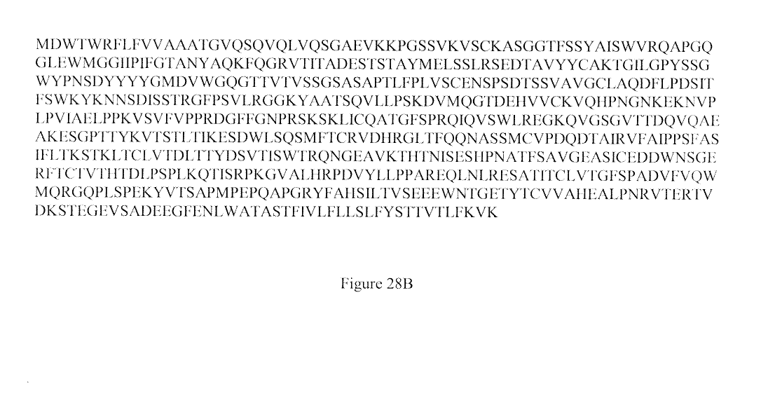

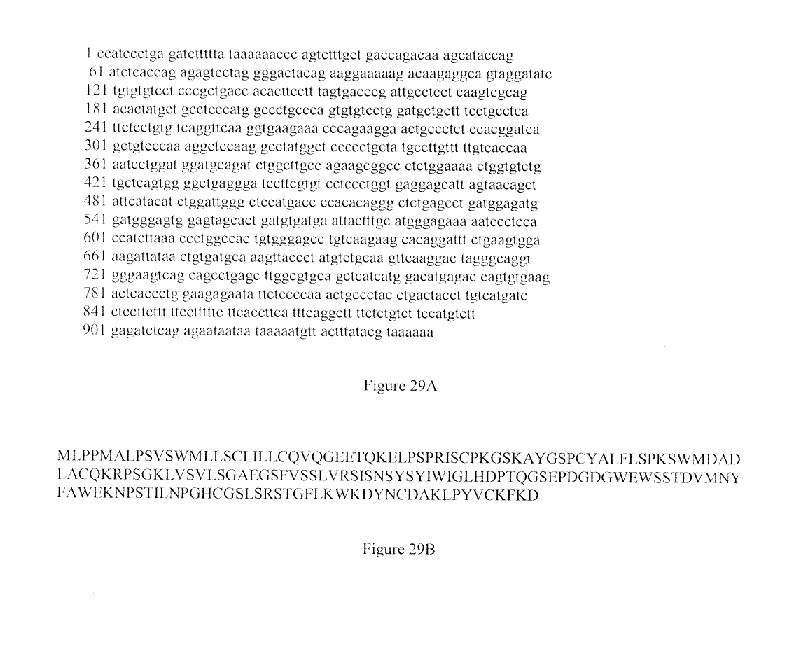

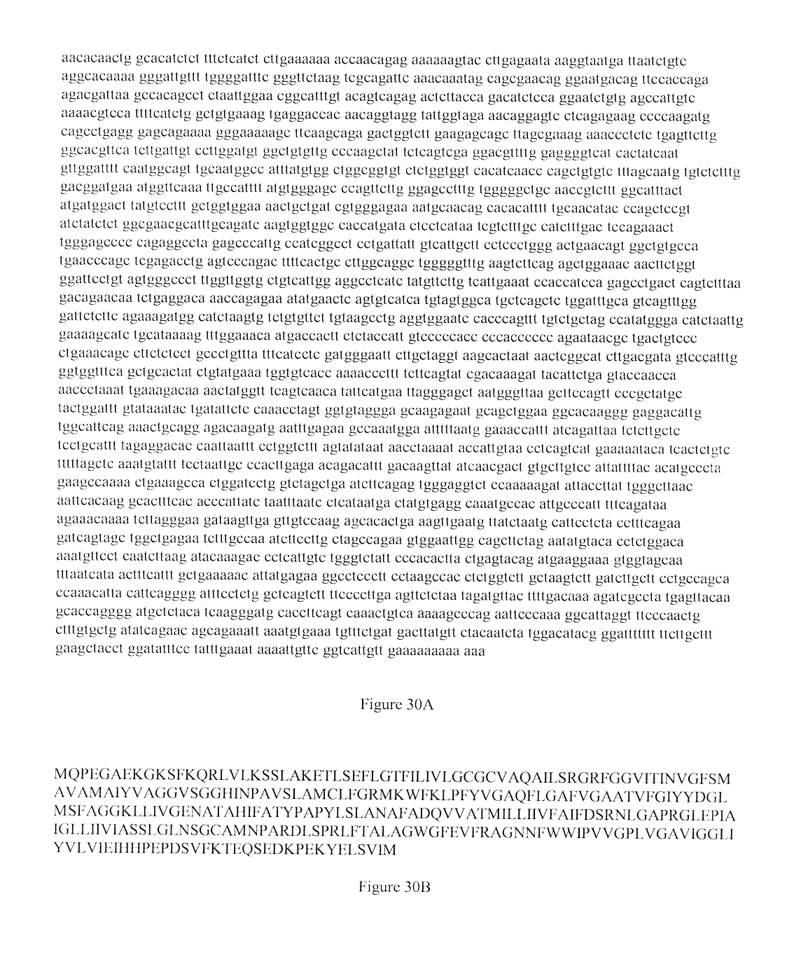

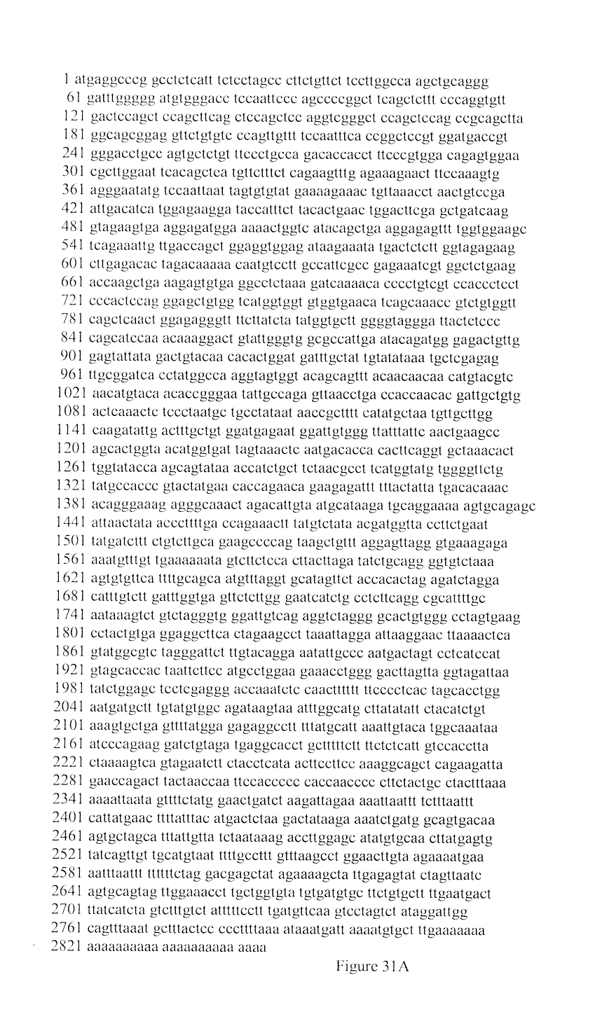

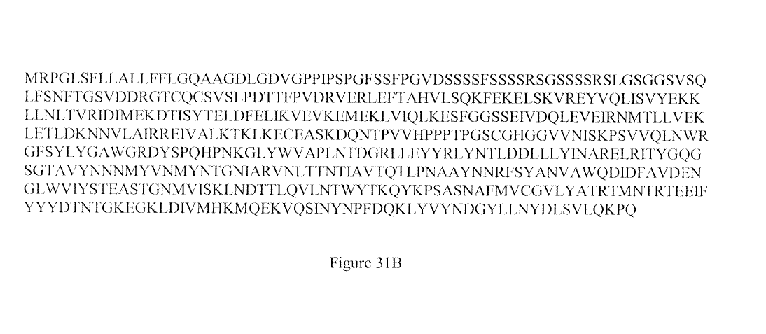

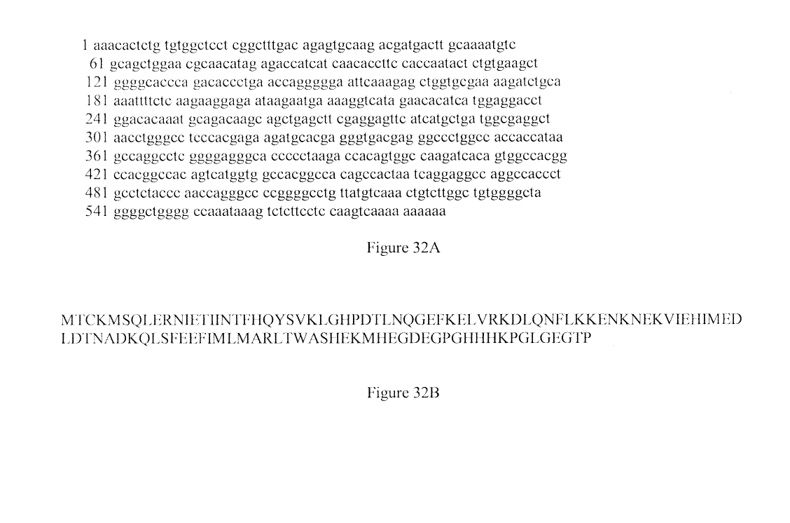

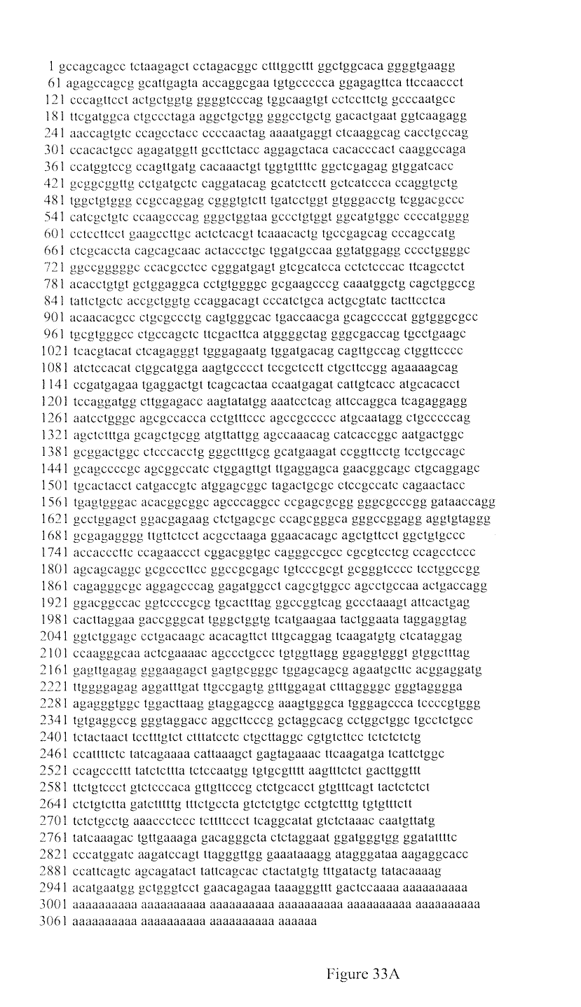

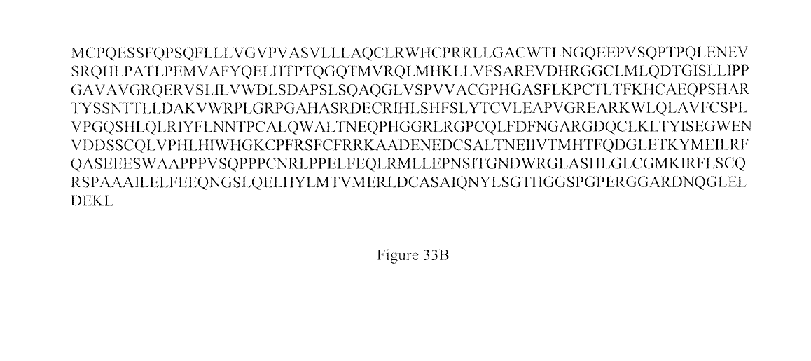

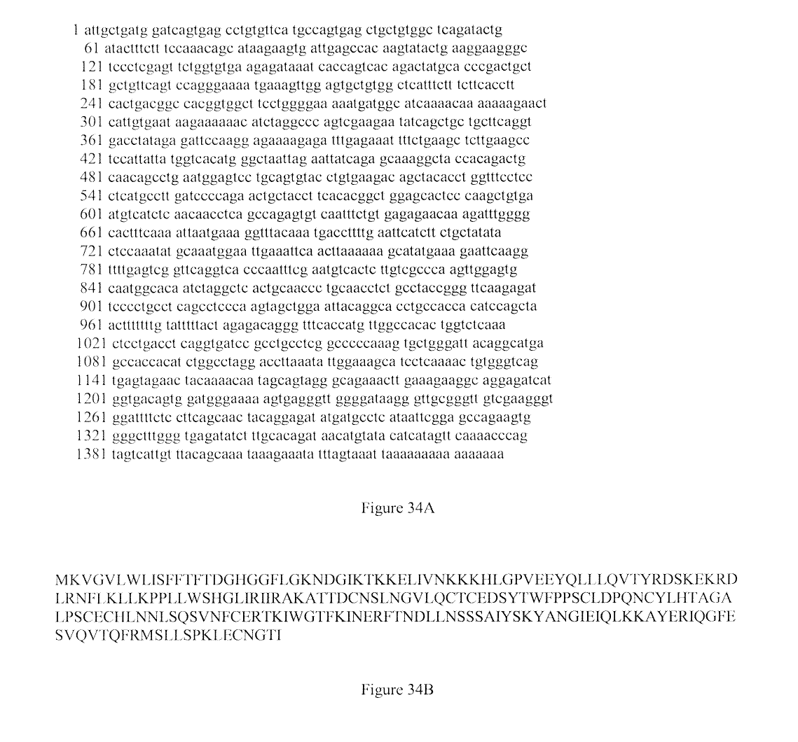

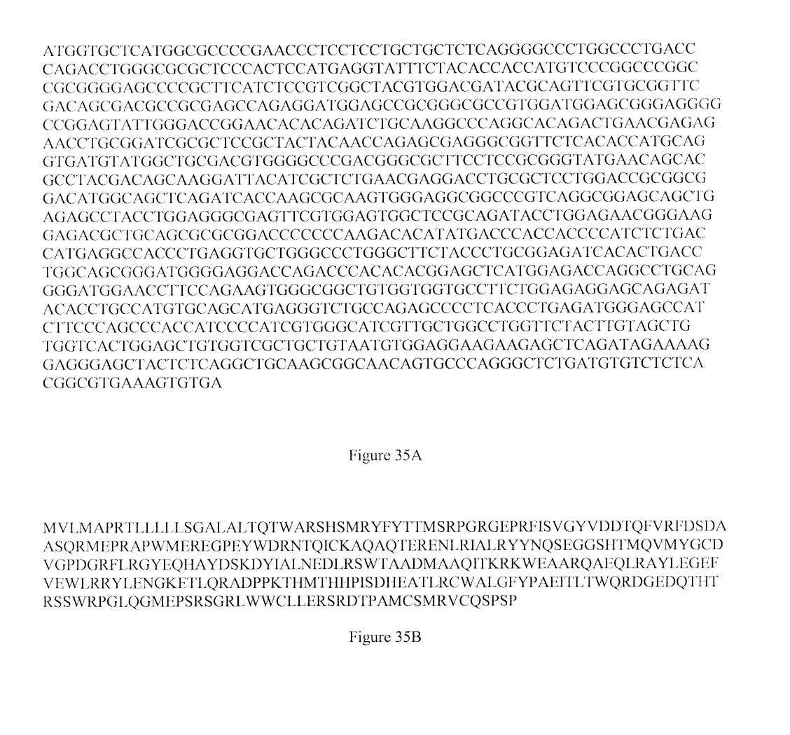

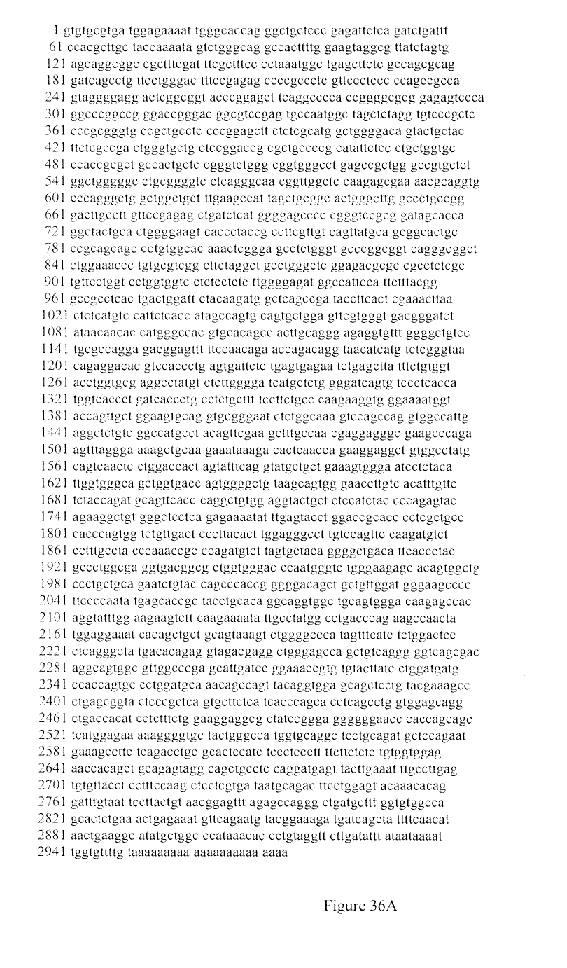

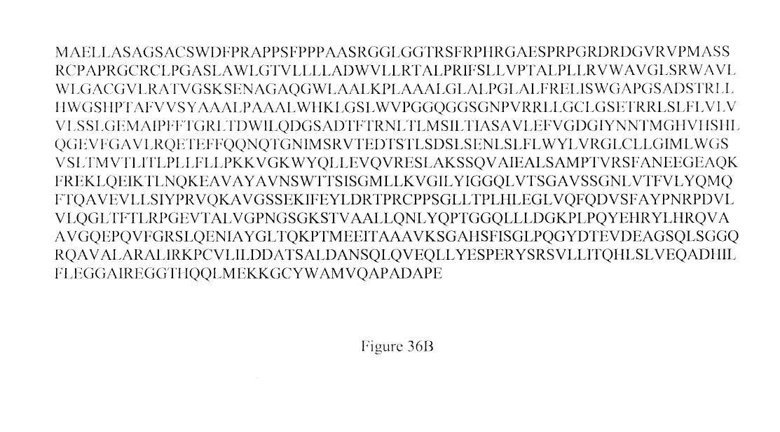

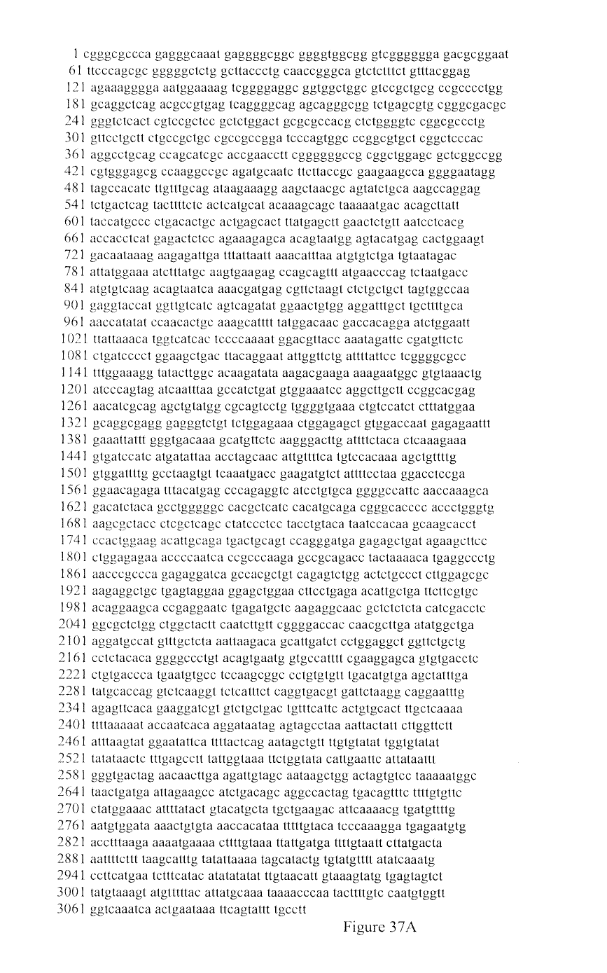

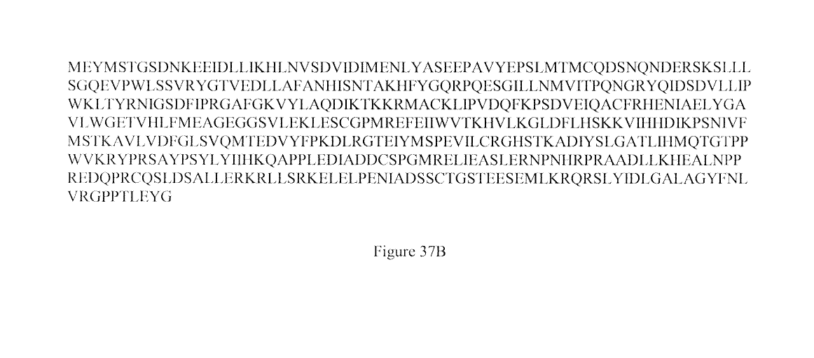

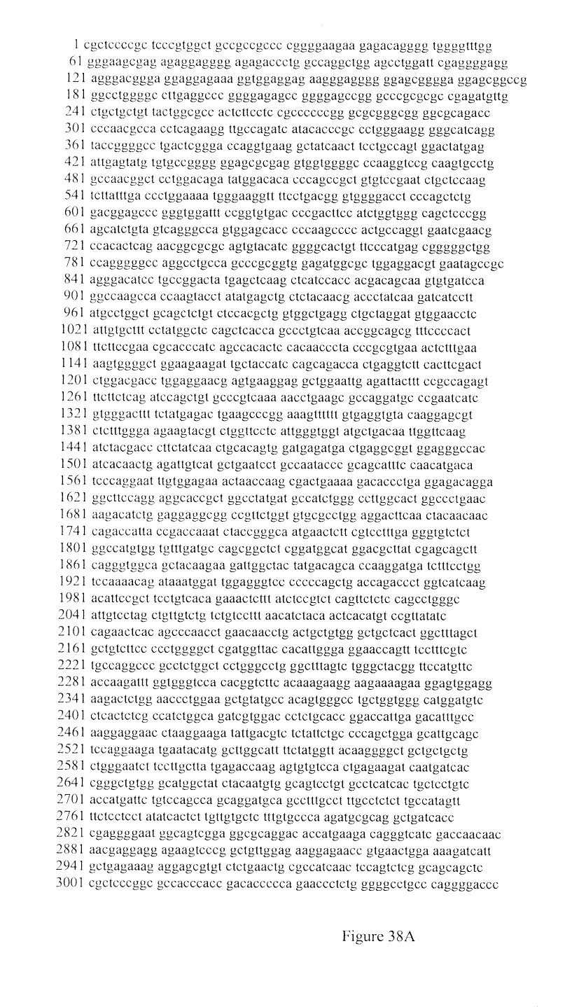

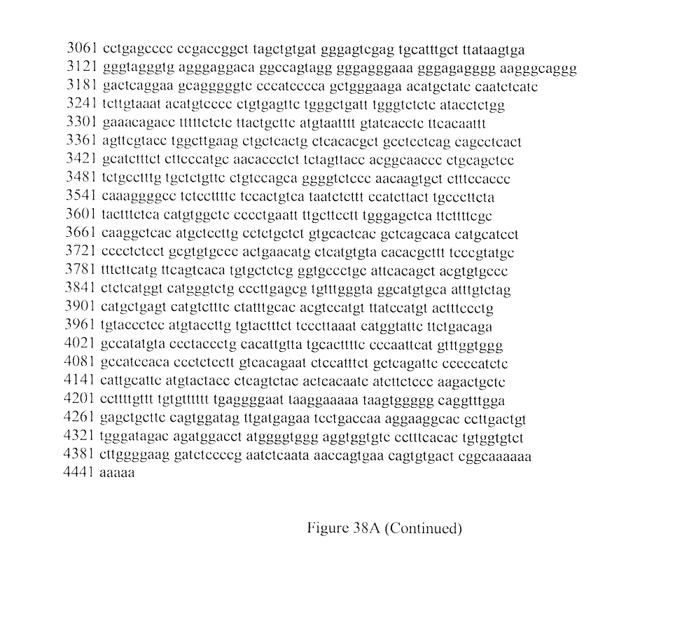

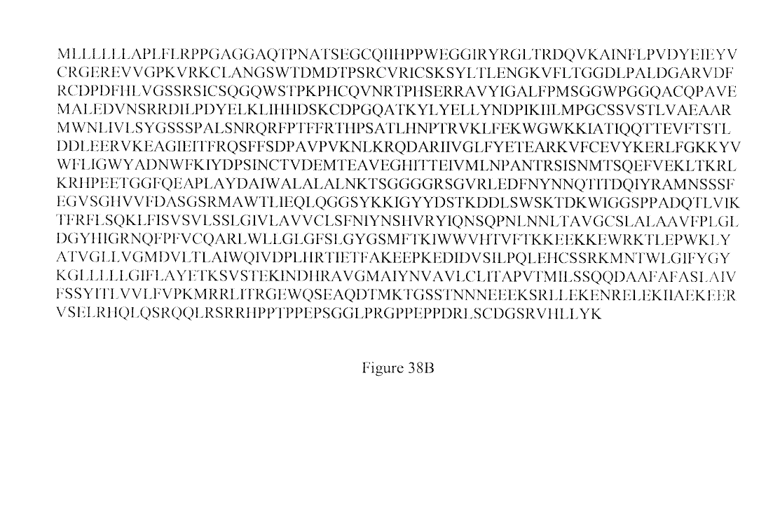

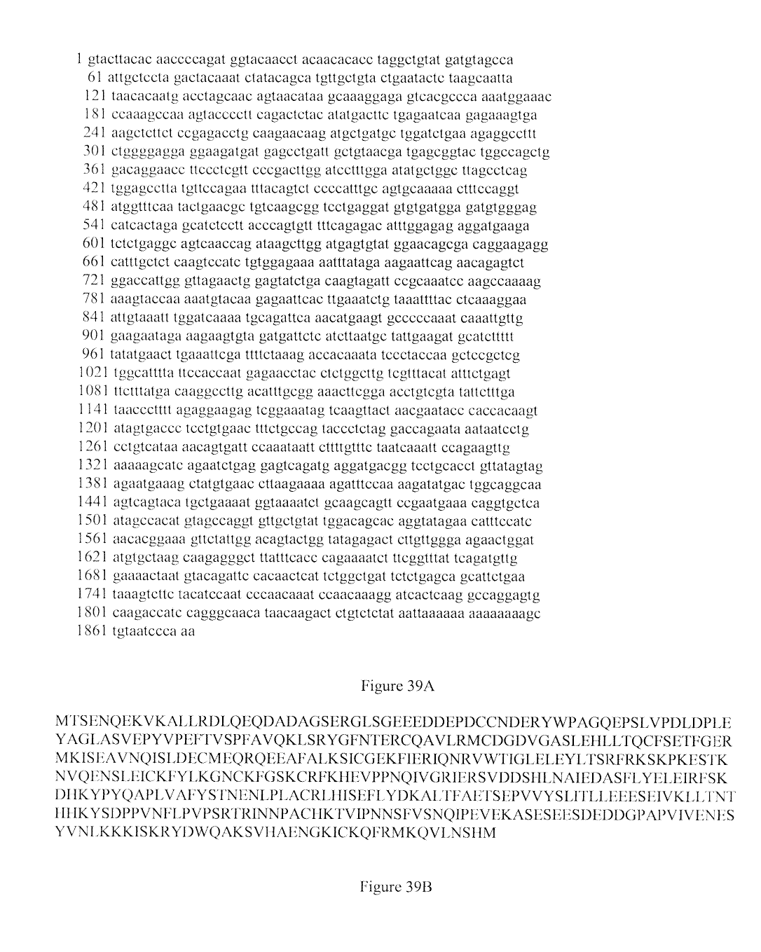

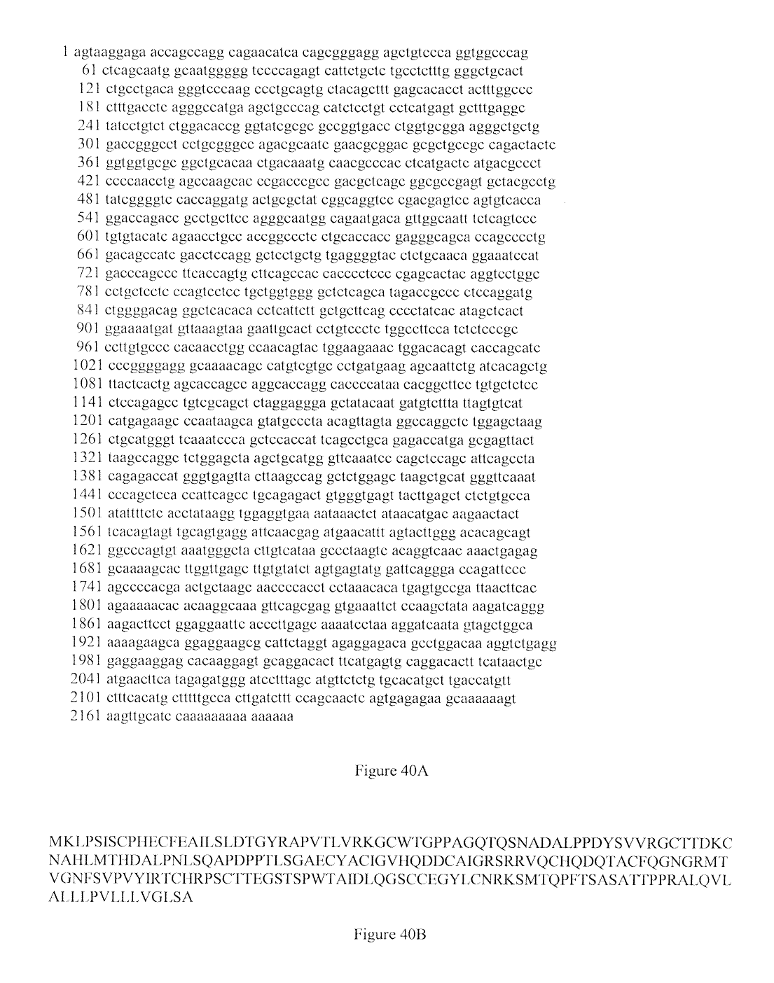

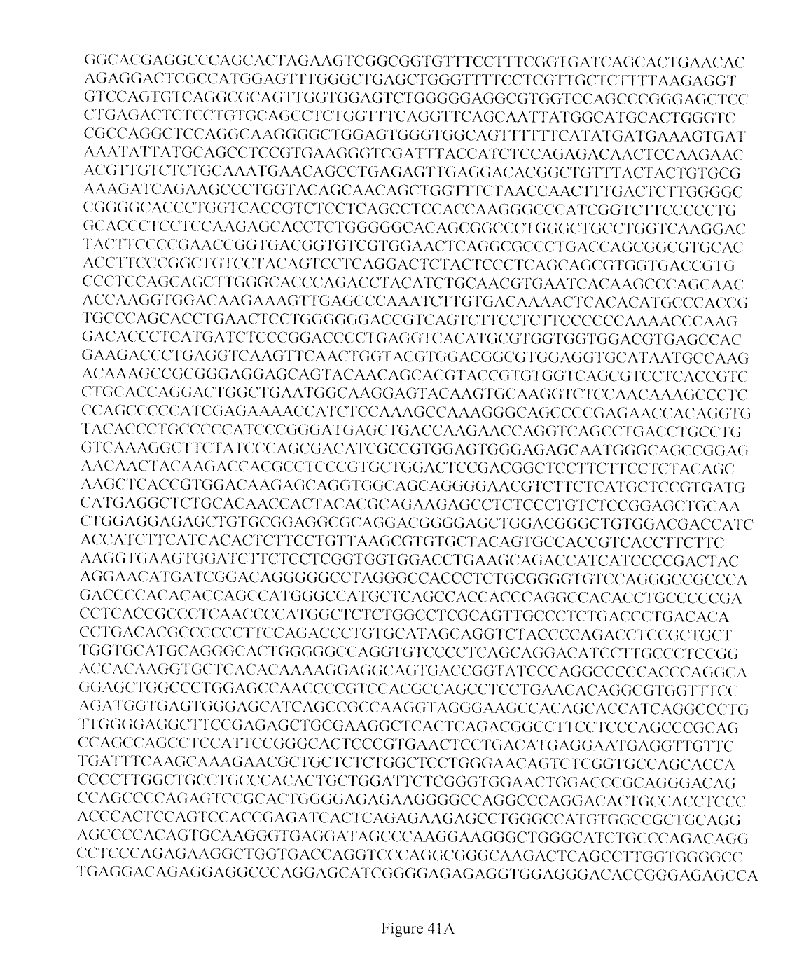

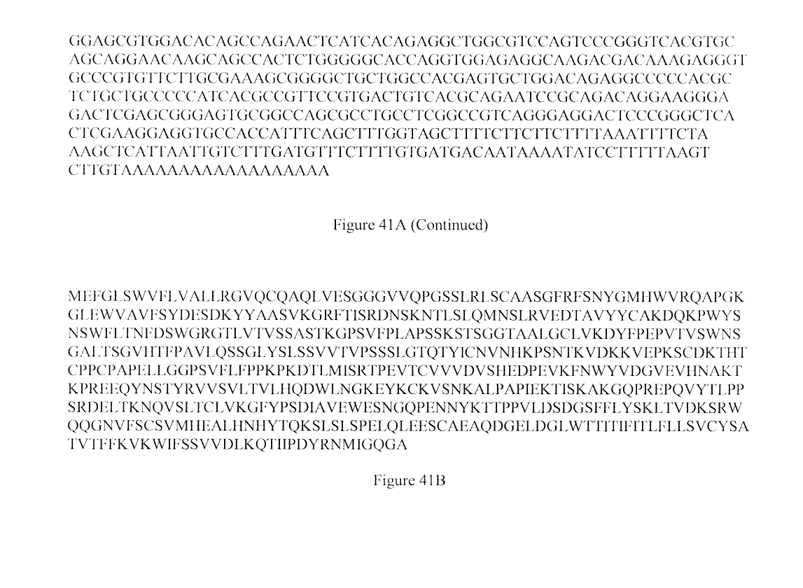

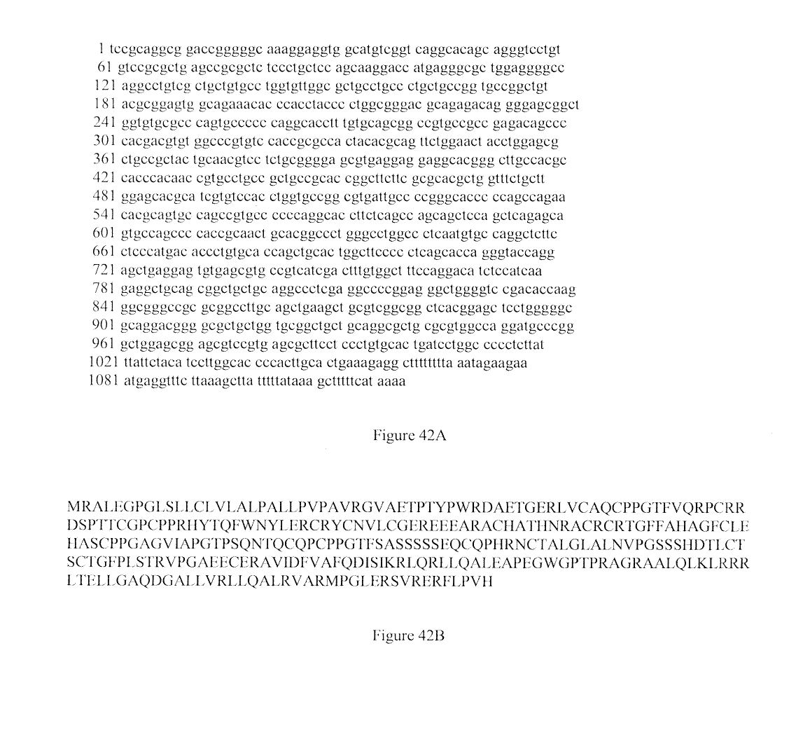

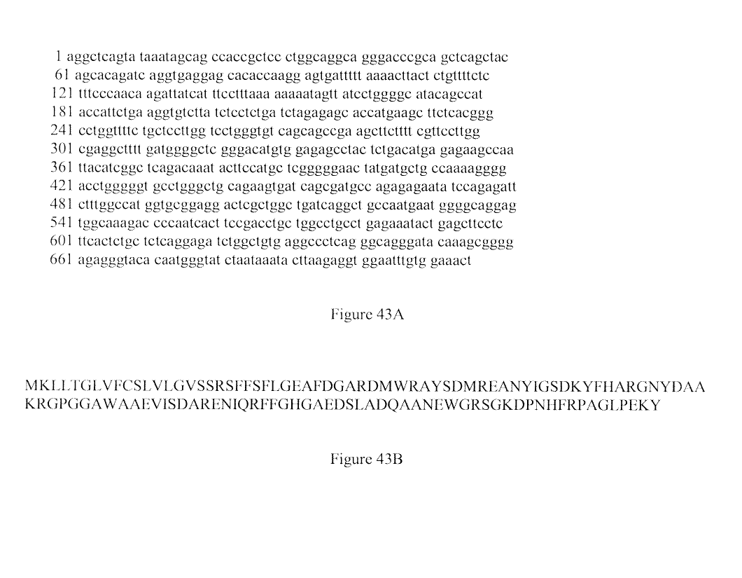

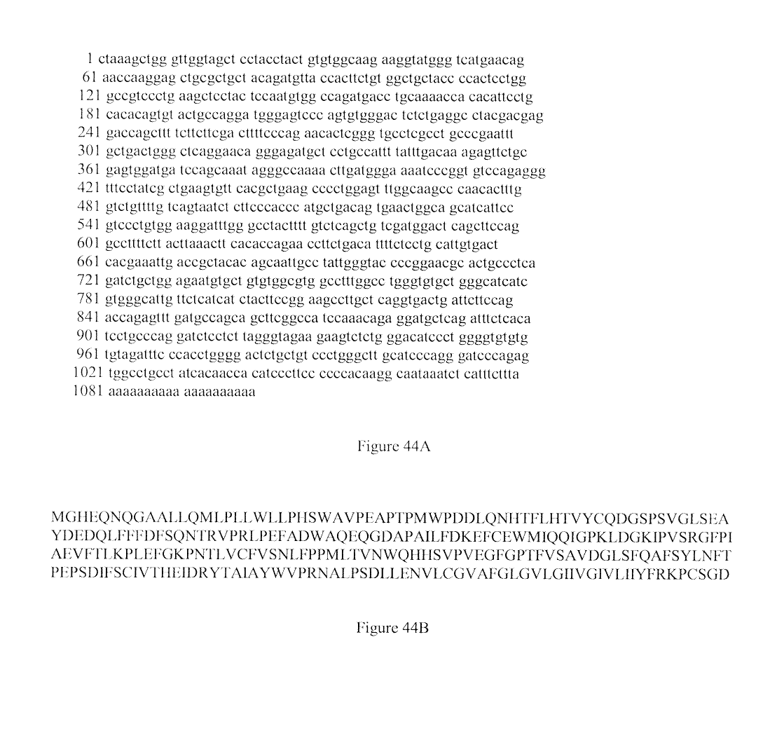

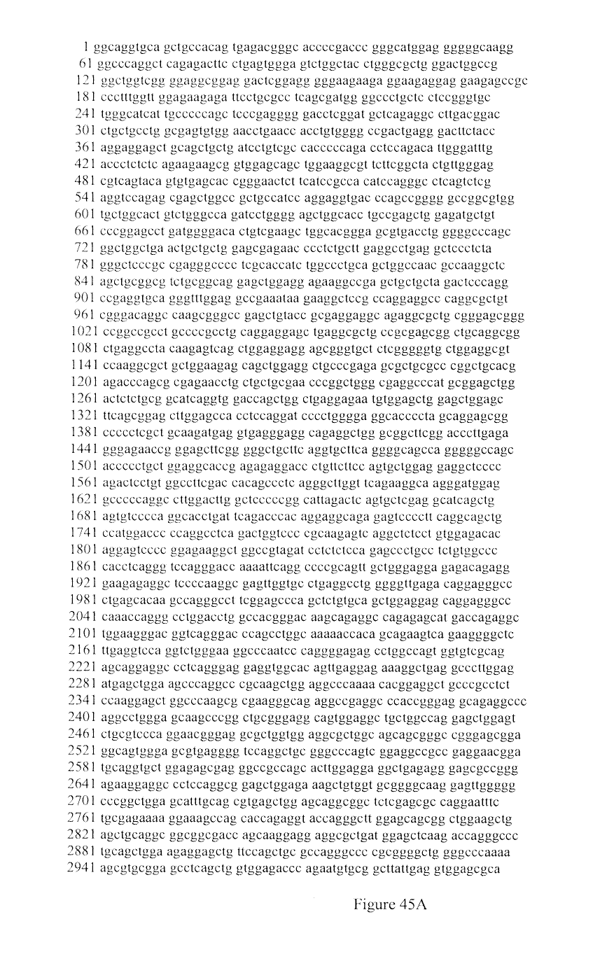

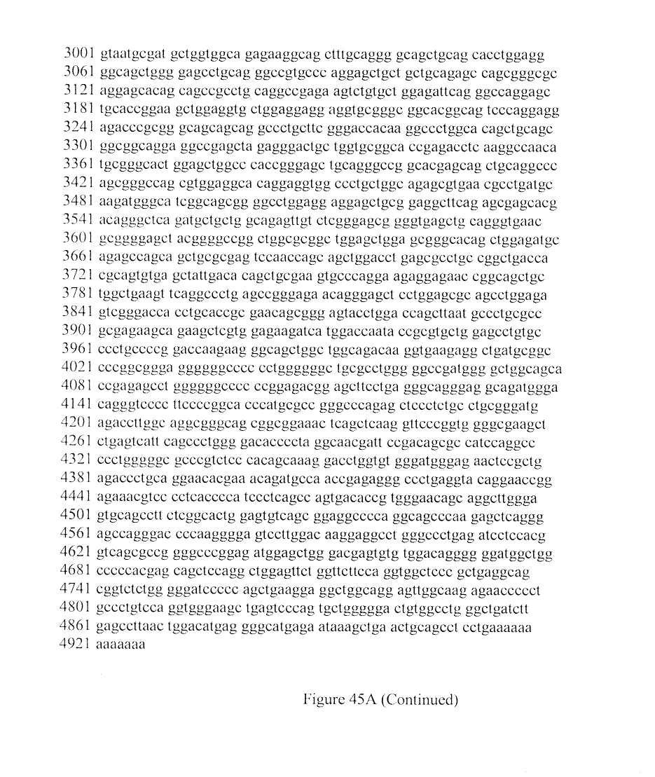

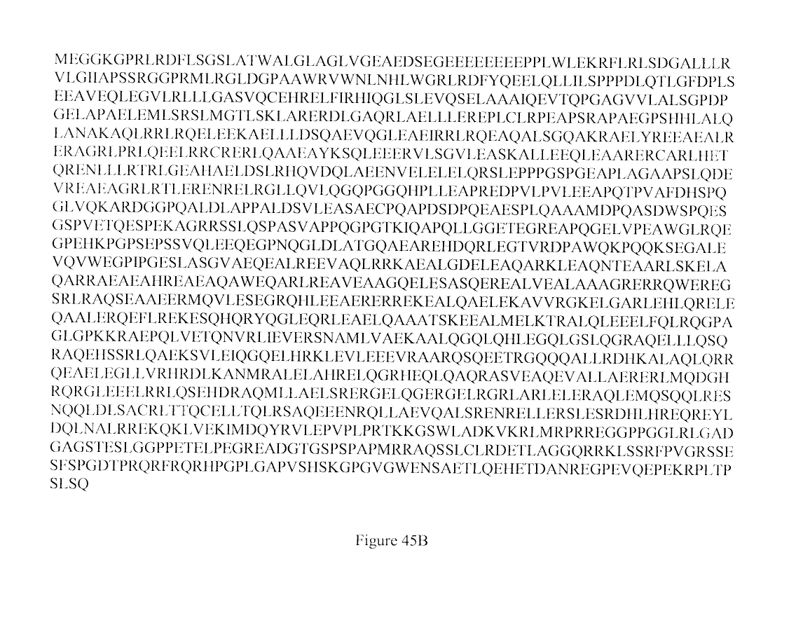

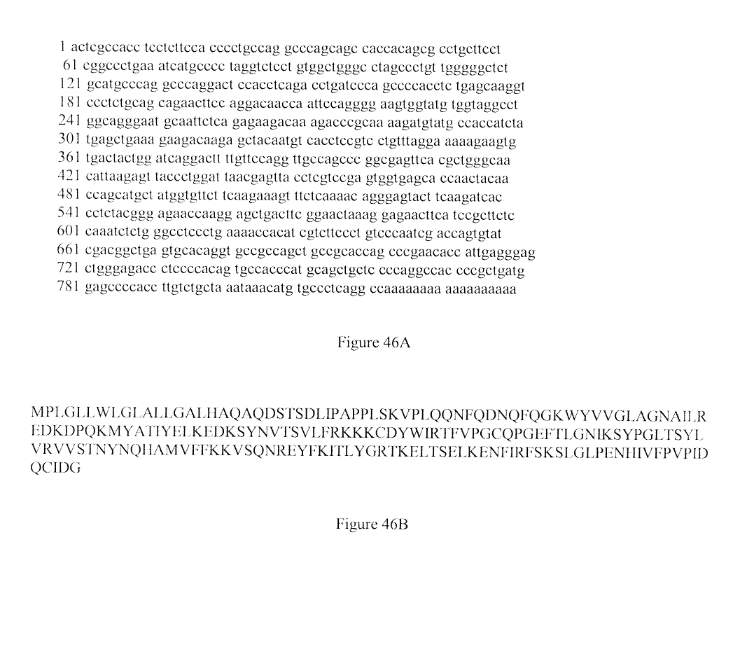

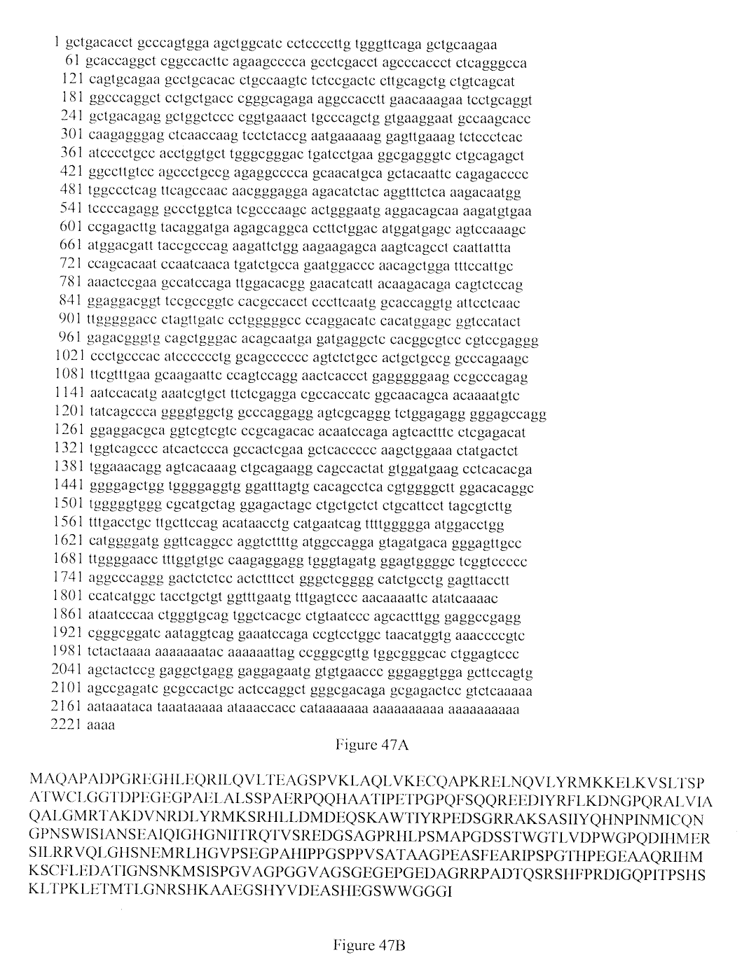

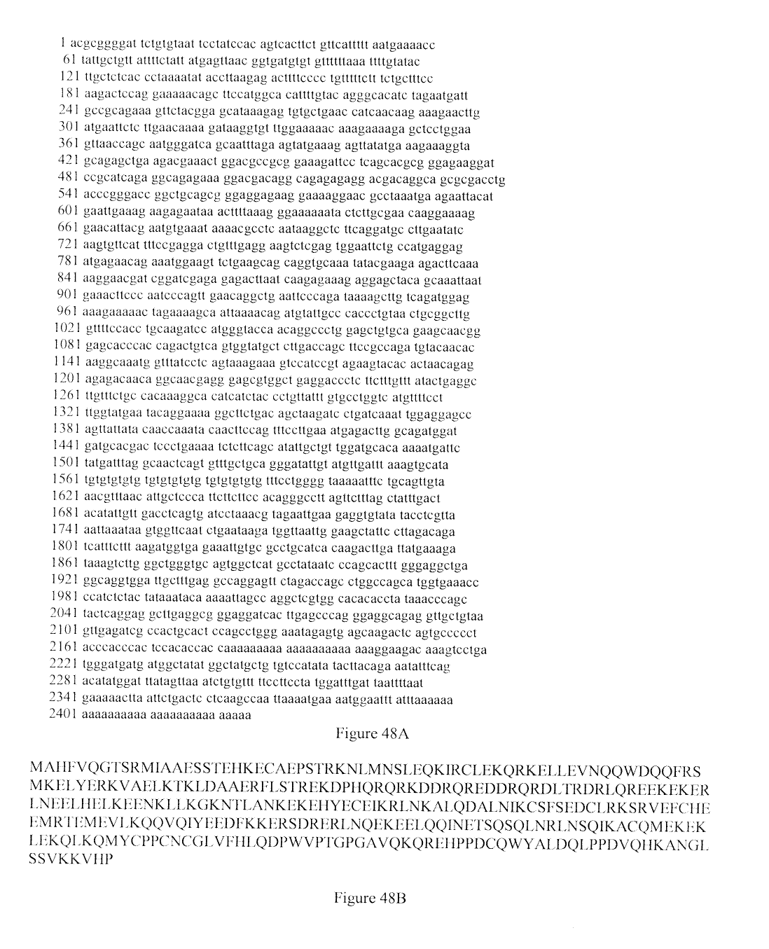

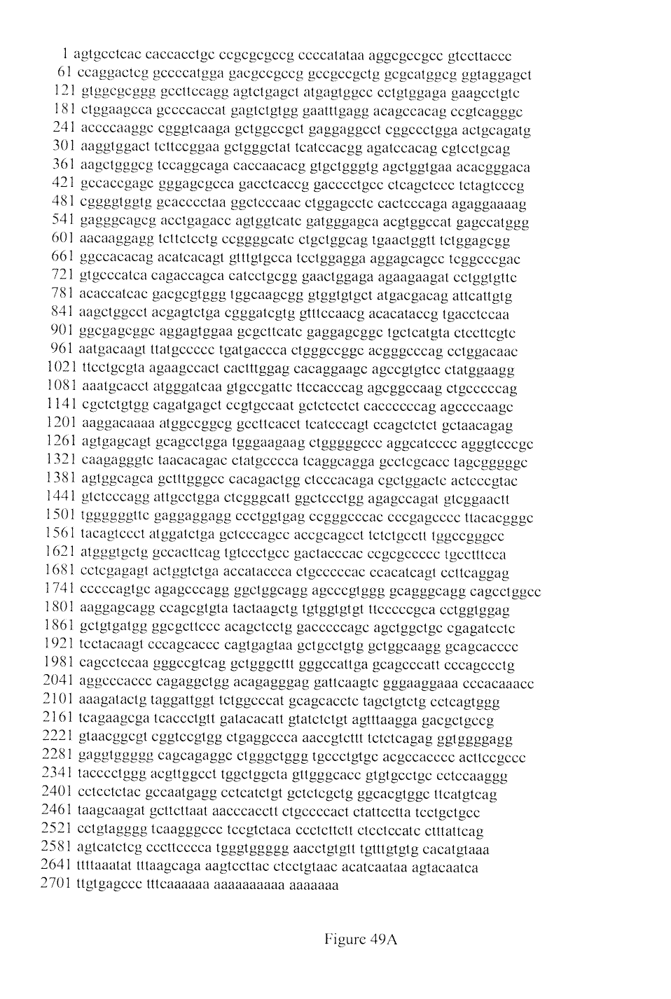

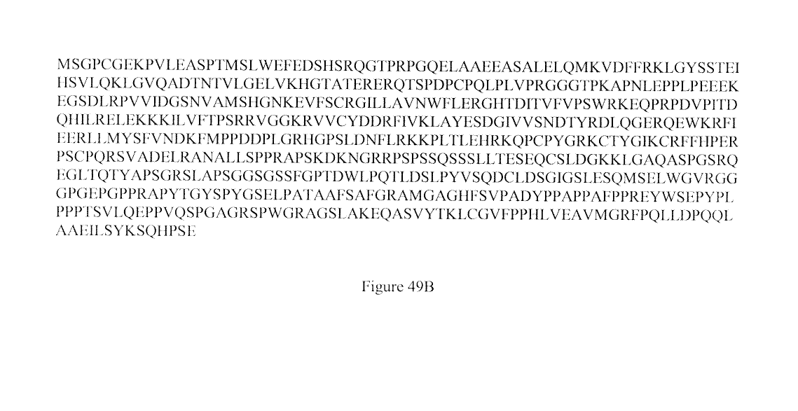

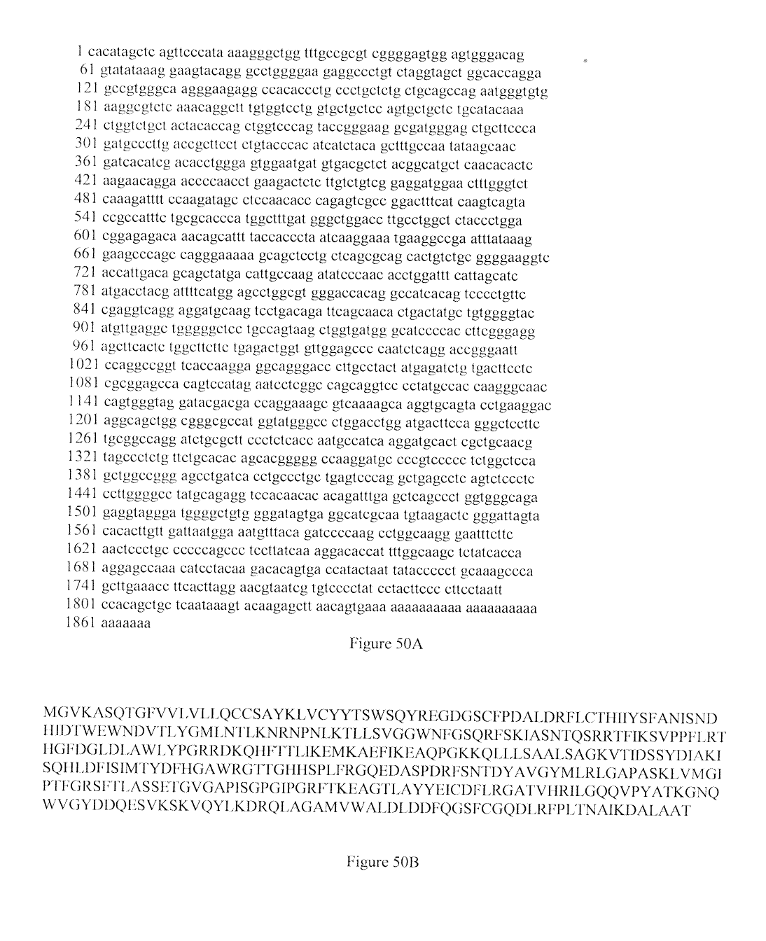

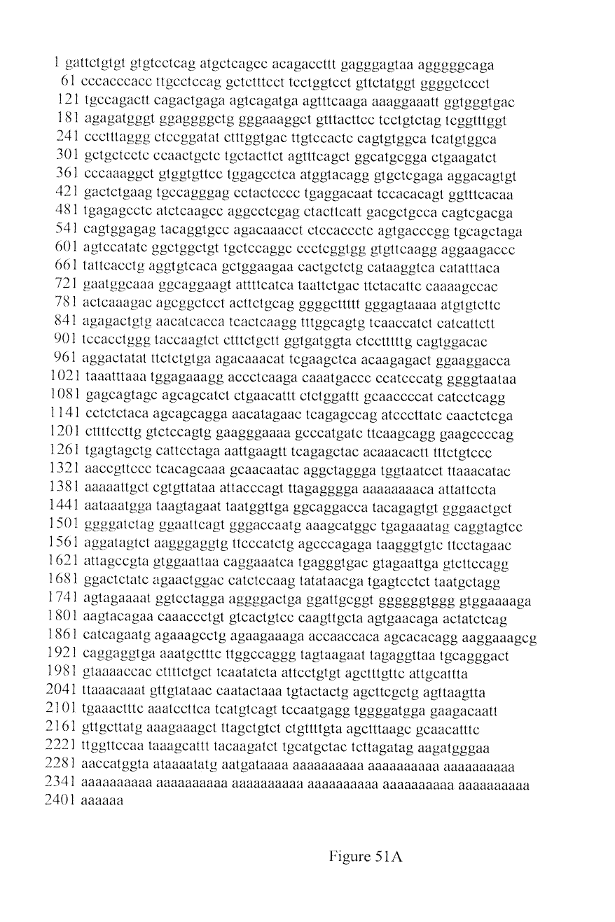

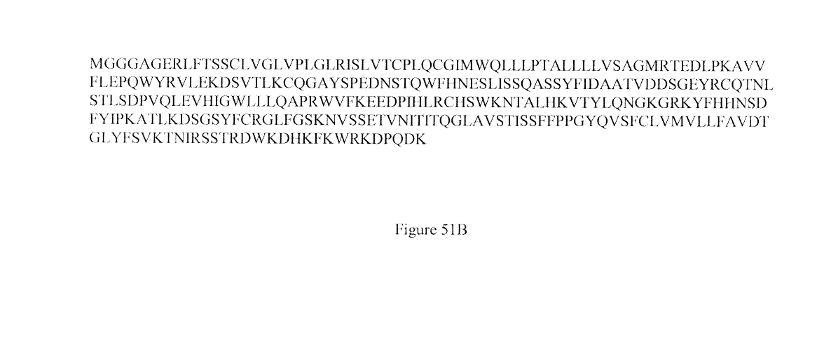

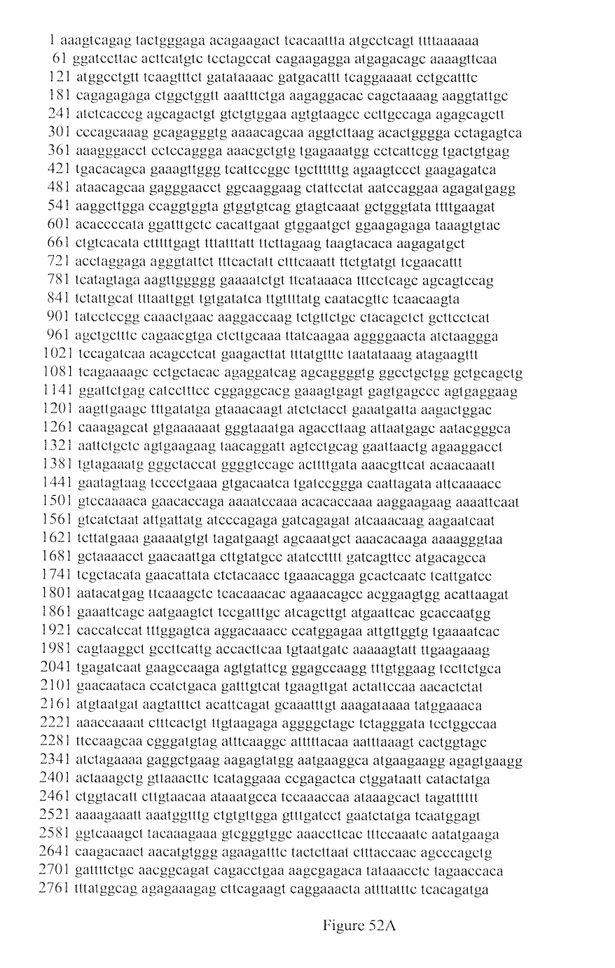

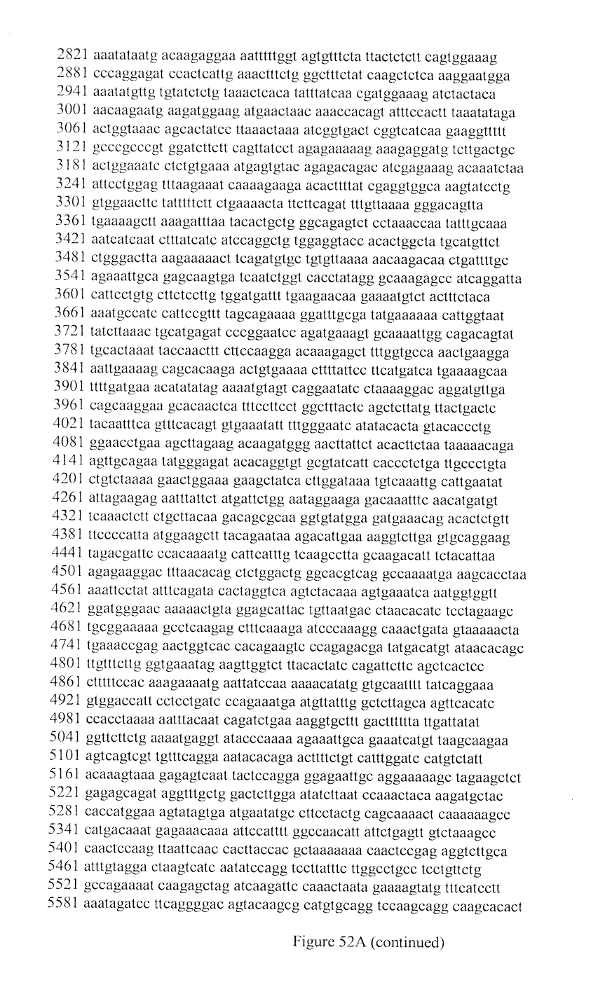

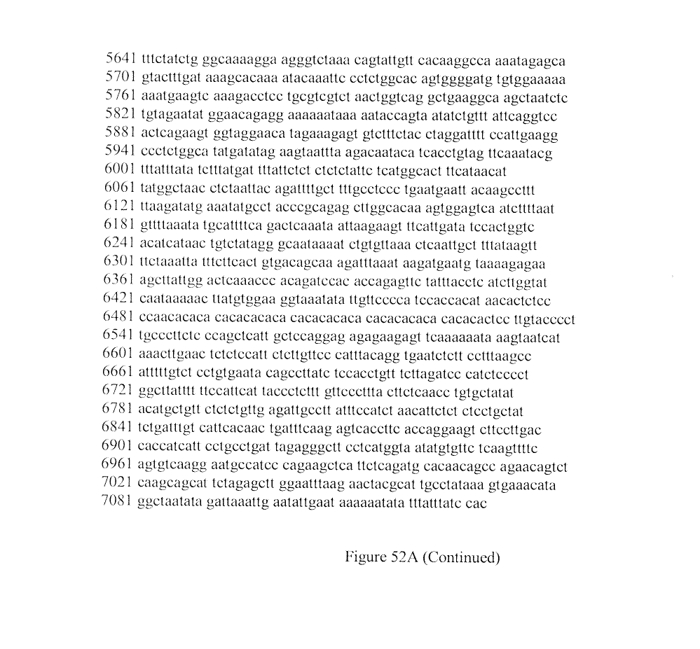

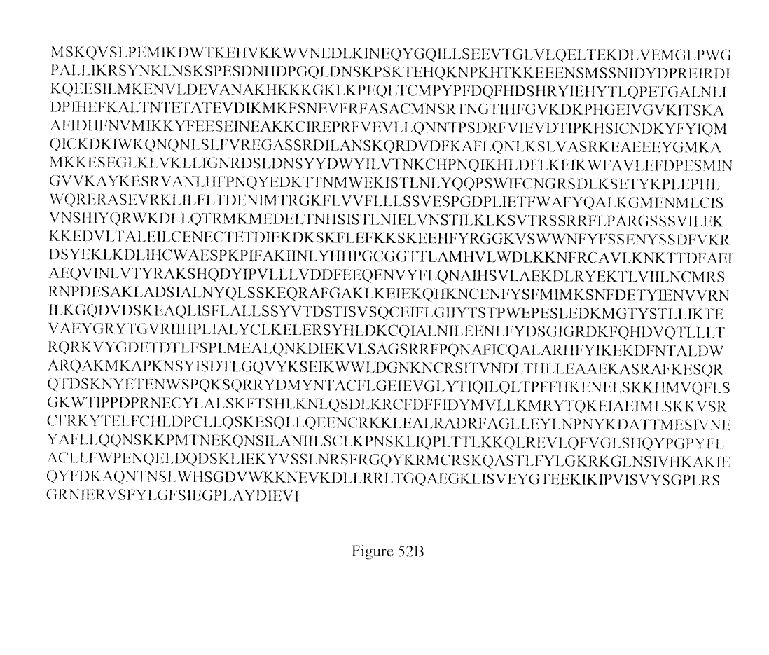

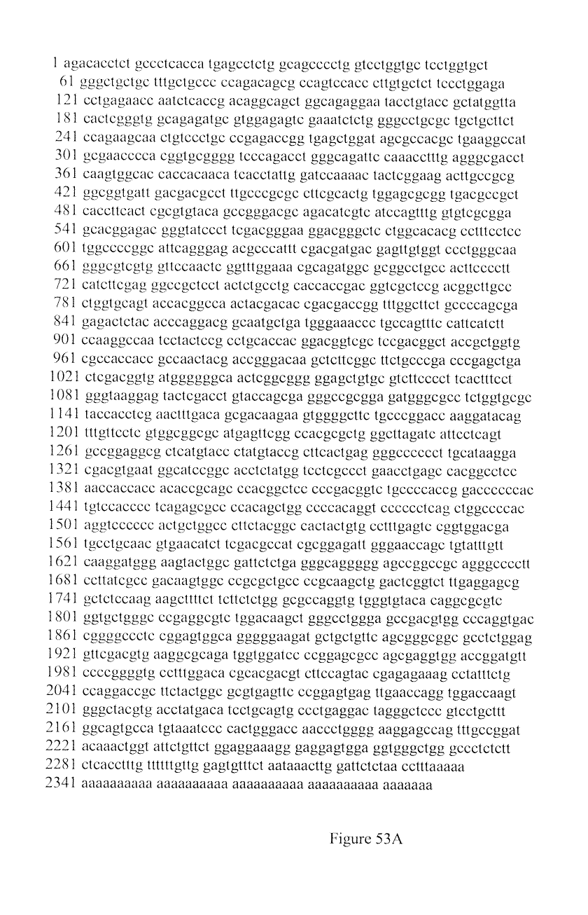

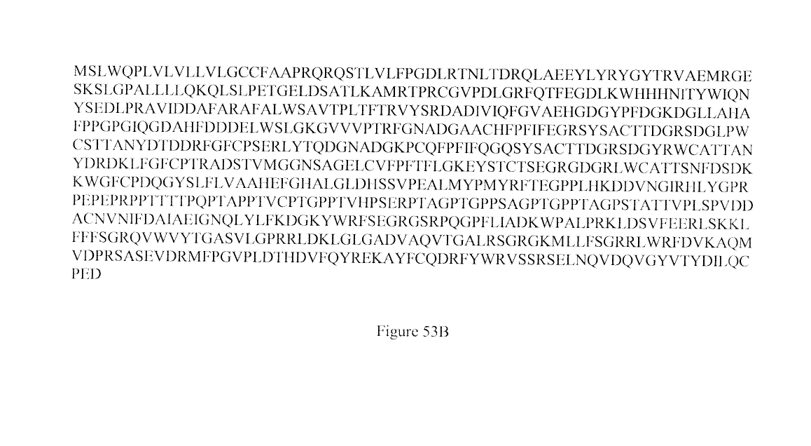

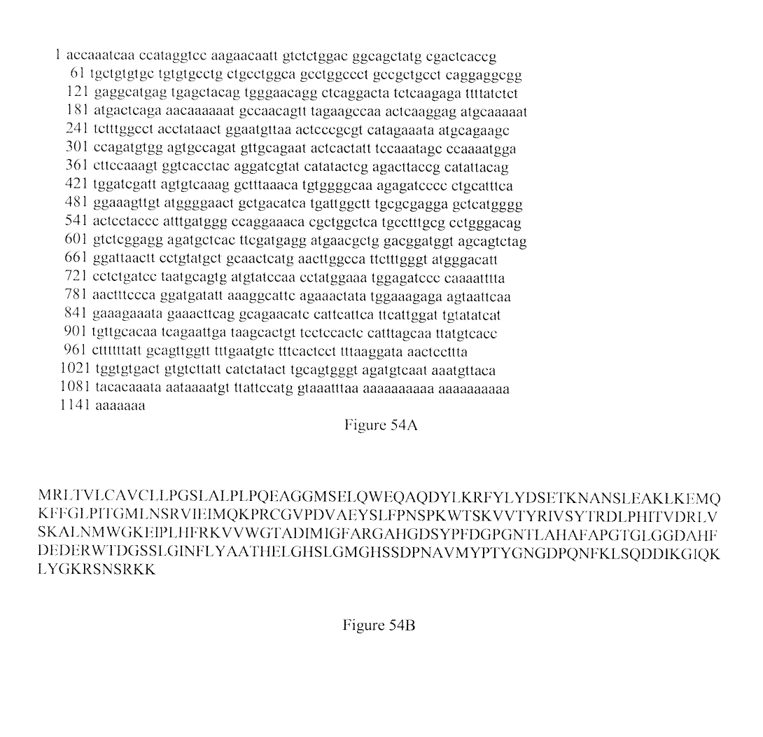

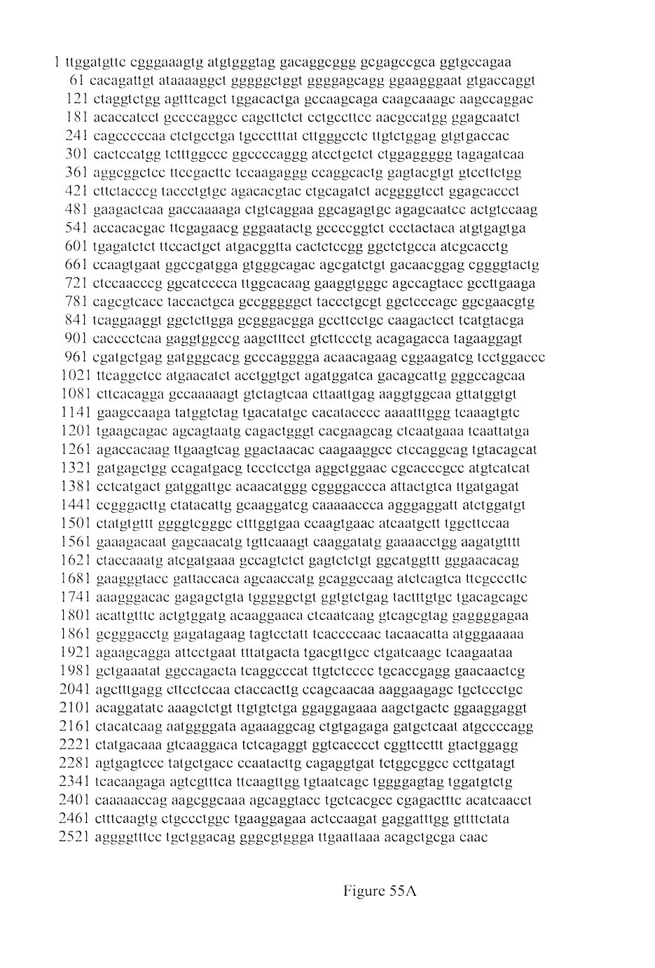

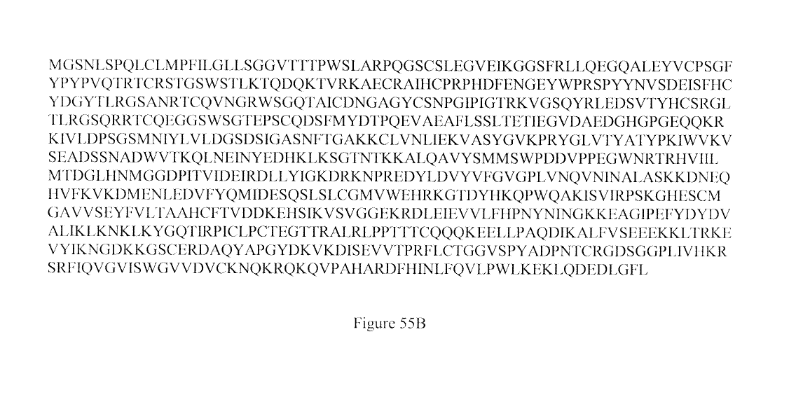

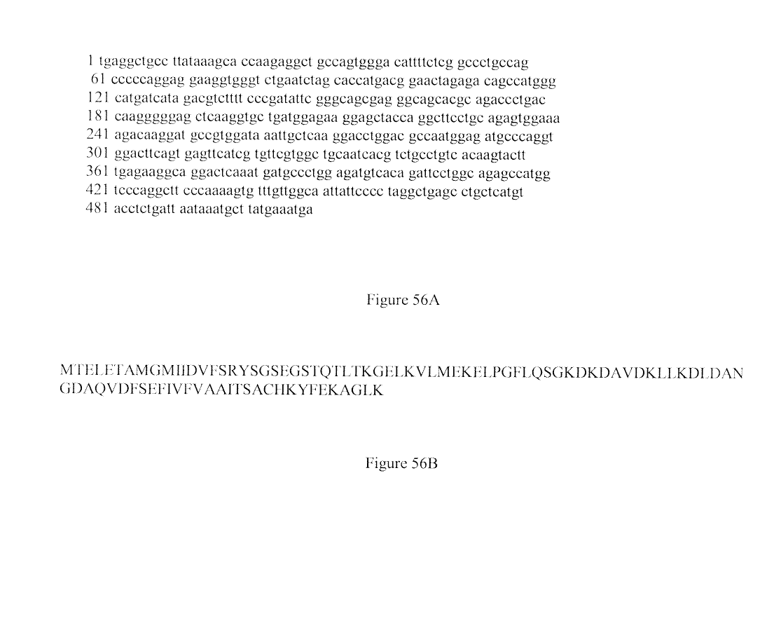

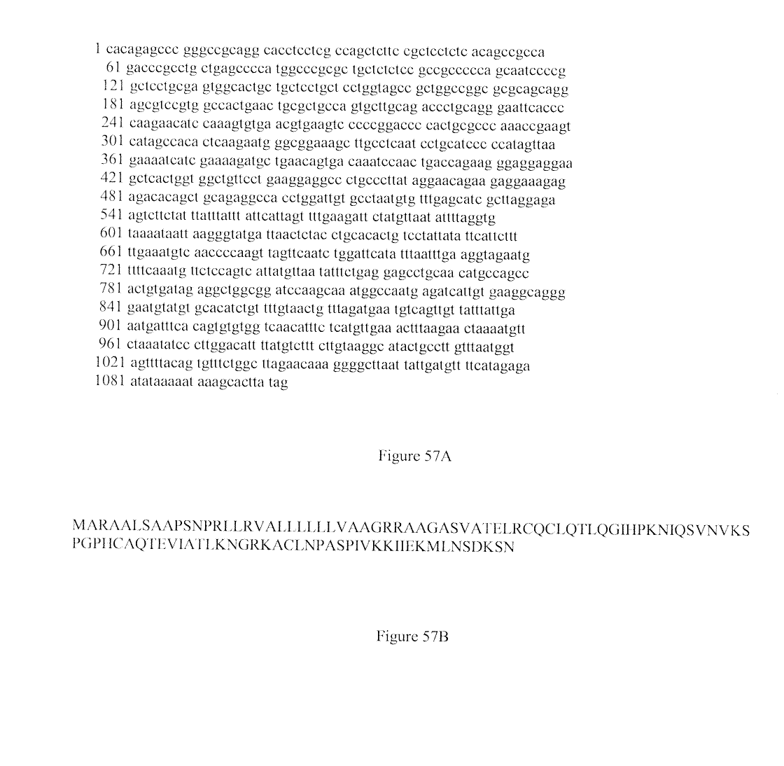

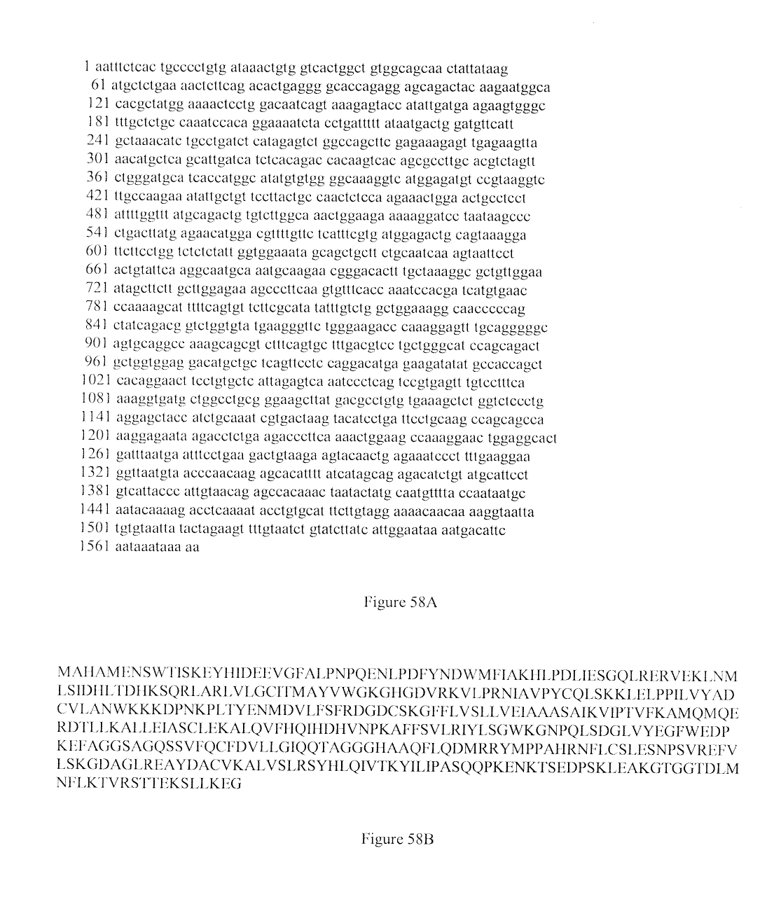

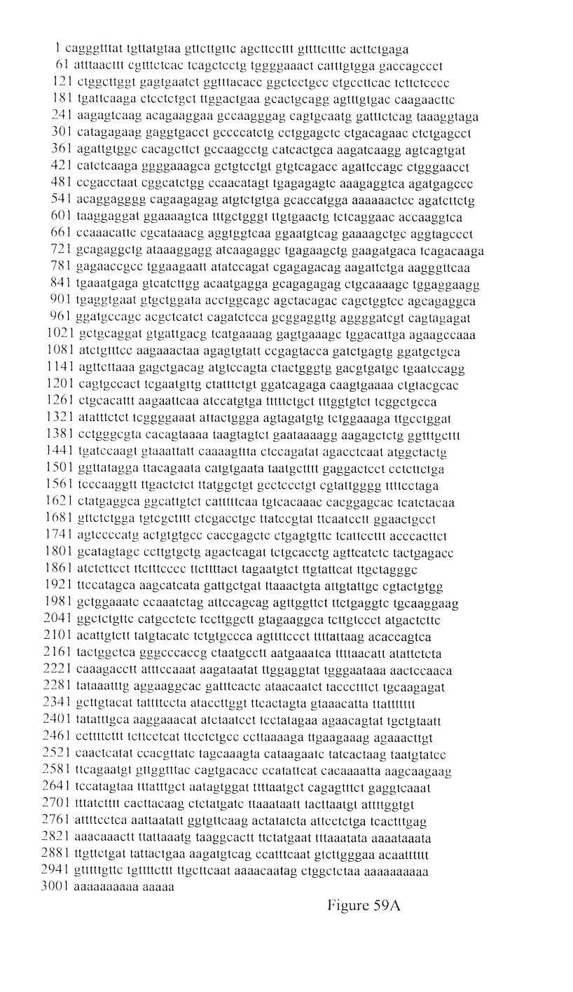

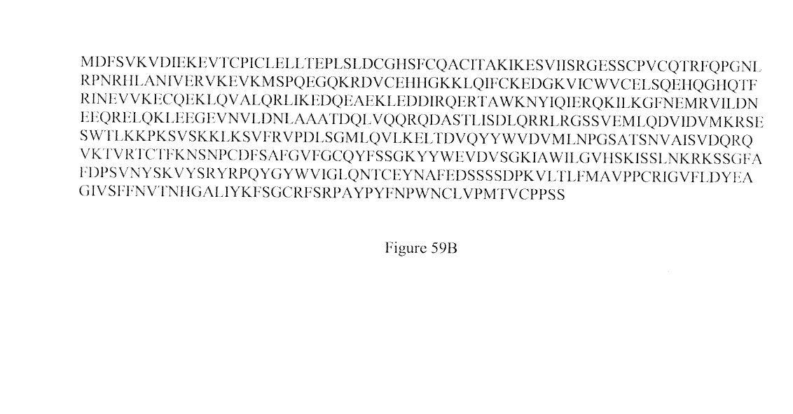

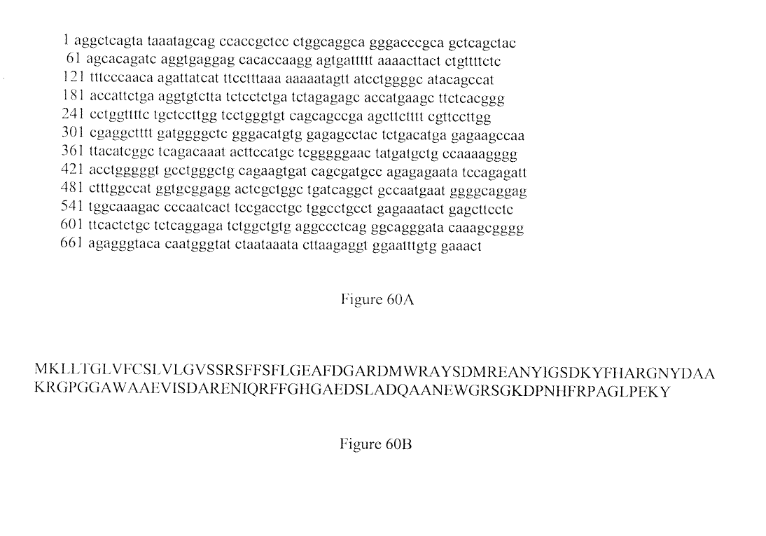

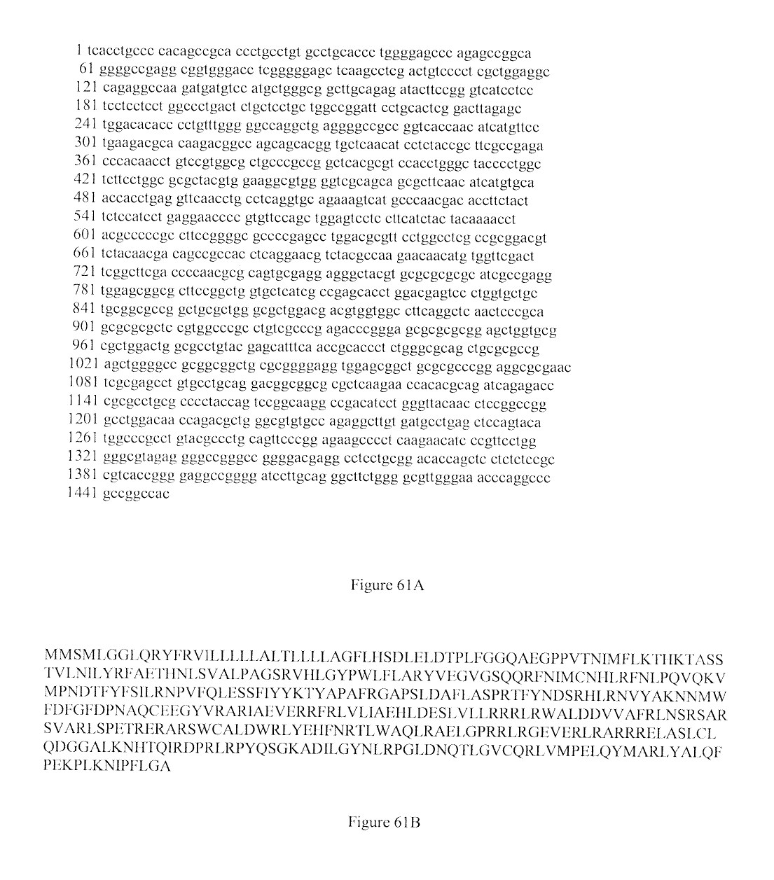

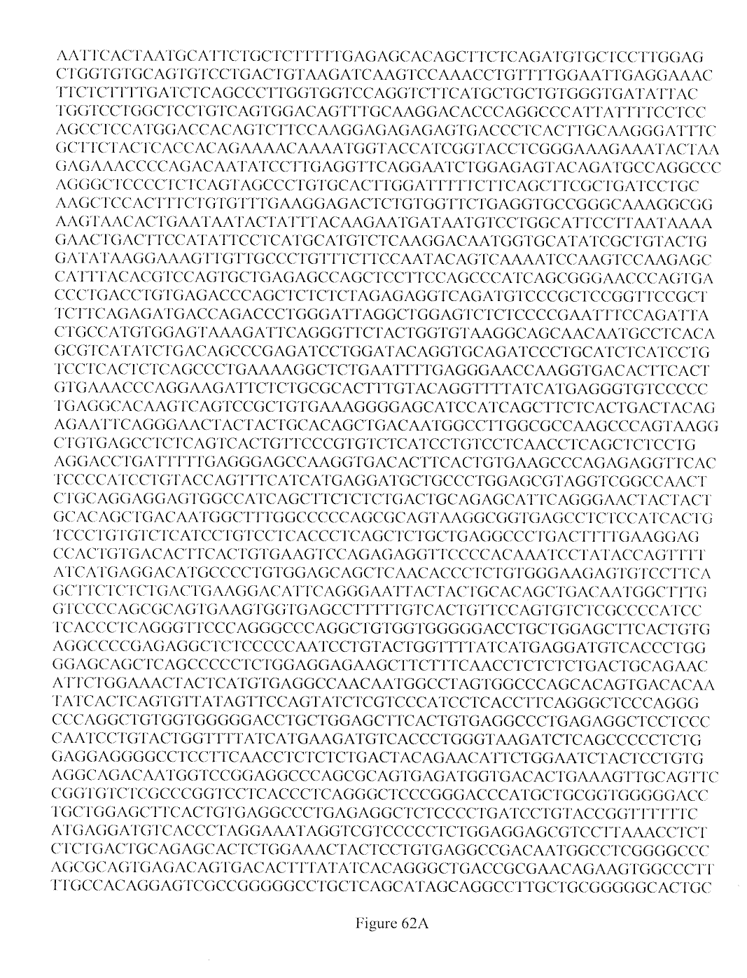

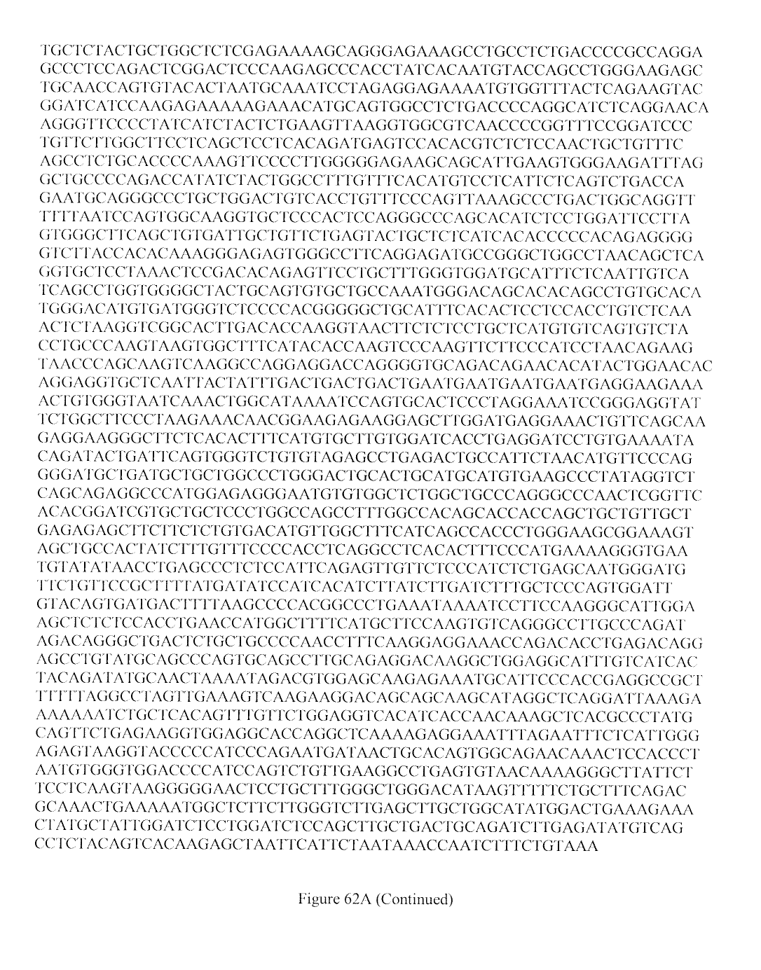

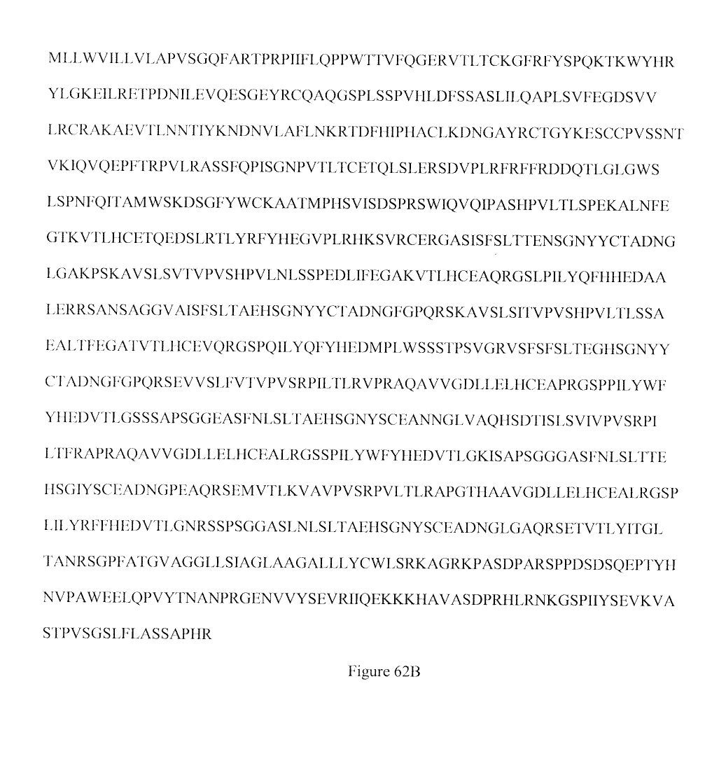

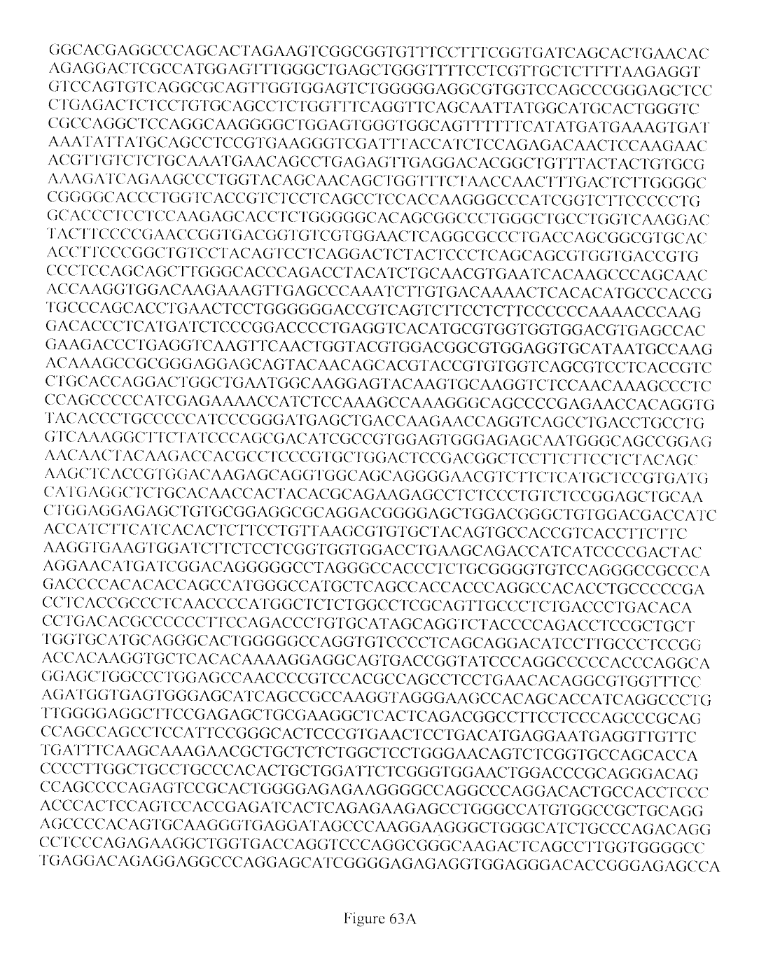

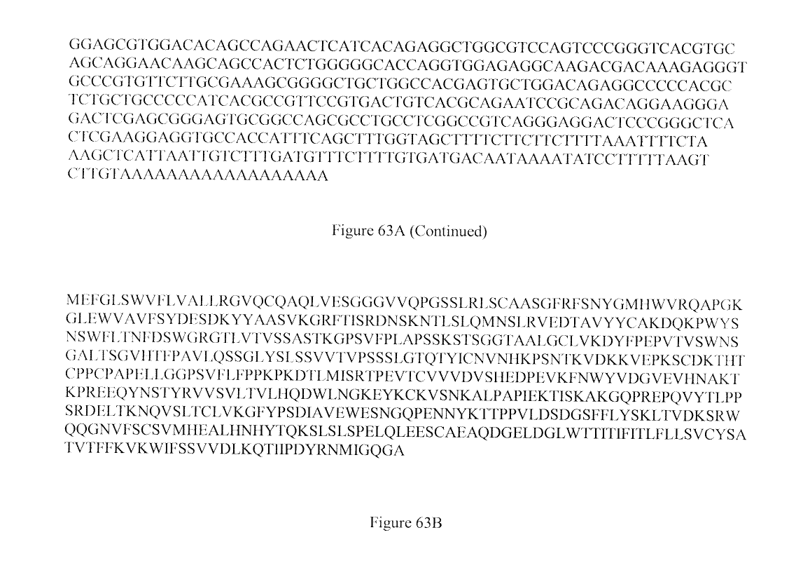

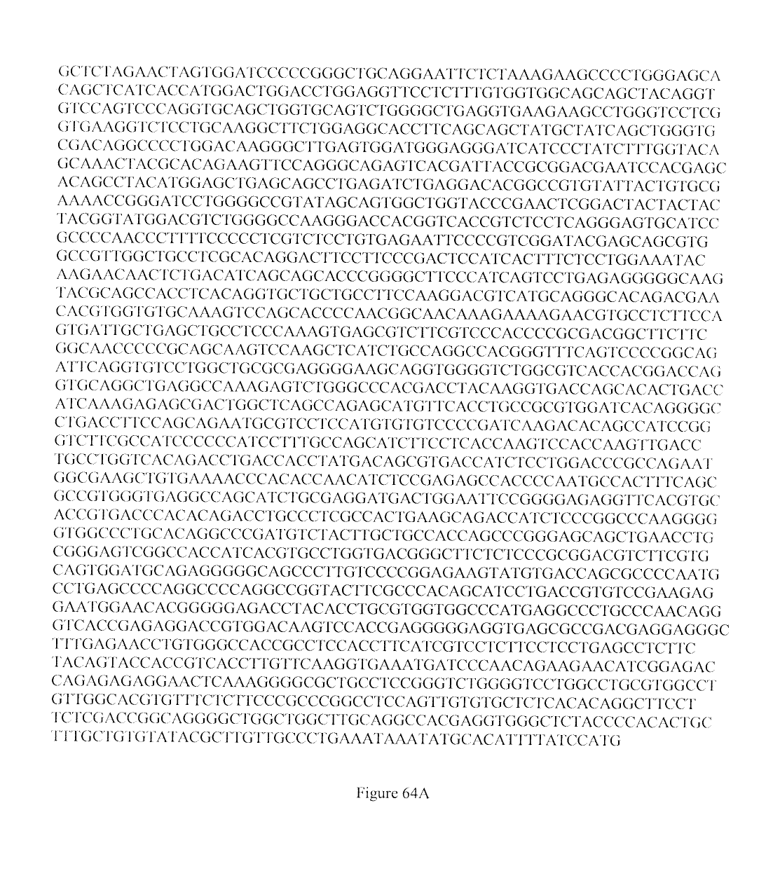

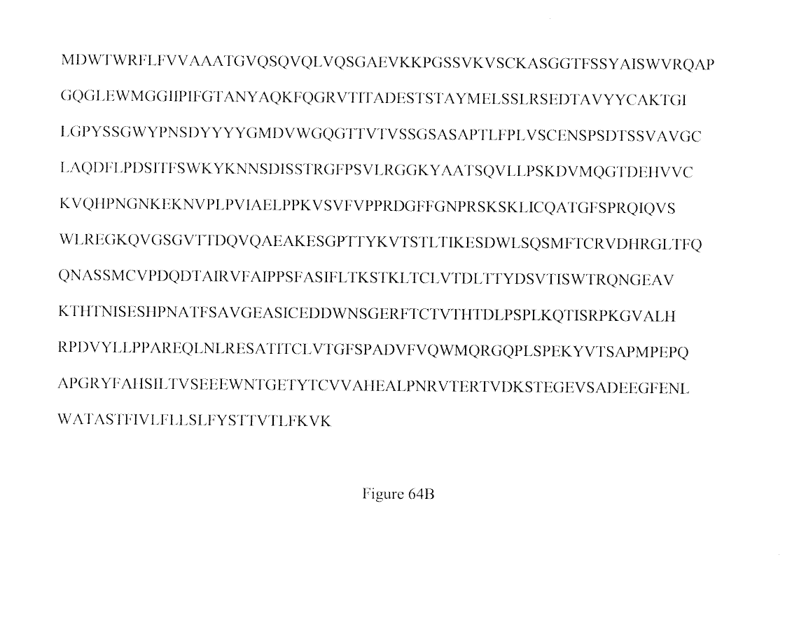

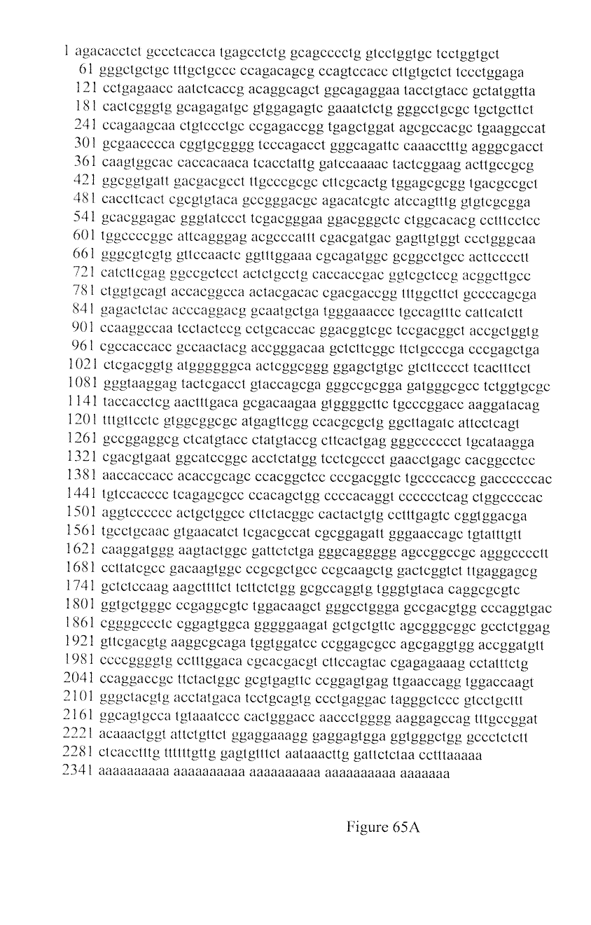

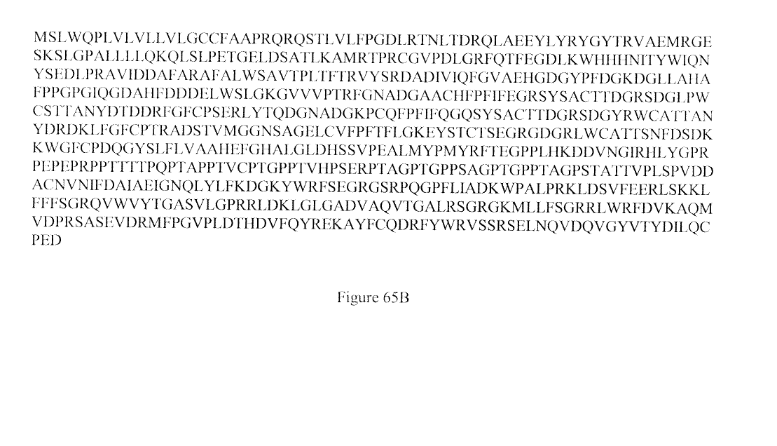

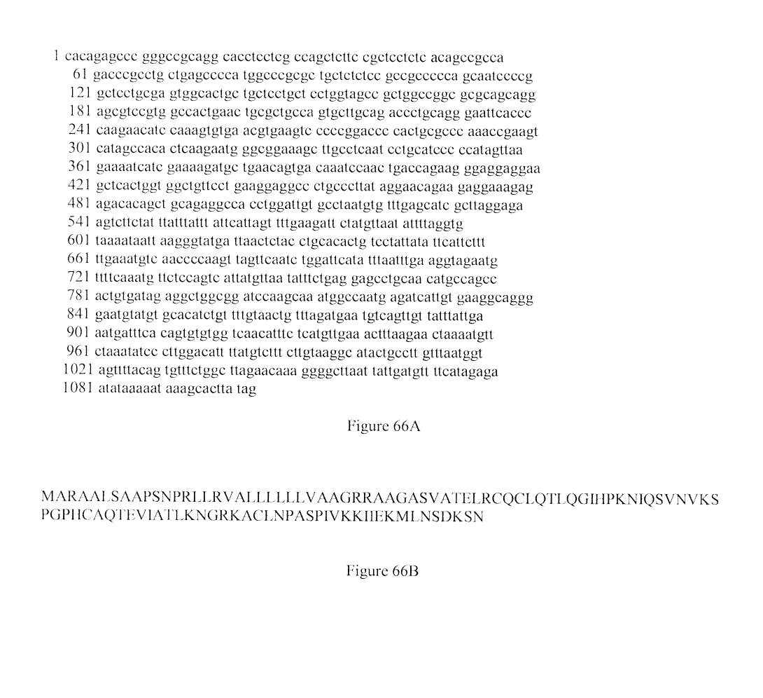

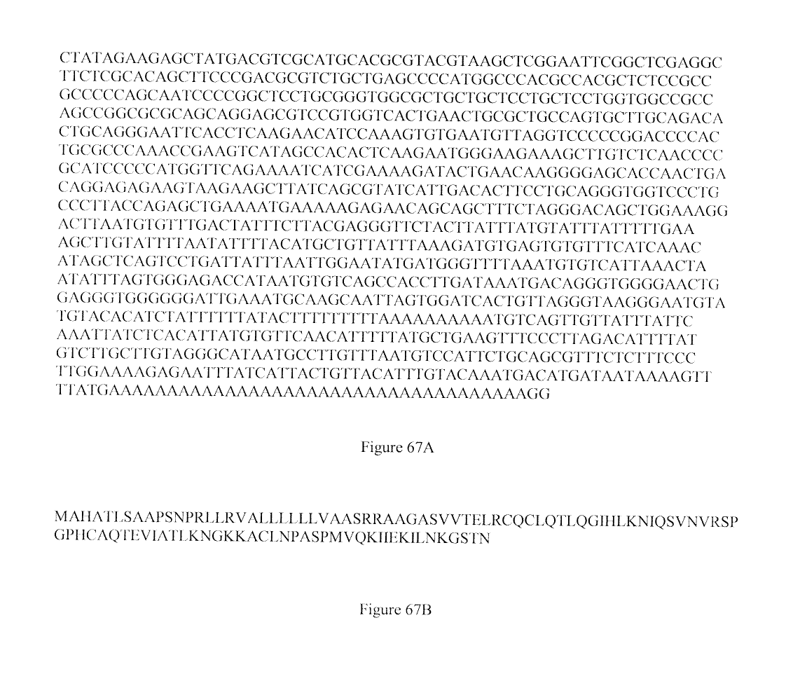

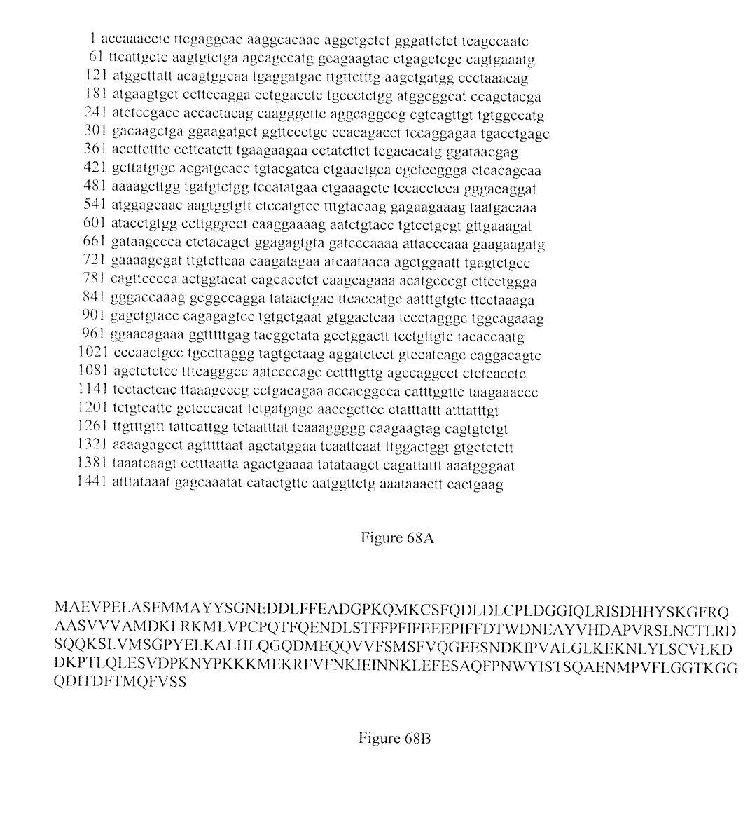

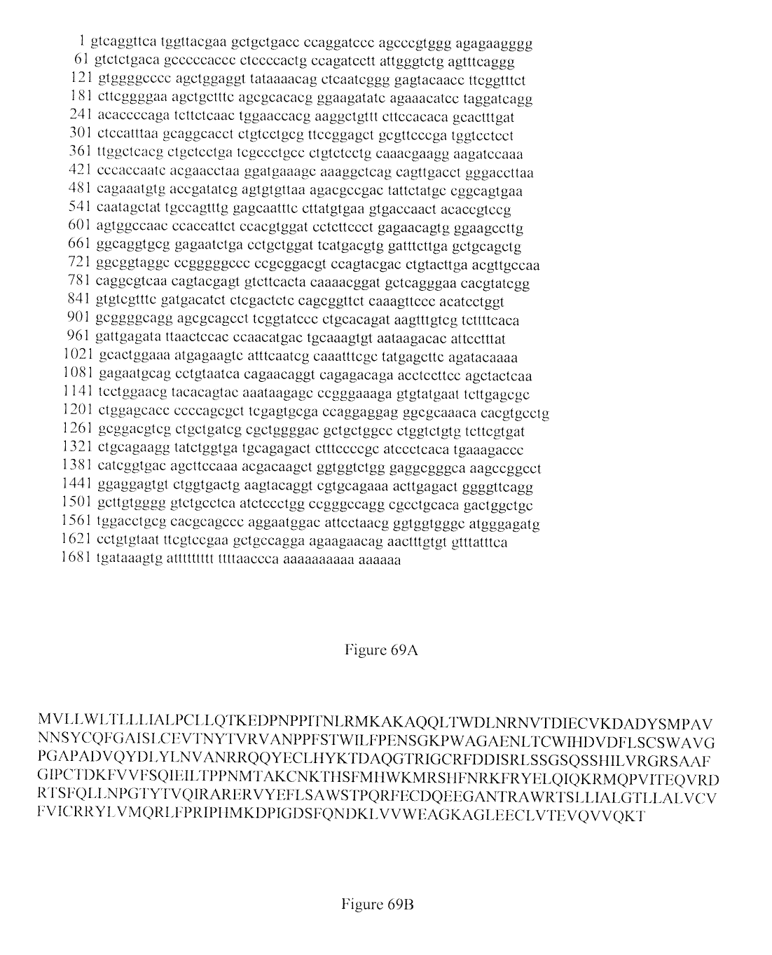

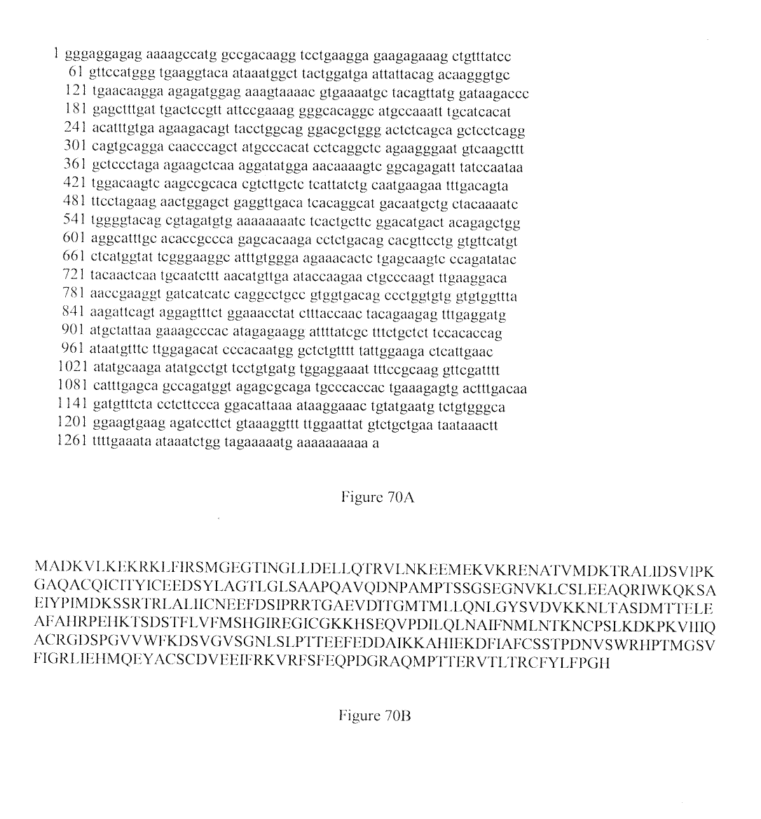

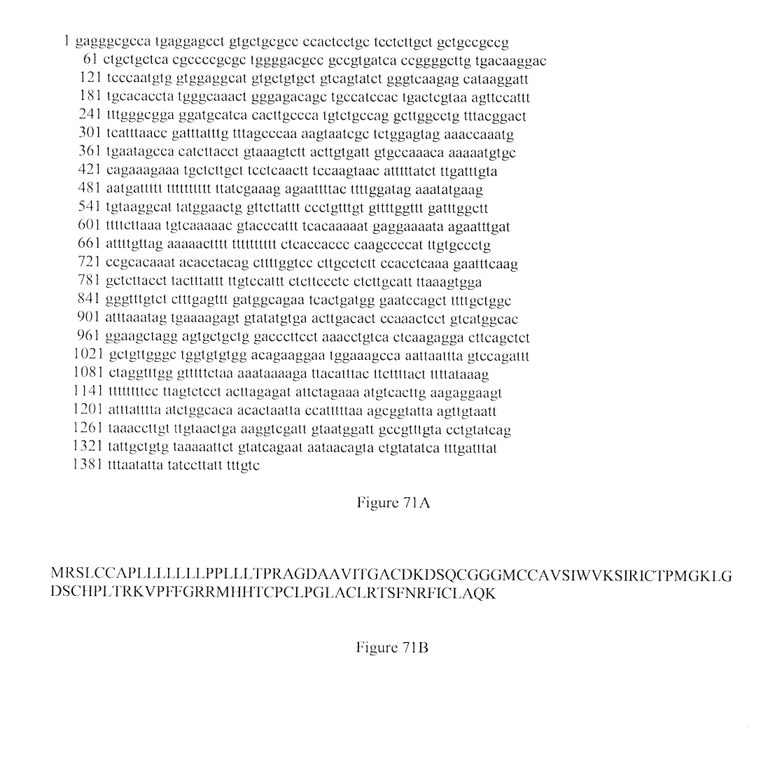

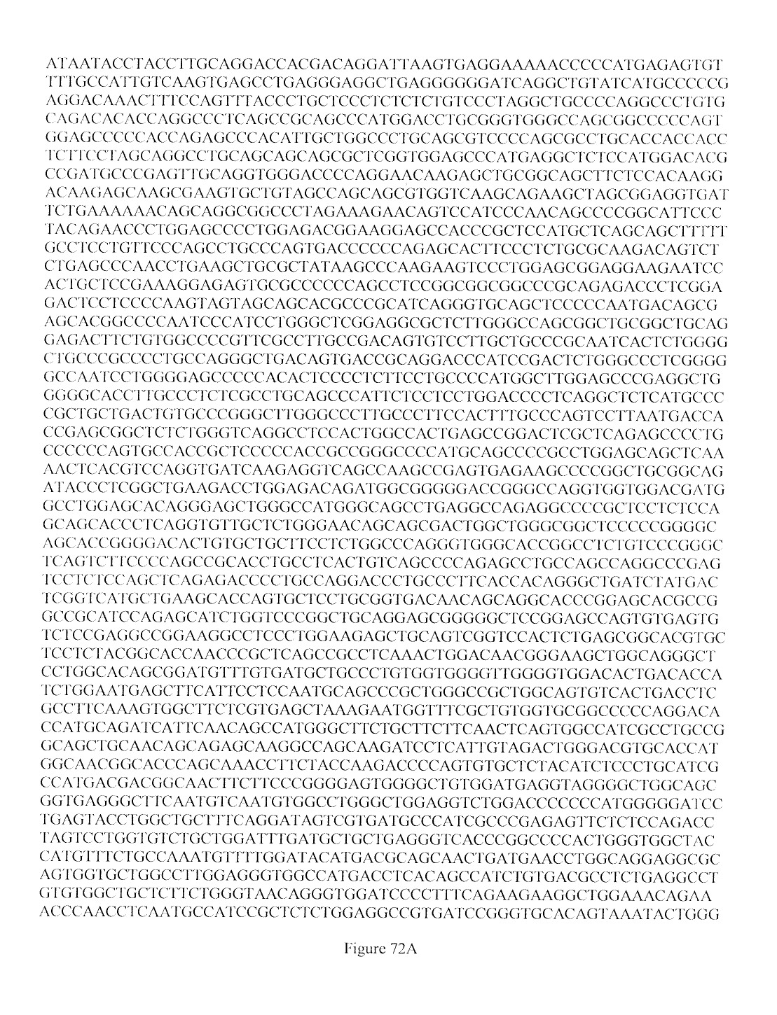

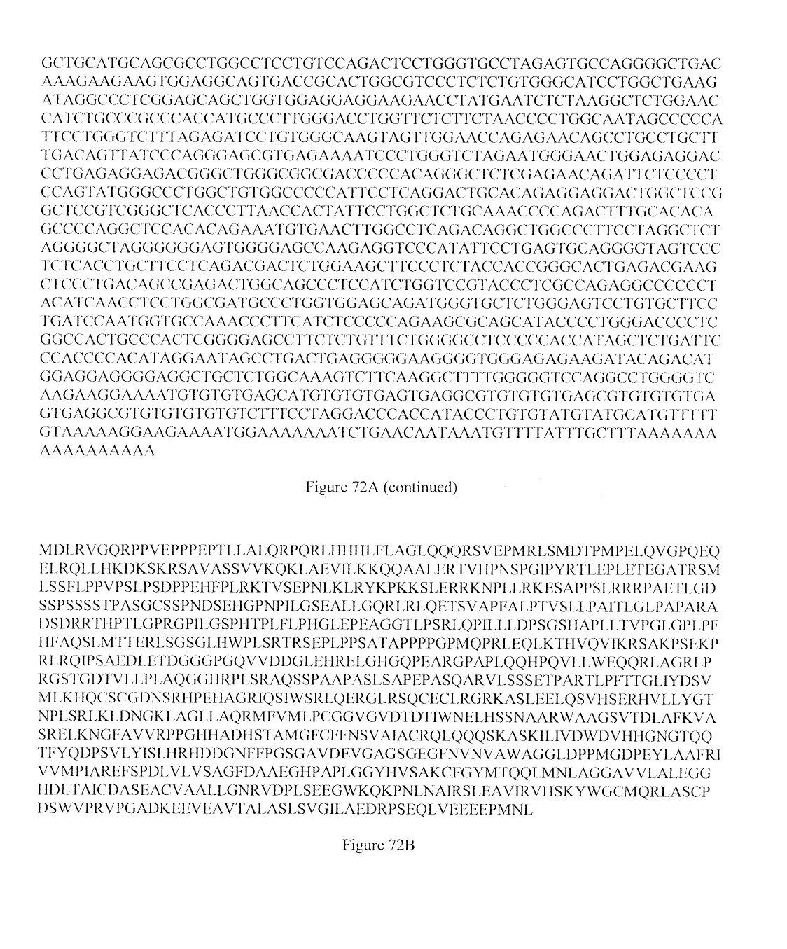

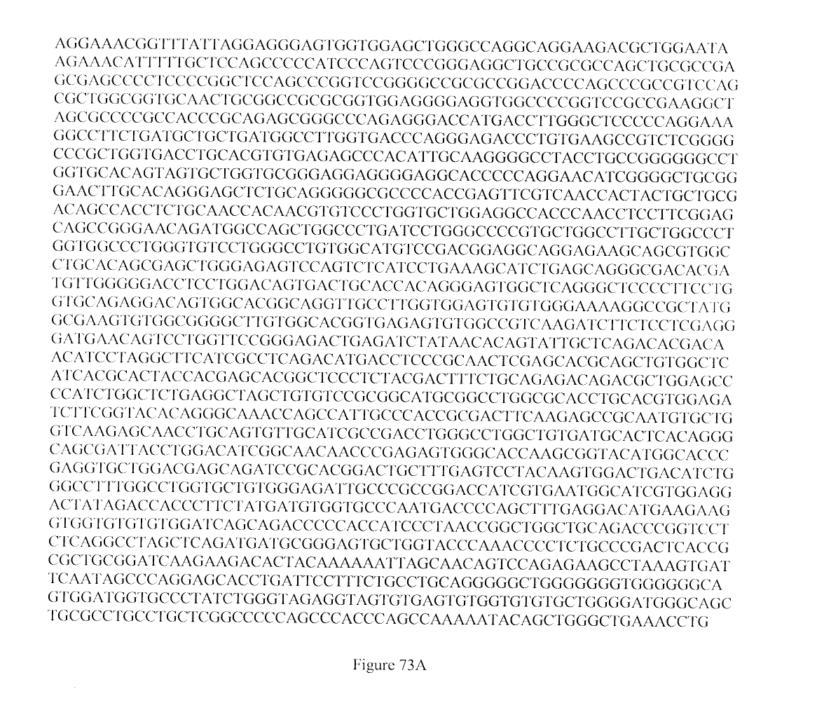

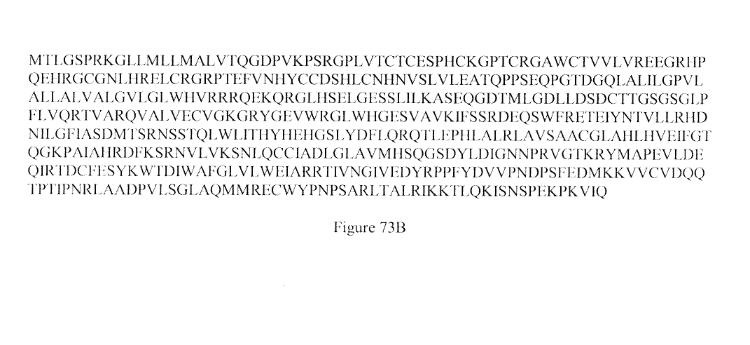

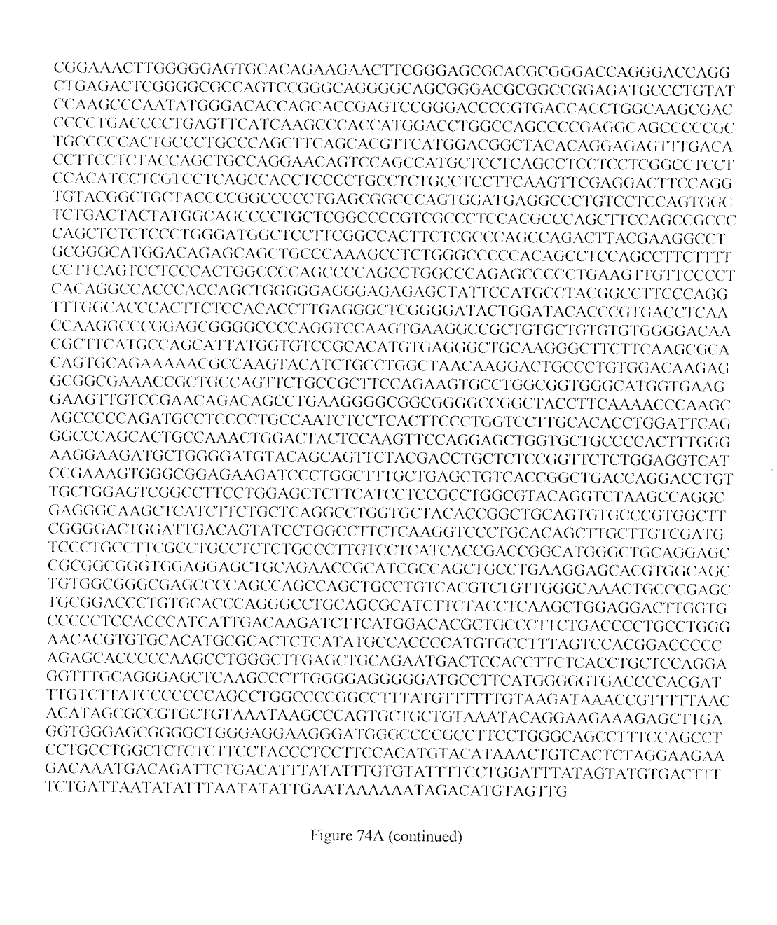

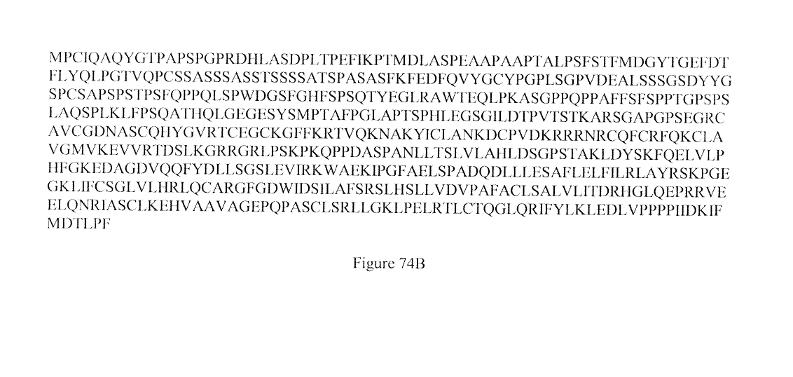

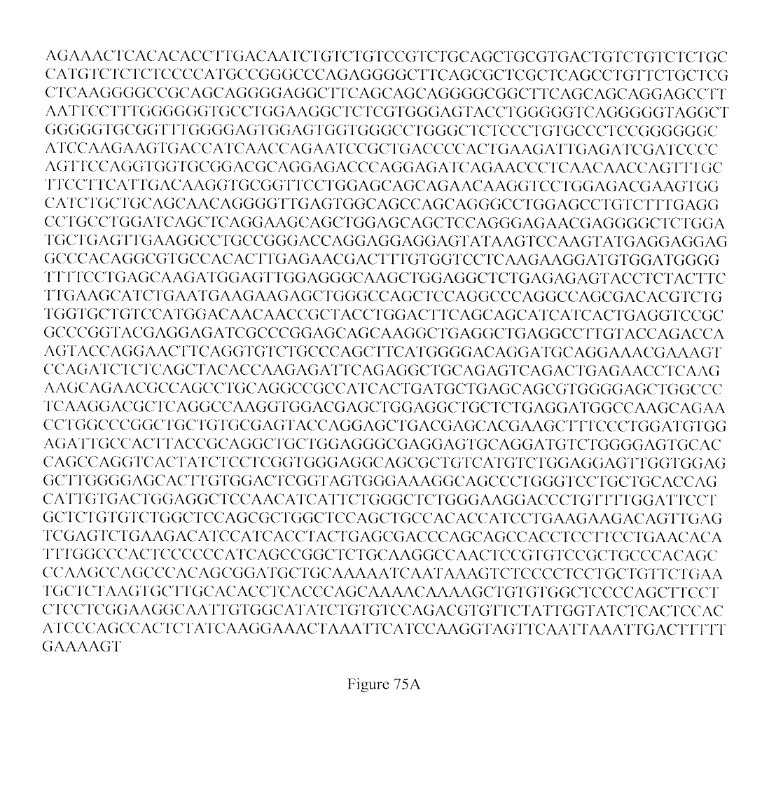

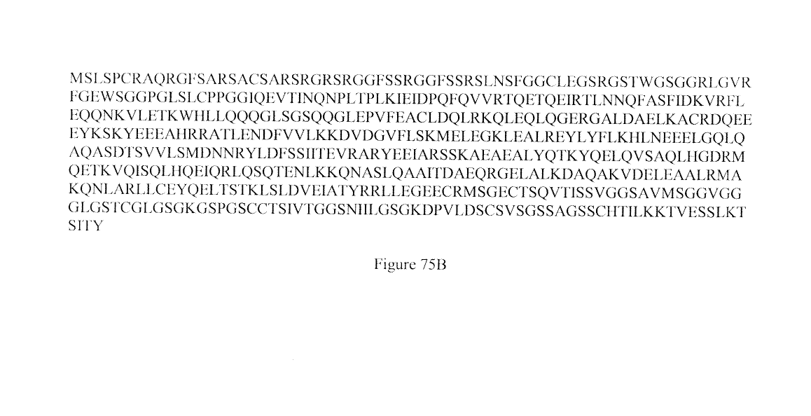

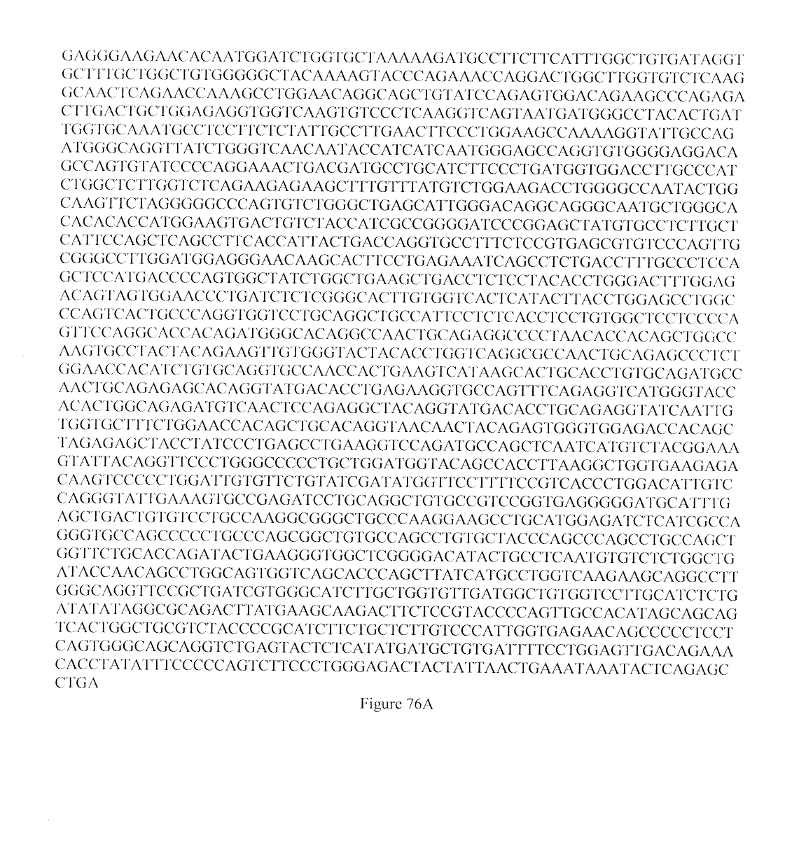

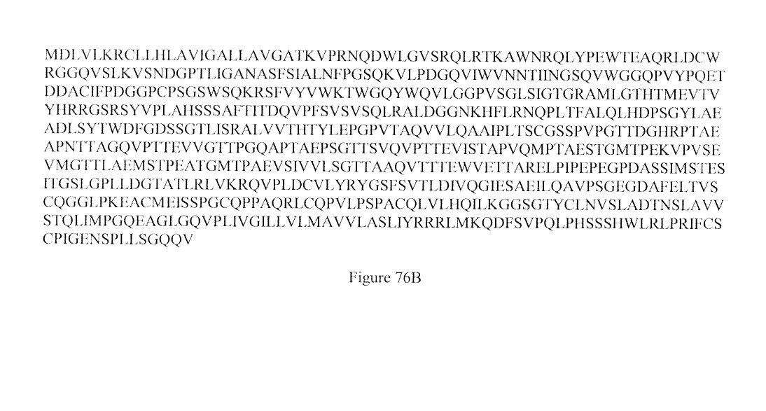

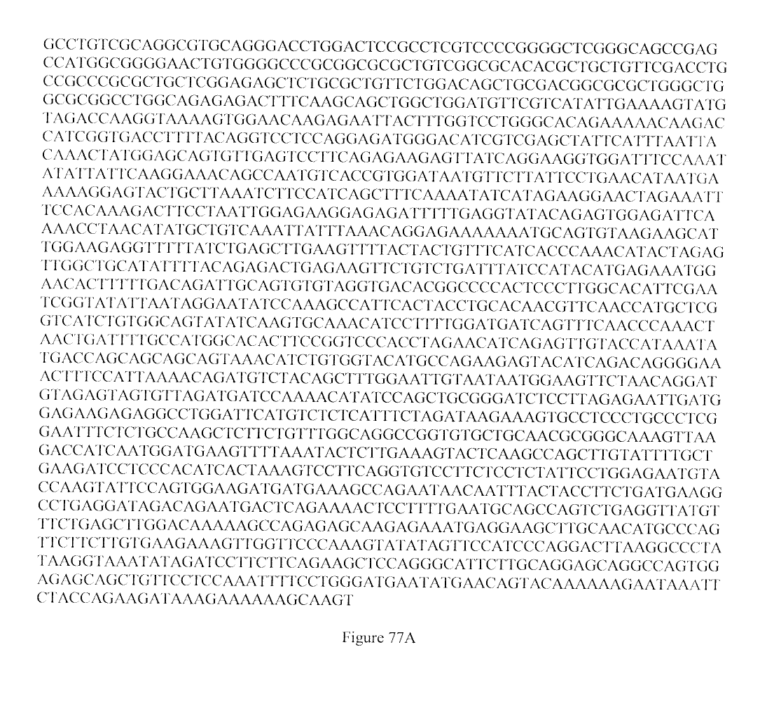

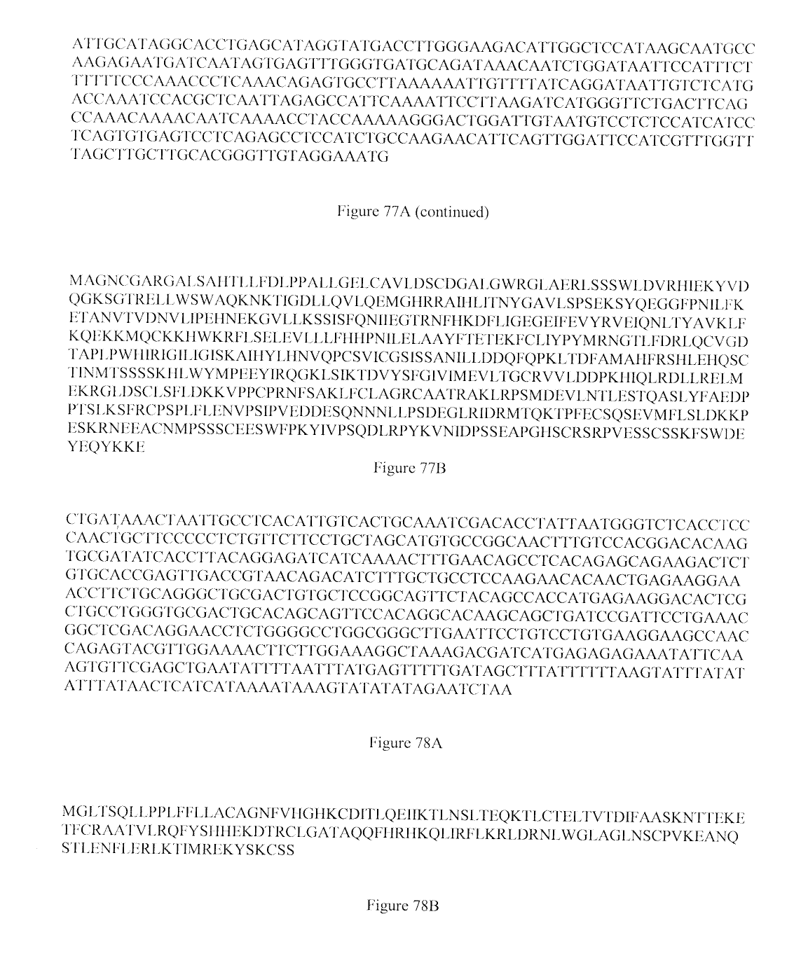

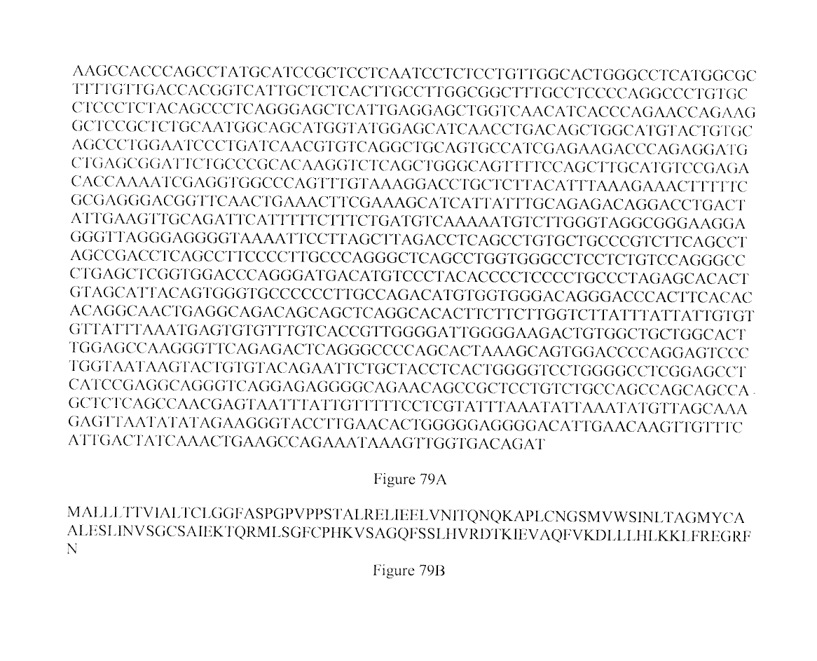

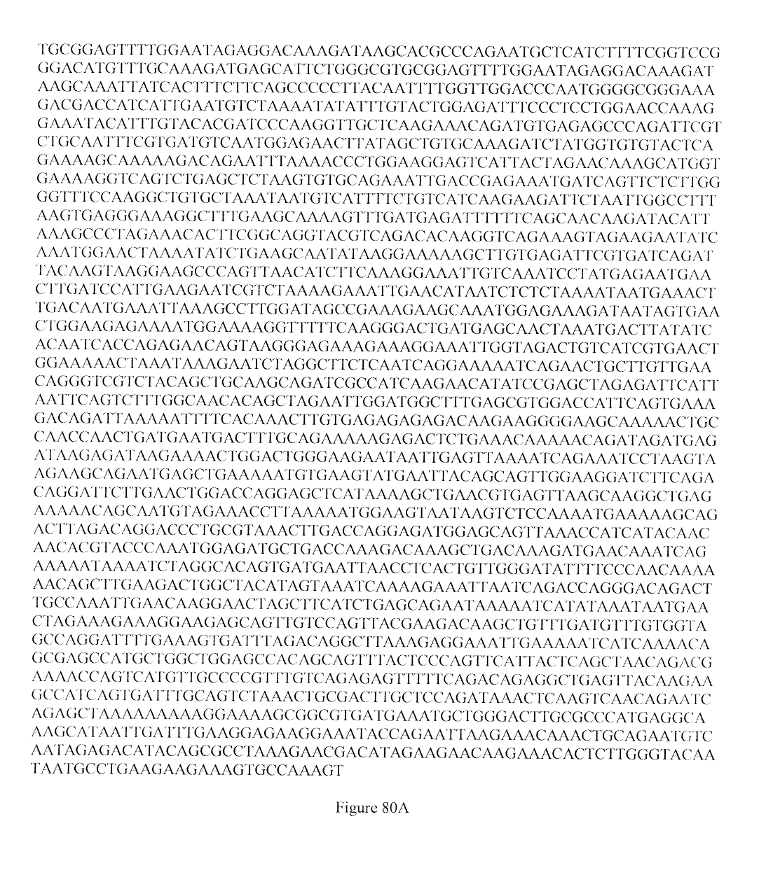

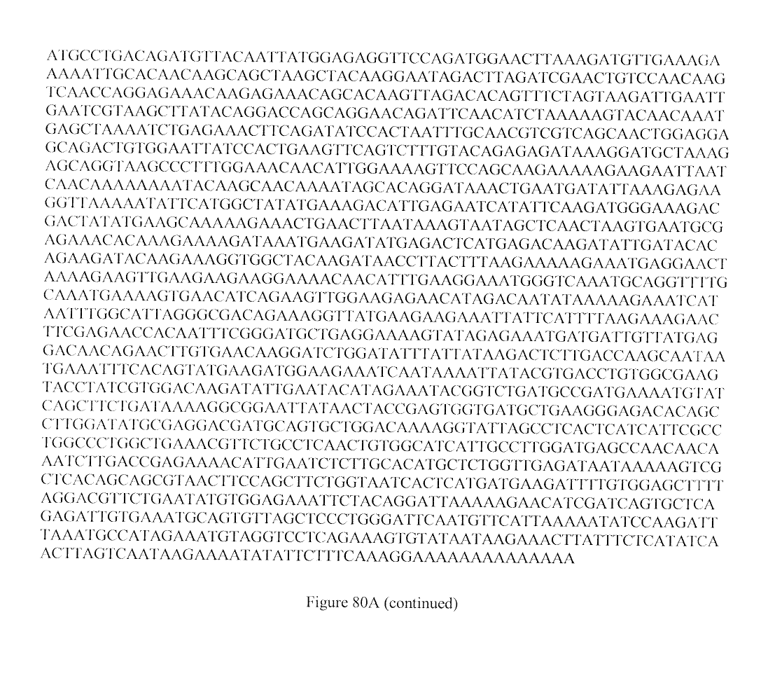

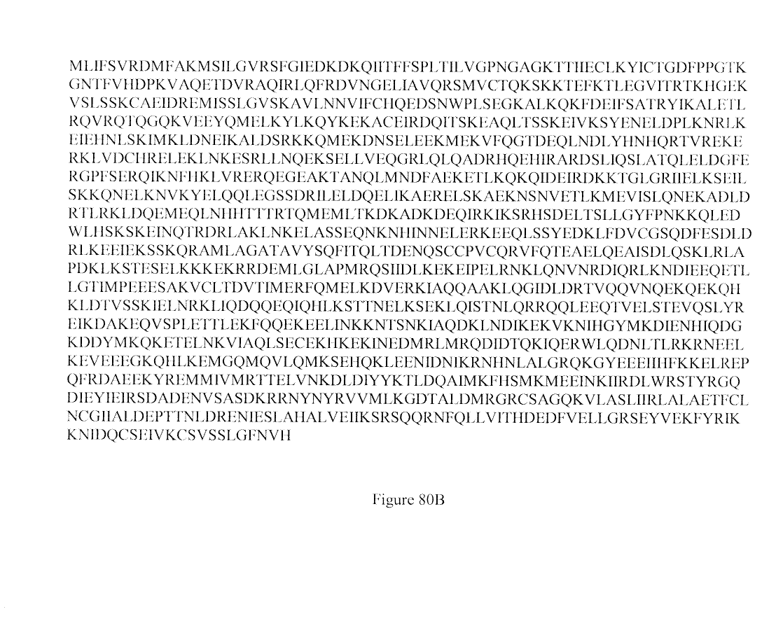

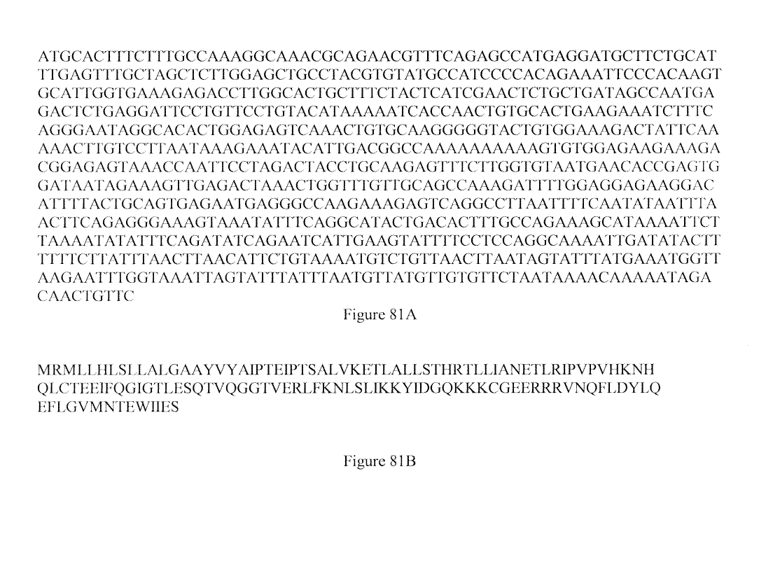

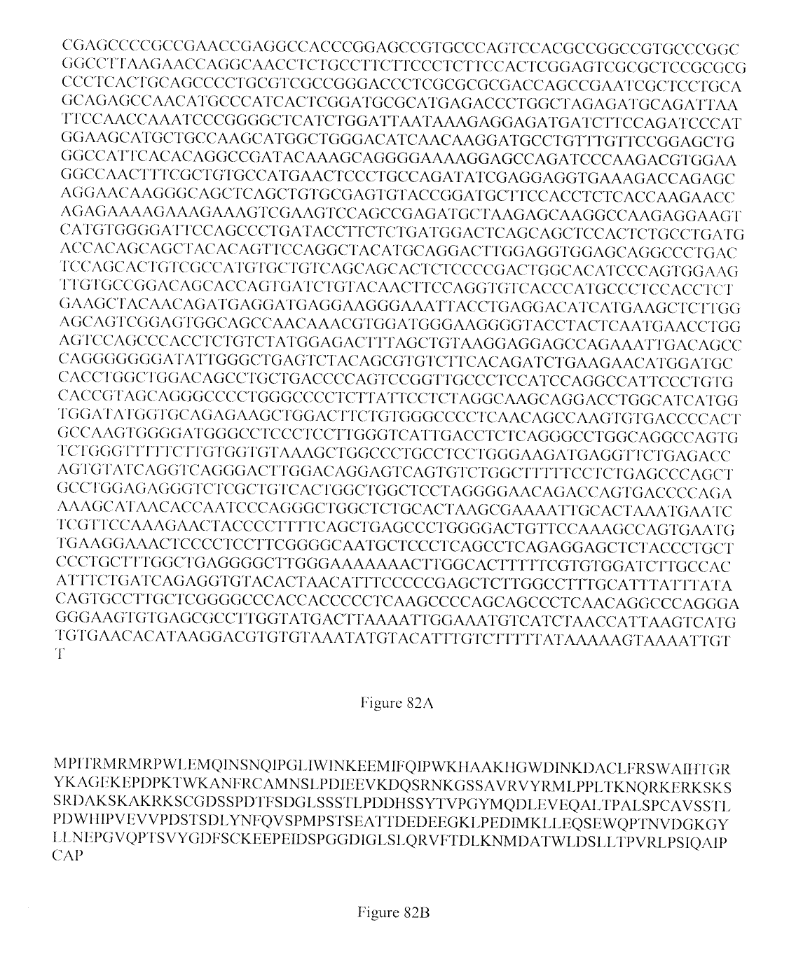

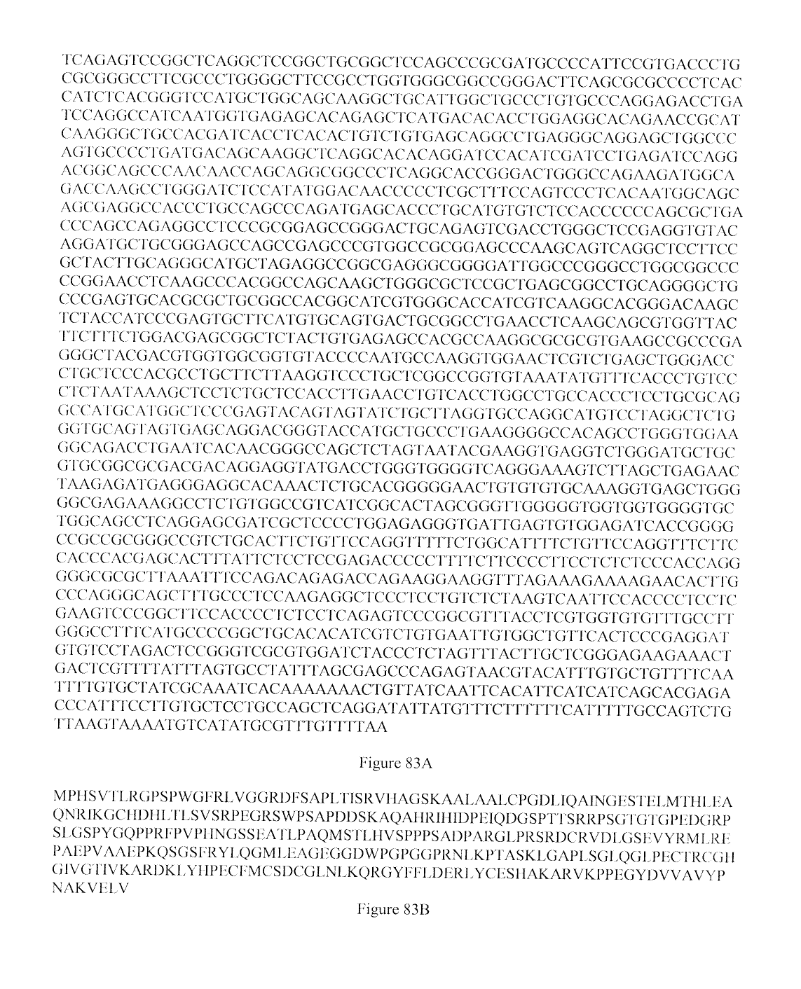

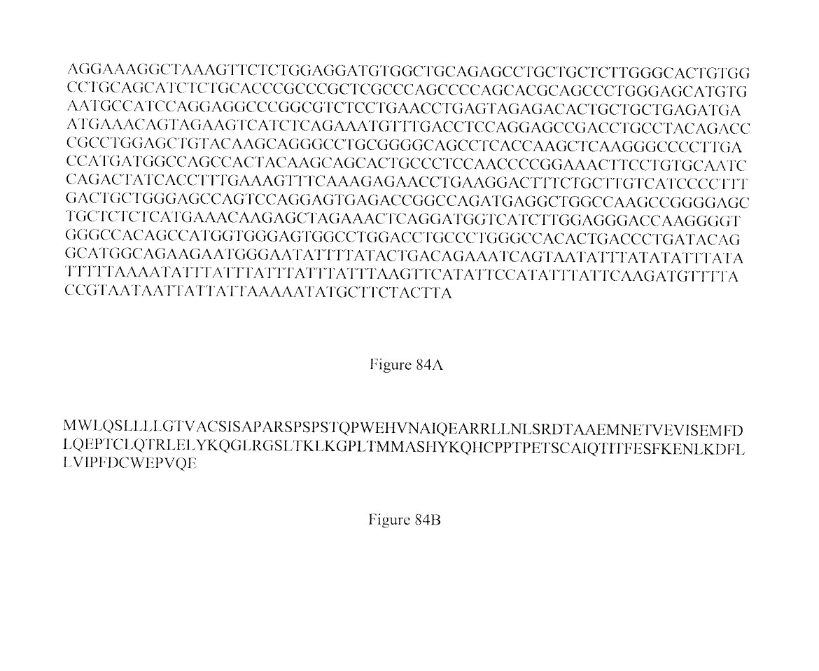

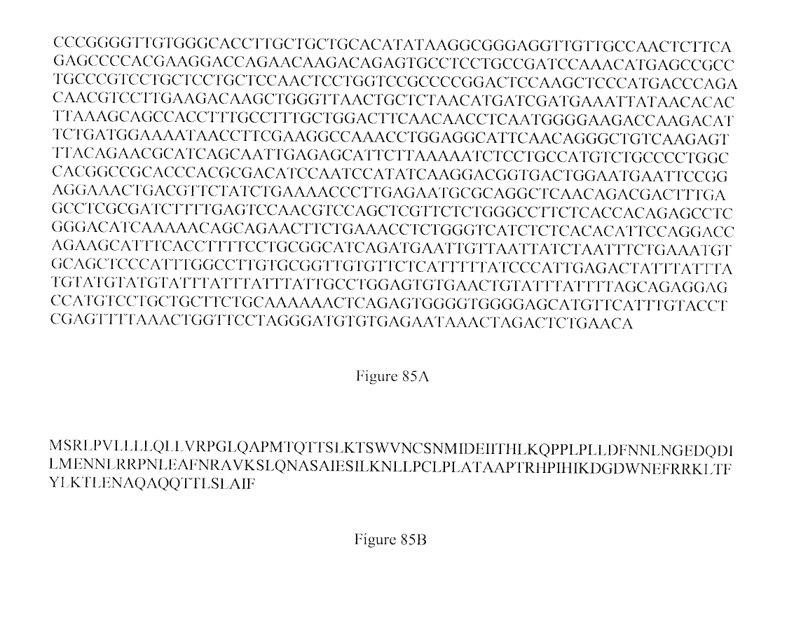

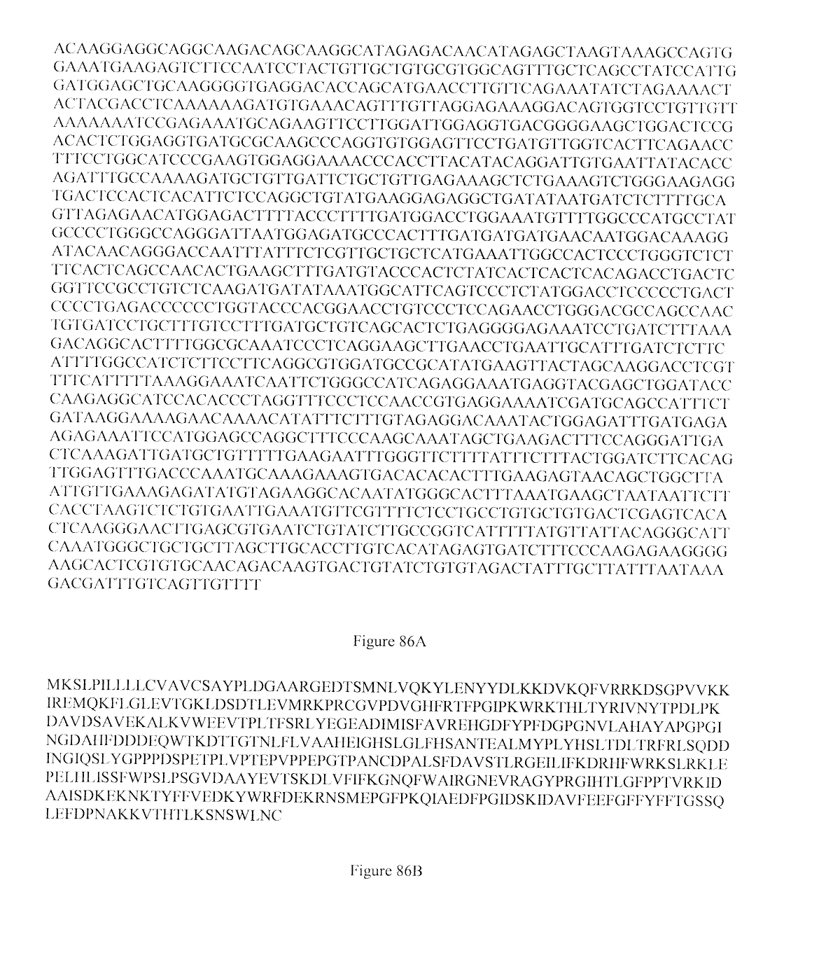

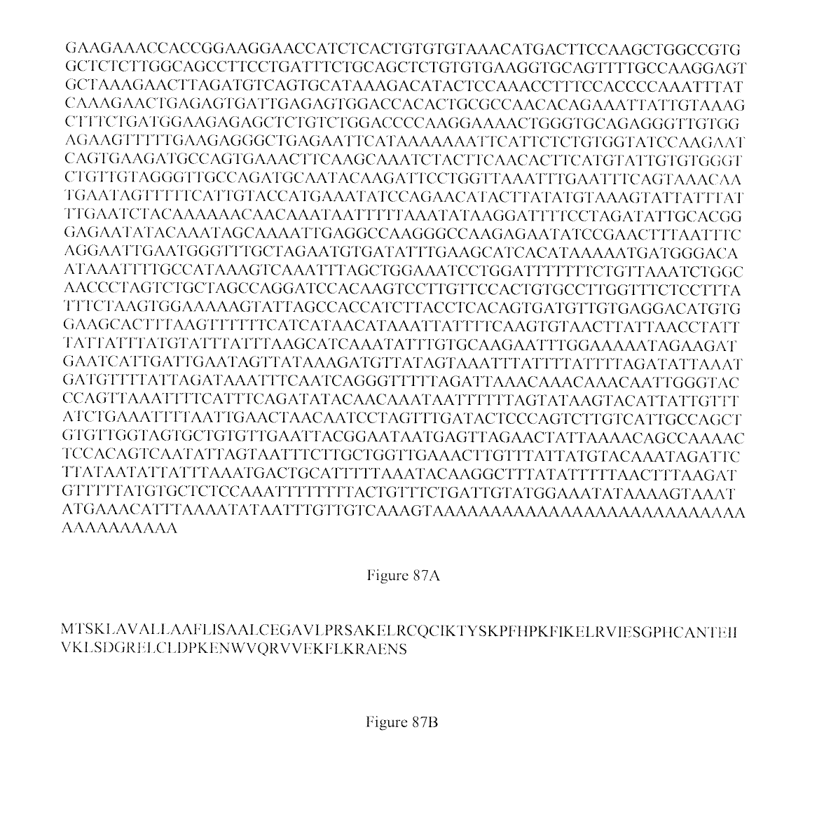

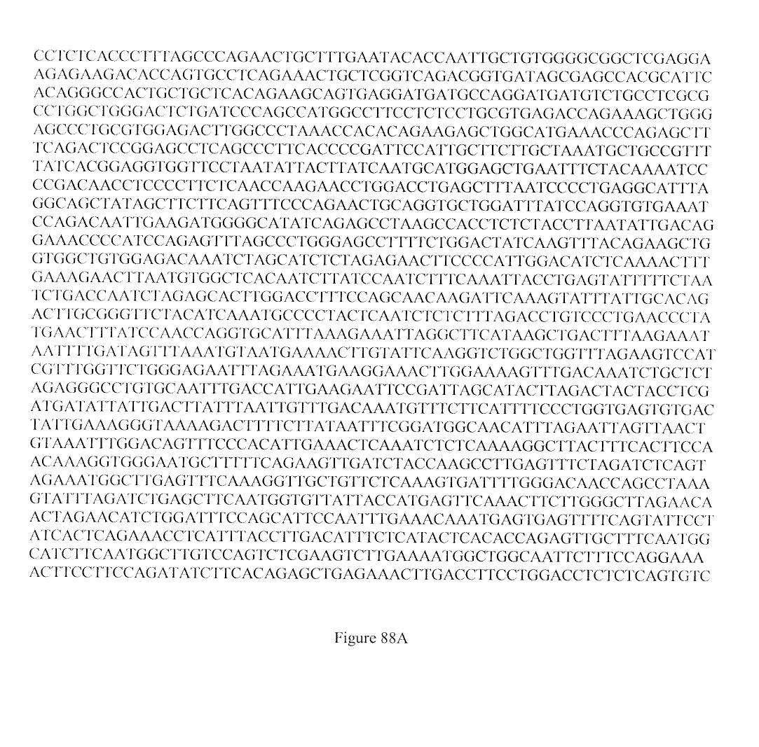

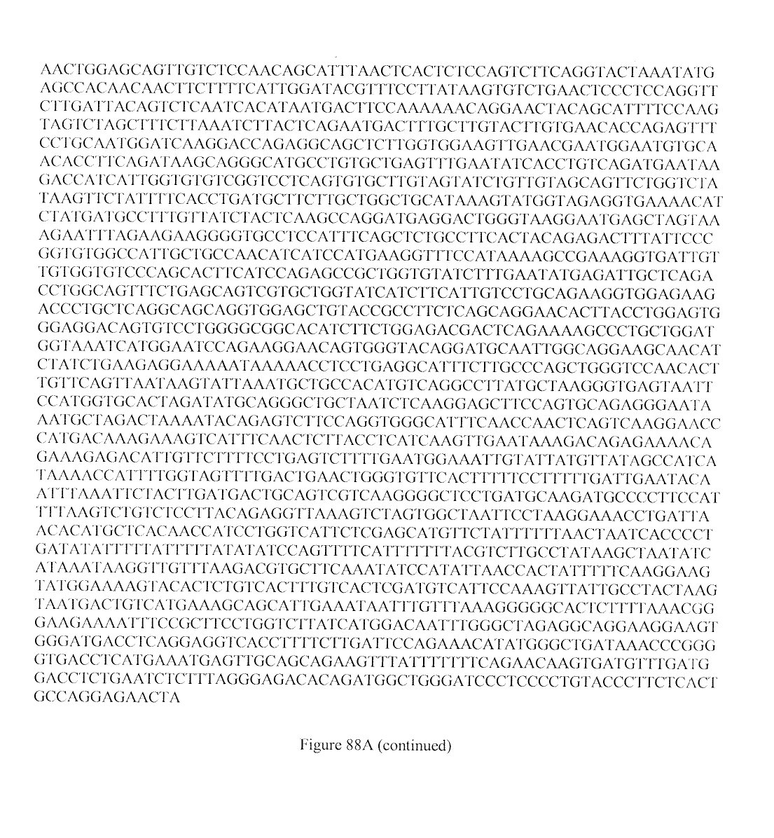

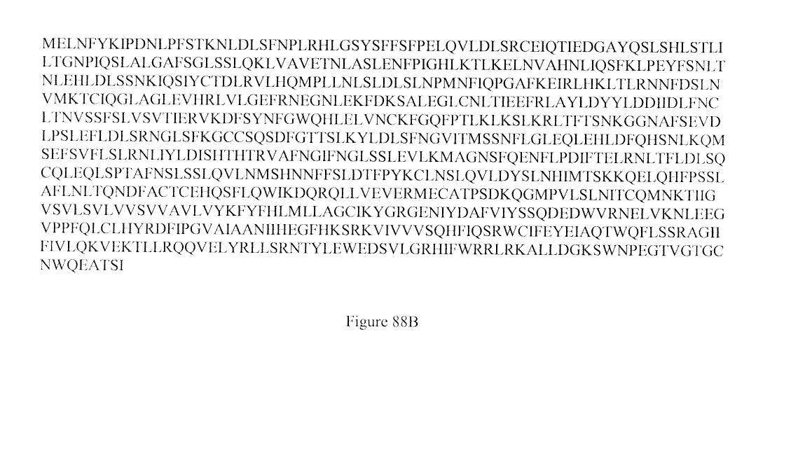

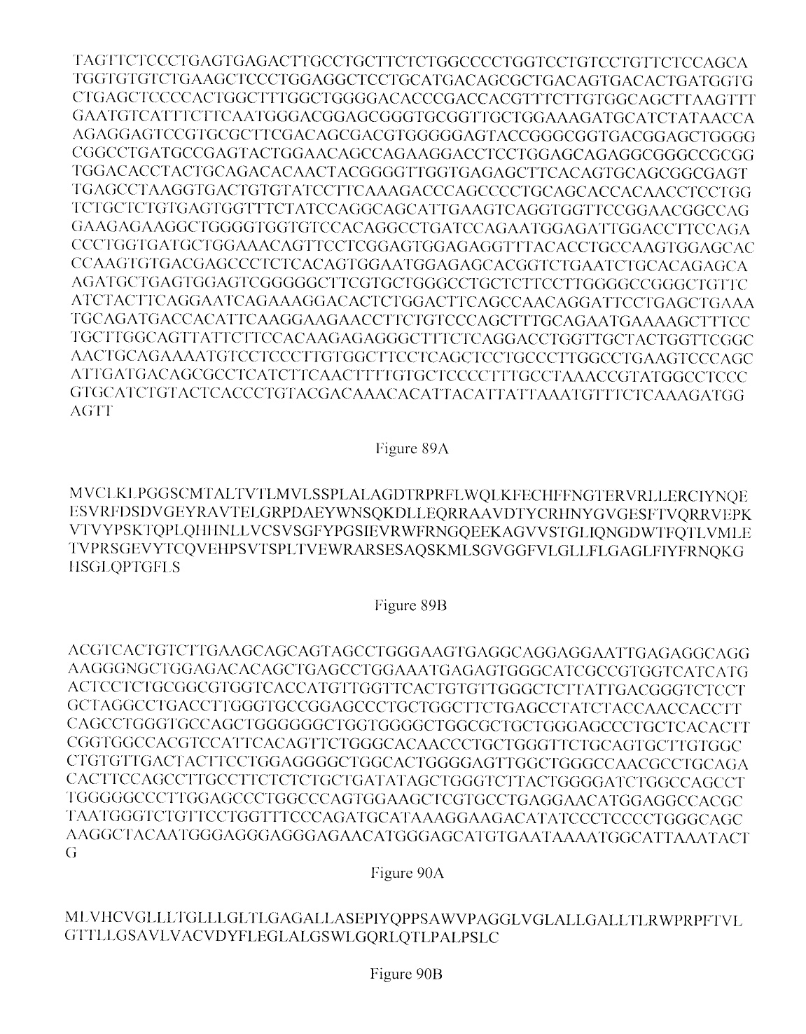

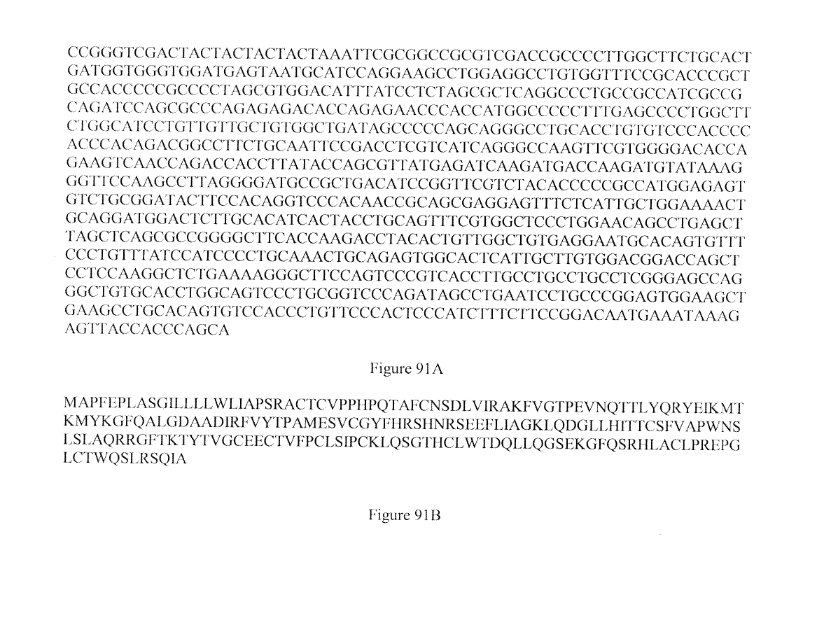

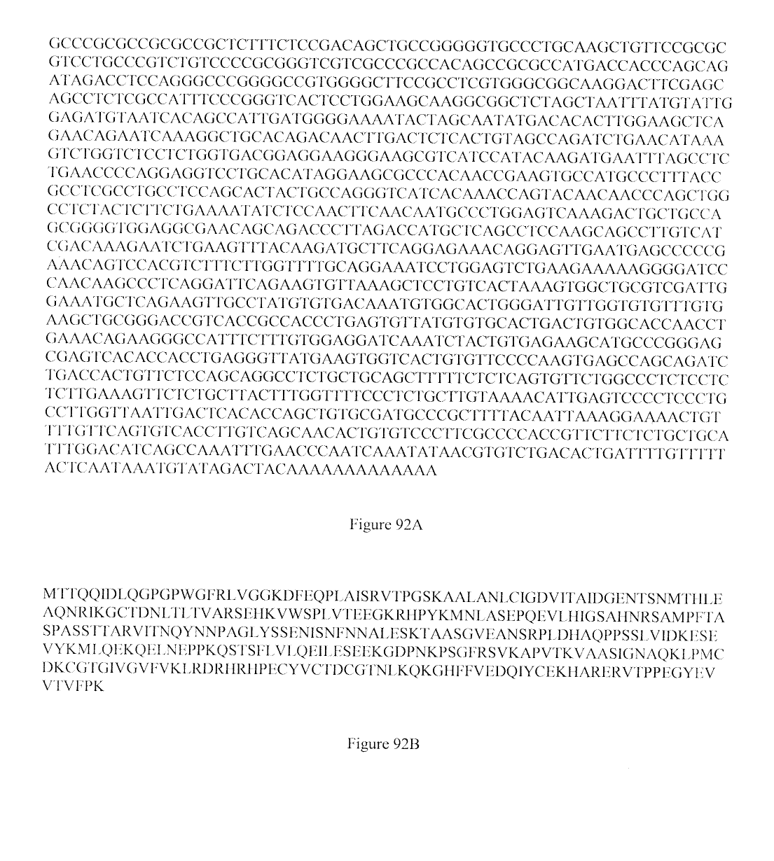

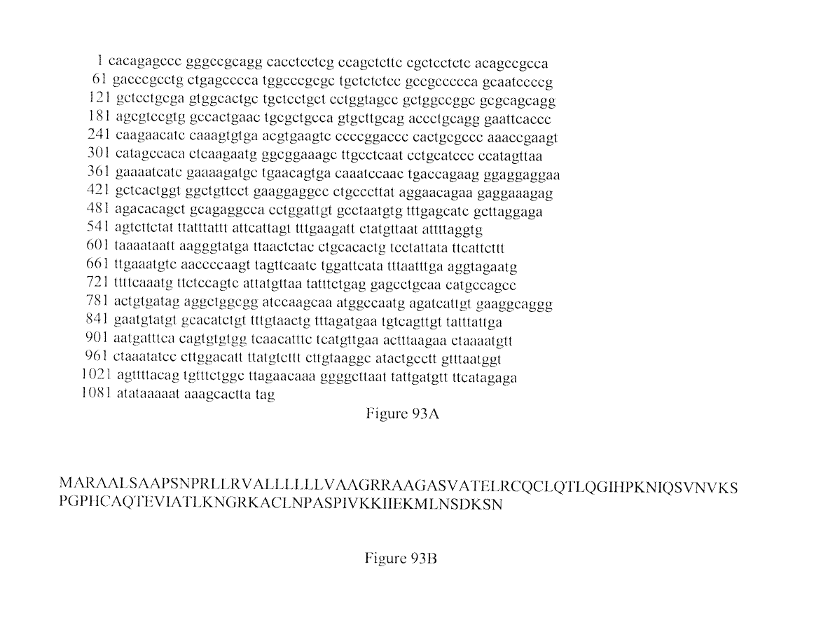

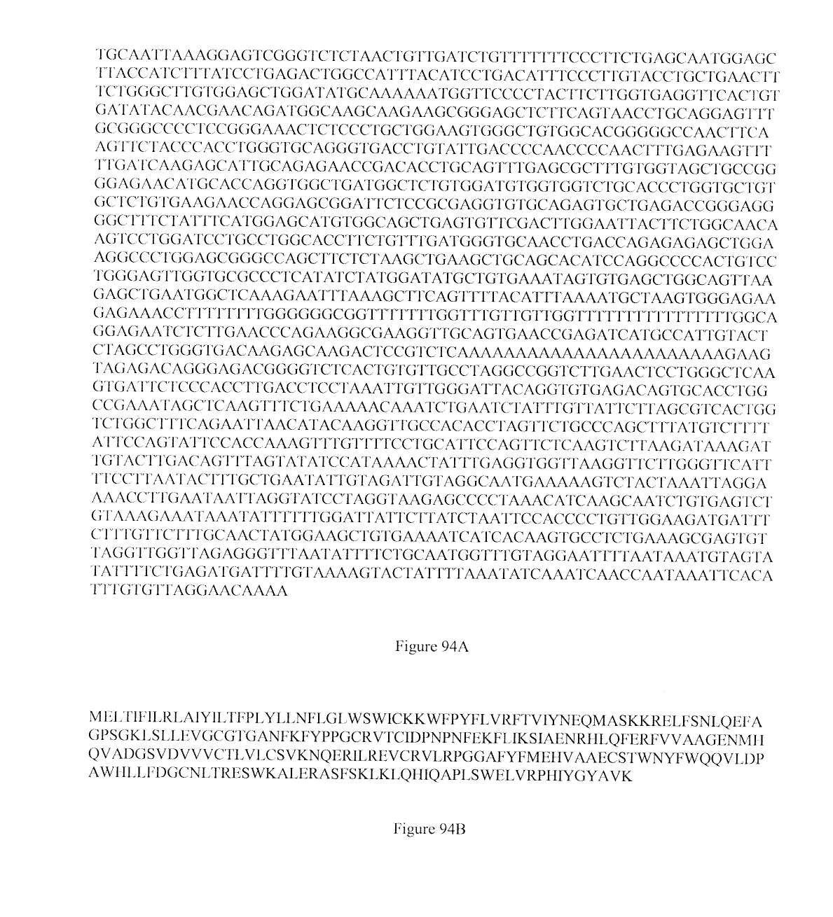

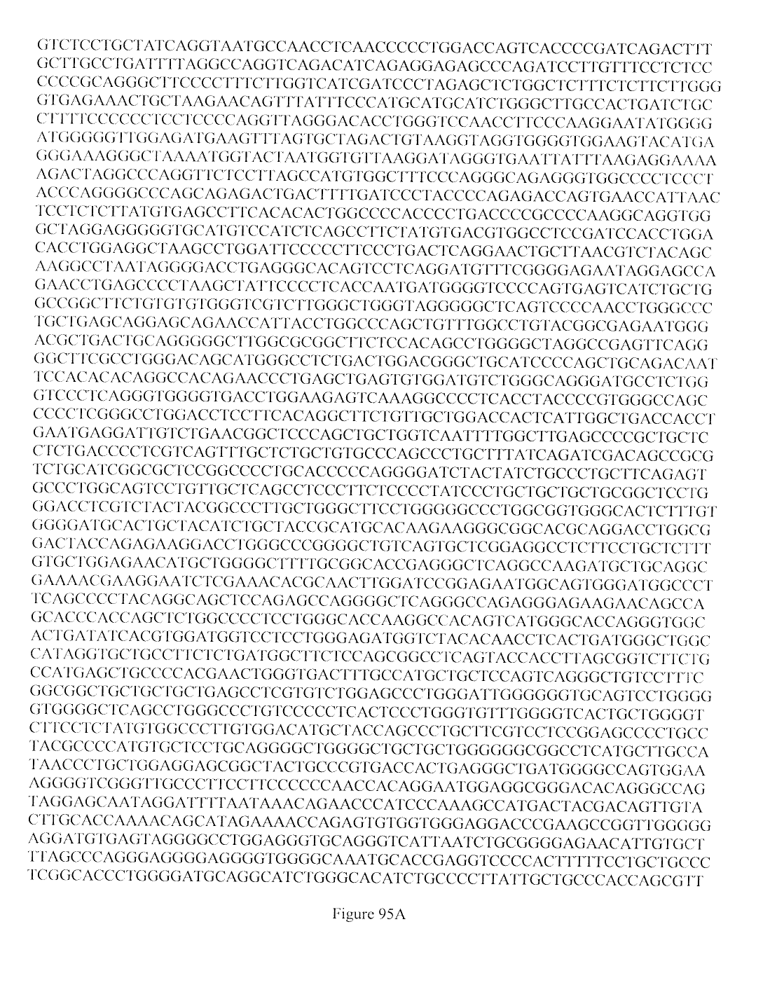

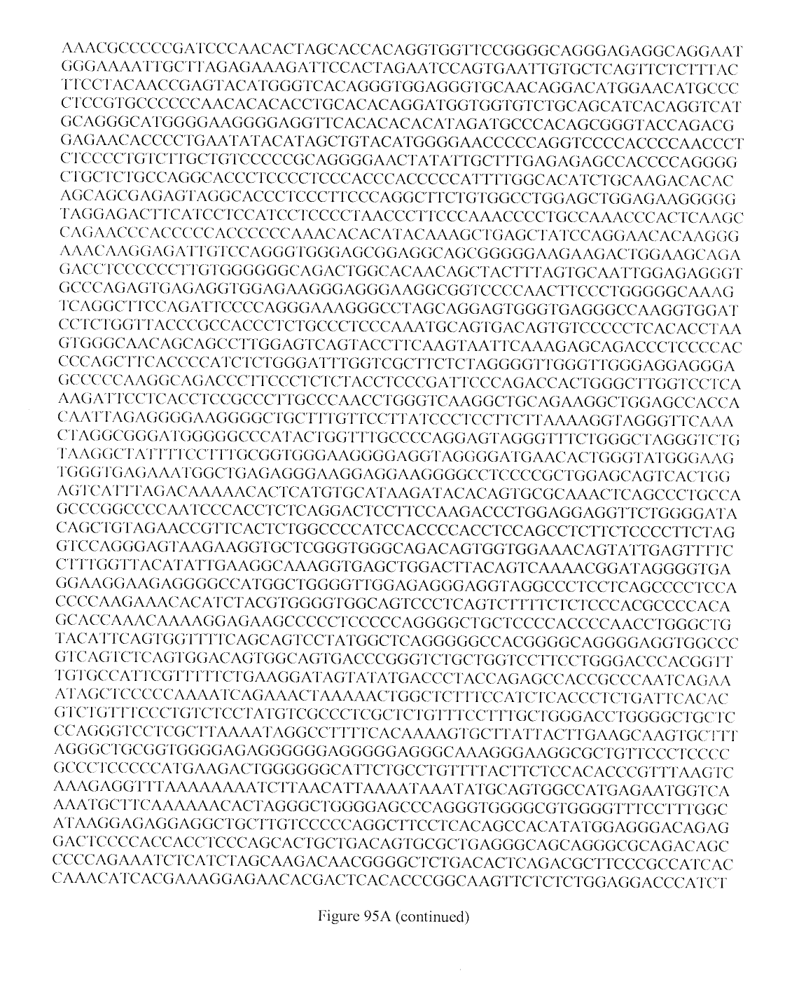

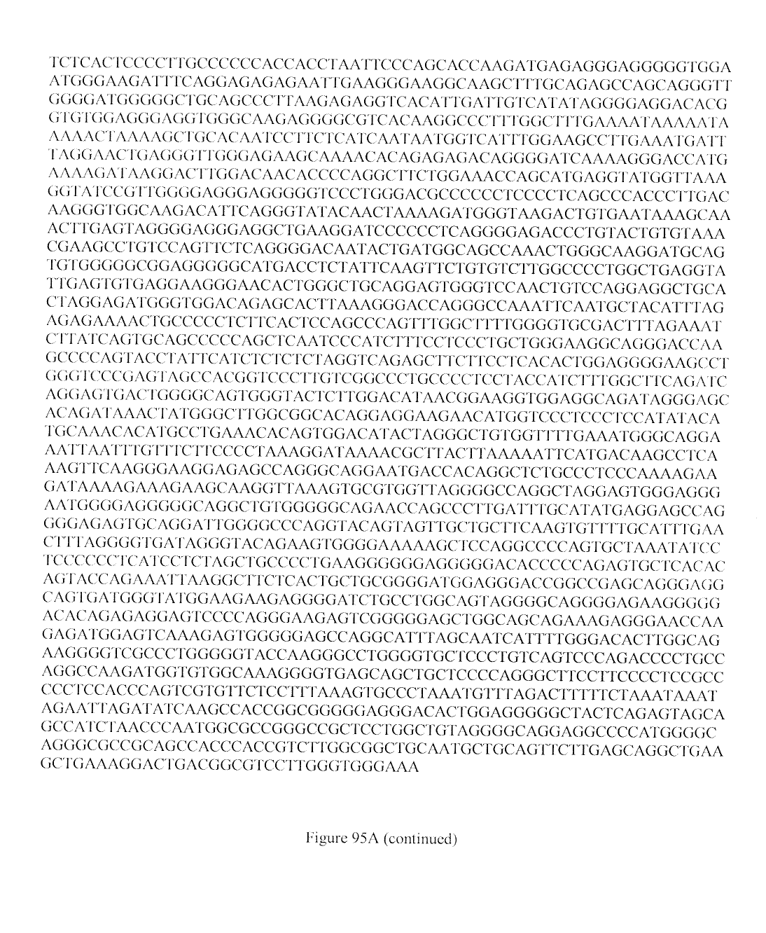

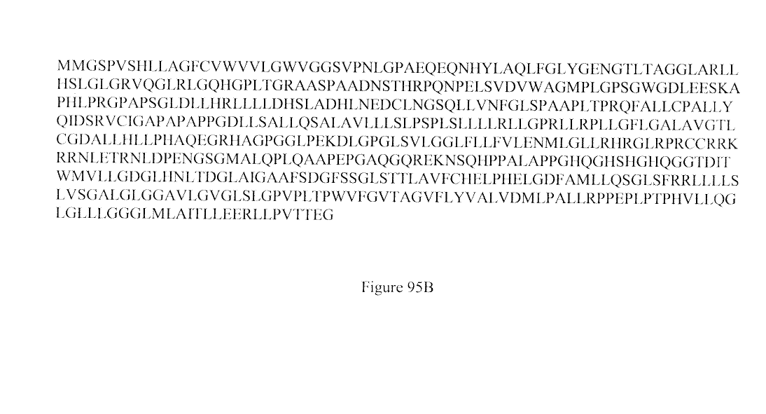

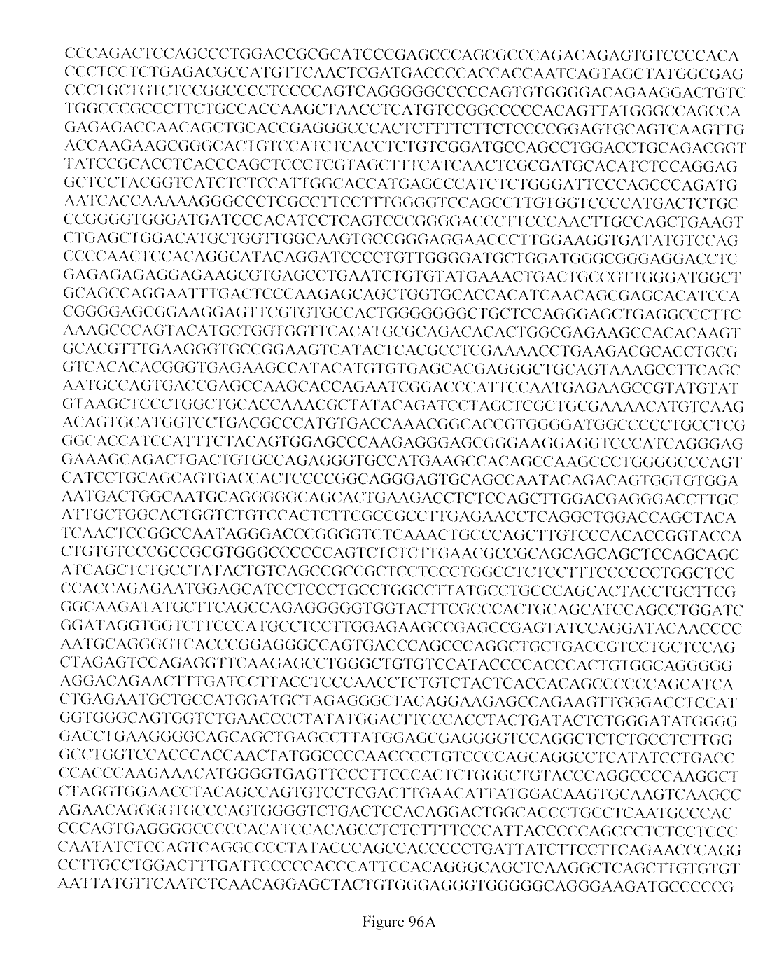

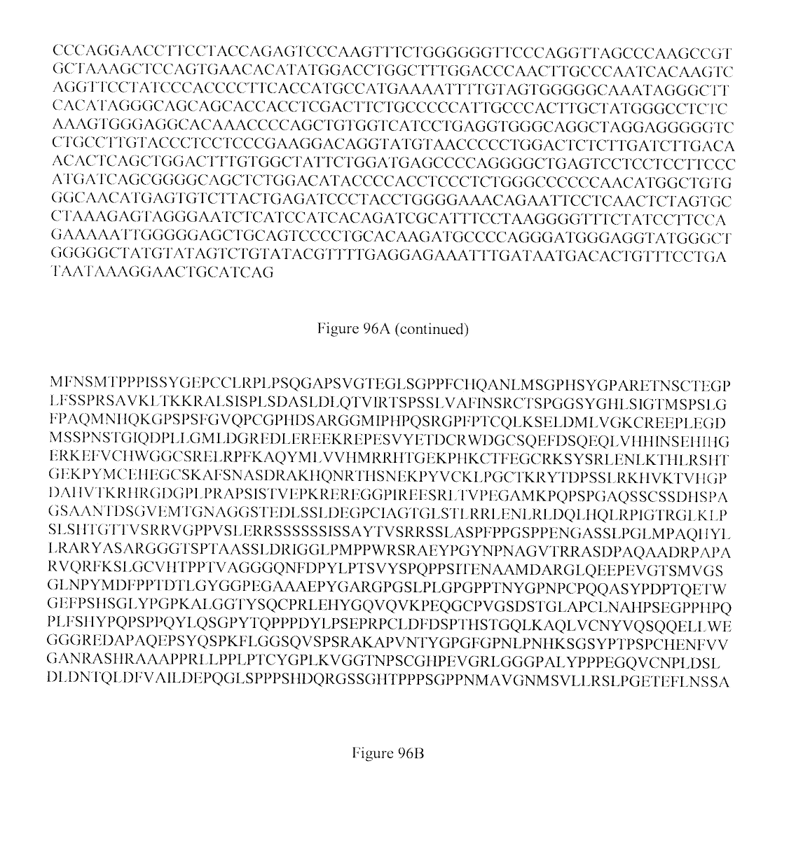

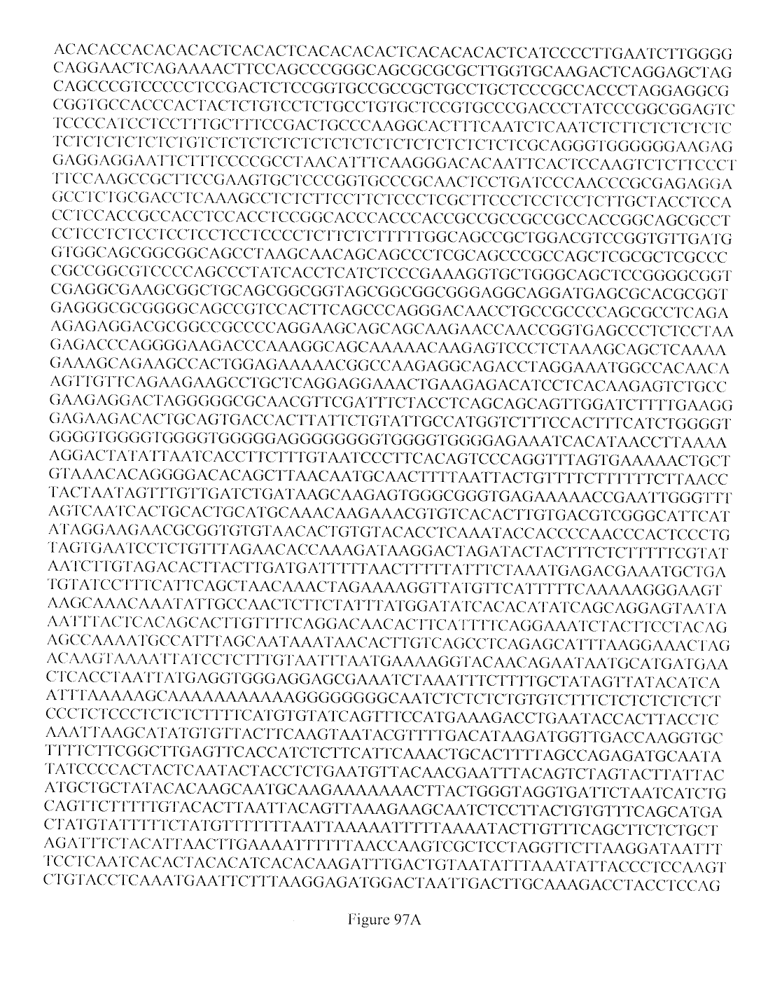

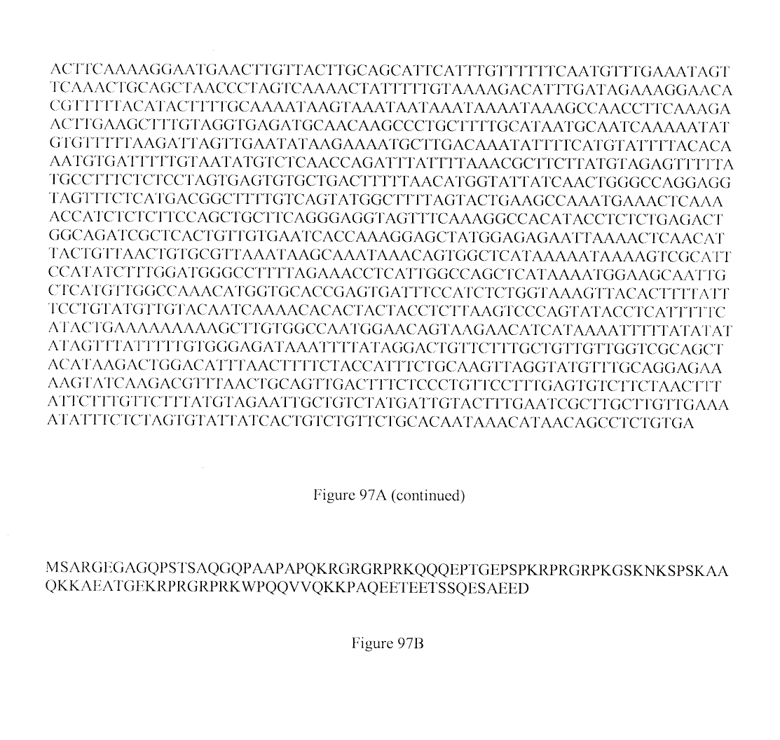

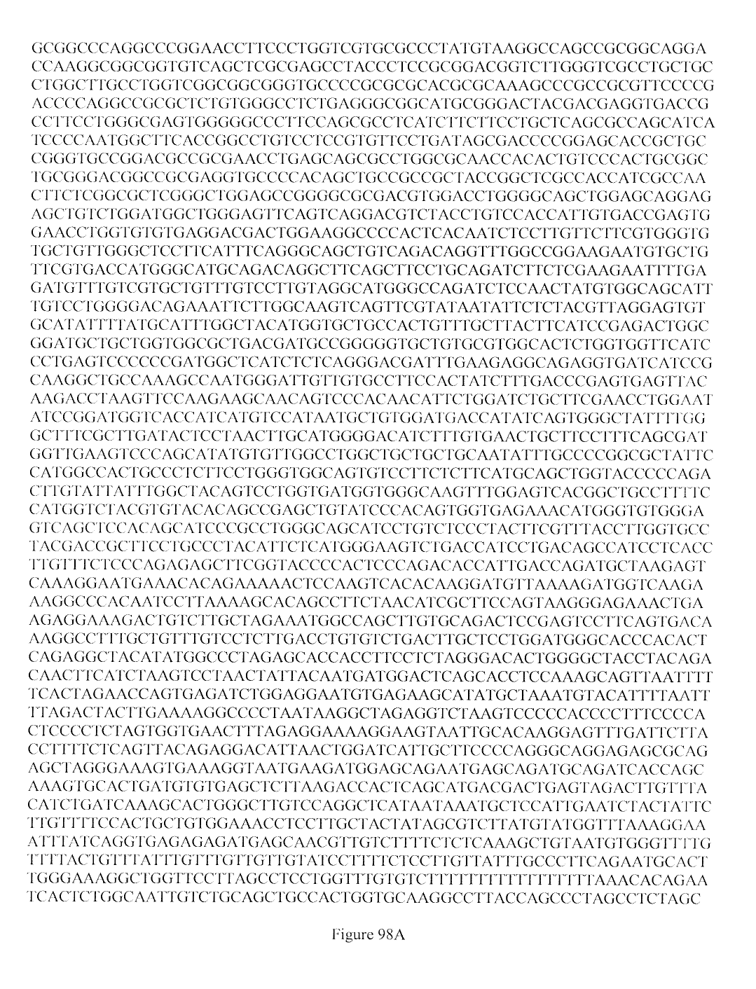

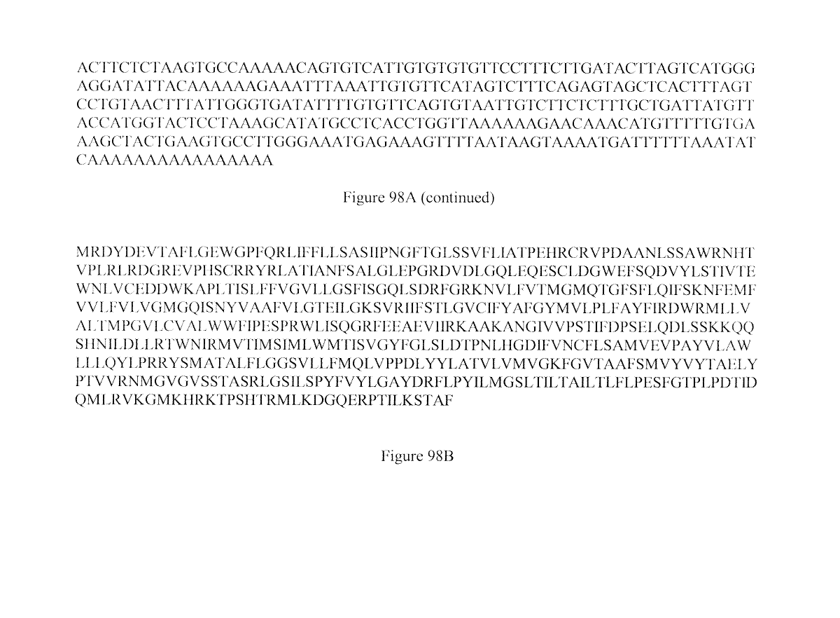

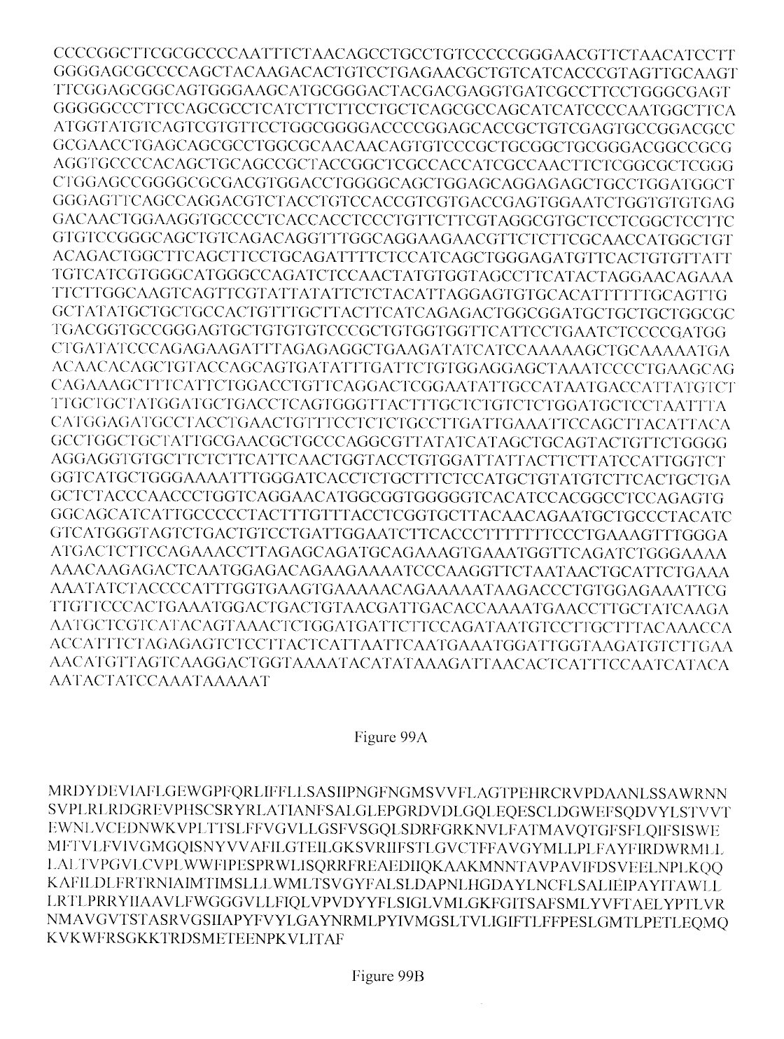

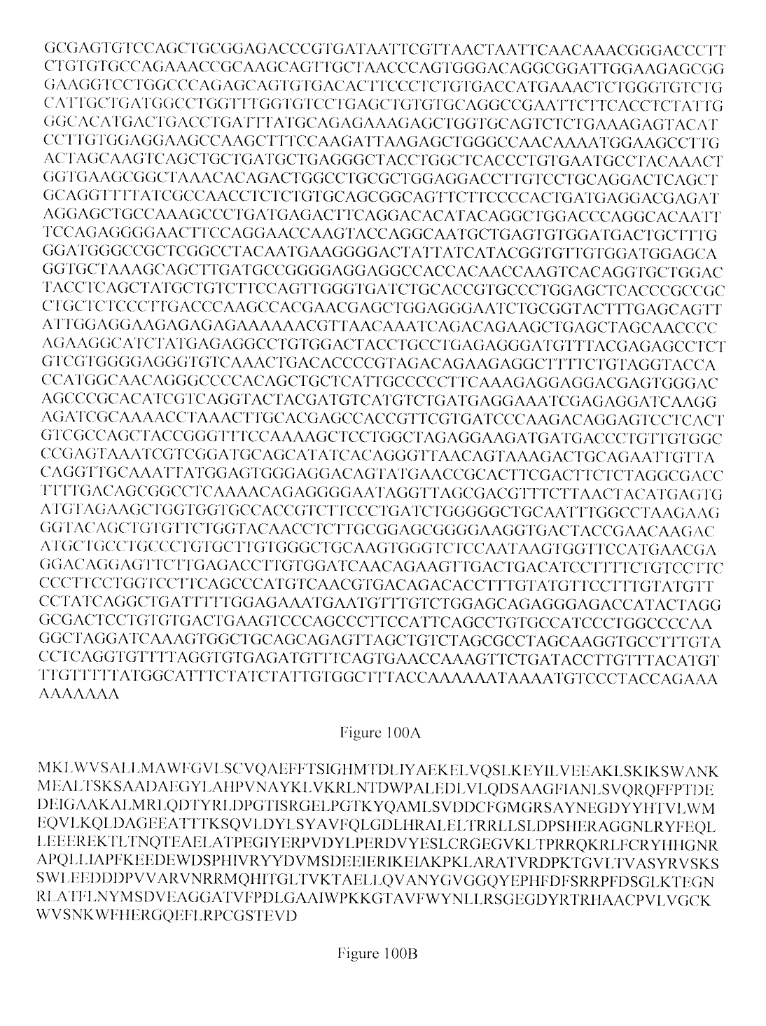

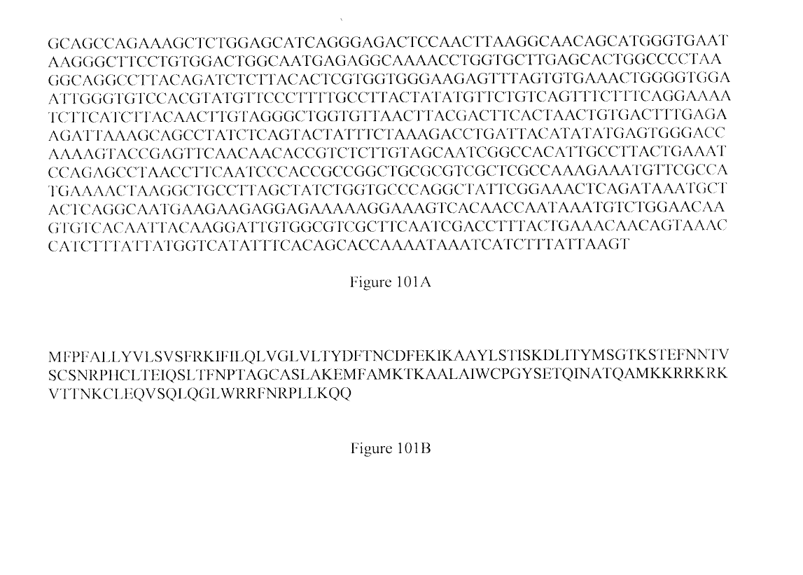

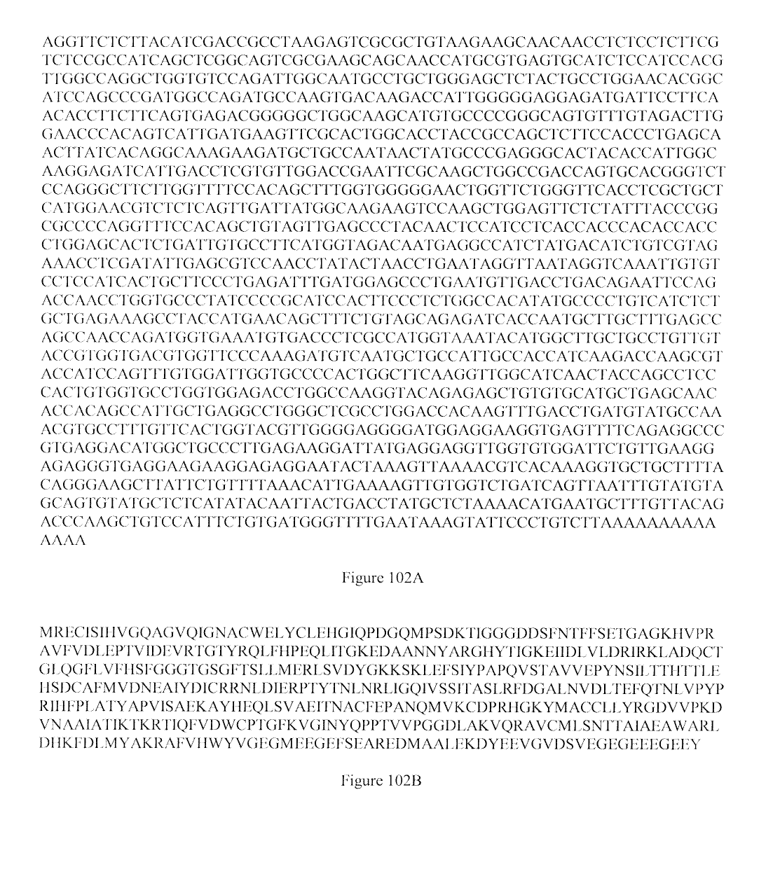

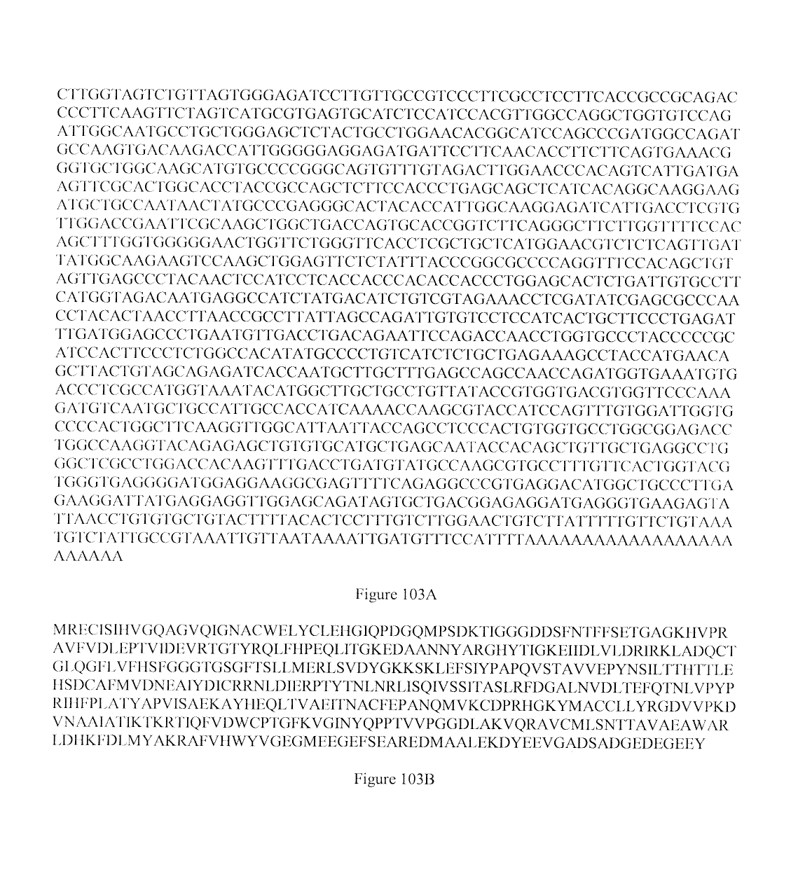

To further investigate the cellular localization of the excess DEFA5& 6 expression in the colon of patients with UC, in-situ hybridization and immunohistochemistry was undertaken in a cohort of biopsies of patients with UC and controls (FIGS. 6 and 7).

FIG. 6 shows the in-situ hybridization of the terminal ileal biopsies for DEFAS showed strong hybridization in the basal crypts consistent with Paneth cell location. In the upper panel terminal ileum (TI), the antisense probe shows strong hybridization in the basal crypts consistent with Paneth cell location. In the lower panel terminal ileum (TI), no significant hybridization was observed with sense control probe. Panel A shows the sigmoid colon biopsy of a non-inflamed control patient. Panels B, C, & D show strong, multifocal hybridization in the basal crypt region of UC sigmoid colon biopsies consistent with Paneth cell metaplasia. In the UC biopsies taken from the sigmoid colon strong, multifocal hybridization in the basal crypt region of these biopsies was observed and this would be consistent with Paneth cell metaplasia. This was not observed in the non-inflamed control biopsies.

FIG. 7 shows the in-situ hybridization of the terminal ileal biopsies for DEFA6. In panel A, terminal ileum immunohistochemistry shows positive staining in the basal crypts consistent with Paneth cell location. In panels B & C, no significant staining was observed in the non-inflamed control patients. In panels D, E, & F, strong, multifocal staining in the basal crypt region of UC sigmoid colon biopsies consistent with Paneth cell metaplasia. Immunohistochemistry for DEFA6 confirmed that in the sigmoid colon UC biopsies, staining was observed in the basal crypt region of these biopsies consistent with Paneth cell metaplasia. Again, this was not observed in the non-inflamed control biopsies (FIG. 7).

Expression of Genes within the IBD2 Locus. Using the markers defining the IBD2 locus we identified 526 Agilent probes representing genes or expressed sequence tags within this locus on chromosome 12. 12 probes had a greater or less than 1.5 fold change in expression with p<0.01 when expression of acute and chronically inflamed UC sigmoid colon biopsies were compared to non-inflamed UC sigmoid colon biopsies (Table 7).

TABLE 7

| | | UC sigmoid | |
|---|---|---|---|
| | | inflamed | |
| | | (35 biopsies) v | |
| | | non-inflamed (25) | |
| Agilent Probe | Gene Symbol | fold change | p value |
| A_23_P98876 | SLC39A5 | −1.52855 | 1.36 × 10⁻⁷ |
| A_24_P647146 | HDAC7A | 1.53876 | 3.15 × 10⁻¹⁶ |
| A_24_P941773 | DKFZP586A0522 | −2.27306 | 2.31 × 10⁻¹¹ |
| A_24_P945113 | ACVRL1 | 1.57956 | 6.33 × 10⁻⁹ |
| A_23_P128230 | NR4A1 | 1.81844 | 0.00005 |
| A_23_P331098 | K5B | 2.3845 | 0.0004 |
| A_24_P246636 | A_24_P246636 | −1.69754 | 7.06 × 10⁻⁸ |
| A_23_P2233 | SILV | 1.51557 | 9.07 × 10⁻⁹ |
| A_23_P105251 | GLI | −1.54447 | 7.73 × 10⁻⁹ |
| A_32_P3783 | HMGA2 | −1.94107 | 2.37 × 10⁻⁹ |
| A_23_P162300 | IRAK3 | 1.7382 | 3.11 × 10⁻¹⁵ |
| A_32_P83256 | IRAKM | 1.70847 | 4.56 × 10⁻¹⁰ |

Analysis of the 526 expression probes located within the IBD2 locus identified 12 probes that were significantly differentially regulated when the inflamed UC sigmoid colon biopsies were compared to the non-inflamed UC sigmoid colon biopsies.

Table 3A (provided above) lists those genes from the IBD2 locus on chromosome 12 that were found to be up-regulated in IBD patients as compared to normal patients.

Table 3B (provided above) lists those genes from the IBD2 locus on chromosome 12 that were found to be down-regulated in IBD patients as compared to normal patients.

Interesting candidate genes that were differentially regulated in the inflamed UC sigmoid samples included keratin 5B (FC +2.38, p=0.0004), MMP19 (FC +1.95, p=0.0084), GLI 1(FC −1.54, p=7.3×10⁻⁹), interleukin-1 receptor-associated kinase 3 (FC +1.74, p=3.1×10⁻¹⁵) and interleukin-1 receptor-associated kinase M (FC +1.71, p=0.0014).

When the acute inflammatory signal was removed and non-inflamed UC sigmoid biopsies and non-inflamed control sigmoid biopsies were compared, no sequences had a fold change greater or less than 1.5. However, notably downregulated genes included tubulin alpha 5 (FC −1.32, p=9.0×10⁻⁶) and tubulin alpha 6 (FC −1.45, p=1.2×10⁻⁵), molecules involved in the microtubule cytoskeleton and barrier integrity of the bowel.

Expression of Genes within the IBDS Locus. Agilent probes representing 11 genes within the with IBDS locus were identified and compared in healthy control, non-inflamed and inflamed UC biopsies (Table 8). Table 8 shows the fold changes in expression of genes within the IBDS locus comparing controls and patients with UC, who have been stratified for the degree of inflammation observed in their sigmoid biopsies. Significant down regulation of the organic cation transporters SLC22A4 and SLC22A5 was observed when inflamed UC sigmoid biopsies were compared to non-inflamed UC sigmoid biopsies.

TABLE 8

| | | | Inflamed sigmoid | | | |
|---|---|---|---|---|---|---|
| | All UC (129) v | | (35) v non-inflamed | | Non-inflamed UC sigmoid | |
| Genes | controls (73) | | sigmoid (22) (UC) | | (22) v non inflamed control | |
| Analyzed | Fold change | p value | Fold change | p value | sigmoid (18) Fold change | p value |
| IL4 | +1.00 | 0.96 | +1.06 | 0.62 | +1.14 | 0.40 |
| IL13 | +1.12 | 0.057 | −1.02 | 0.82 | 1.00 | 0.98 |
| RAD50 | +1.02 | 0.20 | −1.20 | 3.5 × 10⁻⁶ | +1.08 | 0.062 |
| IL5 | +1.09 | 0.037 | +1.10 | 0.17 | +1.04 | 0.53 |
| IRF1 | +1.10 | 0.35 | +1.12 | 2.9 × 10⁻⁶ | −1.01 | 0.62 |
| SLC22A5 | −1.26 | 3.37 × 10⁻⁶ | −1.50 | 2.2 × 10⁻⁶ | +1.02 | 0.75 |
| SLC22A4 | −1.18 | 0.11 | −1.79 | 1.53 × 10⁻⁹ | +1.22 | 0.63 |
| PDLIM4 | +1.10 | 0.0056 | +1.14 | 0.00039 | +1.01 | 0.78 |
| P4HA2 | −1.05 | 0.13 | 1.00 | 0.98 | −1.05 | 0.28 |
| CSF2 | +1.10 | 0.056 | +1.19 | 0.052 | −1.04 | 0.63 |
| IL3 | −1.01 | 0.39 | +1.02 | 0.67 | +1.01 | 0.75 |

Table 3A (provided above) lists those genes from the IBDS locus on chromosome 5 that were found to be up-regulated.

vNon significant, but consistent fold increases in expression were observed when IRF1 and PDLIM4 expression was compared in inflamed and non-inflamed UC sigmoid colon biopsies, (FC +1.12, p=2.9×10⁻⁶ and FC +1.14, p=0.00039, respectively).

Table 3B (provided above) lists those genes from the IBDS locus on chromosome 5 that were found to be down-regulated in IBD patients as compared to normal patients. SLC22A5 (OCTN2) was downregulated in UC biopsies compared to controls (FC −1.26 p=3.37×10⁻⁶) and when inflamed UC sigmoid colon biopsies were compared to non-inflamed UC sigmoid colon biopsies (FC −1.50, p=2.2×10⁻⁶). Expression of SLC22A4 (OCTN1) was also downregulated in inflamed UC sigmoid colon biopsies compared to non-inflamed UC sigmoid colon biopsies (FC −1.79, p=1.5×10⁻⁹), however when all UC biopsies were compared to controls, no change was observed (FC −1.18, p=0.11).

Gene Expression and Disease Activity. Sigmoid colon biopsies from newly diagnosed UC patients who were undergoing endoscopy (8 biopsies) and sigmoid biopsies from patients with established active UC (18 biopsies) were compared. In the newly diagnosed UC sigmoid biopsies, 861 sequences were upregulated with a fold change of greater than 1.5 and p<0.01, and 373 sequences were downregulated with a fold change of less than 1.5 and p<0.01 (data not shown).

The 3 most upregulated genes in the newly diagnosed patients were Selectin E (FC +10.95, p=1.8×10⁻⁶), CCL19 (FC +7.42, p=4.7×10⁻¹⁵) and IL-8 (FC +7.32, p=2.9×10⁻⁷), and down regulated genes included S100P (FC −6.4, p=2.0×10⁻⁹).

Sigmoid colon biopsies from patients from UC patients with a simple clinical colitis activity index (SCCAI) of more than 2 (27 biopsies) were compared to sigmoid colon biopsies from UC patients with a SCCAI of 2 or less (30 biopsies). 813 sequences were upregulated and 444 sequences were downregulated. Among the most upregulated genes were those that were involved in the acute inflammatory response IL-8 (FC +8.86, p=4.4×10⁻¹²), MMP3 (FC +6.5, p=3.2×10⁻¹⁹), MMP7 (FC +5.3, p=3.4×10⁻²¹) and DEFA6 (FC +4.5, p=2.7×10⁻¹²). Down regulated genes included UGT2B15 (FC −4.5, p=0.0013), UGT2B17 (FC −2.8, p=0.0059) and UGT2B10 (FC −2.2, p=0.0038), all members of the UGT family which are involved in cellular detoxification and excretion.

Table 3A (provided above) lists additional genes identified in the present study observed to be up-regulated in UC patients as compared to normal patients, while Table 3B (provided above) lists additional genes identified in the present study observed to be down-regulated in UC patients as compared to normal patients.

Discussion

The present study represents the most rigorous microarray analysis yet reported comparing intestinal gene expression in patients with UC, with healthy controls as well as in patients with other causes of colonic inflammation. The data provide important information on anatomical pattern of gene expression in the healthy colon, with intriguing differences in the right and left colon. Moreover, strong evidence for dysregulated gene expression characteristic of UC has been provided and overall these data provide valuable insight into gene expression in normal physiological homeostasis and during the pathological process of UC.

The strengths of this data set are the size of the study undertaken, the meticulous assessment of disease phenotype, the documenting of anatomical location for each biopsy and the avoidance of confounding effects due to pooling of samples. Thereby, we have been able to remove a considerable amount of background variability that has hampered previous studies. (Marshall E. Science 2004; 306:630-631; Ioannidis J P. Lancet 2005; 365:454-455).

Moreover, the present study has addressed the potentially critical confounding effect of the strength of the non-specific acute inflammatory signature. Quiescent biopsies from patients with UC and controls were compared and from these analysis we were able to gain valuable insight into the pathogenesis of UC. In addition, the present study provided access to a proportion of patients with newly diagnosed disease, overcoming treatment related alterations in gene expression. A further strength of our study has been the fact that real time PCR analysis consistently confirmed the significant changes of in expression in all but one of the candidate genes of interest, strongly validating the microarray data and increasing substantially the confidence associated with the interpretation of the data.

This is the first microarray study to show a gradient in expression of a number of genes along the healthy adult colon. These results contrast with data from Costello and colleagues where no significant differences in expression patterns were observed when comparing biopsies from caecum, transverse colon, descending colon and sigmoid colon (Costello C M, et al. PLoS Med 2005; 2:e199). The observed differences in these data sets may be explained by our analysis looking only at non-inflamed control samples where as Costello investigated control and diseased patients.

Genes involved in developmental pathways—the HOX family and the hedgehog signaling pathway appeared to be the most differentially regulated along the anatomical length of the healthy colon. HOXA13 has been shown play a crucial role in the development of the tail gut and mutations in the gene result in urogenital abnormalities, (De Santa et. al. Development. 2002; 129:551-561) and interestingly it has been shown that HOXB13 expression is down regulated in colorectal tumours from the distal left colon. (Jung et. al. Br J. Cancer. 2005; 92:2233-2239)

The hedgehog signaling pathway is also crucial to the normal growth and development of the human gastrointestinal system and a number of congenital diseases that affect the gut are due to mutations in genes involved in this pathway. (Lees et. al. Gastroenterology. 2005; 129:1696-1710) GLI-1 is one of the major effector molecules of the hedgehog signaling pathway and the GLI-1 gene lies within the IBD2 locus, an area that has previously been shown to be associated with UC. (Parkes et. al. Am J Hum Genet. 2000; 67:1605-1610; Satsangi, et. al, Nat. Genet. 1996; 14:199-202) Data from the present study would also suggest that GLI-1 is downregulated in inflamed UC biopsies compared to non inflamed biopsies from patients with UC. Recent data from our unit has shown a strong association between mutations in the GLI-1 gene and UC. (Lees et. al. Gastroenterology. 2006; 130:A52)

Further recent data published by Varnat and colleagues have suggested that PPARβ negatively regulates Paneth cell differentiation by downregulating the expression of Indian hedgehog, another of the major effector molecules in the hedgehog signaling pathway. (Varnat et. al. Gastroenterology. 2006; 131:538-553) Our data have shown upregulation of the alpha defensins 5 and 6 in the colon of patients with inflamed UC compared to non-inflamed biopsies from patients with UC and controls, and immunohistochemistry and in-situ hybridization have shown that this is largely mediated by Paneth cell metaplasia. It is intriguing to speculate that in patients with UC, further as yet undetermined defects in the Hedgehog signaling pathway may result in unregulated Paneth cell differentiation, Paneth cell metaplasia, increased alpha defensin 5 and 6 expression, and mucosal inflammation.

Indeed among the most stimulating observations in the present study were the data showing the upregulation of the alpha defensins 5 and 6 in patients with UC. Alpha defensins 5 and 6 are small cationic proteins that are part of the innate immune response and they have potent antimicrobial properties against gram positive and gram negative bacteria. (Ouellette A J. Springer Semin Immunopathol. 2005; 27:133-146) They are stored as pro-molecules in Paneth cells which in the healthy colon are largely restricted to the terminal ileum and on release into the mucosa they are cleaved by trypsin to the active antimicrobial peptide. (Ghosh et. al. Nat Immunol 2002; 3:583-590)

In our data set, high levels of alpha defensin expression were observed in the terminal ileal biopsies of non-inflamed controls and patients with UC. Levels of expression in the control patients and patients with quiescent UC fell as the location that the biopsies were retrieved from became more distal in the colon—ascending colon, descending colon and sigmoid colon. However, in the inflamed UC biopsies increased expression of both alpha defensins 5 and 6 was observed at each anatomical location that was biopsied. Lawrance and colleagues also noted the defensins alpha 5 and 6 were upregulated in patients with UC compared to controls, (Lawrance et. al. Hum Mol. Genet. 2001; 10:445-456) although, RNA was extracted from surgical resections and no details about the anatomical location of these specimens were given.

Recent data examining the expression of the alpha defensins 5 and 6 in patients with CD in the terminal ileum would suggest that expression levels are reduced irrespective of the degree of inflammation when compared to control terminal ileal tissue and this may be responsible for the terminal ileal phenotype observed in CD. (Wehkamp et. al. Proc Natl Acad Sci USA. 2005; 102:18129-18134) Whether the observed increase in alpha defensin 5 and 6 expression in our data set is a primary phenomenon related to disease pathogenesis, or whether it is secondary phenomenon protecting a previously damaged epithelial surface to microbial invasion may be resolved by looking for critical variants within these genes that are associated with disease susceptibility and altered function of these peptides.

It is of interest that many but not all of our results are broadly in line with two of the landmark microarray papers in IBD. Consistent with data from Lawrance and colleagues (Lawrance et. al. Hum Mol. Genet. 2001; 10:445-456) we have shown upregulation of S100A8 & A9 and the alpha defensins 5 & 6 in UC. However, no overlap was observed in the differentially expressed probes in the IBD2 locus in the present study and in that of Lawrance. Dieckgraefe and colleagues (Dieckgraefe et. al. Physiol Genomics. 2000; 4:1-11) observed upregulation of a number of the MMP genes, consistent with our data and interestingly members of the REG family were shown to be upregulated in the colon of patients with UC, probably as a result of Paneth cell metaplasia.

The downregulation of ABCB1 in our dataset is of significant interest, and consistent with earlier microarray data from Langman, Dieckgraefe and Lawrance and colleagues. (Dieckgraefe et. al. Physiol Genomics. 2000; 4:1-11; Lawrance et. al. Hum Mol. Genet. 2001; 10:445-456; Langmann et. al. Gastroenterology. 2004; 127:26-40) In addition to this, we have observed that the expression in ABCB1 displayed a decreasing gradient, with gene expression lowest in the sigmoid colon in UC. This pattern of expression is perhaps consistent with the hypothesis associating loss-of-function of P-glycoprotein (gene product of ABCB1) in UC, and in line with the clinical presentation of UC. The current data illustrate the importance of ABCB1 in the disease pathogenesis of UC, as highlighted in recent genetic and animal functional data. (Moodie et. al. Glucocorticoid access and action in the rat colon: expression and regulation of multidrug resistance 1a gene (mdr1a), glucocorticoid receptor (GR), mineralocorticoid receptor (MR) and 11-beta-hydroxysteroid dehydrogenase type 2 (11BHDS2). 197 ed. 2003)

ABCB1 encodes P-glycoprotein 170, an efflux epithelial transporter involved in gut barrier defence and xenobiotic metabolism. It is pertinent that when the entire class of proteins sharing homology with ABCB1 were analyzed, a further 6 out of 48 genes (12.5%) of the ABC transporters were significantly dysregulated in UC; suggesting an important role in this class of protein in the aetiopathogenesis of UC.

In contrast to data produced by Langmann we did not observe any changes in expression of the transcriptional regulator Pregnane-X receptor. These negative data are consistent with genetic studies carried out in the IBD population in Edinburgh—using a haplotype tagging approach, there was an association between the ABCB1 gene and UC, (Ho et. al. Hum Mol. Genet. 2006; 15:797-805) but no association between the Pregnane-X receptor and UC. (Ho G T et. al. Gut. 2006; 55:1676-1677) Aspects of study design may explain the differences observed in this context between our data and those of Langmann and colleagues, as our analysis took in to consideration the inflammation status of the biopsies and anatomical location. In our data set we did not pool samples, thus carrying out a large number of microarrays, where as Langmann and colleagues pooled terminal ileal and colon biopsies and this difference in methodology may also account for the different results.

The matrix metalloproteinases (MMPs) comprise of a family of greater than 23 zinc-dependent enzymes that are end stage effecter molecules involved in the degradation of extra cellular matrix components during morphogenesis and tissue remodeling. (Brinckerhoff et. al. Nat Rev Mol Cell Biol. 2002; 3:207-214) Consistent with our data, previous studies in patients with IBD have shown a marked increase in expression of MMP3 in the inflamed biopsies when compared to non inflamed biopsies and control biopsies. (Heuschkel et. al. Gut. 2000; 47:57-62; von LBet. al. Gut. 2000; 47:63-73) Data for MMP3−/− mice have also demonstrated delayed clearance of bacteria, a compensatory increase in MMP7 and reduced CD4⁺ T lymphocyte recruitment to the lamina propria. (L1 et. al. J Immunol 2004; 173:5171-5179) Furthermore, recent data have also shown a proinflammatory effect of MMP3 mediated through CXCL7 causing dose dependant neutrphil recruitment in colonic cell lines. (Kruidenier et. al. Gastroenterology. 2006; 130:127-136)

In our data set we observed a decrease in MMP3 expression in our inflamed controls compared to our non-inflamed controls suggesting that the changes observed in MMP3 and MMP7 expression in UC may be disease specific. Transmission disequilibrium testing of the 5A variant of MMP3 in the German population showed an association with CD and not UC that was not replicated in the English population. (Pender et. al. J Med. Genet. 2004; 41:e112)

We have also used this dataset to analyse the relative expression of genes mapping to susceptibility loci implicated by genome wide analysis, notably within IBD2 and IBD5. The present data will be of use in gene identification, complementing results of fine-mapping studies, and genome-wide case-control studies currently underway. In this context it is of considerable interest to review our data concerning the IBD5 locus which spans a cytokine cluster containing a number of attractive candidate genes was initially discovered by genome wide scanning in 2000. (Rioux et. al. Am J Hum Genet. 2000; 66:1863-1870; Ma et. al. Inflamm Bowel Dis. 1999; 5:271-278) The tight linkage disequilibrium spanning the IBD5 linkage interval, has limited previous genetic studies, and these have not been sufficiently powered to identify the susceptibility gene within this region. (Waller, et. al. Gut. 2006; 55:809-814; Fisher et. al. Hum Mutat. 2006; 27:778-785; Noble et. al. Gastroenterology. 2005; 129:1854-1864)

Downregulation of the positional candidate gene encoding the organic cation transporter SLC22A5 (OCTN2) was observed when UC biopsies were compared to control biopsies and in both SLC22A4 (OCTN1) and SLC22A5 down regulation was observed in the inflamed UC sigmoid colon biopsies compared to non-inflamed sigmoid biopsies. These provocative data would suggest that decreased expression of these genes may after all be involved in the pathogenesis of UC, and these data thereby may sustain interest in these genes. Peltekova and colleagues suggested that two variants in these genes—SLC22A4 (1672C→T) and the SLC22A5 variant (−207G→C) conferred disease susceptibility to CD, (Peltekova et. al. Nat. Genet. 2004; 36:471-475) however, when expression was compared in the small number of patients in this study who had been genotyped for these mutations no change in expression was observed between wild type and TC homozygote patients (data not shown). Expression of IRF-1 and PDLIM4, both plausible candidates within IBDS was also dysregulated, emphasizing the uncertainties pertaining to this locus at present. Within the IBD2 locus, a series of dysregulated genes were identified in the present dataset, of which only GLI 1 has been subjected to detailed mutation analysis.

In conclusion these data have rigorously characterized expression of the whole genome in the terminal ileum and colon of patients which UC and controls. The studies provide new insights into regional variation of gene expression in the healthy colon, and also considerably extend previous studies in UC. These data identify a number of key regulators of intestinal inflammation, notably the alpha defensin family, hedgehog signaling molecules, and matrix metalloproteinases.

### Example 3

### Immunohistochemistry

### DefA6 Expression in IBD Biopsies

Defensin alpha 6 is normally expressed by Paneth cells in the small intestine crypt epithelium and not in colon epithelial cells. We had observed increased DefA6 expression at the RNA level in ulcerative colitis and Crohn's disease patients using Agilent microarray and in taqman on biopsy lysates. This experiment evaluated if increased DefA6 protein expression could be seen in formalin fixed colon biopsies.

Our studies show, as expected, that there was no DefA6 staining in colon biopsies from non-IBD control patients with no histologic evidence of inflammation. We also evaluated one non-IBD control patient with a diagnosis of microscopic colitis. In that patient, DefA6 was present in sigmoid colon crypt epithelial cells.

In ulcerative colitis patients, 21 patients had scattered or clustered DefA6 staining in crypt epithelial cells of the sigmoid colon, descending colon, transverse colon, or rectum. Twenty of 21 positive patients had histologic evidence of chronic or chronic-active inflammation in their biopsy tissue. The remaining patient had predominantly acute (neutrophilic) inflammation. No patients with positive DefA6 staining in the colon had uninflamed biopsies.

There were 18 ulcerative colitis patients with no evidence of DefA6 staining in colon epithelium. The majority of these patients (10) had no histologic evidence of inflammation in the biopsy tissue. Six of the remaining patients had predominantly neutrophilic inflammation (acute inflammation) and two had chronic/chronic-active inflammation.

In summary, DefA6 expression in ulcerative colitis appears to correlated with the local inflammation status observed in the biopsy. None of the uninflamed biopsies had DefA6 staining. In addition, patients with chronic or chronic-active inflammation were more likely to have positive DefA6 staining than patients with acute inflammation.

Experimental Design: The scoring of inflammation status was based on inflammatory cell type predominance: neutrophil predominance=acute inflammation; neutrophils and mononuclear inflammatory cells=chronic active; and predominantly mononuclear inflammatory cells=chronic.

Table 9 shows the histologic findings. The expression of DefA6 is shown as “−”, “+”, or “++” and the corresponding inflammation score is provided in some cases.

TABLE 9

| Patient | HP # | Tissue | DefA6 | Inflammation status |
|---|---|---|---|---|
| 115 | HP-19891 | sigmoid | + | microscopic colitis |
| 119 | HP-19893 | descend- | − | |
| | | ing | | |
| 122 | HP-19898 | sigmoid | − | |
| 124 | HP-19899 | sigmoid | − | |
| 125 | HP-19900 | sigmoid | NA | this is a section of |
| | | | | terminal ileum |
| 125 | HP-19901 | sigmoid | − | |
| 130 | HP-19905 | rectum | − | |
| 131 | HP-19906 | sigmoid | − | |
| 135 | HP-19908 | sigmoid | − | |
| 136 | HP-19909 | sigmoid | − | |
| 222 | HP-19912 | rectum | + | chronic active |
| 223 | HP-19914 | sigmoid | + | chronic |
| 224 | HP-19916 | sigmoid | + | minimal chronic active |
| 225 | HP-19919 | sigmoid | − | no inflammation |
| 226 | HP-19920 | descend- | − | no inflammation |
| | | ing | | |
| 227 | HP-19922 | sigmoid | + | minimal chronic active |
| 230 | HP-19924 | sigmoid | − | no inflammation |
| 230 | HP-19925 | rectum | − | no inflammation |
| 231 | HP-19926 | descend- | − | acute inflammation |
| | | ing | | |
| 233 | HP-19927 | sigmoid | − | no inflammation |
| 234 | HP-19928 | descend- | − | minimal chronic active |
| | | ing | | HP |
| 235 | HP-19929 | rectum | − | acute inflammation |
| 238 | HP-19930 | sigmoid | + | chronic active |
| 239 | HP-19932 | rectum | + | minimal chronic |
| 240 | HP-19933 | rectum | − | minimal acute |
| 241 | HP-19934 | rectum | − | no inflammation |
| 244 | HP-19935 | sigmoid | − | no inflammation |
| 246 | HP-19936 | sigmoid | + | minimal chronic |
| 247 | HP-19937 | rectum | + | minimal chronic |
| 248 | HP-19938 | rectum | + | minimal chronic |
| 249 | HP-19939 | rectum | + | chronic active |
| 250 | HP-19940 | sigmoid | + | chronic active |
| 251 | HP-19941 | rectum | + | chronic active |
| 253 | HP-19942 | sigmoid | + | chronic active |
| 255 | HP-19943 | sigmoid | NA | fragmented section |
| 256 | HP-19944 | sigmoid | + | chronic active |
| 257 | HP-19947 | rectum | ++ | chronic active |
| 258 | HP-19948 | rectum | + | chronic |
| 259 | HP-19949 | sigmoid | ++ | chronic active |
| 260 | HP-19950 | sigmoid | + | chronic active |
| 261 | HP-19951 | sigmoid | − | mild acute |
| 262 | HP-19952 | sigmoid | − | acute inflammation |
| 262 | HP-19953 | rectum | − | no inflammation |
| 263 | HP-19954 | rectum | − | no inflammation |
| 265 | HP-19955 | sigmoid | + | chronic active |
| 266 | HP-19956 | rectum | + | acute inflammation |
| 267 | HP-19957 | trans- | ++ | Chronic active |
| | | verse | | |
| 270 | HP-19958 | rectum | − | Chronic active |
| 245 | HP-19965 | sigmoid | − | no inflammation |
| 245 | HP-19966 | rectum | − | acute inflammation |

### Example 4

### Analysis of Germline GLI1 Variation

Ulcerative colitis (UC) and Crohn's disease (CD) are polygenic chronic inflammatory bowel diseases (IBD) of high prevalence that are associated with considerable morbidity. The hedgehog (HH) signalling pathway plays vital roles in gastrointestinal tract development, homeostasis and malignancy. We identified a germline variation in GLI1 (within the IBD2 linkage region, 12q13) in patients with IBD. Since this IBD-associated variant encodes a GLI1 protein with reduced function, we tested whether mice with reduced Gli1 activity are susceptible to chemically induced colitis. Using a gene-wide haplotype-tagging approach, germline GLI1 variation was examined in three independent populations of IBD patients and healthy controls from Northern Europe (Scotland, England and Sweden) totaling over 5000 individuals. On log-likelihood analysis, GLI1 was associated with IBD, predominantly UC, in Scotland and England (p<0.0001). A non-synonymous SNP (rs2228226C→G), in exon 12 of GLI1 (Q1100E) was strongly implicated, with pooled odds ratio of 1.194 (C.I.=1.09-1.31, p=0.0002). GLI1 variants were tested in vitro for transcriptional activity in luciferase assays. Q1100E falls within a conserved motif near the C-terminus of GLI1; the variant GLI protein exhibited reduced transactivation function in vitro. In complementary expression studies, we noted the colonic HH response, including GLI1, PTCH and HHIP, to be down-regulated in patients with UC. Finally, Gli1+/− mice were tested for susceptibility to DSS-induced colitis. Clinical response, histology and expression of inflammatory cytokines were recorded. Gli1+/− mice rapidly developed severe intestinal inflammation, with considerable morbidity and mortality compared with wild-type. Local myeloid cells were shown to be direct targets of HH signals and cytokine expression studies revealed robust up-regulation of IL-12, IL-17 and IL-23 in this model. HH signalling through GLI1 is required for proper modulation of the intestinal response to acute inflammatory challenge. Reduced GLI1 function predisposes to IBD pathogenesis, suggesting novel therapeutic avenues.

Ulcerative colitis (UC; MIM 191390) and Crohn's disease (CD; MIM 266600) are chronic, relapsing, inflammatory bowel diseases (IBD) of high prevalence (200-400 cases per 100,000 in N Europe and N America [Loftus E V, Jr. (2004) Gastroenterology 126: 1504-1517]) and are associated with considerable morbidity. Precise aetio-pathogenetic mechanisms are not understood but several lines of evidence implicate the central importance of a dysregulated host response to intestinal bacteria [Xavier R J, Podolsky D K (2007) Nature 448: 427-434]. Epidemiological data, detailed molecular studies, and recent genome-wide association studies strongly suggest that UC and CD are related polygenic diseases that share some susceptibility loci (IL-23R, IL-12B, and NKX2.3 [Wellcome Trust Case Control Consortium (2007) Nature 447: 661-678; Duerr et al. (2006) Science 314: 1461-1463; Parkes et al. (2007) Nat Genet July; 39(7):830-2. Epub 2007 Jun. 6]), but differ at others: NOD2, ATG16L1 and IRGM are specific CD genes; the ECM1 locus is associated with UC [Parkes et al. (2007) Nat Genet July; 39(7):830-2. Epub 2007 Jun. 6; Fisher et al. (2008). Nat Genet. 40(6):710-2. Epub 2008 Apr. 27; Hampe et al. (2007) Nat Genet. 39: 207-211; Hugot et al. (2001). Nature 411: 599-603; Ogura Y et al. (2001). Nature 411: 603-606]. The IBD2 locus (OMIM 601458) on chromosome 12q13 was first identified in a UK genome-wide scan (peak LOD score 5.47 at D12583) [Satsangi et al. (1996). Nat Genet. 14: 199-202] involving both UC and CD patients. Later studies showed that IBD2 contributes significantly to UC, notably extensive disease, but perhaps in a more minor way to CD susceptibility [Achkar et al. (2006). Am J Gastroenterol 101: 572-580; Parkes M et al. (2000) Am J Hum Genet. 67: 1605-1610]. A strong candidate gene that maps to the IBD2 locus is GLI1, one of three related GLI transcription factors that transduce secreted hedgehog (HH) signals. HH signalling is key in gut development, homeostasis and malignancy, but has not been carefully studied in IBD [Lees et al. (2005) Gastroenterology 129: 1696-1710]. In developing intestine, Sonic (SHH) and Indian Hedgehog (1HH) provide a paracrine signal from epithelium to the mesenchymal receptor patched (PTCH). PTCH controls HH signal transduction through the membrane protein smoothened (SMO) and zinc finger transcription factors GLI1, GLI2, and GLI3 to direct tissue pattern and cell fate [Madison et al. (2005) Development 132: 279-289]. Chronic injury, inflammation and repair are critical aspects of IBD, and thus it is pertinent that the HH pathway is centrally involved in these processes in several other tissues, including muscle [Pola et al. (2003) Circulation 108: 479-485], liver [Jung et al. (2008) Gastroenterology 134: 1532-1543; Omenetti et al. (2008) Gut May; 134(5):1532-43. Epub 2008 Feb. 14.], and lung [Stewart et al. (2003) J Pathol 199: 488-495; Watkins et al. (2003) Nature 422: 313-317]. Indeed, HH signalling may play a central role in the inflammatory response since SHH is critical for T lymphocyte development [El Andaloussi et al. (2006). Nat Immunol 7: 418-426], adult human CD4+ T cell activation [Lowrey et al. (2002) J Immunol 169: 1869-1875; Stewart et al. (2002). J Immunol 169: 5451-5457], and myeloid cell maturation in the spleen [Varas et al., (2008) J Leukoc Biol June; 83(6):1476-83. Epub 2008 Mar. 11]. Dysregulation of components of the HH pathway has also been noted in inflammatory diseases of the gut, including Barrett's esophagus, chronic gastritis and IBD [Nielsen et al., (2004) Lab Invest 84: 1631-1642]. Using microarray gene expression analyses of colonoscopic biopsies, we recently demonstrated that GLI1 is down-regulated in the intestinal mucosa in inflamed UC compared with non-inflamed samples [Noble et al. (2008) Gut. October; 57(10):1398-405. Epub 2008 Jun. 3]. To further explore the possible association between GLI1 and IBD susceptibility, we examined haplotype variation at the GLI1 locus in several Northern European populations using a gene-wide haplotype-tagging approach. We identified a significant association with IBD strongly implicating a non-synonymous SNP in the C-terminal region of GLI1. Functional analysis of the associated variant in vitro demonstrated a 50% reduction in GLI1 transcriptional activation, evidence that this may be a functional variant increasing disease risk. These findings together led us to hypothesize that a reduced dosage of functional GLI1 protein might play an important role in colonic inflammation. To test this directly, we challenged Gli1+/− mice and their wild type (WT) littermates with dextran sodium sulphate (DSS) to induce acute intestinal inflammation. Gli1+/− mice rapidly developed severe colitis, suggesting that functional Gli1 activity is crucial to response to inflammatory stimuli in mouse and man.

Subjects and Samples. Table 10 provides detailed demographics and phenotypic data on Scottish and Cambridge IBD population. (HC—healthy control; CD—Crohn's disease; UC—ulcerative colitis; IC—indeterminate colitis.

TABLE 10

| | Scottish Panel | Cambridge Panel |
|---|---|---|
| Total number | 2183 | 2337 |
| | (1374 HC; 474 UC; | (589 HC; 928 UC; |
| | 35 CD) | 737 CD; 83 IC) |
| Sex - % male | HC 48.7%; UC 52.0%; | HC 45.0%; UC 52.6%; |
| | CD 39.6% | CD 37.0% |
| Median age at | HC 50.0 (43.0-55.0) | HC 60.0 (53.0-69.0) |
| diagnosis/re- | UC 34.1 (25.2-49.9) | UC 36.7 (26.84-50.35) |
| cruitment -years | CD 27.8 (20.8-41.1) | CD 26.1 (20.3-37.2) |
| (IQR) | | |
| CD location | | |
| Terminal ileum (L1) | 38.4% | 31.8% |
| Colon (L2) | 36.5% | 36.5% |
| Ileocolon (L3) | 25.1% | 31.7% |
| UC location | | |
| Proctitis (E1) | 17.3% | 14.8% |
| Left-sided colitis (E2) | 40.5% | 34.1% |
| Extensive colitis (E3) | 42.2% | 51.1% |
| CD 5 year behaviour | | |
| Inflammatory (B1) | 64.8% | 52.3% |
| Stricturing (B2) | 14.8% | 35.2% |
| Penetrating (B3) | 20.3% | 12.5% |
| Perianal involvement (p) | 17.4% | 24.4% |
| Surgery CD | CD 59.3% | CD 52.0% |
| | UC 18.5% | UC 11.8% |
| Smoking at diagnosis of | YES 40.7% | YES 41.8% |
| CD | NO 47.4% | NO 45.0% |
| | EX 11.9% | EX 13.2% |
| Smoking at diagnosis of | YES 19.0% | YES 12.1% |
| UC | NO 49.5% | |
| | EX 31.5% | |

Genotyping.

Scotland and Sweden: Genomic DNA was extracted from blood samples using a modified salting-out technique as previously described, and Nucleon kits. Genotypes were derived using the Taqman system for allelic discrimination; the assays were available from Applied Biosystems as Taqman SNP Genotyping Assays (7900HT sequence detection system; Applied Biosystems), except for SNPs rs10783819, rs3809114, rs507562, rs542278, rs730560, rs1669296, rs775322 which were genotyped on the Illumina platform. The accuracy of each Taqman assay was checked by repeat analysis in 5% of cases, with 100% concordance. Genotype distributions in control populations were consistent with Hardy-Weinberg Equilibrium (p>0.01) for all SNPs. Genotypes, in cases for the four tSNPs that could not be derived by Taqman were obtained by direct sequencing. Cambridge: DNA was extracted using Nucleon kits. Genotyping of CD cases and controls was performed using the Taqman biallelic discrimination system using an ABI 7900HT analyser (Applied Biosystems). Genotyping of UC cases was performed using a 1536 SNP Golden Gate bead array (Illumina). Concordance between platforms was assessed by genotyping 92 UC cases for SNPs rs2228224 and rs2228226 with concordance rates of 100% and 97.9% respectively and no evidence of systematic bias between platforms. Genotype distributions in case and control populations were consistent with Hardy-Weinberg Equilibrium (p>0.01) for all SNPs.

Gene Expression by Microarray and QPCR.

The cohort of patients used in the microarray studies consisted of 67 patients with UC, 53 with CD and 31 healthy controls (HC). For demographics, generation of microarray dataset and QPCR methodology [see Noble et al. (2008) Gut. October; 57(10):1398-405. Epub 2008 Jun. 3].

Induction of DSS-Induced Colitis and Histological Analysis.

Groups of two to four wild type Gli1+/lacZ or wild type littermate controls in mixed cages (on a C57BL/6 background) were administered 3% DSS in drinking water for 4-6 days. The amount of DSS consumed was not significantly different between WT and Gli1+/− animals (data not shown). Gli1+/− animals tended to weigh more than their WT littermates (mean=28 g for Gli1+/− animals and 24 g for WT animals). Therefore, DSS-treated weight matched WT C57B1/6 animals (n=4) were also tested along with Gli1+/− animals and WT littermates. No differences were evident between WT littermates and weight matched C57B1/6. All animals were monitored daily for diarrhoea, bloody stool, and weight loss. Clinical scoring was as follows: 0=no symptoms, 1=diarrhoea, 2=bloody stool, 4=severe rectal bleeding and morbidity to the point of immobility/death. For histology, a segment of large intestine tissue of equal length and location for all animals was fixed overnight in 4% paraformaldehyde, dehydrated, infiltrated with paraffin, and sectioned at 5 μm. Slides were stained with hematoxylin/eosin and scored histologically by a gastrointestinal pathologist (HA) blinded to the source of the tissue.

Protein Mutagenesis and Luciferase Assay

GLI1 E1100 was amplified from Image Clone #3531657, and cloned into pCMVTag2b. GLI1 Q1100 was obtained by inducing a point mutation using the QuikChange II Site-Directed Mutagenesis Kit (Stratagene) following the manufacturer's protocol. Plasmids encoding 8xGli-Luciferase, m8x Gli-Luciferase (mutated Gli sites) and Gli2ΔN were gifts of Dr. Andrzej Dlugosz. 293T cells were plated in 12 well plates, transfected with 0.7 μg/well transcription factor, 0.4 m/well reporter plasmid, and 2 ng/well pRL-TK Renilla (Promega) and analyzed for luciferase expression 36 hours after transfection using the Dual-Luciferase Reporter Kit (Promega) following the manufacturer's protocol. Firefly luciferase expression was normalized by well to Renilla, and fold changes were calculated by comparing to 8xGli-Luciferase transfected alone.

Cytokine Expression QPCR Whole colonic mRNA was collected using Trizol, followed by RNA clean-up with DNase digestion using the RNeasy Mini Kit (Qiagen). cDNA was synthesized using the iScript cDNA synthesis kit (Biorad), and SybrGreen QPCR was performed on a Biorad iCycler machine. Expression levels were normalized to GAPDH, and statistical analysis was performed using the Student's T-test.

Statistical analysis. Haplotype frequencies of the tSNPs were inferred using the expectation-maximization algorithm and these used to test whether haplotype frequencies were different in cases and controls as implemented in the EH and PM programmes. The test statistic 2*(In(Lcase)+In(Lcontrol)−In(Lcase/Lcontrol)), which has a χ² distribution with n−1 degrees of freedom (where n=number of possible haplotypes) was calculated and empirical p values obtained by permuting the data 10,000 times. Haplotypes were examined using the Haploview programme v3.2 (www.hapmap.org). Individual SNP analysis was performed using χ² or Fisher's exact test, where appropriate, with twotailed p-values given and odds ratios (OR) presented with 95% confidence intervals (C.I.). The meta-analysis of SNP rs2228226 was performed using the Mantel-Haenszel method using a fixed effects model (R-software package). Details for calculation of false positive report probability are provided in supplementary methods. Expression profiles were analysed using Mann-Whitney U test and Kruskal-Wallis test, assuming a non-parametric distribution of all datasets (GraphPad Prism 4, GraphPad Software Inc.).

Results

Gene-wide variation in GLI1 is associated with IBD and attributable to a nonsynonymous SNP (rs2228226) in the Scottish population

Four multi-marker tagging single nucleotide polymorphisms (tSNPs; r²≧0.8) were identified (rs3817474, rs2228225, rs2228224, and rs2228226) to describe haplotypic variation of GLI1, detecting haplotypes of a frequency >1%. We genotyped these 4 tSNPs in a Scottish IBD population consisting of 474 UC and 335 CD cases, and 1364 well-matched healthy controls (Table 10). We then used a model-free analysis [Zhao et al. (2000) Hum Hered 50: 133-139] to test the association of GLI1 and IBD susceptibility. In the Scottish population, we demonstrate a highly significant association in IBD (p<0.0001) and UC (p<0.0001), and an association with CD of borderline significance (p=0.03). On analysis of individual estimated haplotype frequencies in Haploview, 3 common haplotypes were described (DATA NOT SHOWN). We confirmed that this effect was confined to the GLI1 gene by genotyping an additional 7 haplotypetagging SNPs, chosen from Phase II HapMap data, to tag neighbouring blocks, in 166 CD and 170 UC patients. This confirmed the presence of a GLI1 spanning haplotype block that did not extend into neighbouring genes (INHBE and ARHGAP9) (DATA NOT SHOWN).

Table 11 shows minor allelic frequencies for GLI1 non-synonymous SNP rs2228226 (tSNP4) in Scottish, English, and Swedish healthy controls (HC), inflammatory bowel disease (IBD), Crohn's disease (CD) and ulcerative colitis (UC).

TABLE 11

| | | | IBD | | | UC | | | CD | | |
|---|---|---|---|---|---|---|---|---|---|---|---|
| | HC | | | | p value | | | p value | | | p value |
| | N | % | N | % | OR (C.I.) | N | % | OR (C.I.) | N | % | OR (C.I.) |
| Scotland | 1374 | 30.3 | 884 | 34.8 | 0.0026 | 474 | 33.9 | 0.042 | 335 | 36.1 | 0.0053 |
| | | | | | 1.23 | | | 1.19 | | | 1.30 |
| | | | | | (1.07-1.40) | | | (1.01-1.39) | | | (1.08-1.55) |
| Cambridge | 589 | 26.4 | 1737 | 29.6 | 0.042 | 928 | 30.8 | 0.017 | 737 | 27.9 | 0.40 |
| | | | | | 1.17 (1.0-136) | | | 1.21 | | | 1.08 |
| | | | | | | | | (1.03-1.42) | | | (0.90-1.28) |
| Sweden | 281 | 30.6 | 493 | 35 | 0.27 | 288 | 34.4 | 0.43 | 205 | 35.9 | 0.24 |
| | | | | | 1.14 | | | 1.11 | | | 1.19 |
| | | | | | (0.90-1.45) | | | (0.90-1.45) | | | (0.90-1.58) |

Odds ratios and two-tailed p-values are given for x2 analysis of IBD vs. HC, UC vs. HC and CD vs. HC in each of these three populations. Meta-analysis of these data are presented in FIG. 1. Frequencies of estimated haplotypes in all three populations and the full genotype data for the Scottish population are detailed in supplementary materials (Supplemental Tables 1 and 2).

The association on haplotype testing and log-likelihood analysis was largely attributable to a non-synonymous SNP in exon 12 of GLI1 (rs2228226C→G; tSNP4). rs2228226 was associated with IBD (allelic frequency OR=1.23, C.I. 1.07-1.40, p=0.0026; homozygotes OR=1.56, C.I. 1.15-2.11, p=0.0047), CD (allelic frequency OR=1.30, C.I. 1.08-1.55, p=0.0053, homozygotes OR=1.79, C.I. 1.21-2.65, p=0.0048) and UC (allelic frequency OR=1.19, C.I. 1.01-1.39, p=0.04) (Table 11 and DATA NOT SHOWN). These data suggest an allele specific dose response with a greater odds ratio for homozygotes than heterozygote patients. Mutation screening of the GLI1 coding regions by direct sequencing failed to identify any novel SNPs. There was no association between 7 additional GLI1 variants from dbSNP and IBD (DATA NOT SHOWN).

Replication of GLI1 association in two independent North European IBD cohorts and meta-analysis. We then sought to replicate these findings in other populations. In a large IBD panel from Cambridge, England (n=928 UC, 737 CD, 83 indeterminate colitis and 589 HC) association with GLI1 was replicated by log-likelihood analysis in IBD (p=0.009) and UC (p<0.0001). rs2228226 was associated with IBD (OR 1.17, C.I. 1.00-1.36, p=0.042) and UC(OR 1.21, C.I. 1.03-1.42, p=0.017) but not CD in this population (Table 11). As in Scotland, there was no association with tSNPs1-3 (DATA NOT SHOWN). In the smaller Swedish cohort (n=770), there was a non-significant trend to association of rs2228226 with IBD (OR 1.14, C.I. 0.90-1.45) (Table 11).

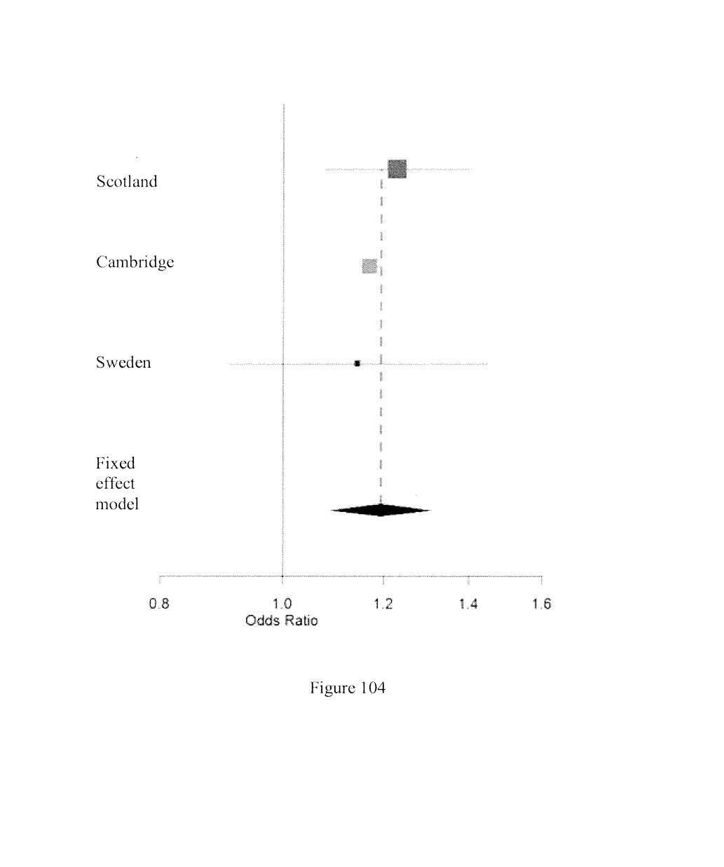

FIG. 104 shows a meta-analysis, using the Mantel-Haenszel method with a fixed effects model on the IBD cases and healthy controls in Scotland, England and Sweden confirmed the association with rs2228226 (OR 1.194, C.I. 1.089-1.309, p=0.0002). The meta-analysis is of non-synonymous GLI1 SNP rs2228226 (tSNP4) in Scotland, Cambridge and Sweden using Mantel-Haenszel method (n=5352 individuals). There was no evidence of heterogeneity in the contribution of rs2228226 between the 3 cohorts (p=0.825). Recognising the current problem with the publication of false positive findings in genetic association studies we estimated the probability that the association with disease risk found in the meta-analysis of GLI1 SNP rs2228226 represents a true (rather than false positive) association by adopting the false positive report probability (FPRP) approach described by Wacholder et al. (2004) J Natl Cancer Inst 96: 434-442. This gives an estimated probability that these findings represent a true finding of at least 92% (FPRP<0.08). This method is designed to avoid overinterpretation of statistically significant findings that are not likely to signify a true positive but in our study gives clear support to our interpretation of these data.

The GLI1 variant encoded by rs2228226 is functionally deficient in activating GLI-responsive transcription in vitro rs2228226C→G is a mis-sense mutation in exon 12 of GLI1, encoding a change from glutamine to glutamic acid (Q1100E).

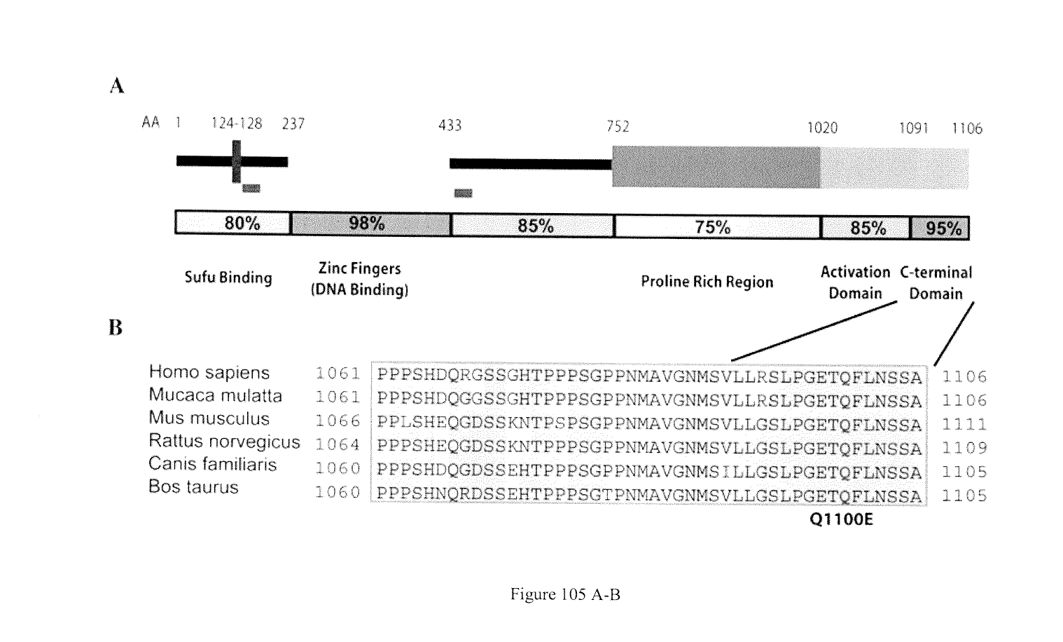

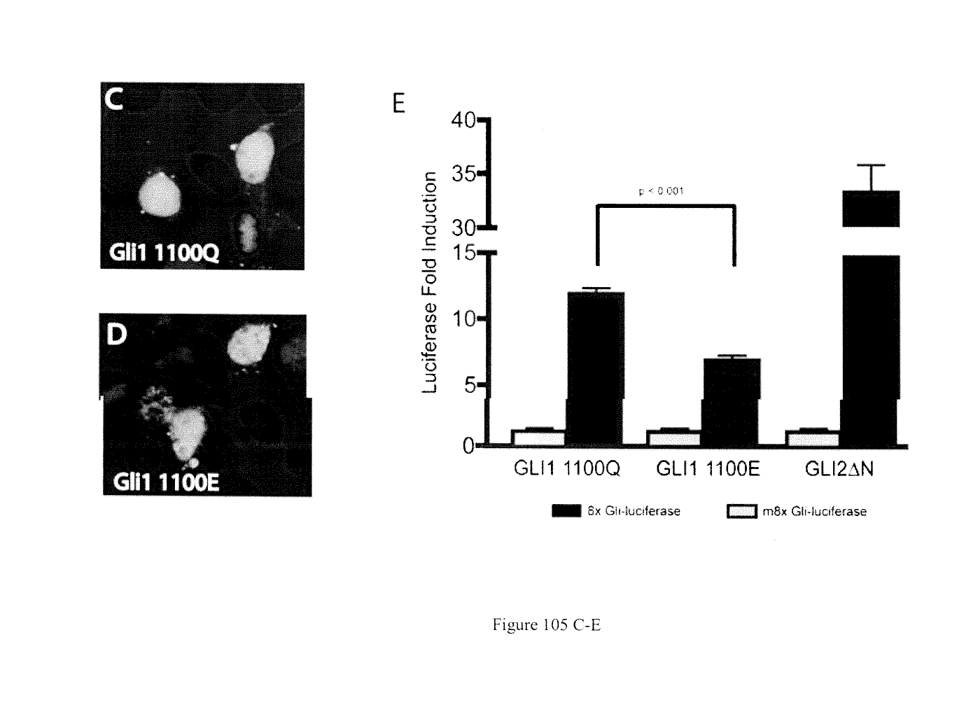

FIG. 105 shows Q1100E disrupts a conserved region of the GLI1 protein and reduces GLI1 transcriptional activity. A) Conservation of known functional domains in the Gli 1 protein. Previously described Sufu binding, DNA binding, and transactivation domains [Yoon et al. (1998) J Biol Chem 273: 3496-3501; 27 Kinzler et al. (1988) Nature 332:371-374; Kogerman et al. (1999) Nat Cell Biol 1: 312-319] are shown schematically. Amino acid conservation of each domain is represented numerically and by shading of the bar below the domain. Red boxes indicate regions known to regulate GLI1 protein stability [Huntzicker et al. (2006) Genes Dev 20: 276-281]. The conserved C-terminal domain that includes Q1100E is adjacent to a known transactivation domain. B) Alignment of the C-terminus of mammalian Gli1 proteins. This region (AA 1080-1106) is highly conserved in mammalian lineages. C, D) GLI1 Q1100 and E1100 have similar cellular localization in 293T cells. E) GLI1 E1100 is deficient in driving activation of the 8xGli-Luciferase reporter compared to GLI1 Q1100. Gli2ΔN is a strong activator of 8xGli-Luciferase and serves as a positive control for GLI1 activation. The m8xGli-Luciferase construct contains only mutant Gli binding sites and serves as a negative control. Data shown from 6 triplicate experiments done using two different plasmid preparations (N=18).

The mutation falls within a well conserved motif at the C-terminus of mammalian GLI1 proteins, near a recognized transactivation domain (FIG. 105a) [Yoon et al. (1998) J Biol Chem 273: 3496-3501]. The Q1100 residue is itself 100% conserved in all mammals examined (FIG. 105b). In order to evaluate the functional consequences of the Q1100E mutation, we transfected either GLI1 Q1100 or the variant GLI1 E1100 into 293T cells. No differences in level of expression or cellular localization were detected between these GLI1 variants; both proteins were readily detectable in the nucleus of transfected cells (FIG. 105c-d). We further evaluated the ability of each variant to activate the well-characterized GLI reporter 8xGli-Luciferase [Saitsu et al. (2005) Dev Dyn 232: 282-292]. We utilized GliΔ2N, a very strong activator of 8xGli-Luciferase, as a positive control for GLI1 transcriptional activity [Roessler et al. (2005) Hum Mol Genet. 14: 2181-2188]. While both GLI1 variants activated 8xGli-Luciferase above baseline, WT GLI1 Q1100 was two-fold more efficient as a transcriptional activator than the variant GLI1 E1100 (FIG. 105e).

Hedgehog pathway activity is dysregulated in colonic inflammation. We have previously reported that GLI1 expression is greater in the distal compared with the proximal colon in man [Noble et al. (2008) Gut. October; 57(10):1398-405. Epub 2008 Jun. 3].

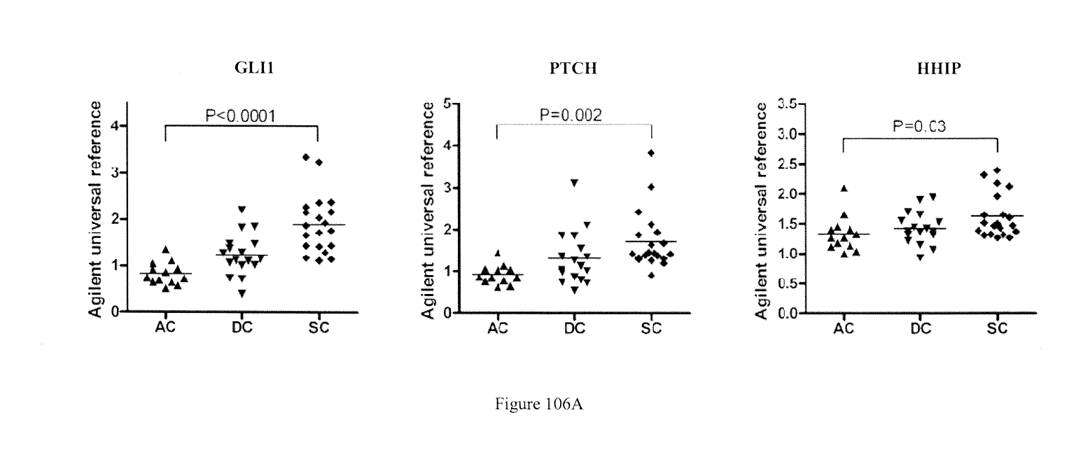

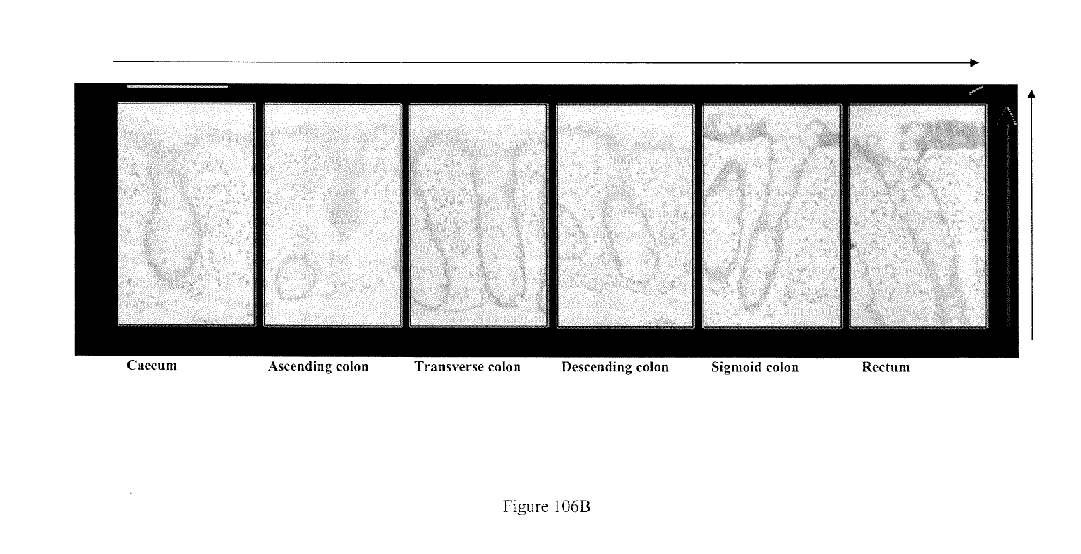

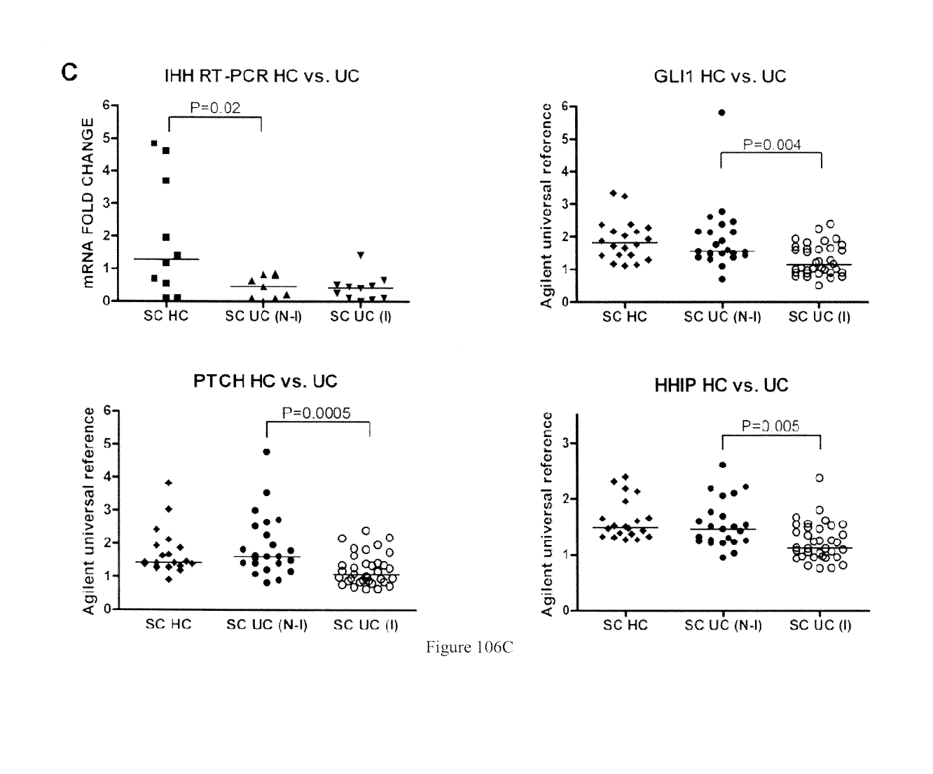

FIG. 106 shows expression of hedgehog (HH) signalling components in the healthy human adult colon (HC) and ulcerative colitis (UC). A) Patched (PTCH), Hedgehog-interacting protein (HHIP), and GLI1 mRNA levels increase along the length of the healthy adult colon, from ascending colon (AC) to descending colon (DC) and sigmoid colon (SC). B) HH protein expression in terminally differentiated enterocytes at the luminal surface, is greater in the distal colon compared with the proximal. C) Quantitative analysis of mRNA levels of Indian hedgehog (IHH), PTCH, GLI1, and HHIP in UC compared with non-inflamed HC. To account for the gradients identified along the length of the healthy colon (a-b), the data from SC only are shown. QPCR data is presented for IHH as this gene was not present on the Agilent microarray chip. Disease specimens are sub-categorised into non-inflamed (N−1) and inflamed (I) tissues. There was no change in levels of DHH, PTCH2, GLI2, GLI3, SUFU or DISP1 in either UC or CD compared with HC, or in non-IBD inflammation (data not shown). Analysis of SHH mRNA demonstrated a mild increase in expression levels related to inflammation that is consistent with the known expression of SHH in inflammatory cells (data not shown) [Lowrey et al. (2002) J Immunol 169: 1869-1875].

Individual data points are plotted with horizontal lines representing the medians for each dataset. P-values presented are derived from Kruskal-Wallis test, comparing levels in AC, DC and SC, and from Mann-Whitney U-tests (UC vs. HC(N−1)).

Extended in silico analysis of this microarray dataset now demonstrates that mRNA transcripts of PTCH and HHIP, along with HH protein, mirror this expression gradient (FIG. 106a-b). GLI1, PTCH and HHIP are pathway response elements whose expression levels predict pathway activity. GLI1 (p=0.0003), PTCH (p=0.002), and HHIP (p=0.0003) were lower in inflamed UC compared with HC from equivalent location (FIG. 106c). IHH was lower in UC regardless of inflammation (p=0.02). GLI1 expression was lower in CD than HC (p=0.004) irrespective of inflammatory status (DATA NOT SHOWN), a noteworthy finding given that GLI1 variation was also associated with CD in Scotland. PTCH was lower in inflamed CD compared with non-inflamed CD and HC (p=0.007). GLI1 and PTCH were both lower in non-IBD inflammation versus HC (DATA NOT SHOWN). These data demonstrate overall down-regulation of HH pathway activity, including GLI1, PTCH, and HHIP, in areas of colonic inflammation.

Gli1+/− animals exhibit mortality and heightened morbidity in response to intestinal inflammation induced by 3% DSS treatment. In vitro analysis of the GLI1 1100E variant demonstrated a 50% deficiency in transactivation function compared to WT GLI1, and our genetic analysis demonstrates an allele-specific dosage response, suggesting that a moderate reduction in GLI1 function was sufficient to predispose to intestinal inflammatory disease. To specifically test this hypothesis, we treated Gli1+/− mice [Park et al. (2000) Development 127: 1593-1605], and their WT littermates with 3% DSS for 6 days to induce acute intestinal inflammation. Gli1+/− animals were rapidly and severely affected by DSS treatment.

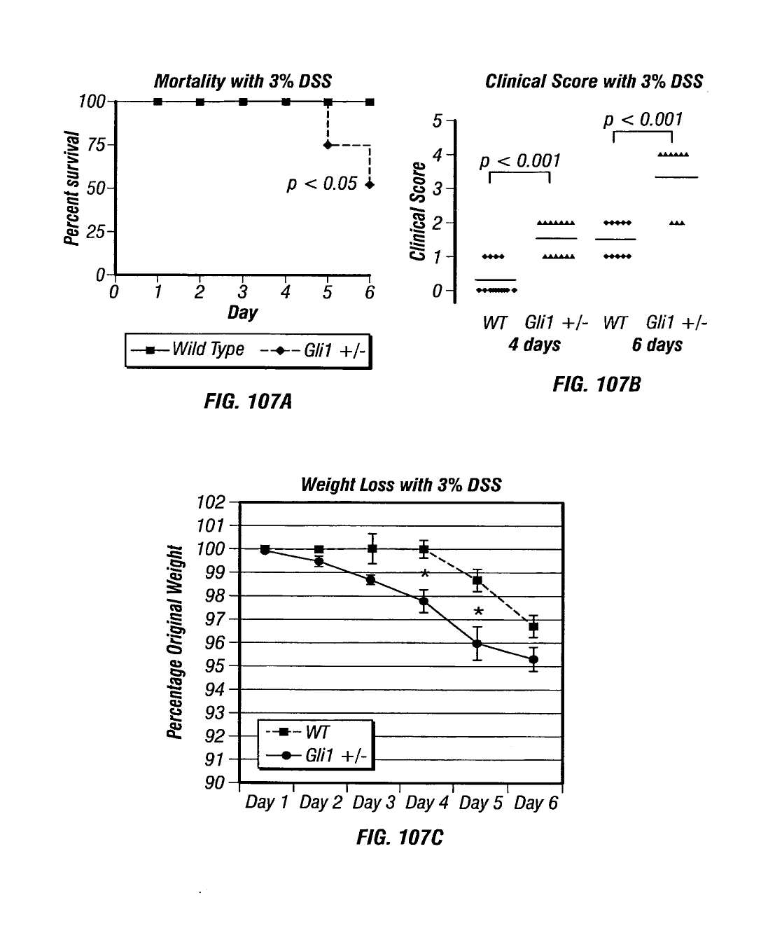

FIG. 107 shows the results in which Gli1+/− animals show mortality, severe clinical symptoms, and profound weight loss after DSS treatment. A) WT animals are 100% viable over the 6 day treatment period (N=14). Nearly 50% of Gli1+/− animals (4/9) die in response to 3% DSS treatment for 6 days. B) Gli1+/− animals display markedly more severe symptoms than WT animals after 4 or 6 days of 3% DSS treatment. 1=diarrhoea, 2=bloody diarrhoea, 4=severe bleeding/death. Each dot represents an individual animal and the solid line shows the mean observation in each cohort. C) G/i/+/− animals (N=9) have lose weight more rapidly than their WT littermates (N=10). *=p<0.05

After 6 days, 4/9 had died, and 3 of the survivors demonstrated severe morbidity, with significant rectal bleeding and almost complete immobility (FIG. 107a). In contrast, no WT animals (N=14) died, all were mobile and showed less morbidity on days 5 and 6 after treatment. Gli1+/− animals developed bloody diarrhoea and significant weight loss by day 4, whereas WT animals did not develop clinical signs or measurable weight loss until day 6 (FIG. 107b-c).

Gli1+/− animals develop more severe colonic pathology than WT littermates in response to DSS treatment. We examined colonic tissue from Gli1+/− and WT animals taken after 4 and 6 days of DSS treatment. Gli1+/− animals developed severe tissue lesions more rapidly than WT.

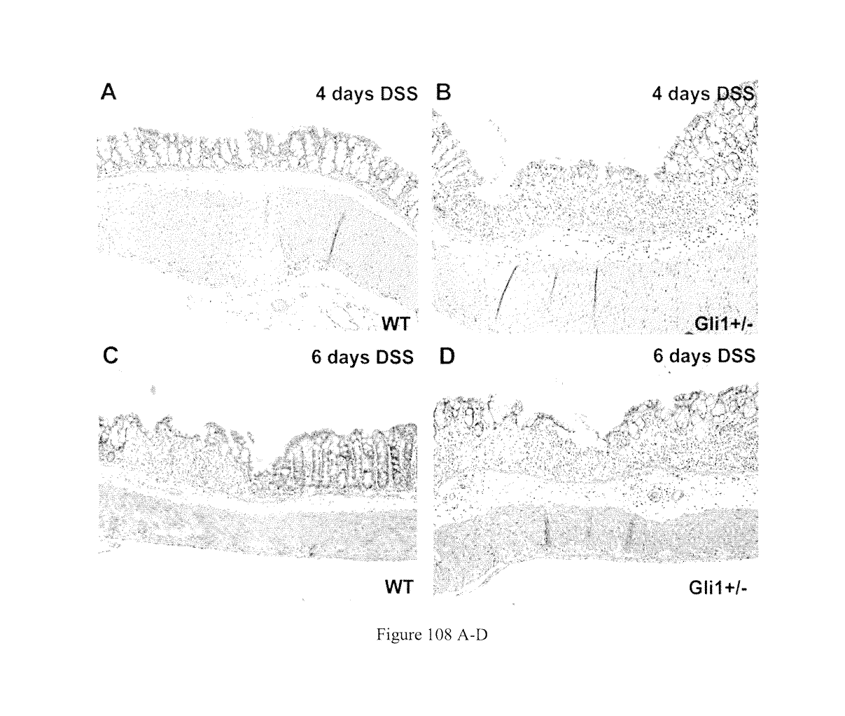

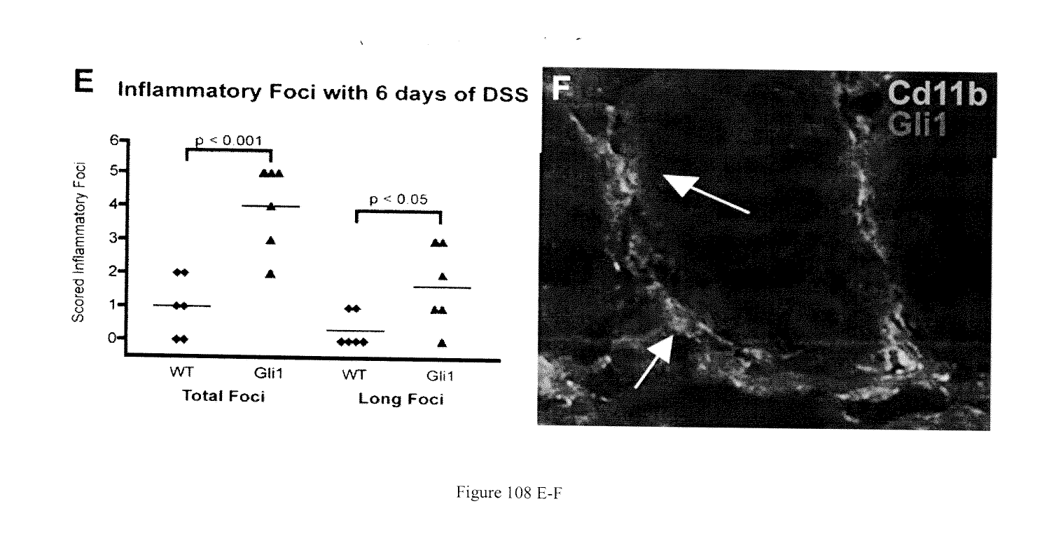

FIG. 108 shows Gli1+/− animals demonstrate more severe intestinal inflammation than WT littermates in response to DSS treatment. A) WT animals (N=4) exhibit mild colonic inflammation but do not develop substantial epithelial or ulcerative inflammatory pathology within 4 days of DSS treatment. B) Gli1+/− animals (N=4) develop significant inflammatory infiltration, epithelial damage, and ulceration within 4 days of DSS treatment. C-D) Gli1+/− animals develop profound intestinal inflammation in response to 3% DSS treatment, with severe epithelial damage in long stretches of their colonic mucosa (N=9). E) Blinded histological scoring of colonic damage after 6 days of DSS treatment. Standard lengths of tissue from the mid colon and distal descending colon were scored in each animal. Gli1+/− animals (N=6) have more overall inflammatory foci and more long foci (10+ crypt units affected) than WT animals (N=6). Each dot represents the number of observed foci in an individual animal; the solid line shows the mean observation in each cohort. Red dots indicate the animals that were analyzed for cytokine expression. F) Resident mucosal myeloid cells respond directly to Hh signalling and express LacZ in the homeostatic colon of Gli1+/− animals. Arrows indicate cells with LacZ-positive nuclei and Cd11b membranes.

After 4 days, WT colons showed evidence of inflammatory change but with few destructive lesions FIG. 108a), while extensive inflammatory infiltration and destructive colonic ulcers were prominent in Gli1+/− mice (FIG. 108b). After 6 days, inflammation in surviving Gli1+/− animals was markedly more severe. The number (FIG. 108e), size and invasiveness of inflammatory lesions (FIG. 108c-e) were significantly greater in Gli1+/− animals. Taken together, these results demonstrate that the loss of a single Gli1 allele leads to increased sensitivity to DSS treatment as reflected by severe intestinal inflammatory pathology and obvious clinical signs.

Intestinal myeloid cells respond directly to HH signals. We have demonstrated that HH signalling is exclusively paracrine in murine intestine and colon [Madison et al. (2005) Development 132: 279-28914]. Here we confirm that Gli1 is expressed in inflammatory cells in mouse, utilizing G/i/+/lacZ animals, which allow Hh-responsive cells to be easily visualized [Park et al. (2000) Development 127: 1593-1605]. In resting adult colon, lamina propria resident CD11b-positive myeloid cells express LacZ and are therefore responding directly to Hh signals (FIG. 108e).

Gli1+/− animals have increased IL-23p19 and pro-inflammatory cytokine expression.

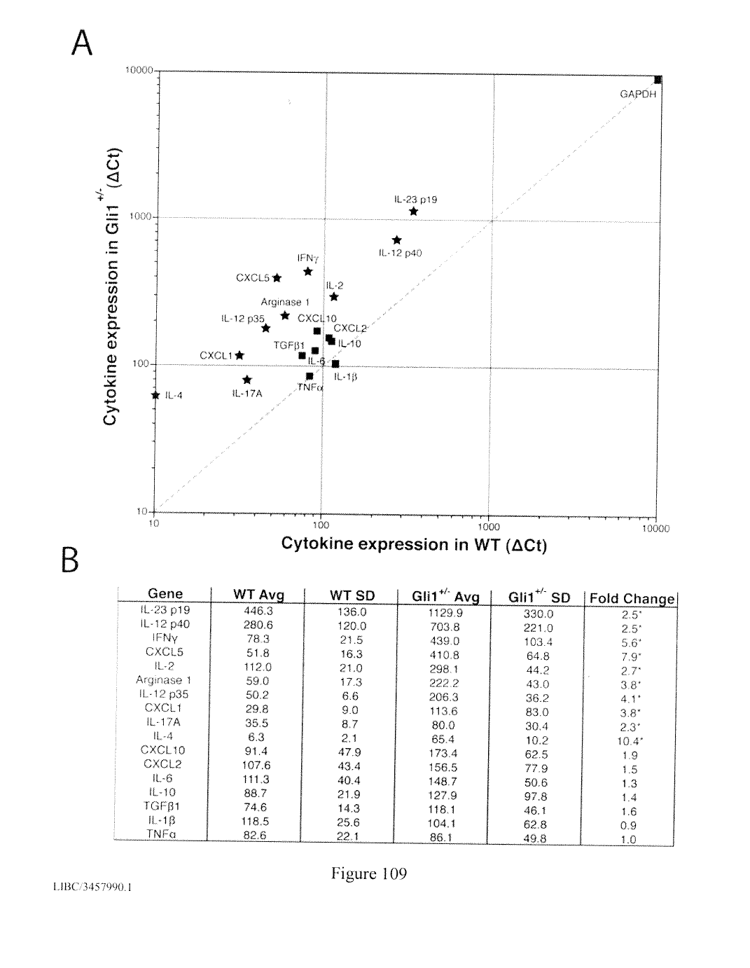

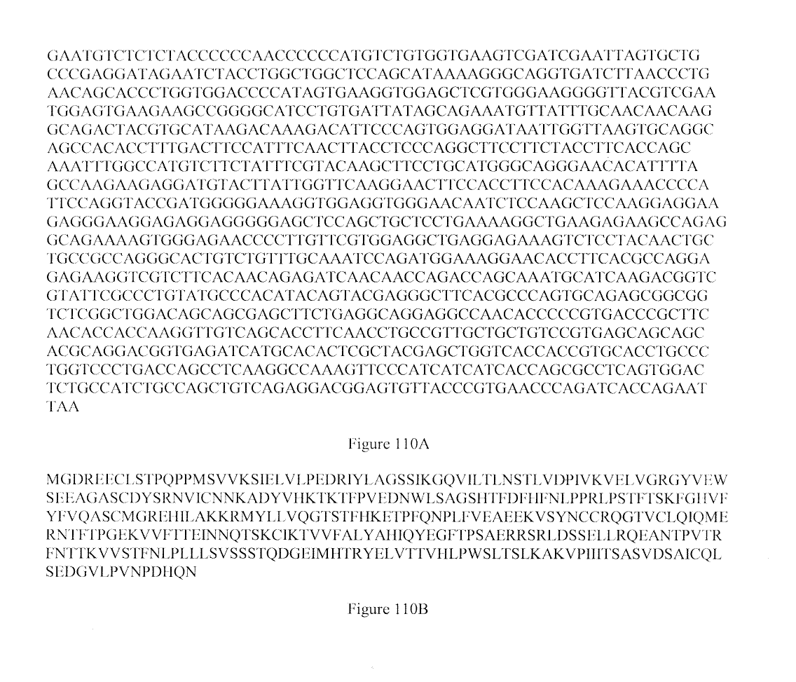

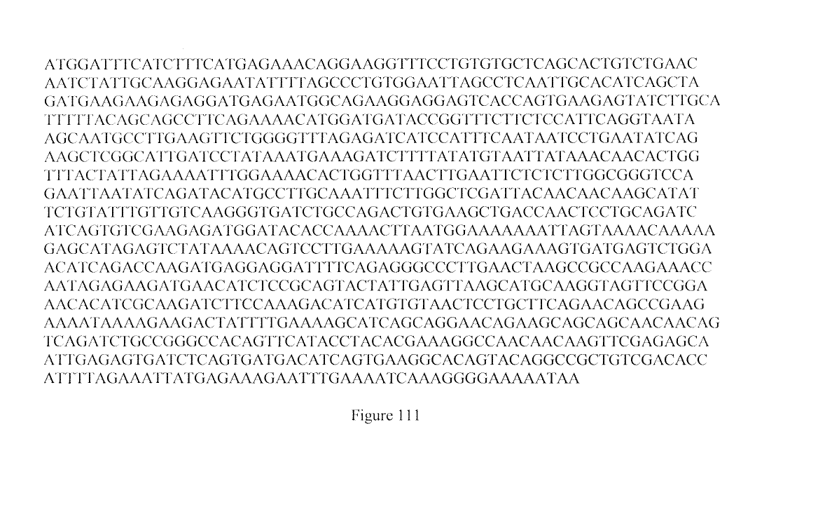

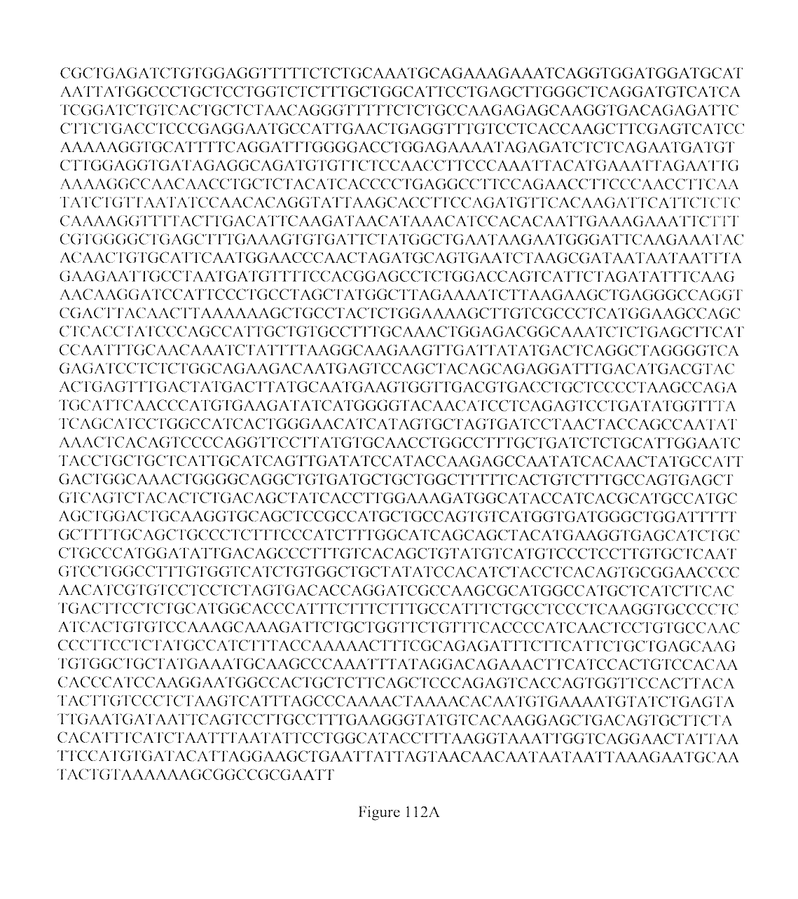

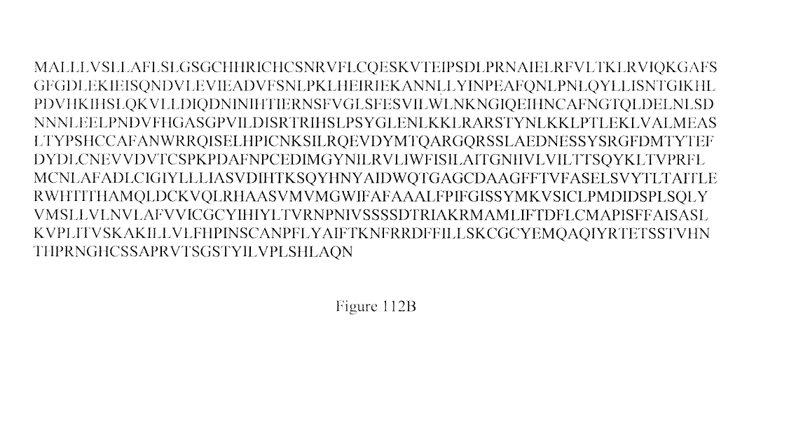

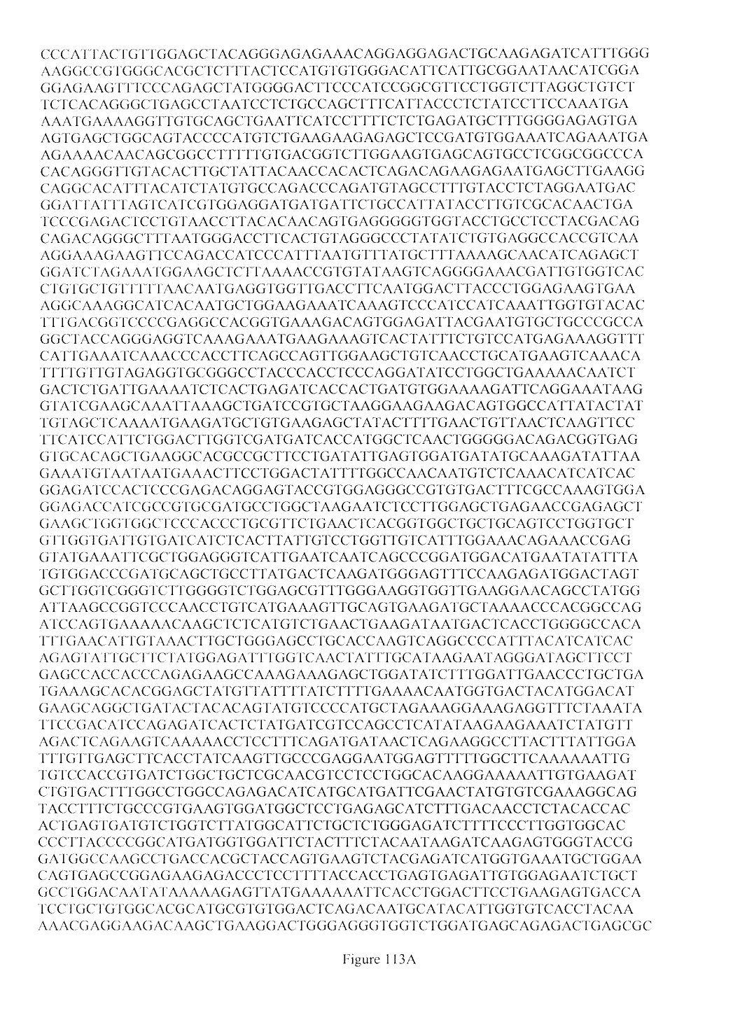

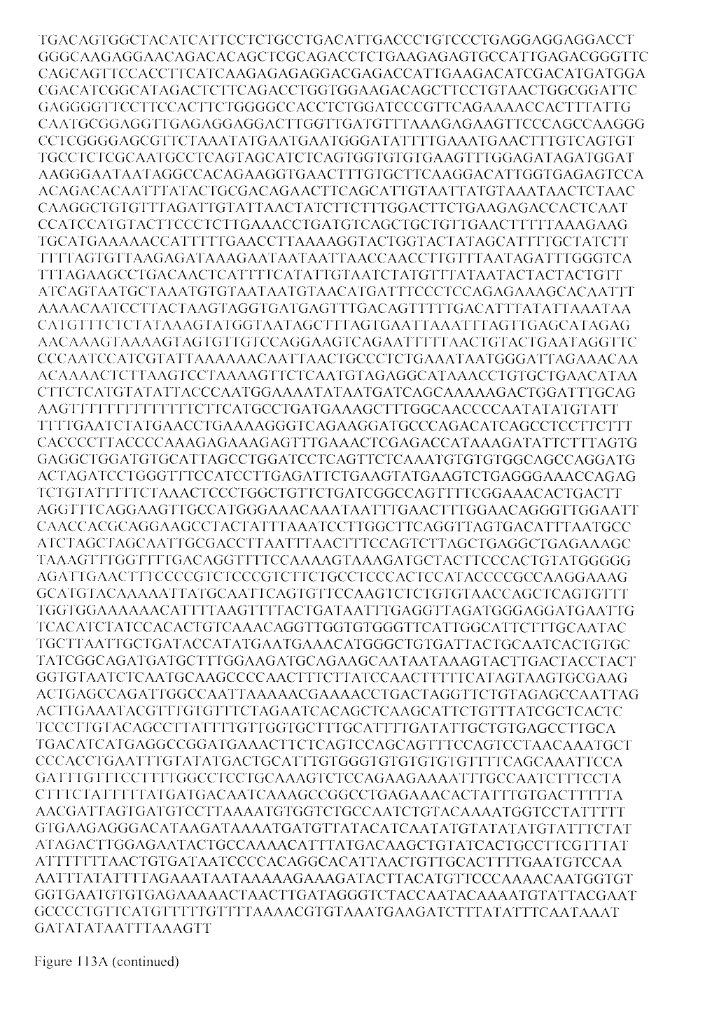

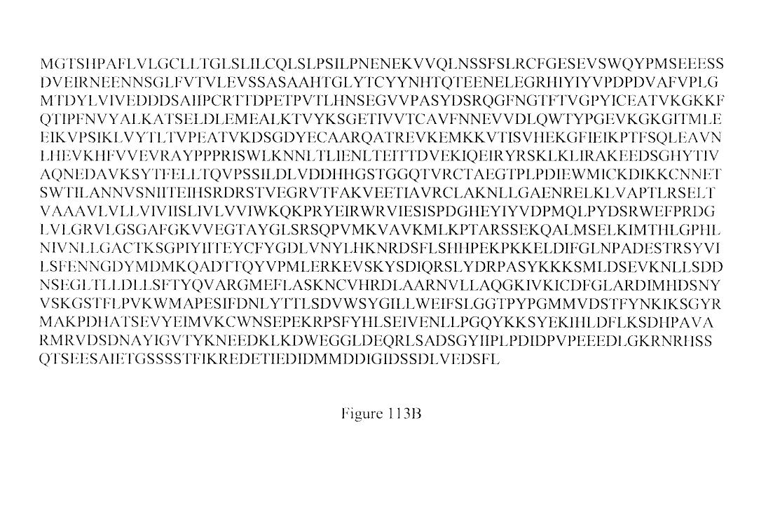

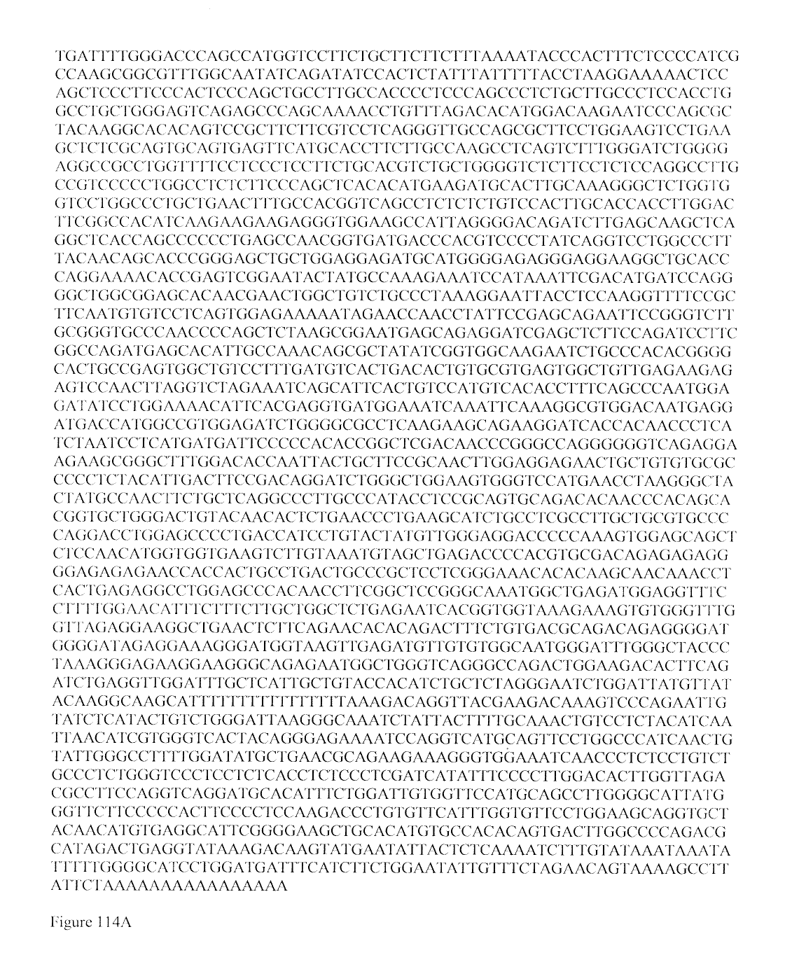

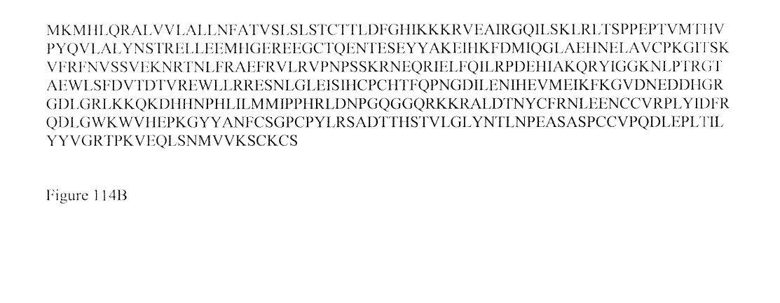

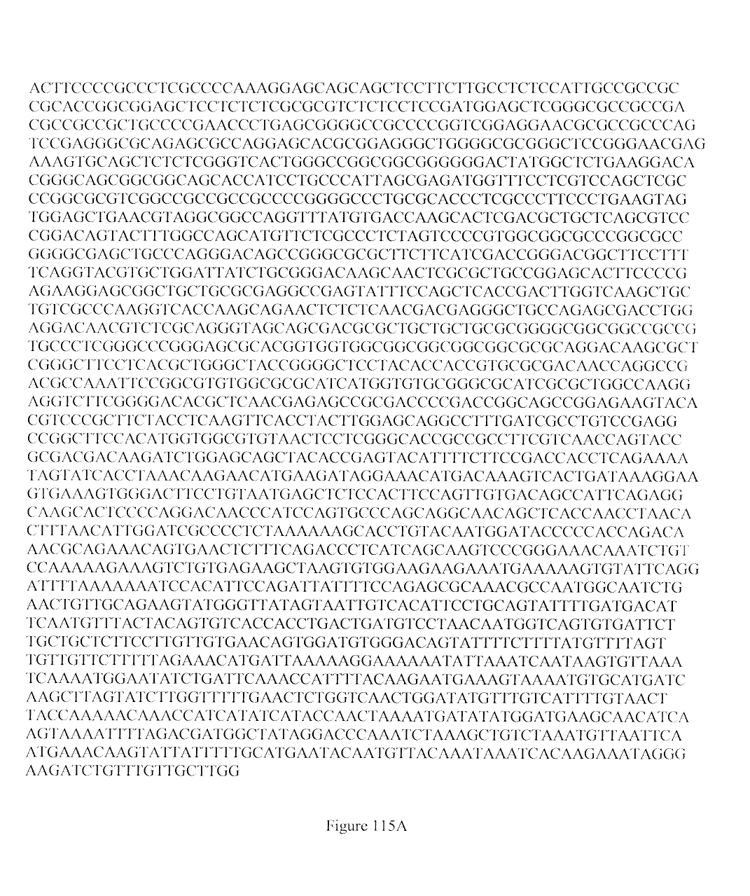

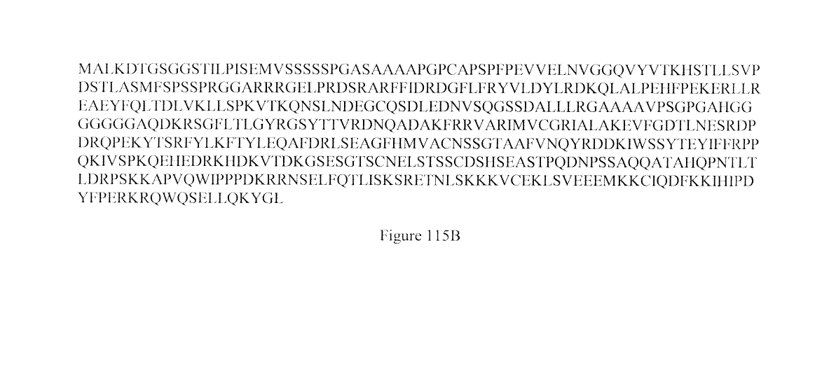

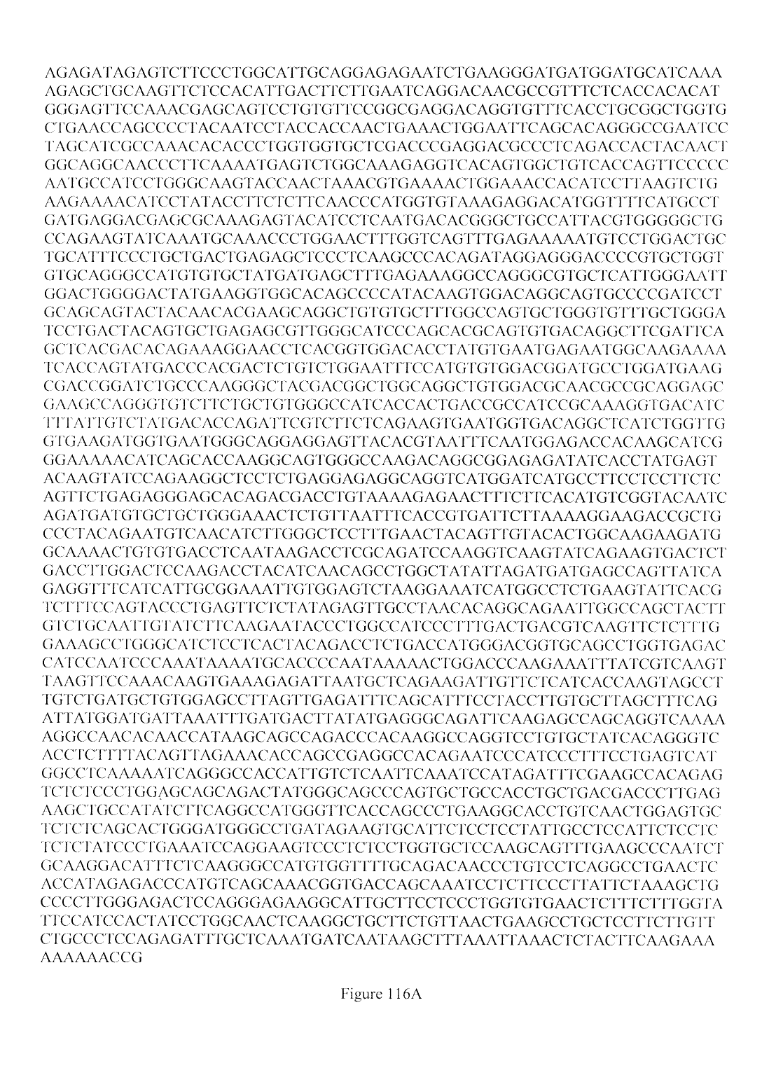

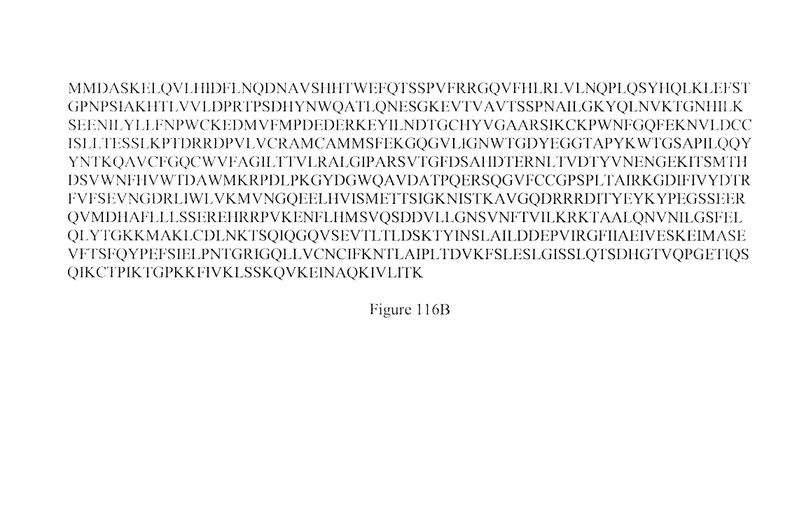

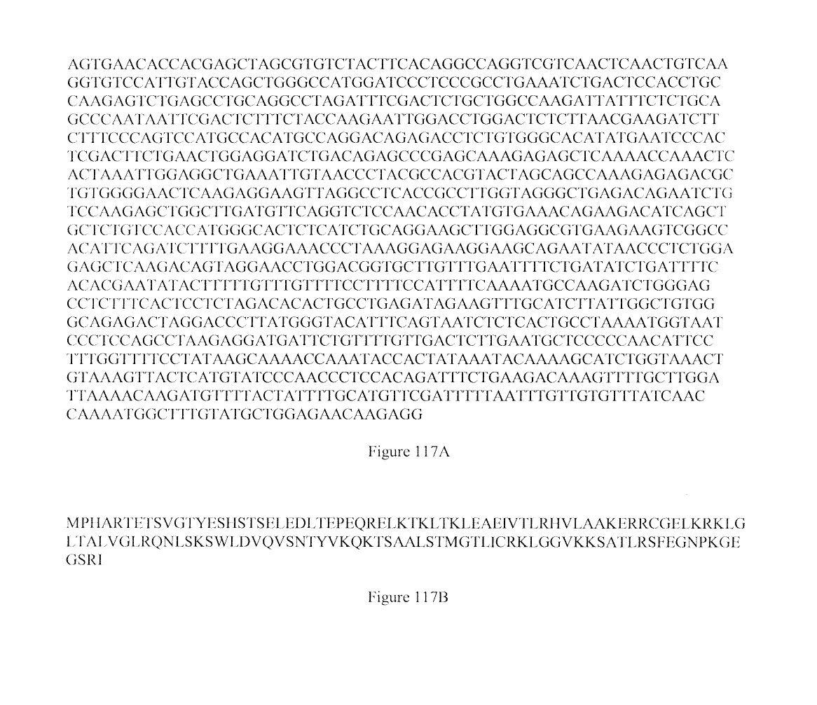

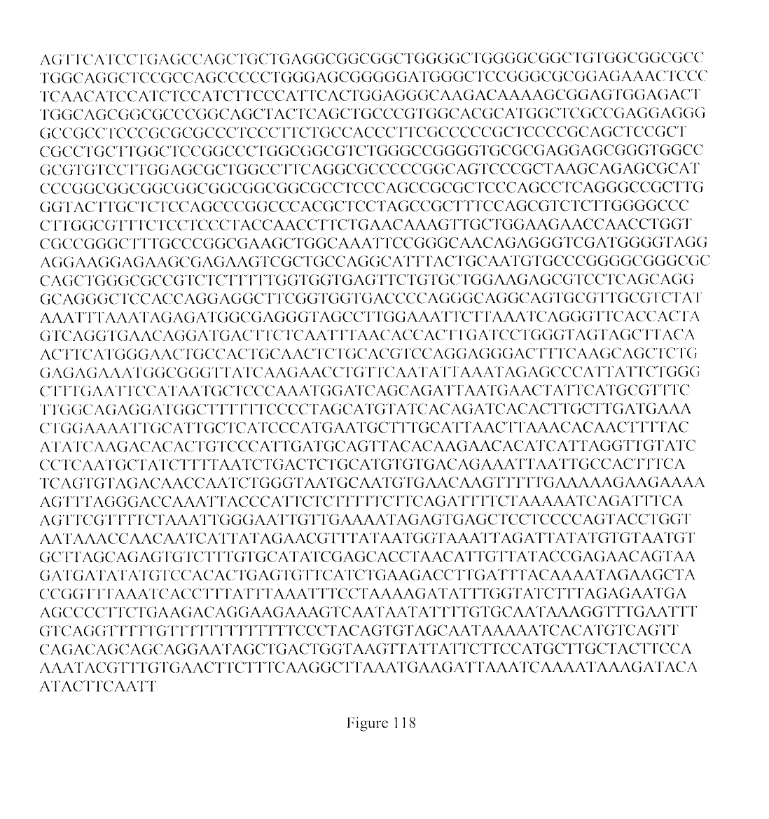

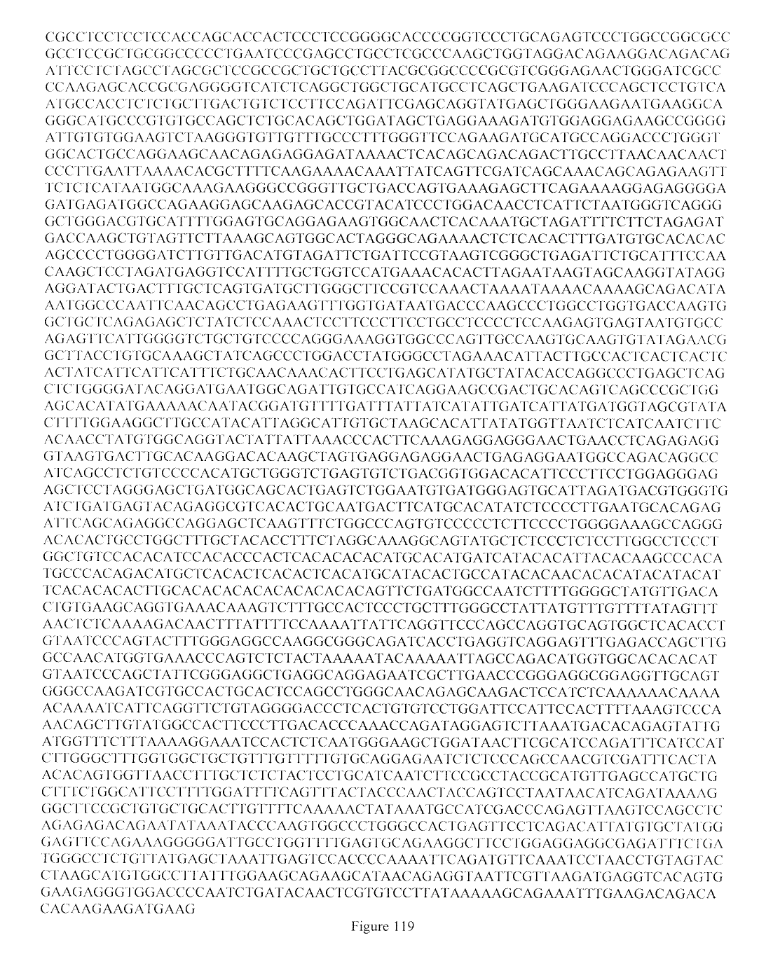

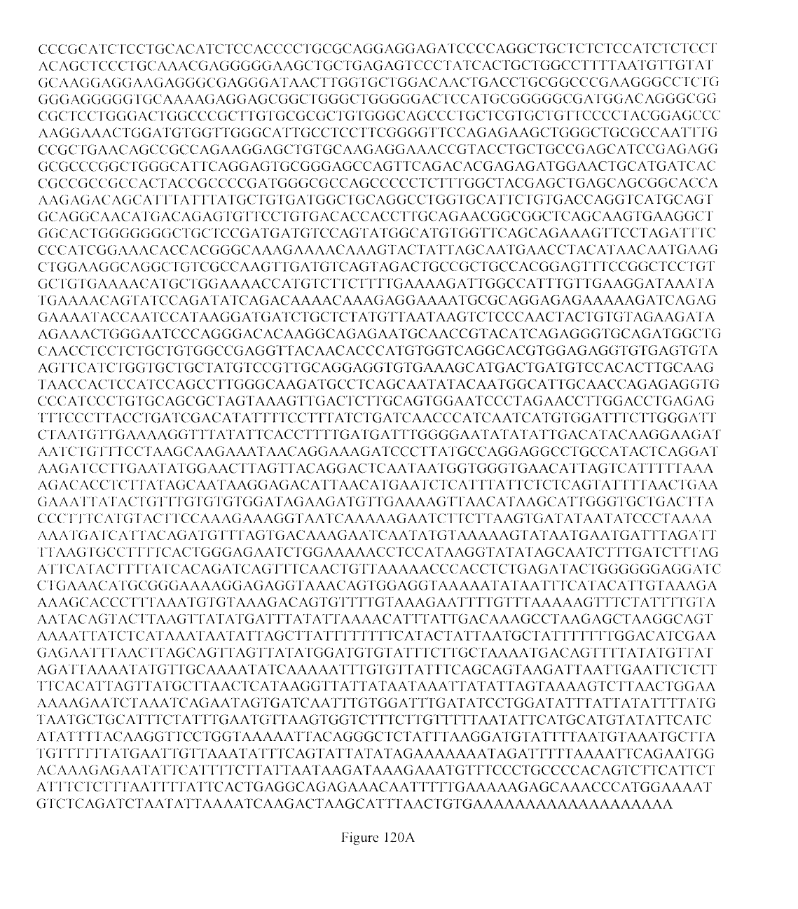

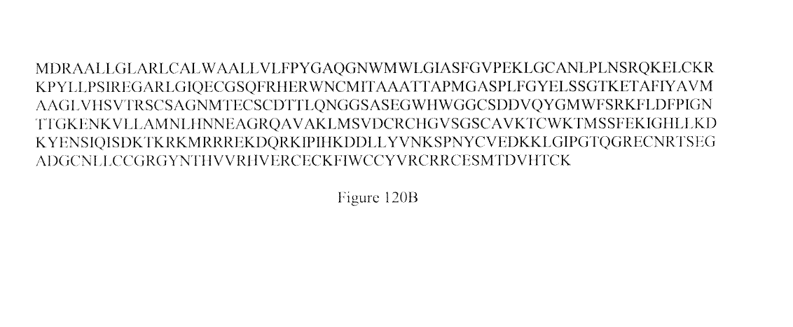

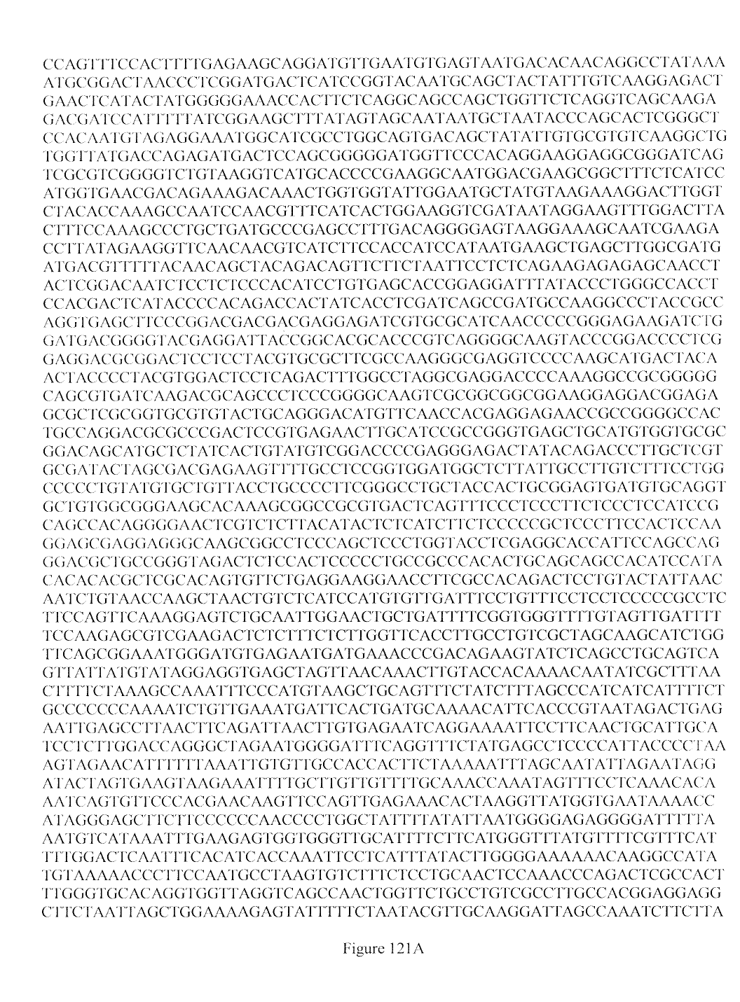

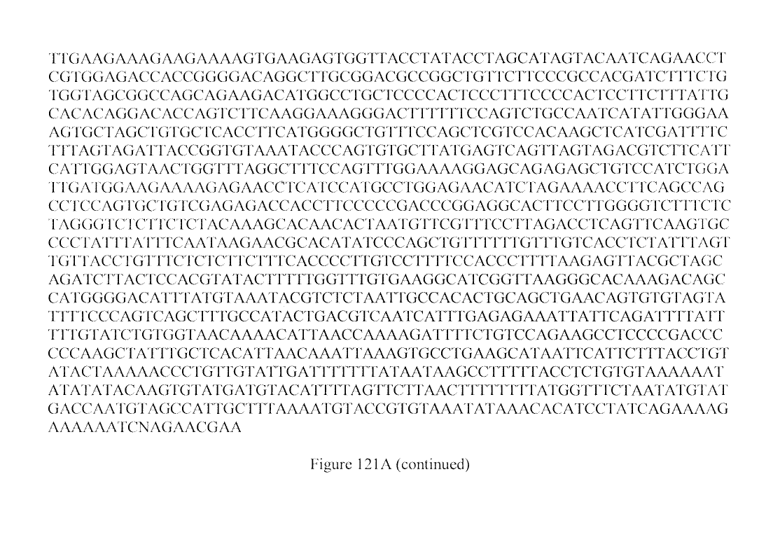

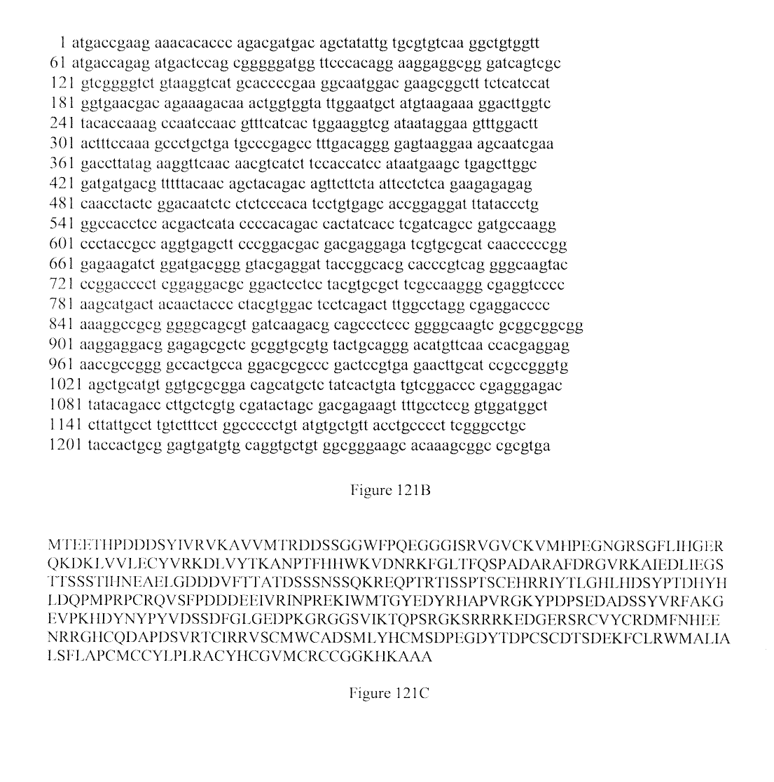

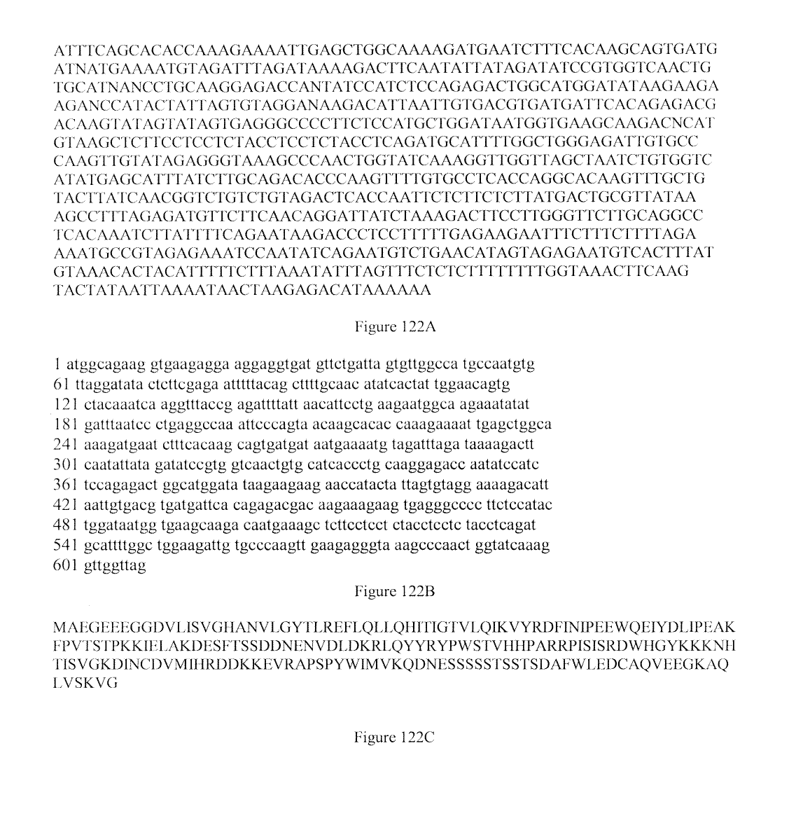

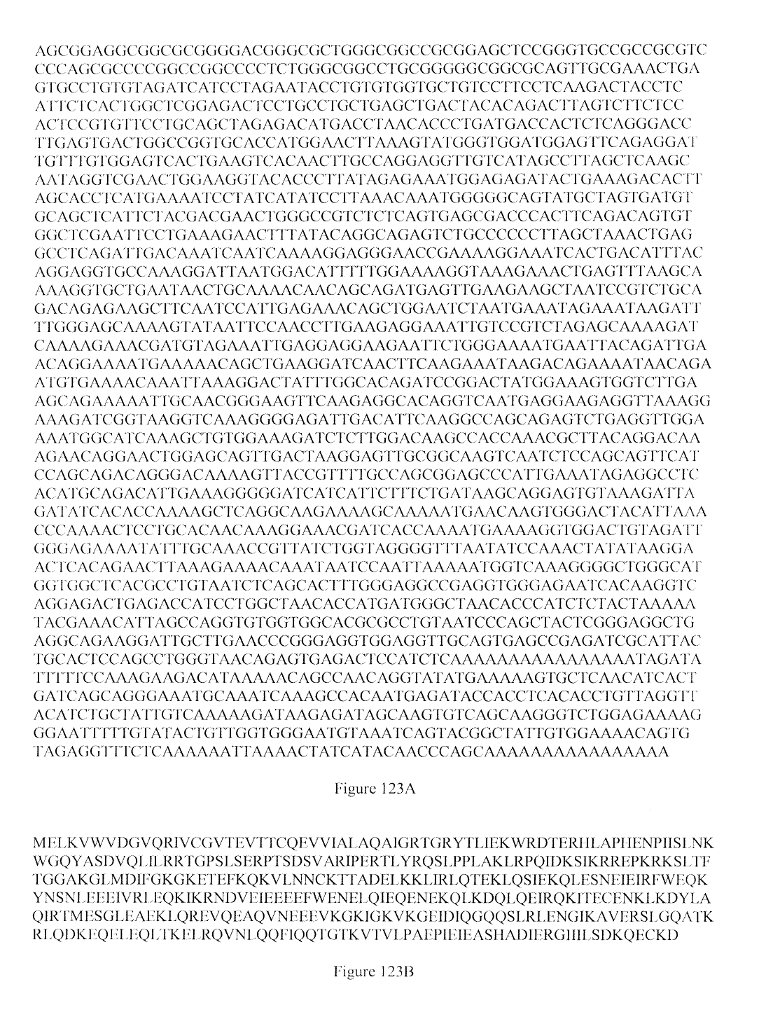

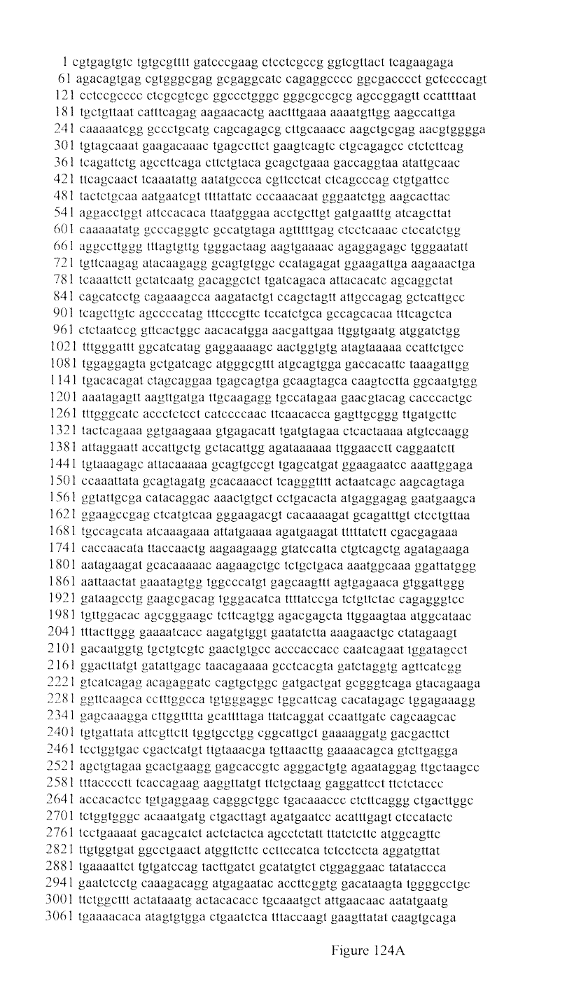

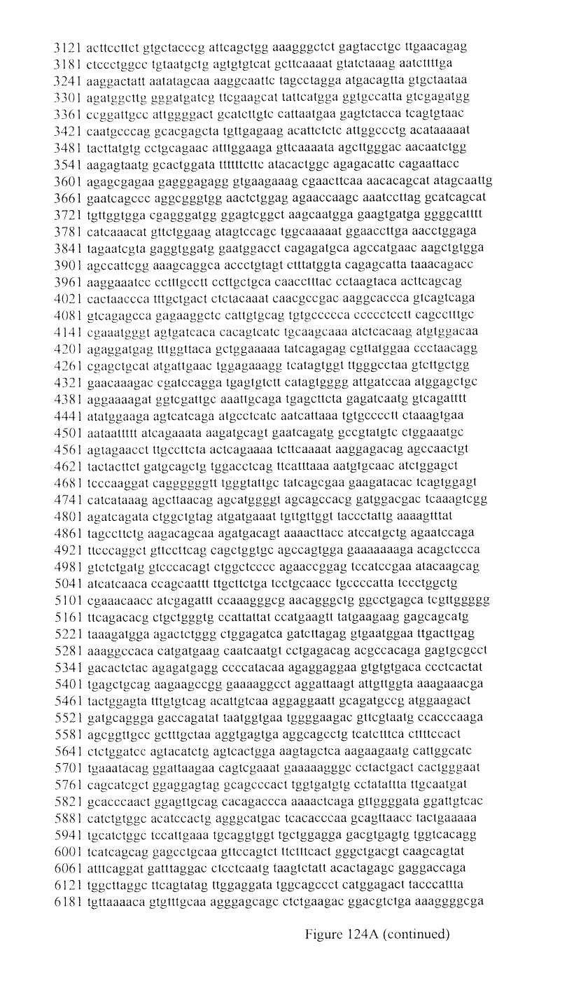

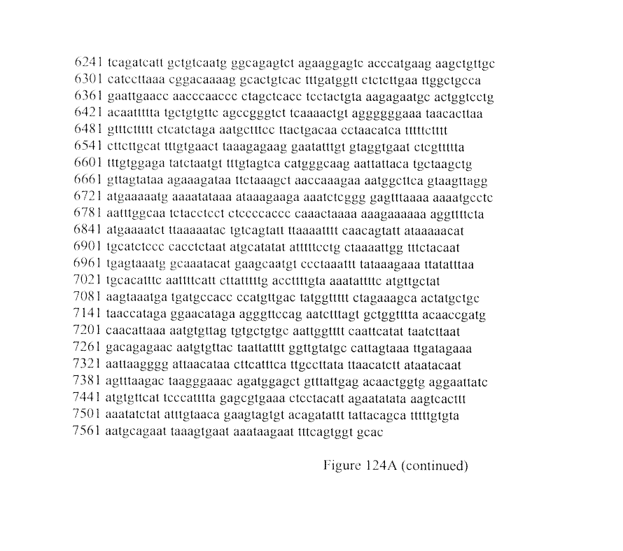

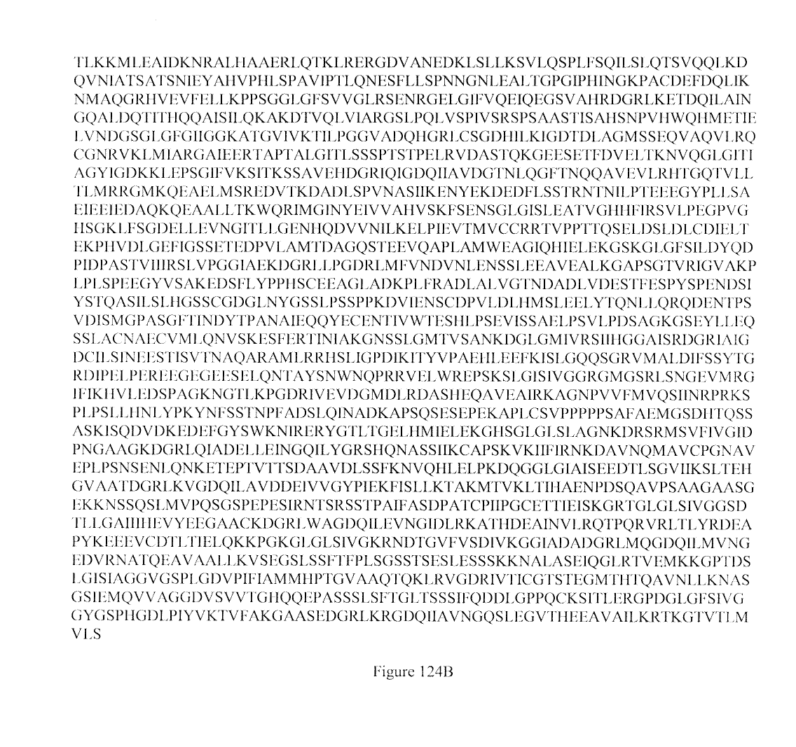

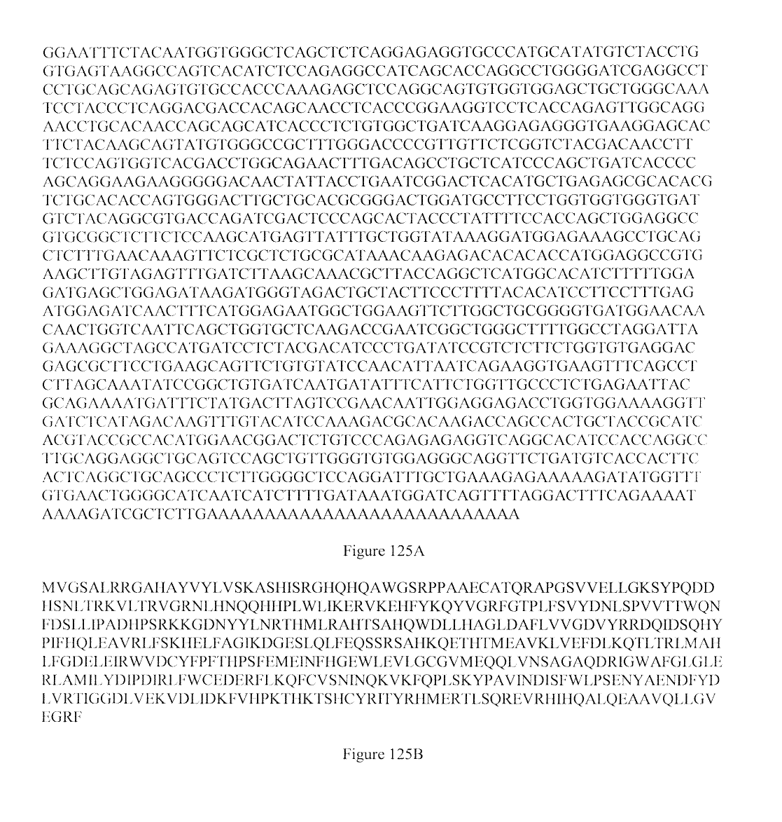

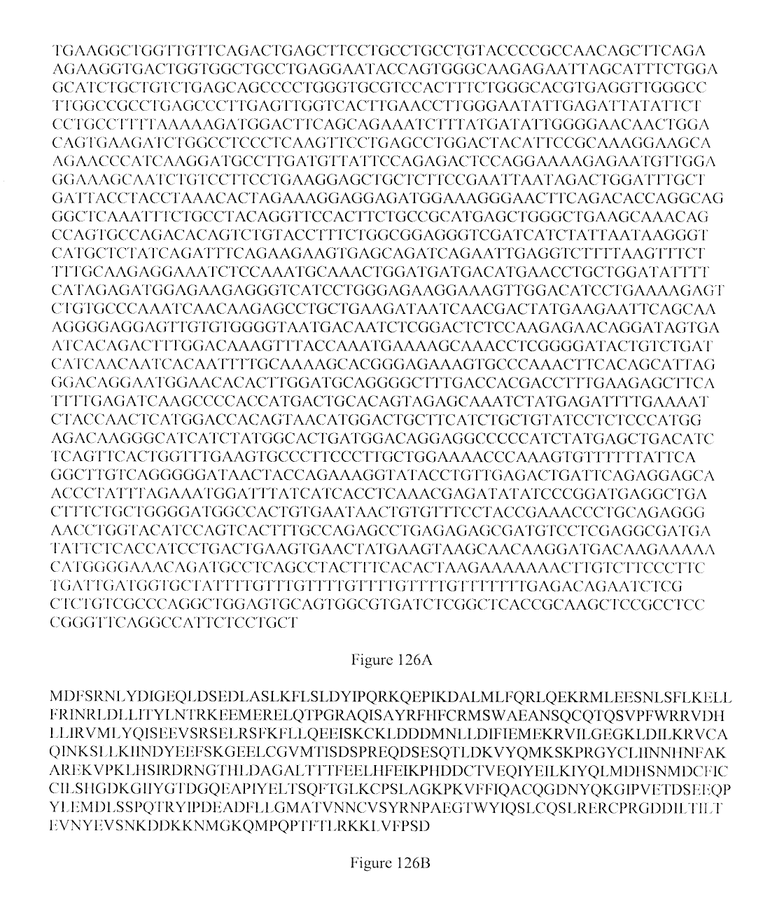

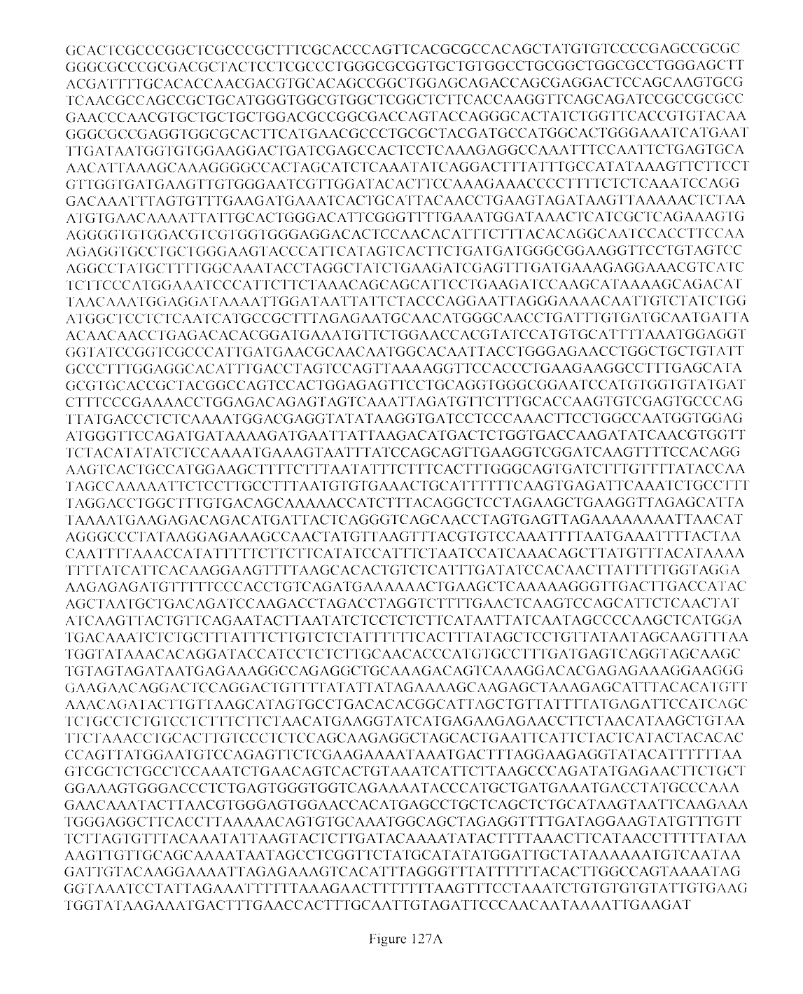

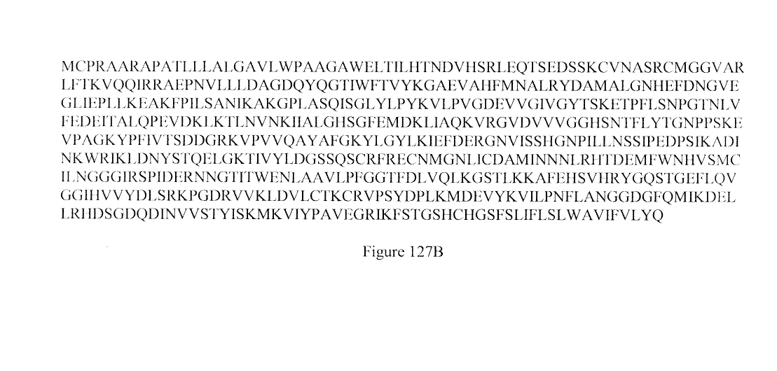

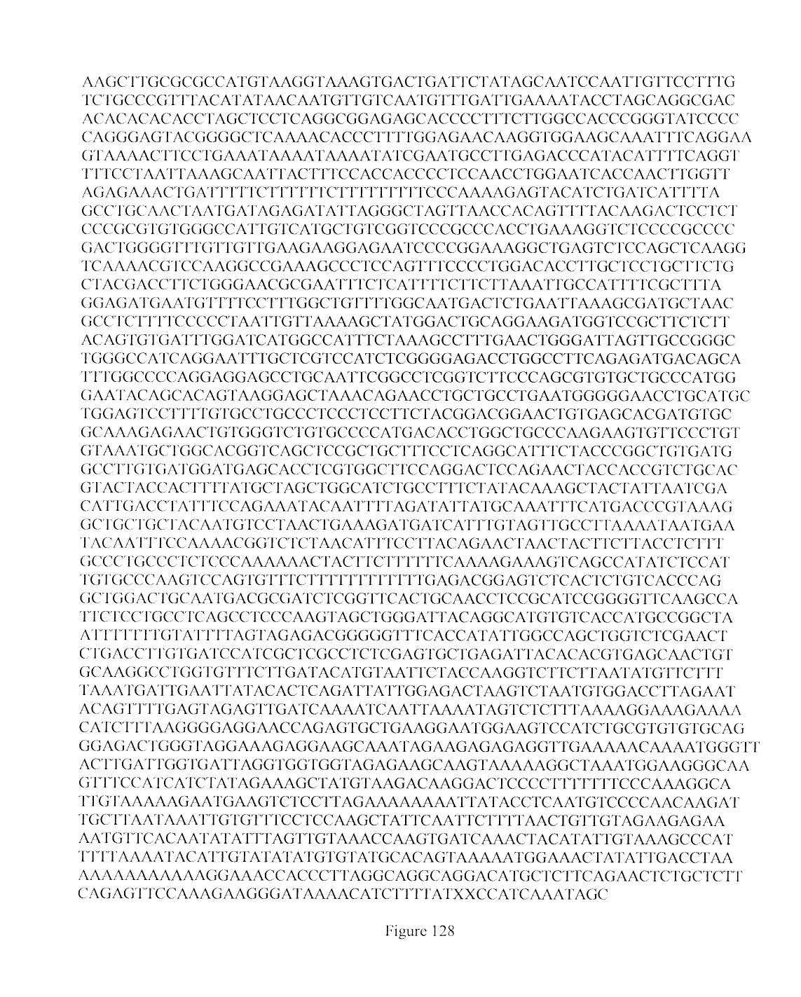

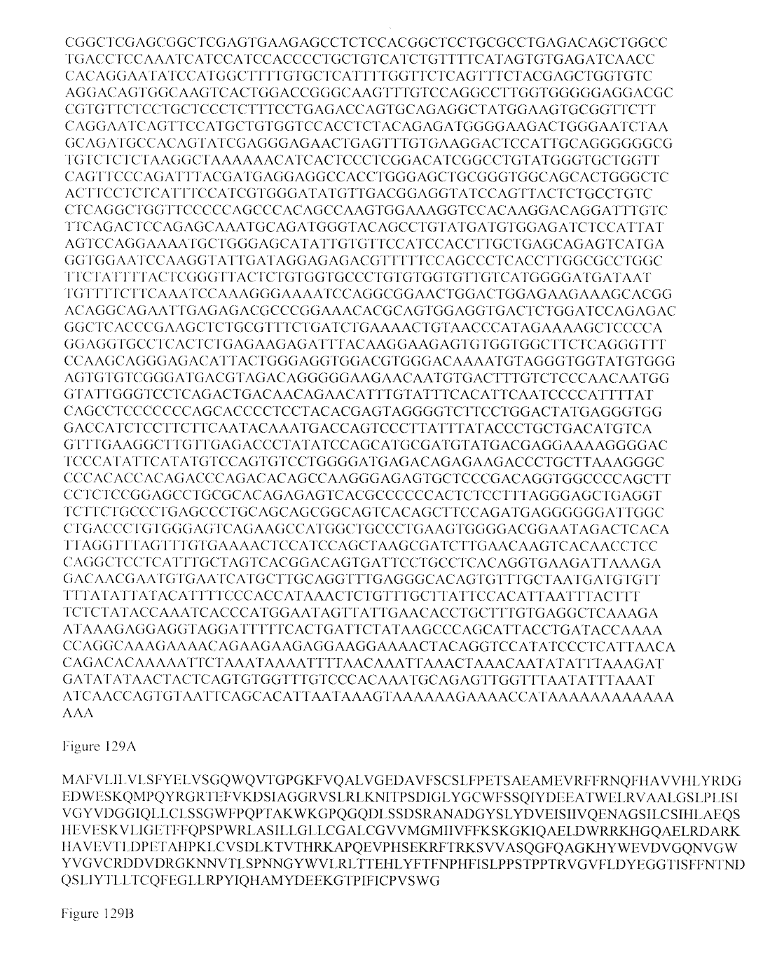

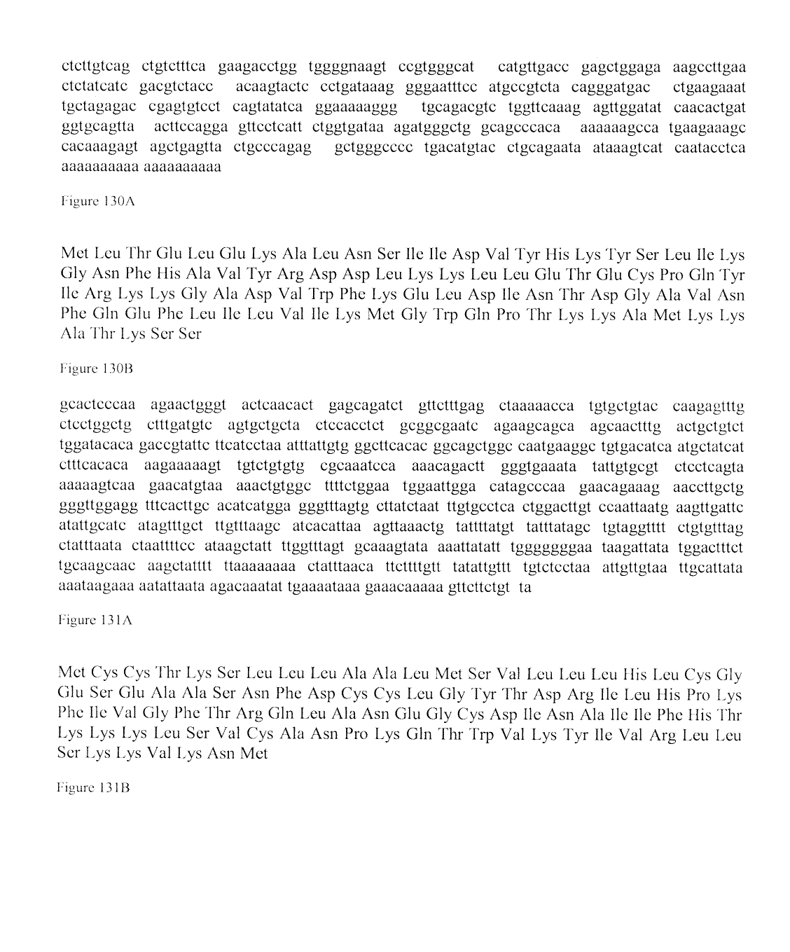

FIG. 109 shows cytokine analysis of Gli1+/− and WT mice after DSS treatments demonstrates robust pro-inflammatory cytokine activation. A) Cytokine expression in WT and Gli1+/− animals (N=4) after 6 days of 3% DSS treatment. Cytokine expression normalized to GAPDH is plotted on the Y-axis for Gli1+/− mice, and on the X-axis for WT animals. Gene expression levels that are changed in a statistically significant manner are shown with stars. The dotted diagonal trendline indicates identical expression levels between WT and Gli1+/− mice. B) Table showing the average cytokine expression, standard deviation, and fold change in Gli1+/− animals compared to WT controls (*=p<0.05). Several pro-inflammatory cytokines are upregulated in Gli1+/− animals, but anti-inflammatory cytokines are largely unchanged.

QPCR examination of cytokine expression on whole colonic tissue taken after 6 days of DSS confirms the histological and clinical data; Gli1+/− animals demonstrate very significant inflammation compared to WT (FIG. 109). We detect robust expression of TH1 cytokines, including IFNγ, in Gli1+/− animals, not surprising given the severity of the inflammation in these animals and the prominence of TH1 cells in DSS-induced colitis. We did not detect a significant difference in TGF13 and IL-10 between Gli1+/− and WT animals, suggesting that down-regulation of anti-inflammatory cytokines was not the primary mechanism of increased inflammation in this model. The most highly expressed cytokine in Gli1+/− animals was IL-23p19, which is known to drive differentiation of TH17 lymphocytes, key mediators of inflammation in several systems, including IBD [Hue et al. (2006) J Exp Med 203: 2473-2483; Yen et al. (2006) J Clin Invest 116: 1310-1316]. IL-12 and IL-17, cytokines closely associated with IL-23, were also upregulated in Gli1+/− animals. These data are particularly significant since the IL-23 pathway has recently been strongly implicated in IBD pathogenesis both in humans [Duerr et al. (2006) Science 314: 1461-1463] and mice [Yen et al. (2006) J Clin Invest 116: 1310-1316]. Our data provide a potential link between this key inflammatory pathway and the robust inflammation seen with reduced GLI1 dosage or function.

Discussion

The data presented here provide the first evidence that intact HH signalling is critical in the mammalian gut response to inflammatory challenge, and that reduced GLI1 function is implicated in IBD pathogenesis. We confirm that the HH signaling pathway is downregulated in colonic inflammation in man. We identify a specific GLI1 variant that is highly associated with UC/IBD, and demonstrate that the variant protein is functionally deficient as a transcriptional activator in vitro. Finally, we demonstrate that a 50% reduction in murine Gli1 results in a heightened intestinal inflammatory response to DSS with significant upregulation of the IL-23 pathway. Not only do these findings have clear implications for the understanding of IBD pathogenesis with potential for therapeutic intervention, they are the first clear description of a functional role for HH signalling and GLI1 in bowel inflammation. The inherited variation in the GLI1 gene that we have detected is associated with IBD and UC, in both Scotland and England, with findings for rs2228226 confirmed by meta-analysis of over 5000 individuals, with odds ratio of 1.19. Evidence for an effect in CD is seen in the present study, but the predominant effect is clearly related to UC. The magnitude of this association is entirely in line with the effect size noted in a number of recent studies of complex disease genetics, including CD, colo-rectal cancer [Tenesa et al. (2008) Nat Genet. 40: 631-637], and coeliac disease [Hunt et al. (2008) Nat Genet. 40: 395-402]. The level of significance attained satisfies suggested criteria of p<10⁻⁴-10⁻⁶ for gene-centric studies [Burton et al. (2007) Nat Genet. 39: 1329-1337; Thomas et al. (2004) J Natl Cancer Inst 96: 421-423]. The three Northern European populations studied have previously demonstrated similar contribution of other IBD susceptibility genes/loci, including NOD2 and IBDS [Gaya et al. (2006) Lancet 367: 1271-1284]. Whilst the minor allelic frequencies for this SNP are very similar in Scotland and Sweden (30.3% and 30.6%), they differ by 3.9% between Scotland and Cambridge (30.3% and 26.4%). This difference is in keeping with that noted for a number of SNPs analysed for population stratification in the recent WTCCC study [Wellcome Trust Case Control Consortium (2007) Nature 447: 661-678]. Whilst our resequencing efforts identify rs2228226 as the only coding variant associated with IBD, the haplotype analysis and log-likelihood analyses raise the possibility that other germ-line variants may also contribute to IBD risk. These need be explored formally—specifically the role of intronic variants, long-range promoter effects and/or copy number variation. In this context, several complex disease genes, including NOD2 [Hugot et al. (2001). Nature 411: 599-603; Ogura Y et al. (2001). Nature 411: 603-606], have multiple independent mutations conferring disease risk, some disease genes have no causative mutations within coding sequences (e.g. IRGM in CD [Parkes et al. Am J Hum Genet. 2000; 67:1605-16105]), and synonymous SNPs may be associated with functional effects [Kimchi-Sarfaty et al. (2007) Science 315: 525-528]. rs2228226C→G encodes a change from glutamine to glutamic acid (Q1100E). Our in vitro data demonstrates that GLI1 1100E is a subfunctional transcriptional activator compared to WT GLI1, though it is synthesized and localized appropriately. The Q1100E mutation causes a significant charge change in a conserved region directly adjacent to the known transactivation region of GLI1; this change could directly modify transactivation activity, disrupt the structure of the transactivation domain, or affect protein stabilization [Huntzicker et al. (2006) Genes Dev 20: 276-281] decreasing activity. Our data suggest that reductions in GLI1 activity or amount also produce a robust phenotype. Gli1+/− animals, which have only 50% of the WT level of Gli1, develop severe inflammation rapidly in response to moderate stimuli. These data, to our knowledge the first description of a phenotype for reduced Gli 1 function [Park et al. (2000) Development 127: 1593-1605], demonstrate the key role that a full complement of Hh response plays in protection from inflammatory disease. In addition, our in vitro data demonstrates that GLI1 E1100 is capable of activating some Gli response, suggesting that under homeostatic conditions, GLI1 E1100 could perform adequately. Similar to the situation in Gli1+/− animals, however, under conditions of inflammatory stress, GLI1 E1100 can only function at 50% of the level of WT GLI1. Whether the predisposition to inflammation in these systems is a direct result of lowered HH signal transduction within inflammatory cells or reflects the effect of lower HH signals on stromal target cells that, in turn, release signals that impact the integrity of the epithelial layer is not yet clear. Addressing this question will be a key avenue of future investigation. We have shown that the HH pathway may directly modify the innate immune response through signalling to myeloid target cells. This finding is in accordance with recent data demonstrating a crucial role for Hh signalling in myeloid cell maturation in the spleen [Varas et al., (2008) J Leukoc Biol June; 83(6):1476-83. Epub 2008 March 1123]. Interestingly, myeloid cell populations can differentially modify the intestinal inflammatory milieu through a significant impact on the IL-23/IL-17 pathway [Denning et al. (2007) Nat Immunol 8: 1086-1094]. Together, our findings demonstrating direct HH response by innate immune populations and increased IL-23 after DSS treatment in Gli1+/− animals suggest that HH signalling may normally promote a tolerogenic phenotype in mucosal myeloid cells; reduced HH signal transduction may instead trigger an inflammatory response in these cells.

In conclusion, we demonstrate here for the first time the altered expression of a developmental signalling pathway in IBD, opening up novel lines of investigation. Furthermore, we show that these effects are in part genetically determined, with evidence implicating GLI1 as an IBD2 gene, and identification of a specific variant with reduced transcriptional activity. The functional relevance of Gli1 is demonstrated by the severe intestinal inflammation that develops in the face of a 50% reduction in Gli 1 concentration in an established mouse model of colitis. Taken together, these data strongly argue for the importance of a robust HH pathway activation in the protective response of the intestinal mucosa to inflammatory stimuli. These findings have important implications for the pathogenesis of UC and potentially for other forms of chronic inflammation. [Lees et al. (2006) Gastroenterology 131: 1657-1658].

### Example 5

### IBD Gene Expression Profiles from Whole Blood Samples

Genome wide expression profiles from human endoscopic colonic biopsies have started to help dissect out the pathogenic inflammatory pathways at the cellular level in IBD (Noble C L et al. Gut, 2008 Jun. 3. [Epub ahead of print]). Whole blood genome wide expression profiles may complement conventional endoscopic techniques to help in the diagnosis of IBD- Crohn's disease (CD) and ulcerative colitis (UC). The aim of this study was to investigate whole blood gene expression profiles to try to differentiate patients with IBD- CD, UC, and controls (HC).

Patients. 21 UC, 19 CD, and 10 controls (HC) were studied. 2 of the UC patients were newly diagnosed, 15 had quiescent disease and 4 had active disease. In CD 1 new diagnosis, 11 quiescent disease and 7 active disease patients were investigated.

Methods. 41058 expression sequence tags (representing 33296 genes) were analyzed in 50 whole blood samples using the Agilent platform. Total RNA was extracted from the blood using the micro total RNA isolation from animal tissues protocol (Qiagen, Valencia, Calif.). A T7 RNA polymerase single round of linear amplification was carried out to incorporate Cyanine-3 and Cyanine-5 label into cRNA. The samples were hybridized for 18 hours at 60° C. with constant rotation. Microarrays were washed, dried and scanned on the Agilent scanner according to the manufacturer's protocol. Microarray image files were analysed using Agilent's Feature Extraction software version 7.5. The genes were normalized using the Stratagene Universal Human Reference.

Using clustering analysis with all the IBD and HC patients, and probes that had a > or < than 1.5 fold change in expression, HC patients were more prevalent on one side of the dendrogram 1/21 v 9/29 (p=0.02 OR 1.8) (data not shown). No difference was observed in the distribution of the CD and UC samples. When all of the IBD samples were compared to controls 493 sequences had a fold change of greater than 1.5 (1.7×10⁻⁴¹<p<0.01) and 595 sequences had a fold change of less than 1.5 (4.0×10⁻⁴⁰<p<0.01). When CD and UC were compared, 293 sequences had a fold change of greater than 1.5 (5.4×10⁻²⁷<p<0.01) and 301 sequences had a fold change of less than 1.5 (5.2×10⁻¹⁸<p<0.01).

By using a panel of 10 of the up regulated and down regulated genes sequences we were able to predict with a >90% sensitivity IBD samples from controls. Table 12 below lists 20 differentially regulated genes in IBD when compared to controls.

TABLE 12

| Sequence Name | Sequence Code | Fold Change | P-value |
|---|---|---|---|
| LOC342959 (AARDC5) | A_32_P30271 | 4.92527 | 8.62E−18 |
| A_24_P910246 | A_24_P910246 | 2.92405 | 1.41E−07 |
| (ATXN3L) | | | |
| LOC92552 (FSHR) | A_23_P361744 | 2.78494 | 4.12E−13 |
| PDGFRA | A_23_P332536 | 2.7178 | 0.00004 |
| TGFB3 | A_24_P373096 | 2.67234 | 5.89E−09 |
| KCTD8 | A_23_P94902 | 2.65574 | 0.00005 |
| TGM4 | A_23_P41241 | 2.64262 | 0.0005 |
| NYD-SP25 | A_24_P309216 | 2.62905 | 7.76E−15 |
| FLJ33651 | A_24_P306032 | 2.62873 | 0.0034 |
| EMX2OS | A_24_P892472 | 2.55683 | 0.00029 |
| WNT16 | A_23_P134601 | −2.96773 | 3.46E−10 |
| SPRED2 | A_24_P315535 | −2.97751 | 3.12E−16 |
| MGC50721 (C16orf65) | A_23_P412508 | −3.06148 | 0.0088 |
| C12orf2 | A_24_P78556 | −3.17916 | 0.0129 |
| MPDZ | A_23_P396328 | −3.19437 | 1.87E−40 |
| FARS2 | A_24_P456422 | −3.35391 | 0.00097 |
| CASP8 | A_24_P157087 | −3.41412 | 3.90E−09 |
| NT5E | A_24_P316430 | −3.63046 | 0 |
| TDGF3 | A_24_P179646 | −3.71928 | 7.31E−23 |
| BTNL3 | A_23_P158297 | −5.22514 | 2.09E−12 |

Whole blood genome wide expression signature provides a starting point for differentiating between patients with IBD and controls and this may provide complimentary diagnostic evidence of the diagnosis of IBD.

1. A method of diagnosing the presence of an inflammatory bowel disease (IBD) in a mammalian subject, comprising determining that the level of expression of a nucleic acid encoding a polypeptide shown as any one of SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68. 70, 72, 74, 76, 78, 80, 82, 84, 86, 88, 90, 92, 94, 96. 98, 100, 102, 104, 106, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 194, 197, 199, 201, 203, 205, 207, and 230 in a test sample obtained from said subject is higher relative to the level of expression in a control, wherein said higher level of expression is indicative of the presence of an IBD in the subject from which the test sample was obtained.

2. (canceled)

3. The method of claim 1 wherein said mammalian subject is a human patient.

4. The method of claim 3 wherein evidence of said expression level is obtained by a method of gene expression profiling.

5. The method of claim 3 wherein said method is a PCR-based method.

6. The method of claim 4 wherein said expression levels are normalized relative to the expression levels of one or more reference genes, or their expression products.

7. The method of claim 1 comprising determining evidence of the expression levels of at least two of said genes, or their expression products.

8. The method of claim 1 comprising determining evidence of the expression levels of at least three of said genes, or their expression products.

9. The method of claim 1 comprising determining evidence of the expression levels of at least four of said genes, or their expression products.

10. The method of claim 1 comprising determining evidence of the expression levels of at least live of said genes, or their expression products.

11. The method of claim 1 further comprising the step of creating a report summarizing said IBD detection.

12. The method of claim 1, wherein said IBD is ulcerative colitis.

13. The method of claim 1, wherein said IBD is Crohn's disease.

14. The method of claim 1, wherein said IBD is ulcerative colitis and Crohn's disease.

15. The method of claim 1, wherein said test sample is from a colonic tissue biopsy.

16. The method of claim 15, wherein said biopsy is from a tissue selected from the group consisting of the terminal ileum, the ascending colon, the descending colon, and the sigmoid colon.

17. The method of claim 15, wherein said biopsy is from an inflamed colonic area.

18. The method of claim 15, wherein said biopsy is from a non-inflamed colonic area.

19. The method of claim 1, wherein said determining step is indicative of a recurrence of an IBD in said mammalian subject, and wherein said mammalian subject was previously diagnosed with an IBD and treated for said previously diagnosed IBD.

20. The method of claim 19, wherein said treatment comprised surgery.

21. The method of claim 1, wherein said determining step is indicative of a flare-up of said IBD in said mammalian subject.

22. A method of treating an inflammatory bowel disorder (IBD) in a mammalian subject in need thereof, the method comprising the steps of (a) determining that the level of expression of a nucleic acid encoding a polypeptide shown as any one of SEQ ID NOS: 2, 4, 6, 8, 10, 12, 14, 16, 18, 20, 22, 24, 26. 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 194, 197, 199, 201, 203, 205, 207, and 230 in a test sample obtained from said subject is higher relative to the level of expression in a control, wherein said higher level of expression is indicative of the presence of an IBD in the subject from which the test sample was obtained; and (b) administering to said subject an effective amount of an IBD therapeutic agent.

23. (canceled)

24. The method of claim 22 wherein said mammalian subject is a human patient.

25. The method of claim 24 wherein evidence of said expression level is obtained by a method of gene expression profiling.

26. The method of claim 24 wherein said method is a PCR-based method.

27. The method of claim 25 wherein said expression levels are normalized relative to the expression levels of one or more reference genes, or their expression products.

28. The method of claim 22 comprising determining evidence of the expression levels of at least two of said genes, or their expression products.

29. The method of claim 22 comprising determining evidence of the expression levels of at least three of said genes, or their expression products.

30. The method of claim 22 comprising determining evidence of the expression levels of at least four of said genes, or their expression products.

31. The method of claim 22 comprising determining evidence of the expression levels of at least live of said genes, or their expression products.

32. The method of claim 22 further comprising the step of creating a report summarizing said IBD detection.

33. The method of claim 22, wherein said IBD is ulcerative colitis.

34. The method of claim 22, wherein said IBD is Crohn's disease.

35. The method of claim 22 wherein said IBD is ulcerative colitis and Crohn's disease.

36. The method of claim 22 wherein said test sample is from a colonic tissue biopsy.

37. The method of claim 36, wherein said biopsy is from a tissue selected from the group consisting of the terminal ileum, the ascending colon, the descending colon, and the sigmoid colon.

38. The method of claim 36, wherein said biopsy is from an inflamed colonic area.

39. The method of claim 36, wherein said biopsy is from a non-inflamed colonic area.

40. The method of claim 22, wherein said determining step is indicative of a recurrence of an IBD in said mammalian subject, and wherein said mammalian subject was previously diagnosed with an IBD and treated for said previously diagnosed IBD.

41. The method of claim 40, wherein said treatment comprised surgery.

42. The method of claim 22, wherein said determining step is indicative of a flare-up of said IBD in said mammalian subject.

43. The method of claim 22, wherein said IBD therapeutic agent is an aminosalicylate.

44. The method of claim 22, wherein said IBD therapeutic agent is a corticosteroid.

45. The method of claim 22, wherein said IBD therapeutic agent is an immunosuppressive agent.
